SEQUENCE LISTING (252 sequences)

SEQ ID NO: 1 - protein, 386 aa, Human alphaherpesvirus 1
KTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGA

SEQ ID NO: 2 - protein, 389 aa, Human alphaherpesvirus 1
GTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGA

SEQ ID NO: 3 - protein, 552 aa, Human alphaherpesvirus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 4 - protein, 382 aa, Human alphaherpesvirus 2
RTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 5 - protein, 545 aa, Human alphaherpesvirus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 6 - protein, 431 aa, Human alphaherpesvirus 1
ETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHH

SEQ ID NO: 7 - protein, 433 aa, Human alphaherpesvirus 1
GSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHH

SEQ ID NO: 8 - protein, 511 aa, Human alphaherpesvirus 1
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 9 - protein, 400 aa, Human alphaherpesvirus 2
ASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 10 - protein, 400 aa, Human alphaherpesvirus 2
ASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 11 - protein, 399 aa, Human alphaherpesvirus 2
SPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 12 - protein, 399 aa, Human alphaherpesvirus 2
SPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 13 - protein, 401 aa, Human alphaherpesvirus 2
SASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 14 - protein, 401 aa, Human alphaherpesvirus 2
SASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 15 - protein, 480 aa, Human alphaherpesvirus 2
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 16 - protein, 306 aa, Human alphaherpesvirus 1
KYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPY

SEQ ID NO: 17 - protein, 394 aa, Human alphaherpesvirus 1
MGGAAARLGAVILFVVIVGLHGVRGKYALADASLKLADPNRFRRKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEAPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 18 - protein, 306 aa, Human alphaherpesvirus 2
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 19 - protein, 302 aa, Human alphaherpesvirus 2
ADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 20 - protein, 301 aa, Human alphaherpesvirus 2
DPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 21 - protein, 393 aa, Human alphaherpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 22 - RNA, 1161 nt, Human alphaherpesvirus 1
  source 1..1161: mol_type = mRNA
aagacctcctggcgccgcgtgtccgtgggcgaggacgtgtccctgctgcccgcccccggccccaccggccgcggccccacccagaagctgctgtgggccgtggagcccctggacggctgcggccccctgcacccctcctgggtgtccctgatgccccccaagcaggtgcccgagaccgtggtggacgccgcctgcatgcgcgcccccgtgcccctggccatggcctacgccccccccgccccctccgccaccggcggcctgcgcaccgacttcgtgtggcaggagcgcgccgccgtggtgaaccgctccctggtgatctacggcgtgcgcgagaccgactccggcctgtacaccctgtccgtgggcgacatcaaggaccccgcccgccaggtggcctccgtggtgctggtggtgcagcccgcccccgtgcccacccccccccccacccccgccgactacgacgaggacgacaacgacgagggcgagggcgaggacgagtccctggccggcacccccgcctccggcaccccccgcctgcccccctcccccgcccccccccgctcctggccctccgcccccgaggtgtcccacgtgcgcggcgtgaccgtgcgcatggagacccccgaggccatcctgttctcccccggcgaggccttctccaccaacgtgtccatccacgccatcgcccacgacgaccagacctacaccatggacgtggtgtggctgcgcttcgacgtgcccacctcctgcgccgagatgcgcatctacgagtcctgcctgtaccacccccagctgcccgagtgcctgtcccccgccgacgccccctgcgccgcctccacctggacctcccgcctggccgtgcgctcctacgccggctgctcccgcaccaacccccccccccgctgctccgccgaggcccacatggagcccttccccggcctggcctggcaggccgcctccgtgaacctggagttccgcgacgcctccccccagcactccggcctgtacctgtgcgtggtgtacgtgaacgaccacatccacgcctggggccacatcaccatcaacaccgccgcccagtaccgcaacgccgtggtggagcagcccctgccccagcgcggcgccgacctggccgagcccacccacccccacgtgggcgcctaa

SEQ ID NO: 23 - RNA, 1149 nt, Human alphaherpesvirus 2
  source 1..1149: mol_type = mRNA
cgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 24 - RNA, 1149 nt, synthetic construct
  source 1..1149: mol_type = other RNA
agaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 25 - RNA, 1152 nt, synthetic construct
  source 1..1152: mol_type = other RNA
agaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 26 - RNA, 1149 nt, synthetic construct
  source 1..1149: mol_type = other RNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 27 - RNA, 1149 nt, synthetic construct
  source 1..1149: mol_type = other RNA
cgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 28 - RNA, 1152 nt, synthetic construct
  source 1..1152: mol_type = other RNA
cgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctgataa

SEQ ID NO: 29 - RNA, 1149 nt, synthetic construct
  source 1..1149: mol_type = other RNA
agaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 30 - RNA, 1152 nt, synthetic construct
  source 1..1152: mol_type = other RNA
agaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 31 - RNA, 1149 nt, synthetic construct
  source 1..1149: mol_type = other RNA
cggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 32 - RNA, 1296 nt, Human alphaherpesvirus 1
  source 1..1296: mol_type = mRNA
gagaccgcctccaccggccccaccatcaccgccggcgccgtgaccaacgcctccgaggcccccacctccggctcccccggctccgccgcctcccccgaggtgacccccacctccacccccaaccccaacaacgtgacccagaacaagaccacccccaccgagcccgcctccccccccaccacccccaagcccacctccacccccaagtccccccccacctccacccccgaccccaagcccaagaacaacaccacccccgccaagtccggccgccccaccaagccccccggccccgtgtggtgcgaccgccgcgaccccctggcccgctacggctcccgcgtgcagatccgctgccgcttccgcaactccacccgcatggagttccgcctgcagatctggcgctactccatgggcccctccccccccatcgcccccgcccccgacctggaggaggtgctgaccaacatcaccgccccccccggcggcctgctggtgtacgactccgcccccaacctgaccgacccccacgtgctgtgggccgagggcgccggccccggcgccgacccccccctgtactccgtgaccggccccctgcccacccagcgcctgatcatcggcgaggtgacccccgccacccagggcatgtactacctggcctggggccgcatggactccccccacgagtacggcacctgggtgcgcgtgcgcatgttccgccccccctccctgaccctgcagccccacgccgtgatggagggccagcccttcaaggccacctgcaccgccgccgcctactacccccgcaaccccgtggagttcgactggttcgaggacgaccgccaggtgttcaaccccggccagatcgacacccagacccacgagcaccccgacggcttcaccaccgtgtccaccgtgacctccgaggccgtgggcggccaggtgcccccccgcaccttcacctgccagatgacctggcaccgcgactccgtgaccttctcccgccgcaacgccaccggcctggccctggtgctgccccgccccaccatcaccatggagttcggcgtgcgccacgtggtgtgcaccgccggctgcgtgcccgagggcgtgaccttcgcctggttcctgggcgacgacccctcccccgccgccaagtccgccgtgaccgcccaggagtcctgcgaccaccccggcctggccaccgtgcgctccaccctgcccatctcctacgactactccgagtacatctgccgcctgaccggctaccccgccggcatccccgtgctggagcaccactaa

SEQ ID NO: 33 - RNA, 1203 nt, Human alphaherpesvirus 2
  source 1..1203: mol_type = mRNA
gcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 34 - RNA, 1200 nt, Human alphaherpesvirus 2
  source 1..1200: mol_type = mRNA
tcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 35 - RNA, 1206 nt, synthetic construct
  source 1..1206: mol_type = other RNA
agcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 36 - RNA, 1200 nt, synthetic construct
  source 1..1200: mol_type = other RNA
tctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 37 - RNA, 1203 nt, synthetic construct
  source 1..1203: mol_type = other RNA
agccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 38 - RNA, 1203 nt, synthetic construct
  source 1..1203: mol_type = other RNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 39 - RNA, 1200 nt, synthetic construct
  source 1..1200: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 40 - RNA, 1203 nt, synthetic construct
  source 1..1203: mol_type = other RNA
gcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 41 - RNA, 1200 nt, synthetic construct
  source 1..1200: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 42 - RNA, 1203 nt, synthetic construct
  source 1..1203: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 43 - RNA, 1200 nt, synthetic construct
  source 1..1200: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 44 - RNA, 1200 nt, synthetic construct; identical to SEQ ID NO: 39
  source 1..1200: mol_type = other RNA

SEQ ID NO: 45 - RNA, 1203 nt, synthetic construct
  source 1..1203: mol_type = other RNA
agccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 46 - RNA, 1203 nt, synthetic construct
  source 1..1203: mol_type = other RNA
gcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 47 - RNA, 1200 nt, synthetic construct
  source 1..1200: mol_type = other RNA
tcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 48 - RNA, 1203 nt, synthetic construct
  source 1..1203: mol_type = other RNA
tctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 49 - RNA, 1200 nt, synthetic construct
  source 1..1200: mol_type = other RNA
tctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 50 - RNA, 1200 nt, synthetic construct
  source 1..1200: mol_type = other RNA
agtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 51 - RNA, 1200 nt, synthetic construct
  source 1..1200: mol_type = other RNA
tccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 52 - RNA, 1200 nt, synthetic construct
  source 1..1200: mol_type = other RNA
tctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 53 - RNA, 1200 nt, synthetic construct
  source 1..1200: mol_type = other RNA
tctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 54 - RNA, 921 nt, Human alphaherpesvirus 1
  source 1..921: mol_type = mRNA
aagtacgccctggccgacgcctccctgaagatggccgaccccaaccgcttccgcggcaaggacctgcccgtgctggaccagctgaccgacccccccggcgtgcgccgcgtgtaccacatccaggccggcctgcccgaccccttccagcccccctccctgcccatcaccgtgtactacgccgtgctggagcgcgcctgccgctccgtgctgctgaacgccccctccgaggccccccagatcgtgcgcggcgcctccgaggacgtgcgcaagcagccctacaacctgaccatcgcctggttccgcatgggcggcaactgcgccatccccatcaccgtgatggagtacaccgagtgctcctacaacaagtccctgggcgcctgccccatccgcacccagccccgctggaactactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgccaagggctcctgcaagtacgccctgcccctgcgcatccccccctccgcctgcctgtccccccaggcctaccagcagggcgtgaccgtggactccatcggcatgctgccccgcttcatccccgagaaccagcgcaccgtggccgtgtactccctgaagatcgccggctggcacggccccaaggccccctacacctccaccctgctgccccccgagctgtccgagacccccaacgccacccagcccgagctggcccccgaggaccccgaggactccgccctgctggaggaccccgtgggcaccgtggccccccagatcccccccaactggcacatcccctccatccaggacgccgccaccccctactaa

SEQ ID NO: 55 - RNA, 921 nt, Human alphaherpesvirus 2
  source 1..921: mol_type = mRNA
aagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 56 - RNA, 921 nt, synthetic construct
  source 1..921: mol_type = other RNA
aaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 57 - RNA, 921 nt, synthetic construct
  source 1..921: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 58 - RNA, 921 nt, synthetic construct
  source 1..921: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 59 - RNA, 924 nt, synthetic construct
  source 1..924: mol_type = other RNA
aaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactgataa

SEQ ID NO: 60 - RNA, 921 nt, synthetic construct
  source 1..921: mol_type = other RNA
aaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 61 - RNA, 906 nt, synthetic construct
  source 1..906: mol_type = other RNA
gatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 62 - RNA, 1626 nt, synthetic construct
  source 1..1626: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccaagacctcctggcgccgcgtgtccgtgggcgaggacgtgtccctgctgcccgcccccggccccaccggccgcggccccacccagaagctgctgtgggccgtggagcccctggacggctgcggccccctgcacccctcctgggtgtccctgatgccccccaagcaggtgcccgagaccgtggtggacgccgcctgcatgcgcgcccccgtgcccctggccatggcctacgccccccccgccccctccgccaccggcggcctgcgcaccgacttcgtgtggcaggagcgcgccgccgtggtgaaccgctccctggtgatctacggcgtgcgcgagaccgactccggcctgtacaccctgtccgtgggcgacatcaaggaccccgcccgccaggtggcctccgtggtgctggtggtgcagcccgcccccgtgcccacccccccccccacccccgccgactacgacgaggacgacaacgacgagggcgagggcgaggacgagtccctggccggcacccccgcctccggcaccccccgcctgcccccctcccccgcccccccccgctcctggccctccgcccccgaggtgtcccacgtgcgcggcgtgaccgtgcgcatggagacccccgaggccatcctgttctcccccggcgaggccttctccaccaacgtgtccatccacgccatcgcccacgacgaccagacctacaccatggacgtggtgtggctgcgcttcgacgtgcccacctcctgcgccgagatgcgcatctacgagtcctgcctgtaccacccccagctgcccgagtgcctgtcccccgccgacgccccctgcgccgcctccacctggacctcccgcctggccgtgcgctcctacgccggctgctcccgcaccaacccccccccccgctgctccgccgaggcccacatggagcccttccccggcctggcctggcaggccgcctccgtgaacctggagttccgcgacgcctccccccagcactccggcctgtacctgtgcgtggtgtacgtgaacgaccacatccacgcctggggccacatcaccatcaacaccgccgcccagtaccgcaacgccgtggtggagcagcccctgccccagcgcggcgccgacctggccgagcccacccacccccacgtgggcgcctaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 63 - RNA, 1614 nt, synthetic construct
  source 1..1614: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 64 - RNA, 1779 nt, synthetic construct
  source 1..1779: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatggccatctccggcgtgcccgtgctgggcttcttcatcatcgccgtgctgatgtccgcccaggagtcctgggccgagaccgcctccaccggccccaccatcaccgccggcgccgtgaccaacgcctccgaggcccccacctccggctcccccggctccgccgcctcccccgaggtgacccccacctccacccccaaccccaacaacgtgacccagaacaagaccacccccaccgagcccgcctccccccccaccacccccaagcccacctccacccccaagtccccccccacctccacccccgaccccaagcccaagaacaacaccacccccgccaagtccggccgccccaccaagccccccggccccgtgtggtgcgaccgccgcgaccccctggcccgctacggctcccgcgtgcagatccgctgccgcttccgcaactccacccgcatggagttccgcctgcagatctggcgctactccatgggcccctccccccccatcgcccccgcccccgacctggaggaggtgctgaccaacatcaccgccccccccggcggcctgctggtgtacgactccgcccccaacctgaccgacccccacgtgctgtgggccgagggcgccggccccggcgccgacccccccctgtactccgtgaccggccccctgcccacccagcgcctgatcatcggcgaggtgacccccgccacccagggcatgtactacctggcctggggccgcatggactccccccacgagtacggcacctgggtgcgcgtgcgcatgttccgccccccctccctgaccctgcagccccacgccgtgatggagggccagcccttcaaggccacctgcaccgccgccgcctactacccccgcaaccccgtggagttcgactggttcgaggacgaccgccaggtgttcaaccccggccagatcgacacccagacccacgagcaccccgacggcttcaccaccgtgtccaccgtgacctccgaggccgtgggcggccaggtgcccccccgcaccttcacctgccagatgacctggcaccgcgactccgtgaccttctcccgccgcaacgccaccggcctggccctggtgctgccccgccccaccatcaccatggagttcggcgtgcgccacgtggtgtgcaccgccggctgcgtgcccgagggcgtgaccttcgcctggttcctgggcgacgacccctcccccgccgccaagtccgccgtgaccgcccaggagtcctgcgaccaccccggcctggccaccgtgcgctccaccctgcccatctcctacgactactccgagtacatctgccgcctgaccggctaccccgccggcatccccgtgctggagcaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 65 - RNA, 1668 nt, synthetic construct
  source 1..1668: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 66 - RNA, 1386 nt, synthetic construct
  source 1..1386: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccaagtacgccctggccgacgcctccctgaagatggccgaccccaaccgcttccgcggcaaggacctgcccgtgctggaccagctgaccgacccccccggcgtgcgccgcgtgtaccacatccaggccggcctgcccgaccccttccagcccccctccctgcccatcaccgtgtactacgccgtgctggagcgcgcctgccgctccgtgctgctgaacgccccctccgaggccccccagatcgtgcgcggcgcctccgaggacgtgcgcaagcagccctacaacctgaccatcgcctggttccgcatgggcggcaactgcgccatccccatcaccgtgatggagtacaccgagtgctcctacaacaagtccctgggcgcctgccccatccgcacccagccccgctggaactactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgccaagggctcctgcaagtacgccctgcccctgcgcatccccccctccgcctgcctgtccccccaggcctaccagcagggcgtgaccgtggactccatcggcatgctgccccgcttcatccccgagaaccagcgcaccgtggccgtgtactccctgaagatcgccggctggcacggccccaaggccccctacacctccaccctgctgccccccgagctgtccgagacccccaacgccacccagcccgagctggcccccgaggaccccgaggactccgccctgctggaggaccccgtgggcaccgtggccccccagatcccccccaactggcacatcccctccatccaggacgccgccaccccctactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 67 - RNA, 1386 nt, synthetic construct
  source 1..1386: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgacccgcctgaccgtgctggccctgctggccggcctgctggcctcctcccgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 68 - protein, 25 aa, Human alphaherpesvirus 2
MGRLTSGVGTAALLVVAVGLRVVCA

SEQ ID NO: 69 - protein, 27 aa, Human alphaherpesvirus 2
MGRLTSGVGTAALLVVAVGLRVVCAKY

SEQ ID NO: 70 - protein, 28 aa, Human alphaherpesvirus 2
MGRLTSGVGTAALLVVAVGLRVVCAKYA

SEQ ID NO: 71 - protein, 29 aa, Human alphaherpesvirus 2
MGRLTSGVGTAALLVVAVGLRVVCAKYAL

SEQ ID NO: 72 - protein, 30 aa, Human alphaherpesvirus 2
MGRLTSGVGTAALLVVAVGLRVVCAKYALA

SEQ ID NO: 73 - protein, 25 aa, Human alphaherpesvirus 1
MGGAAARLGAVILFVVIVGLHGVRG

SEQ ID NO: 74 - protein, 27 aa, Human alphaherpesvirus 1
MGGAAARLGAVILFVVIVGLHGVRGKY

SEQ ID NO: 75 - protein, 27 aa, Human alphaherpesvirus 2
MALGRVGLAVGLWGLLWVGVVVVLANA

SEQ ID NO: 76 - protein, 20 aa, Human alphaherpesvirus 1
MDRGAVVGFLLGVCVVSCLA

SEQ ID NO: 77 - protein, 23 aa, Human alphaherpesvirus 2
MARGAGLVFFVGVWVVSCLAAAP

SEQ ID NO: 78 - protein, 26 aa, Human alphaherpesvirus 2
MARGAGLVFFVGVWVVSCLAAAPRTS

SEQ ID NO: 79 - protein, 23 aa, Human alphaherpesvirus 2
MARGAGLVFFVGVWVVSCLVVVP

SEQ ID NO: 80 - protein, 20 aa, Human alphaherpesvirus 1
MDRGAVVGFLLGVCVVSCLA

SEQ ID NO: 81 - protein, 22 aa, Human alphaherpesvirus 2
MRGGGLICALVVGALVAAVASA

SEQ ID NO: 82 - protein, 30 aa, Human alphaherpesvirus 1
MHQGAPSWGRRWFVVWALLGLTLGVLVASA

SEQ ID NO: 83 - protein, 32 aa, Human alphaherpesvirus 1
MHQGAPSWGRRWFVVWALLGLTLGVLVASAAP

SEQ ID NO: 84 - protein, 20 aa, Human alphaherpesvirus 2
MPGRSLQGLAILGLWVCATG

SEQ ID NO: 85 - protein, 21 aa, Human alphaherpesvirus 2
MPGRSLQGLAILGLWVCATGL

SEQ ID NO: 86 - protein, 18 aa, Human alphaherpesvirus 2
MPGRSLQGLAILGLWVCA

SEQ ID NO: 87 - protein, 20 aa, Human alphaherpesvirus 1
MPCRPLQGLVLVGLWVCATS

SEQ ID NO: 88 - protein, 19 aa, Homo sapiens
MRMQLLLLIALSLALVTNS

SEQ ID NO: 89 - protein, 19 aa, Homo sapiens
MTRLTVLALLAGLLASSRA

SEQ ID NO: 90 - protein, 25 aa, Homo sapiens
MAISGVPVLGFFIIAVLMSAQESWA

SEQ ID NO: 91 - protein, 34 aa, Zaire ebolavirus
MGVTGILQLPRDRFKRTSFFLWVIILFQRTFSIP

SEQ ID NO: 92 - RNA, 75 nt, Human alphaherpesvirus 2
  source 1..75: mol_type = mRNA
atgggccgcctgacctccggcgtgggcaccgccgccctgctggtggtggccgtgggcctgcgcgtggtgtgcgcc

SEQ ID NO: 93 - RNA, 75 nt, synthetic construct
  source 1..75: mol_type = other RNA
atgggcagactgacatctggcgtgggaacagctgctctgctggtggttgctgtgggcctgagagtcgtgtgtgcc

SEQ ID NO: 94 - RNA, 75 nt, synthetic construct
  source 1..75: mol_type = other RNA
atggggagactcacatcaggcgtaggaaccgctgccctgttggtcgtggccgttggtctgagagttgtgtgtgcc

SEQ ID NO: 95 - RNA, 75 nt, synthetic construct
  source 1..75: mol_type = other RNA
atgggcagactgacctccggcgtgggcaccgccgccctgctggtggtggccgtgggcctgagagtggtgtgcgcc

SEQ ID NO: 96 - RNA, 84 nt, Human alphaherpesvirus 2
  source 1..84: mol_type = mRNA
atgggccgcctgacctccggcgtgggcaccgccgccctgctggtggtggccgtgggcctgcgcgtggtgtgcgccaagtacgcc

SEQ ID NO: 97 - RNA, 84 nt, synthetic construct
  source 1..84: mol_type = other RNA
atgggcagactgacatctggcgtgggaacagctgctctgctggtggttgctgtgggcctgagagtcgtgtgtgccaaatacgcc

SEQ ID NO: 98 - RNA, 84 nt, synthetic construct
  source 1..84: mol_type = other RNA
atggggagactcacatcaggcgtaggaaccgctgccctgttggtcgtggccgttggtctgagagttgtgtgtgccaaatatgct

SEQ ID NO: 99 - RNA, 84 nt, synthetic construct
  source 1..84: mol_type = other RNA
atgggcagactgacctccggcgtgggcaccgccgccctgctggtggtggccgtgggcctgagagtggtgtgcgccaaatacgcc

SEQ ID NO: 100 - RNA, 87 nt, synthetic construct
  source 1..87: mol_type = other RNA
atggggagactcacatcaggcgtaggaaccgctgccctgttggtcgtggccgttggtctgagagttgtgtgtgccaaatatgctctg

SEQ ID NO: 101 - RNA, 87 nt, synthetic construct
  source 1..87: mol_type = other RNA
atgggcagactgacctccggcgtgggcaccgccgccctgctggtggtggccgtgggcctgagagtggtgtgcgccaaatacgccctg

SEQ ID NO: 102 - RNA, 90 nt, Human alphaherpesvirus 2
  source 1..90: mol_type = mRNA
atgggccgcctgacctccggcgtgggcaccgccgccctgctggtggtggccgtgggcctgcgcgtggtgtgcgccaagtacgccctggcc

SEQ ID NO: 103 - RNA, 90 nt, synthetic construct
  source 1..90: mol_type = other RNA
atgggcagactgacatctggcgtgggaacagctgctctgctggtggttgctgtgggcctgagagtcgtgtgtgccaaatacgccctggcc

SEQ ID NO: 104 - RNA, 90 nt, synthetic construct
  source 1..90: mol_type = other RNA
atggggagactcacatcaggcgtaggaaccgctgccctgttggtcgtggccgttggtctgagagttgtgtgtgccaaatatgctctggca

SEQ ID NO: 105 - RNA, 90 nt, synthetic construct
  source 1..90: mol_type = other RNA
atgggcagactgacctccggcgtgggcaccgccgccctgctggtggtggccgtgggcctgagagtggtgtgcgccaaatacgccctggcc

SEQ ID NO: 106 - RNA, 75 nt, synthetic construct
  source 1..75: mol_type = other RNA
atgggcggagctgctgctagactgggagccgtgatcctgttcgtggttatcgtgggactgcatggcgtgcggggc

SEQ ID NO: 107 - RNA, 75 nt, synthetic construct
  source 1..75: mol_type = other RNA
atgggaggagcagctgccagactcggtgccgtgatcctgttcgtggtcattgttggcctgcacggggtaaggggc

SEQ ID NO: 108 - RNA, 75 nt, synthetic construct
  source 1..75: mol_type = other RNA
atgggaggagctgctgctagattgggagctgtgattctgtttgtggtgattgtgggactgcatggagtgagagga

SEQ ID NO: 109 - RNA, 75 nt, synthetic construct
  source 1..75: mol_type = other RNA
atgggcggcgcagccgctagactgggtgcagtcatcctctttgtggtgatcgtggggctgcatggggtcagaggg

SEQ ID NO: 110 - RNA, 75 nt, synthetic construct
  source 1..75: mol_type = other RNA
atgggcggagctgcggctcgcctcggagccgtcattctgttcgtggtgatcgtgggtctgcatggggtcagagga

SEQ ID NO: 111 - RNA, 81 nt, synthetic construct
  source 1..81: mol_type = other RNA
atgggcggagctgctgctagactgggagccgtgatcctgttcgtggttatcgtgggactgcatggcgtgcggggcaagtat

SEQ ID NO: 112 - RNA, 81 nt, synthetic construct
  source 1..81: mol_type = other RNA
atgggaggagcagctgccagactcggtgccgtgatcctgttcgtggtcattgttggcctgcacggggtaaggggcaagtac

SEQ ID NO: 113 - RNA, 90 nt, synthetic construct
  source 1..90: mol_type = other RNA
atggggagactcacatccggagtgggcactgctgccctgttggtcgtggccgttggtctgagagttgtgtgtgccaaatatgctctggca

SEQ ID NO: 114 - RNA, 81 nt, synthetic construct
  source 1..81: mol_type = other RNA
atgggaggagctgctgctagattgggagctgtgattctgtttgtggtgattgtgggactgcatggagtgagaggaaaatac

SEQ ID NO: 115 - RNA, 81 nt, synthetic construct
  source 1..81: mol_type = other RNA
atgggcggcgcagccgctagactgggtgcagtcatcctctttgtggtgatcgtggggctgcatggggtcagagggaagtat

SEQ ID NO: 116 - RNA, 81 nt, synthetic construct
  source 1..81: mol_type = other RNA
atgggcggagctgcggctcgcctcggagccgtcattctgttcgtggtgatcgtgggtctgcatggggtcagaggaaagtac

SEQ ID NO: 117 - RNA, 81 nt, synthetic construct
  source 1..81: mol_type = other RNA
atggccttggggagagtgggccttgcagtgggtctgtggggactgctctgggttggcgtagtcgtcgtgctggctaacgca

SEQ ID NO: 118 - RNA, 81 nt, synthetic construct
  source 1..81: mol_type = other RNA
atggcactagggagagtgggattagctgtgggtctgtggggactgctctgggtaggagtcgtcgtcgtgctggctaacgca

SEQ ID NO: 119 - RNA, 81 nt, synthetic construct
  source 1..81: mol_type = other RNA
atggccttggggagagtcggccttgcagtgggactgtggggtctgctgtgggttggcgtggtagtcgtgctcgctaacgcc

SEQ ID NO: 120 - RNA, 81 nt, synthetic construct
  source 1..81: mol_type = other RNA
atggctctgggcagagttggactggctgttggactgtggggactgctgtgggttggagtggtggtggtgctggctaatgct

SEQ ID NO: 121 - RNA, 69 nt, synthetic construct
  source 1..69: mol_type = other RNA
atggcacggggagccggattggtgttctttgtgggcgtgtgggtggtgagctgcttggcagccgcacca

SEQ ID NO: 122 - RNA, 69 nt, synthetic construct
  source 1..69: mol_type = other RNA
atggcacggggcgcaggtttggtctttttcgtgggcgtgtgggtggtgagctgcttggcagccgcacca

SEQ ID NO: 123 - RNA, 69 nt, synthetic construct
  source 1..69: mol_type = other RNA
atggcgagaggagccggactcgtgttctttgtgggagtctgggttgtgagctgcctggcagcagctcca

SEQ ID NO: 124 - RNA, 69 nt, synthetic construct
  source 1..69: mol_type = other RNA
atggcgagaggagccgggctcgtgttctttgtgggcgtatgggtcgtttcctgcctggctgccgcaccc

SEQ ID NO: 125 - RNA, 78 nt, synthetic construct
  source 1..78: mol_type = other RNA
atggctagaggtgccggcctggtgttctttgttggcgtgtgggtcgtgtcctgtctggccgctgctcctagaacatct

SEQ ID NO: 126 - RNA, 78 nt, synthetic construct
  source 1..78: mol_type = other RNA
atggcgagaggagccgggctcgtgttctttgtgggcgtatgggtcgtttcctgcctggctgccgcacccaggaccagc

SEQ ID NO: 127 - RNA, 78 nt, synthetic construct
  source 1..78: mol_type = other RNA
atggcaagaggagcaggactggtgtttttcgtgggagtttgggtggtgtcttgcctggctgctgctccaagaacctct

SEQ ID NO: 128 - RNA, 78 nt, synthetic construct
  source 1..78: mol_type = other RNA
atggcgagaggagccggactcgtgttctttgtgggagtctgggttgtgagctgcctggcagcagctccacgcactagc

SEQ ID NO: 129 - RNA, 69 nt, synthetic construct
  source 1..69: mol_type = other RNA
atggcgagaggagccggactcgtgttctttgtgggagtctgggttgtgagctgcctggtagtagttcca

SEQ ID NO: 130 - RNA, 90 nt, synthetic construct
  source 1..90: mol_type = other RNA
atgcatcagggcgctccatcttggggtagacgttggttcgttgtgtgggccctgctgggactgacactgggagttctggttgcctctgct

SEQ ID NO: 131 - RNA, 90 nt, synthetic construct
  source 1..90: mol_type = other RNA
atgcaccagggtgccccttcctggggcagaaggtggttcgtggtgtgggcccttctggggctgaccctcggagtcctggttgcgagcgca

SEQ ID NO: 132 - RNA, 90 nt, synthetic construct
  source 1..90: mol_type = other RNA
atgcaccagggagcaccttcttggggaagaagatggtttgtggtgtgggctctgctgggactgaccctgggagtgctggtggcttctgct

SEQ ID NO: 133 - RNA, 90 nt, synthetic construct
  source 1..90: mol_type = other RNA
atgcatcaaggcgcacccagttggggcagacggtggttcgtagtgtgggccttgctgggactgactctcggtgtcctcgttgcgtctgct

SEQ ID NO: 134 - RNA, 96 nt, synthetic construct
  source 1..96: mol_type = other RNA
atgcatcagggcgctccatcttggggtagacgttggttcgttgtgtgggccctgctgggactgacactgggagttctggttgcctctgctgctcct

SEQ ID NO: 135 - RNA, 96 nt, synthetic construct
  source 1..96: mol_type = other RNA
atgcaccagggtgccccttcctggggcagaaggtggttcgtggtgtgggcccttctggggctgaccctcggagtcctggttgcgagcgcagctccc

SEQ ID NO: 136 - RNA, 96 nt, synthetic construct
  source 1..96: mol_type = other RNA
atgcaccagggagcaccttcttggggaagaagatggtttgtggtgtgggctctgctgggactgaccctgggagtgctggtggcttctgctgctcct

SEQ ID NO: 137 - RNA, 96 nt, synthetic construct
  source 1..96: mol_type = other RNA
atgcatcaaggcgcacccagttggggcagacggtggttcgtagtgtgggccttgctgggactgactctcggtgtcctcgttgcgtctgctgcaccg

SEQ ID NO: 138 - RNA, 60 nt, synthetic construct
  source 1..60: mol_type = other RNA
atgcctggcagatctctgcaaggactggccatcctcggactgtgggtttgcgcaacagga

SEQ ID NO: 139 - RNA, 60 nt, synthetic construct
  source 1..60: mol_type = other RNA
atgcccggcagaagcctccagggactggctatcctggggctgtgggtgtgcgccaccggt

SEQ ID NO: 140 - RNA, 60 nt, synthetic construct
  source 1..60: mol_type = other RNA
atgcctggaagatctctccagggactggcaatcctgggactgtgggtgtgtgcaacagga

SEQ ID NO: 141 - RNA, 60 nt, synthetic construct
  source 1..60: mol_type = other RNA
atgcctgggagaagcctgcaagggctcgcaatcttgggcctgtgggtttgtgccacaggc

SEQ ID NO: 142 - RNA, 63 nt, synthetic construct
  source 1..63: mol_type = other RNA
atgcctggcagatctctgcaaggactggccatcctcggactgtgggtttgcgcaacaggactg

SEQ ID NO: 143 - RNA, 63 nt, synthetic construct
  source 1..63: mol_type = other RNA
atgcccggcagaagcctccagggactggctatcctggggctgtgggtgtgcgccaccggtctt

SEQ ID NO: 144 - RNA, 63 nt, synthetic construct
  source 1..63: mol_type = other RNA
atgcctggaagatctctccagggactggcaatcctgggactgtgggtgtgtgcaacaggactg

SEQ ID NO: 145 - RNA, 63 nt, synthetic construct
  source 1..63: mol_type = other RNA
atgcctgggagaagcctgcaagggctcgcaatcttgggcctgtgggtttgtgccacaggcttg

SEQ ID NO: 146 - RNA, 54 nt, synthetic construct
  source 1..54: mol_type = other RNA
atgcctggcagatctctgcaaggactggccatcctcggactgtgggtttgcgca

SEQ ID NO: 147 - RNA, 54 nt, synthetic construct
  source 1..54: mol_type = other RNA
atgcccggcagaagcctccagggactggctatcctggggctgtgggtgtgcgcc

SEQ ID NO: 148 - RNA, 54 nt, synthetic construct
  source 1..54: mol_type = other RNA
atgcctggaagatctctccagggactggcaatcctgggactgtgggtgtgtgca

SEQ ID NO: 149 - RNA, 54 nt, synthetic construct
  source 1..54: mol_type = other RNA
atgcctgggagaagcctgcaagggctcgcaatcttgggcctgtgggtttgtgcc

SEQ ID NO: 150 - RNA, 57 nt, Homo sapiens
  source 1..57: mol_type = mRNA
atgcgcatgcagctgctgctgctgatcgccctgtccctggccctggtgaccaactcc

SEQ ID NO: 151 - RNA, 57 nt, synthetic construct
  source 1..57: mol_type = other RNA
atgagaatgcagctgctgctcctgatcgccctgtctctggccctggtcaccaatagc

SEQ ID NO: 152 - RNA, 57 nt, synthetic construct
  source 1..57: mol_type = other RNA
atgcgcatgcaactgctcctgctgattgcgttgagccttgccctggtgaccaacagc

SEQ ID NO: 153 - RNA, 57 nt, synthetic construct
  source 1..57: mol_type = other RNA
atgagaatgcagctgctgctgctgatcgccctgtccctggccctggtgaccaactcc

SEQ ID NO: 154 - RNA, 57 nt, Homo sapiens
  source 1..57: mol_type = mRNA
atgacccgcctgaccgtgctggccctgctggccggcctgctggcctcctcccgcgcc

SEQ ID NO: 155 - RNA, 75 nt, Homo sapiens
  source 1..75: mol_type = mRNA
atggccatctccggcgtgcccgtgctgggcttcttcatcatcgccgtgctgatgtccgcccaggagtcctgggcc

SEQ ID NO: 156 - RNA, 102 nt, synthetic construct
  source 1..102: mol_type = other RNA
atgggagtgaccggcattctccagctgcctcgggacagattcaagcggaccagcttcttcctgtgggtcatcatcctgttccagcggaccttcagcatcccc

SEQ ID NO: 157 - RNA, 102 nt, synthetic construct
  source 1..102: mol_type = other RNA
atgggagtgactggcatactccagcttcctagagacaggtttaagcgcacatccttctttctgtgggtcatcatcctgttccaacggaccttcagcattccc

SEQ ID NO: 158 - RNA, 102 nt, synthetic construct
  source 1..102: mol_type = other RNA
atgggagtaaccggaattctccagctgccaagagatcgattcaaaagaacatcatttttcctttgggtaattattctgtttcagagaacattttccatccct

SEQ ID NO: 159 - RNA, 102 nt, synthetic construct
  source 1..102: mol_type = other RNA
atgggagttaccggaattctgcaattgcccagggatcggttcaagcgcacatccttcttcctgtgggtcatcatcctctttcagcgtactttctccataccc

SEQ ID NO: 160 - protein, 419 aa, synthetic construct
MRMQLLLLIALSLALVTNSASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 161 - protein, 418 aa, synthetic construct
MRMQLLLLIALSLALVTNSSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 162 - protein, 425 aa, synthetic construct
MGRLTSGVGTAALLVVAVGLRVVCAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 163 - protein, 424 aa, synthetic construct
MGRLTSGVGTAALLVVAVGLRVVCASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 164 - protein, 428 aa, synthetic construct
MGRLTSGVGTAALLVVAVGLRVVCAKYAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 165 - protein, 427 aa, synthetic construct
MGRLTSGVGTAALLVVAVGLRVVCAKYASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 166 - protein, 429 aa, synthetic construct
MGRLTSGVGTAALLVVAVGLRVVCAKYALASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 167 - protein, 430 aa, synthetic construct
MGRLTSGVGTAALLVVAVGLRVVCAKYALAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 168 - protein, 426 aa, synthetic construct
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 169 - protein, 427 aa, synthetic construct
MALGRVGLAVGLWGLLWVGVVVVLANAASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 170 - protein, 426 aa, synthetic construct
MGGAAARLGAVILFVVIVGLHGVRGKYSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 171 - protein, 427 aa, synthetic construct
MGGAAARLGAVILFVVIVGLHGVRGKYASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 172 - protein, 431 aa, synthetic construct
MHQGAPSWGRRWFVVWALLGLTLGVLVASAAPSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 173 - protein, 432 aa, synthetic construct
MHQGAPSWGRRWFVVWALLGLTLGVLVASAAPASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 174 - protein, 420 aa, synthetic construct
MPGRSLQGLAILGLWVCATGLSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 175 - protein, 421 aa, synthetic construct
MPGRSLQGLAILGLWVCATGLASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 176 - protein, 425 aa, synthetic construct
MARGAGLVFFVGVWVVSCLAAAPRTSSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 177 - protein, 426 aa, synthetic construct
MARGAGLVFFVGVWVVSCLAAAPRTSASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 178 - protein, 433 aa, synthetic construct
MGVTGILQLPRDRFKRTSFFLWVIILFQRTFSIPSPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 179 - protein, 434 aa, synthetic construct
MGVTGILQLPRDRFKRTSFFLWVIILFQRTFSIPASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 180 - protein, 321 aa, synthetic construct
MRMQLLLLIALSLALVTNSADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 181 - protein, 325 aa, synthetic construct
MRMQLLLLIALSLALVTNSKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 182 - protein, 327 aa, synthetic construct
MGRLTSGVGTAALLVVAVGLRVVCAADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 183 - protein, 331 aa, synthetic construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 184 - protein, 329 aa, synthetic construct
MGRLTSGVGTAALLVVAVGLRVVCAKYADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 185 - protein, 401 aa, synthetic construct
MRMQLLLLIALSLALVTNSRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 186 - protein, 407 aa, synthetic construct
MGRLTSGVGTAALLVVAVGLRVVCARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 187 - protein, 410 aa, synthetic construct
MGRLTSGVGTAALLVVAVGLRVVCAKYARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 188 - protein, 412 aa, synthetic construct
MGRLTSGVGTAALLVVAVGLRVVCAKYALARTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 189 - protein, 405 aa, synthetic construct
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 190 - protein, 405 aa, synthetic construct
MARGAGLVFFVGVWVVSCLVVVPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 191 - protein, 383 aa, synthetic construct
MRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 192 - protein, 409 aa, synthetic construct
MGGAAARLGAVILFVVIVGLHGVRGKYRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 193 - RNA, 1260 nt, synthetic construct
  source 1..1260: mol_type = other RNA
atgcgcatgcagctgctgctgctgatcgccctgtccctggccctggtgaccaactccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 194 - RNA, 1260 nt, synthetic construct
  source 1..1260: mol_type = other RNA
atgagaatgcagctgctgctcctgatcgccctgtctctggccctggtcaccaatagcgcttctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 195 - RNA, 1260 nt, synthetic construct
  source 1..1260: mol_type = other RNA
atgcgcatgcaactgctcctgctgattgcgttgagccttgccctggtgaccaacagcgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 196 - RNA, 1260 nt, synthetic construct
  source 1..1260: mol_type = other RNA
atgagaatgcagctgctgctgctgatcgccctgtccctggccctggtgaccaactccgcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 197 - RNA, 1287 nt, synthetic construct
  source 1..1287: mol_type = other RNA
atgggccgcctgacctccggcgtgggcaccgccgccctgctggtggtggccgtgggcctgcgcgtggtgtgcgccaagtacgccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 198 - RNA, 1287 nt, synthetic construct
  source 1..1287: mol_type = other RNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 199 - RNA, 1287 nt, synthetic construct
  source 1..1287: mol_type = other RNA
atggggagactcacatcaggcgtaggaaccgctgccctgttggtcgtggccgttggtctgagagttgtgtgtgccaaatatgctgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 200 - RNA, 1287 nt, synthetic construct; identical to SEQ ID NO: 199
  source 1..1287: mol_type = other RNA

SEQ ID NO: 201 - RNA, 1290 nt, synthetic construct
  source 1..1290: mol_type = other RNA
atgggcagactgacatctggcgtgggaacagctgctctgctggtggttgctgtgggcctgagagtcgtgtgtgccaaatacgccctggcctctcccggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggcccaggcgcaagccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactga

SEQ ID NO: 202 - RNA, 1290 nt, synthetic construct
  source 1..1290: mol_type = other RNA
atggggagactcacatcaggcgtaggaaccgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 203 - RNA, 1290 nt, synthetic construct
  source 1..1290: mol_type = other RNA
atggggagactcacatccggagtgggcactgctgccctgttggtcgtggccgttggtctgagagttgtgtgtgccaaatatgctctggcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggacagacccacatgttatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 204 - RNA, 1290 nt, synthetic construct
  source 1..1290: mol_type = other RNA
atgggcagactgacctccggcgtgggcaccgccgccctgctggtggtggccgtgggcctgagagtggtgtgcgccaaatacgccctggcctcccccggcagaaccatcaccgtgggccccagaggcaacgcctccaacgccgccccctccgcctcccccagaaacgcctccgcccccagaaccacccccacccctccccagcccagaaaagccaccaaatccaaagcctccaccgccaaacccgcccctcctcccaaaaccggccctcccaaaacctcctccgaacccgtgagatgcaacagacacgatcccctggccagatacggctccagagtgcagatcagatgcagattccccaactccaccagaaccgaattcagactgcagatctggagatacgccaccgccaccgatgccgaaatcggcaccgccccctccctggaagaagtgatggtgaacgtgtccgcccctcctggcggccagctggtgtacgattccgcccccaacagaaccgatccccacgtgatctgggccgaaggcgccggccccggcgcctcccccagactgtactccgtggtgggccccctgggcagacagagactgatcatcgaagaactgaccctggaaacccagggcatgtactactgggtgtggggcagaaccgatagaccctccgcctacggcacctgggtgagagtgagagtgttcagacccccttccctgaccatccacccccacgccgtgctggaaggccagcccttcaaagccacctgcaccgccgccacctactaccccggcaacagagccgaattcgtgtggttcgaagatggcagaagggtgttcgatcccgcccagatccacacccagacccaggaaaaccccgacggcttctccaccgtgtccaccgtgacctccgccgccgtgggcggccagggccctcccagaaccttcacctgccagctgacctggcacagagactccgtgtccttctccagaagaaacgcctccggcaccgcctccgtgctgcccagacccaccatcaccatggaattcaccggcgatcacgccgtgtgcaccgccggctgcgtgcccgaaggcgtgaccttcgcctggttcctgggcgatgattcctcccccgccgaaaaagtggccgtggcctcccagacctcctgcggcagacccggcaccgccaccatcagatccaccctgcccgtgtcctacgaacagaccgaatacatctgcagactggccggctaccccgatggcatccccgtgctggaacaccactga

SEQ ID NO: 205 - RNA, 1281 nt, synthetic construct
  source 1..1281: mol_type = other RNA
atggccttggggagagtgggccttgcagtgggtctgtggggactgctctgggttggcgtagtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 206 - RNA, 1281 nt, synthetic construct
  source 1..1281: mol_type = other RNA
atggcactagggagagtgggattagctgtgggtctgtggggactgctctgggtaggagtcgtcgtcgtgctggctaacgcaagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 207 - RNA, 1281 nt, synthetic construct
  source 1..1281: mol_type = other RNA
atggccttggggagagtcggccttgcagtgggactgtggggtctgctgtgggttggcgtggtagtcgtgctcgctaacgccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 208 - RNA, 1281 nt, synthetic construct
  source 1..1281: mol_type = other RNA
atggctctgggcagagttggactggctgttggactgtggggactgctgtgggttggagtggtggtggtgctggctaatgctagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaggaagtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaggaactgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactga

SEQ ID NO: 209 - RNA, 1284 nt, synthetic construct
  source 1..1284: mol_type = other RNA
atgggcggagctgctgctagactgggagccgtgatcctgttcgtggttatcgtgggactgcatggcgtgcggggcaagtatagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 210 - RNA, 1284 nt, synthetic construct
  source 1..1284: mol_type = other RNA
atgggaggagcagctgccagactcggtgccgtgatcctgttcgtggtcattgttggcctgcacggggtaaggggcaagtacagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 211 - RNA, 1284 nt, synthetic construct
  source 1..1284: mol_type = other RNA
atgggaggagctgctgctagattgggagctgtgattctgtttgtggtgattgtgggactgcatggagtgagaggaaaatactctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 212 - RNA, 1281 nt, synthetic construct
  source 1..1281: mol_type = other RNA
atgggcggcgcagccgctagactgggtgcagtcatcctctttgtggtgatcgtggggctgcatggggtcagagggaagtattctccgggacggactataaccgtaggtccaagaggaaacgcctctaacgcagccccgtctgcctcaccacgaaacgcctcagctcccagaaccactcctactccaccccagcctaggaaggcgacgaaatccaaggcttccacggccaaacccgcccctccacccaaaaccggacctcctaagaccagctctgaaccggtgcggtgtaataggcacgacccattggcgcgatatggcagtagggtccagatacggtgcagattcccaaacagcacaagaacagaattccggctgcaaatctggcgatatgcaacggccaccgatgccgaaatcggaacagcacccagtctggaagaagtgatggtgaacgtcagtgctccacctggcggacaactggtgtacgactctgcacccaatcgcacagatccccacgtgatttgggccgagggtgctggacctggggcttcacccaggctgtatagcgttgtagggccacttgggaggcagagactcatcattgaggaactgaccctggaaactcagggcatgtactactgggtatggggccgcacagatcgccccagcgcttatggcacctgggtgcgggtgcgggtgtttcgcccaccctccctcaccattcaccctcatgcggttctggagggacagcctttcaaggcaacttgtaccgcagccacctactatcccggcaatagagcggagttcgtctggtttgaggacggccgtagggtgttcgatcctgcccagattcacacccagacacaggagaatcccgacggctttagcacagtgagcactgtgacgtctgctgccgttggtggtcaagggcctcctcgtaccttcacatgccaattgacctggcaccgcgactcagttagctttagccgccggaatgccagtgggaccgccagtgttctcccaaggccgacaatcaccatggagttcactggcgaccatgcagtgtgcacagctgggtgtgtcccagaaggcgtgactttcgcctggtttctgggtgatgactcctcacccgccgagaaagtagctgtcgcttcccagacttcctgtggacgtcctggaactgcgacaatccgaagcacactgccggtttcctacgagcagacggagtacatatgccgccttgcaggctaccccgatggaattccagtccttgagcaccattga

SEQ ID NO: 213 - RNA, 1299 nt, synthetic construct
  source 1..1299: mol_type = other RNA
atgcatcagggcgctccatcttggggtagacgttggttcgttgtgtgggccctgctgggactgacactgggagttctggttgcctctgctgctcctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 214 - RNA, 1299 nt, synthetic construct
  source 1..1299: mol_type = other RNA
atgcaccagggtgccccttcctggggcagaaggtggttcgtggtgtgggcccttctggggctgaccctcggagtcctggttgcgagcgcagctcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 215 - RNA, 1299 nt, synthetic construct
  source 1..1299: mol_type = other RNA
atgcaccagggagcaccttcttggggaagaagatggtttgtggtgtgggctctgctgggactgaccctgggagtgctggtggcttctgctgctccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 216 - RNA, 1296 nt, synthetic construct
  source 1..1296: mol_type = other RNA
atgcatcaaggcgcacccagttggggcagacggtggttcgtagtgtgggccttgctgggactgactctcggtgtcctcgttgcgtctgctgcaccgagtccaggaaggacgattacggtgggacccagaggtaatgcgtccaatgctgcgccatccgcttctccacggaacgcatctgcacccaggactacaccgacaccacctcagccgcgcaaagccaccaagagcaaggccagcacagccaaacccgctcctccacctaaaaccggaccacctaagaccagctctgaacccgtcagatgcaacaggcacgatccgttggccagatatggcagtcgcgtccagatcaggtgtcgcttccctaacagcacacggaccgagttcaggctgcaaatttggcgctacgctacagccactgacgcagagattggcactgctcccagtctggaggaggtcatggtgaacgtgtctgctccaccaggcggtcagctggtctatgactcagcccctaatcgcacagatcctcacgtgatttgggcagaaggtgcggggcctggggcctccccaaggctctactcagtggttggaccccttgggagacagcggctgatcatcgaggaactgactctcgaaacccaaggtatgtactactgggtatggggcagaacagacagaccttcagcttatggcacctgggtgcgggtgagagtgtttaggcctccctccctgacgatccatccccatgctgtgctggaaggacagccgttcaaggcaacatgcacagcagccacttactatcccggaaaccgtgctgagtttgtgtggttcgaggatgggcgacgtgtattcgaccctgcccagattcacacccagacacaggagaatcccgacgggttttccactgtgagcaccgtgacatcagcggcagtaggagggcagggcccaccccgaacgttcacttgccagcttacttggcatcgggacagtgttagctttagccgccggaatgcctctggcaccgcatccgtccttcctcgcccaaccatcaccatggaattcactggcgatcacgccgtttgtacagccgggtgtgttcccgagggagtgacctttgcttggtttctgggcgatgactcaagcccagccgaaaaggtggccgtcgcctcccaaacgagctgtgggcgacctggcaccgctaccatacgtagcactctgcccgtttcctacgaacagaccgagtatatctgccgattggccggttaccccgatgggataccagtcctggagcaccactga

SEQ ID NO: 217 - RNA, 1266 nt, synthetic construct
  source 1..1266: mol_type = other RNA
atgcctggcagatctctgcaaggactggccatcctcggactgtgggtttgcgcaacaggactgagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 218 - RNA, 1266 nt, synthetic construct
  source 1..1266: mol_type = other RNA
atgcccggcagaagcctccagggactggctatcctggggctgtgggtgtgcgccaccggtcttagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 219 - RNA, 1266 nt, synthetic construct
  source 1..1266: mol_type = other RNA
atgcctggaagatctctccagggactggcaatcctgggactgtgggtgtgtgcaacaggactgtctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 220 - RNA, 1263 nt, synthetic construct
  source 1..1263: mol_type = other RNA
atgcctgggagaagcctgcaagggctcgcaatcttgggcctgtgggtttgtgccacaggcttgtccccgggtcgaacaatcactgttgggcccaggggaaatgccagcaatgctgcaccttcagcaagcccacgaaacgcttcagcacccaggacaacacccactccacctcaaccgcggaaagccaccaagagcaaggcaagtaccgccaaacccgctcctcctcccaagacagggccacccaagacctctagtgagccagtgaggtgtaaccgccatgatccccttgccagatacgggagcagagtgcagattaggtgccggtttccaaactccacgagaaccgaatttcgcctccagatttggcggtatgcgactgccacagacgcagagattggtaccgctcccagcctggaggaggtcatggtgaacgtgtcagcgcctccgggtggccagctggtctacgactctgccccaaatcgaaccgaccctcacgtcatctgggctgaaggagcgggaccaggagcctctccacgcttgtatagcgtagttggccctctggggagacagcgcctgatcattgaggaactgacccttgagacacaggggatgtactactgggtgtggggcaggactgacaggcccagtgcctatggaacttgggttagggtccgcgtctttcggccacccagtctgaccatccatccacatgccgtgctggaaggccagcccttcaaagcgacttgcactgccgccacgtactatccagggaatagagccgagttcgtttggttcgaggatggccggagagtattcgatccagctcagatccacacccagacgcaggaaaacccggacggctttagcacggtgagtaccgtcacctctgctgccgtcggaggccaaggacctccccgtaccttcacatgccagcttacatggcaccgggactcagtaagcttttcacgtcgtaatgcatccggtactgcttctgtgctgcctcgacccaccatcaccatggagttcacaggggatcacgcagtgtgtacggcaggctgcgtgcctgaaggcgtgacattcgcctggtttctcggtgatgactcctctcctgctgaaaaggtggctgtagcctcccaaacaagctgtggtcggcctggaactgccactatacgctccactctcccggtgtcctacgaacagaccgagtacatatgcagactggctggatatcccgatggcattcccgtgctggagcatcactga

SEQ ID NO: 221 - RNA, 1281 nt, synthetic construct
  source 1..1281: mol_type = other RNA
atggctagaggtgccggcctggtgttctttgttggcgtgtgggtcgtgtcctgtctggccgctgctcctagaacatctagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 222 - RNA, 1281 nt, synthetic construct
  source 1..1281: mol_type = other RNA
atggcgagaggagccgggctcgtgttctttgtgggcgtatgggtcgtttcctgcctggctgccgcacccaggaccagcagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 223 - RNA, 1281 nt, synthetic construct
  source 1..1281: mol_type = other RNA
atggcaagaggagcaggactggtgtttttcgtgggagtttgggtggtgtcttgcctggctgctgctccaagaacctcttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 224 - RNA, 1278 nt, synthetic construct
  source 1..1278: mol_type = other RNA
atggcgagaggagccggactcgtgttctttgtgggagtctgggttgtgagctgcctggcagcagctccacgcactagctctccaggcagaactatcacagtgggacccagagggaatgccagcaatgcagccccgagtgccagccctcgtaacgccagcgctcccagaacaaccccaactccaccgcagcctagaaaggcgaccaagtccaaagcatccactgcaaaaccagccccacctcccaaaacgggacctcccaagaccagctccgagcctgtaaggtgcaatcggcatgaccccttggcccgatatggcagtcgcgtgcagattcgatgtcggtttcccaactctacccggactgagttccggttgcagatctggaggtatgcgaccgccactgacgctgagatcggcacagcaccaagcctggaagaagtgatggtgaacgttagtgctcctccgggcgggcaactcgtgtatgactccgcacccaaccgcacagatcctcacgtgatttgggccgaaggagccggacccggtgcgtcacctaggctctactctgtcgtaggaccactgggccgtcaacgcctgataatcgaggagctgactctggagacacagggtatgtactactgggtctggggcagaaccgacaggccatctgcttacgggacatgggtccgcgttcgagtatttcggccaccctcactgaccatacatccccatgccgttcttgaagggcagcctttcaaggcaacctgtactgctgccacatactatcccgggaatagggccgagttcgtctggtttgaagatggccgaagggtgtttgacccggctcagatccacacccagacacaggagaaccccgatggcttcagtacggtgtctaccgtcacaagcgccgctgtgggtggccaaggtcctcccagaactttcacctgtcagctgacgtggcacagggattccgtgagcttttcccgccgcaatgcgtcagggaccgcctccgtgcttcctcggccaaccatcacaatggaattcacgggtgatcacgctgtctgcacagctggctgcgttcctgagggcgtgacattcgcatggtttcttggtgacgactcatctcccgcagagaaggtggctgttgcctcacaaacgagttgtgggcgtccaggcactgccaccattcggtccaccctccccgtaagctacgaacagactgagtatatttgcagactggctggatacccggatgggattcctgtcctggaacatcactga

SEQ ID NO: 225 - RNA, 1305 nt, synthetic construct
  source 1..1305: mol_type = other RNA
atgggagtgaccggcattctccagctgcctcgggacagattcaagcggaccagcttcttcctgtgggtcatcatcctgttccagcggaccttcagcatccccagccctggcagaaccatcacagtgggccctagaggcaacgcctctaatgccgctcctagcgcctctcctagaaacgcctctgctcccagaaccacacctacacctccacagcctagaaaggccaccaagagcaaggccagcacagccaaacctgctcctccacctaagacaggccctccaaagacaagctctgagcccgtgcggtgcaacagacacgatccactggccagatacggcagccgggtgcagatcagatgcagattccccaacagcacccggaccgagttccggctccagatttggagatacgccaccgccacagatgccgagattggaacagcccctagcctggaagaagtgatggtcaacgtttcagcccctcctggcggccagctggtgtatgattctgcccctaaccggaccgatcctcacgtgatatgggctgaaggtgctggccctggcgcttcccctagactgtattctgttgtgggccctctgggcagacagcggctgatcattgaggaactgaccctggaaacccagggcatgtactactgggtctggggcagaaccgatagaccaagcgcctatggcacctgggttcgagtgcgagtgttcagacctcctagcctgaccatccatcctcacgccgttctggaaggccagcctttcaaggccacatgtaccgccgccacctactatcccggaaacagagccgagttcgtttggttcgaggacggcagaagggtgttcgaccccgctcagatccacacacagacccaagagaaccccgacggctttagcaccgtgtccacagtgacatctgccgccgttggaggacagggccctcctagaacctttacctgccagctgacctggcacagagacagcgtgtccttcagcagaagaaacgccagcggcacagccagcgttctgcctagacctaccatcaccatggaattcaccggcgaccacgccgtgtgtacagctggatgtgttcctgagggcgtgaccttcgcttggtttctgggcgacgatagcagccctgccgaaaaagtggctgtggccagccagacaagctgtggcagacctggaaccgccaccatcagaagcacactgcctgtcagctacgagcagaccgagtacatctgtcggctggccggctatcctgatggcatccctgtgctggaacaccactgataa

SEQ ID NO: 226 - RNA, 1305 nt, synthetic construct
  source 1..1305: mol_type = other RNA
atgggagtgactggcatactccagcttcctagagacaggtttaagcgcacatccttctttctgtgggtcatcatcctgttccaacggaccttcagcattcccagccccggcagaaccataacagtagggccacgggggaatgcttccaatgctgcaccttccgcttcaccgaggaatgcttctgccccaagaactacccccactcctcctcaacccaggaaagcgacaaagtccaaggccagcaccgcaaaacccgctcctcctccaaagactgggcccccaaagacaagtagcgaaccagttcggtgcaacaggcatgacccacttgcacgctatgggtcaagagtccagatacggtgtcgcttccctaacagtacaaggactgagtttcggctgcagatctggcgttatgccacagctactgacgcagagattggtaccgcccccagtttggaagaggtgatggtcaacgtgtccgcacccccaggaggacagctggtctatgactcagcgcccaataggaccgatccccacgtgatctgggcagaaggagccggtcctggggcctctccacggctgtactcagttgttggcccgcttggacgacagagactcatcatcgaagagctgacactggagacacaggggatgtactactgggtgtggggccgtactgaccgcccttccgcatatggcacttgggtgagagttcgcgtctttcggcccccttctctcaccatccatcctcatgccgtgctcgaaggccagccctttaaggccacatgcactgctgcgacctactaccctggcaacagagccgagtttgtctggtttgaggatggtcggcgagtattcgatccagcccagattcacacacaaacgcaggaaaatccggacggcttcagcacagtgtccacggtgacctctgctgcagttggtggacaaggaccccctcgaaccttcacctgtcagctgacctggcacagagactccgtaagcttcagccgtagaaacgcctctggaaccgccagtgtgttgccgaggccgactatcacgatggaattcacaggcgatcatgccgtctgtactgccggctgtgtgccagaaggcgtaaccttcgcttggtttctcggggatgactcaagtcctgcagagaaagtggctgtggcctctcagacgagctgcggtcgaccaggaacagctaccattcgcagcactctgcccgtgtcctacgagcagacggagtacatctgcaggctggccggctatcccgatgggattccagtcctggagcaccactgataa

SEQ ID NO: 227 - RNA, 1305 nt, synthetic construct
  source 1..1305: mol_type = other RNA
atgggagtaaccggaattctccagctgccaagagatcgattcaaaagaacatcatttttcctttgggtaattattctgtttcagagaacattttccatcccttctcctggaagaaccatcacagtgggaccaagaggaaatgcaagcaatgcagcaccttctgcttctccaagaaatgcttctgctccaagaaccaccccaacccctcctcagccaagaaaagcaaccaaatccaaagcatccacagcaaaacctgcacctcctccaaaaacaggacctccaaaaacctcctctgaacctgtgagatgcaacagacatgatcctctggcaagatatggatcaagagtgcagatcagatgcagatttccaaattccaccagaacagaattcagactccagatctggagatatgcaacagcaacagatgcagaaattggaacagcaccatctctggaagaagtgatggtgaatgtgtctgctcctcctggaggacagctggtgtatgattctgctccaaacagaacagatcctcatgtgatctgggctgaaggagctggacctggagcttctccaagactgtactctgtggtgggacctctgggaagacagagactgatcattgaagaactgaccctggaaacccagggaatgtactactgggtgtggggaagaacagacagaccttctgcttatggaacctgggtgagagtgagagtgttcagacctccttctctgaccatccaccctcatgctgtgctggaaggacagcctttcaaagcaacctgcacagcagcaacctactaccctggaaacagagctgaatttgtgtggtttgaagatggaagaagggtgtttgatcctgctcagatccacacccagacccaggaaaatcctgatggattttccacagtgtccacagtgacatctgctgctgtgggaggacagggacctccaagaaccttcacctgccagctgacctggcacagagattctgtgtctttttcaagaagaaatgcttctggaacagcttctgtgctgccaagaccaaccatcaccatggaattcacaggagatcatgctgtgtgcacagctggatgtgtgcctgaaggagtgacctttgcttggtttctgggagatgattcttctccagctgaaaaagtggctgtggcttcccagacctcttgtggaagacctggaacagcaaccatcagatccaccctgcctgtgtcttatgaacagacagaatacatttgcagactggctggataccctgatggaatccctgtgctggaacaccactgataa

SEQ ID NO: 228 - RNA, 1302 nt, synthetic construct
  source 1..1302: mol_type = other RNA
atgggagttaccggaattctgcaattgcccagggatcggttcaagcgcacatccttcttcctgtgggtcatcatcctctttcagcgtactttctccataccctctcccggacgaactatcactgtaggcccaaggggcaacgctagtaatgccgctcccagtgcttcaccacgcaatgcgagcgcacccagaactacacccacacctcctcagccgaggaaagccaccaagtccaaagccagcaccgccaaacccgctcctccacctaaaacagggcctcccaagacctcaagcgagcccgttagatgcaatcggcatgatccactggctcgttacggttctcgggtccagatacgctgtaggtttcctaactccacacgaaccgagttcagattgcagatctggagatatgccacagccactgacgctgagattggcactgcacctagtctggaggaggtgatggtgaacgtgagcgcccctcctggcggtcagcttgtgtatgactcagcaccgaatcgcacagatccgcacgtcatatgggccgaaggtgcagggcccggtgcatcccctcggctgtattccgtggtcggaccactcgggcgccagaggcttatcattgaggaactgaccctcgaaacccagggtatgtactactgggtatggggccgtaccgaccggcctagcgcctacggaacttgggtgagagttcgggtgttcagaccgccaagtcttacaattcaccctcatgccgtgctcgaaggtcagccatttaaggccacgtgtactgcggctacgtactatcccgggaatcgggctgaattcgtgtggtttgaggatggcaggagagtgttcgacccagcccaaatccacacccaaacacaggagaacccagacgggttttccaccgtgtcaacggtcacatctgccgccgtcggaggacaagggccacccagaaccttcacatgccagctgacctggcatagggatagcgtaagctttagccggcgaaacgcatctggaacggcgagcgttctgcctcgaccaacaatcaccatggagttcaccggcgatcacgcagtgtgcactgctgggtgtgtacccgaaggcgtgacatttgcctggtttctgggagatgactcctcacccgcagaaaaggtcgccgttgcatctcagaccagttgtggcaggcccgggactgctaccatccgcagcactctgccggtgtcttacgaacagacggagtacatttgccgcttggcgggctatccagacggcattccagttctggagcatcactga

SEQ ID NO: 229 - RNA, 996 nt, synthetic construct
  source 1..996: mol_type = other RNA
atgggccgcctgacctccggcgtgggcaccgccgccctgctggtggtggccgtgggcctgcgcgtggtgtgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 230 - RNA, 996 nt, synthetic construct
  source 1..996: mol_type = other RNA
atgggcagactgacatctggcgtgggaacagctgctctgctggtggttgctgtgggcctgagagtcgtgtgtgccaaatacgccctggccgatcctagcctgaagatggctgaccccaaccggttccggggcaagaatctgcctgttctggaccagctgaccgatcctcctggcgtgaaacgggtgtaccacatccagccaagcctggaagatcccttccagcctcctagcatccccatcaccgtgtactacgccgtgctggaaagggcctgtagaagcgtgctgctgcacgccccatctgaagcccctcaaatcgtcagaggcgcttccgacgaggccagaaagcacacctacaacctgacaatcgcctggtacagaatgggcgacaactgcgccattcctatcaccgtgatggagtacaccgagtgtccctacaacaagagcctgggcgtgtgccccatcagaacacagcctagatggtcctactacgacagcttcagcgccgtgtccgaggacaatctgggcttcctgatgcatgcccctgcctttgagacagccggcacctatctgcggctggtcaagatcaacgactggaccgagatcacccagttcatcctggaacacagagccagagccagctgcaaatacgctctgcccctgagaattcctcctgccgcctgtctgacaagcaaggcctatcagcagggcgtgaccgtggatagcatcggcatgctgcccagattcatccccgagaaccagagaacagtggccctgtactccctgaagatcgccggatggcacggacccaagcctccatacacaagcacactgctgcctccagagctgagcgacaccaccaatgccacacagcctgaactggtgcctgaggacccagaggattctgccctgcttgaagatcctgccggcaccgtgtctagccagattcctcctaactggcacatccccagcatccaggatgtggcccctcatcattga

SEQ ID NO: 231 - RNA, 996 nt, synthetic construct
  source 1..996: mol_type = other RNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgctcttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 232 - RNA, 996 nt, synthetic construct
  source 1..996: mol_type = other RNA
atggggagactcacatcaggcgtaggaaccgctgccctgttggtcgtggccgttggtctgagagttgtgtgtgccaaatatgctctcgctgatccgagcctcaagatggcagatcccaaccgatttcggggaaagaatctgccagtactggaccagctgacggacccacctggcgtcaaacgcgtctaccacatacagcctagtcttgaggacccttttcagccaccgtctatccccattaccgtgtactatgccgtgctggaacgcgcgtgtaggtcagttctgctgcatgccccatccgaagccccccagatcgtcagaggagcttctgatgaagcacgcaaacacacctacaacctcacaatagcgtggtatcgaatgggcgataactgcgcaattcccatcacagtcatggagtacacggagtgcccctacaacaagagcctcggtgtttgccctatcaggacacaacccaggtggagctattacgacagtttcagcgccgtgtctgaggacaatctggggtttctgatgcacgcacccgccttcgagactgccggcacctacttgcggctggtgaagatcaacgactggactgagatcacccagttcatcctggaacatagggccagagccagctgcaagtatgcccttcccctgcggattccgcctgcagcatgtctgacctcaaaagcctaccagcaaggggtgactgtggacagcattggcatgctgcctcgtttcattcccgagaatcaacggacagtggctctgtattccctgaagatcgcaggatggcatgggcccaaaccaccttatacctctacgttgcttccaccagaactcagtgacaccactaatgcgacacagccagaacttgtgcctgaggatcctgaagatagcgctctgttggaggatccagccggtactgtgtcctcccagataccacccaattggcacattccttccattcaggacgtagctccgcatcactga

SEQ ID NO: 233 - RNA, 996 nt, synthetic construct
  source 1..996: mol_type = other RNA
atgggcagactgacctccggcgtgggcaccgccgccctgctggtggtggccgtgggcctgagagtggtgtgcgccaaatacgccctggccgatccctccctgaaaatggccgatcccaacaggttcagaggcaaaaacctgcccgtgctggatcagctgaccgatccccctggcgtgaaaagagtgtaccacatccagccctccctggaagatcccttccagcccccttccatccccatcaccgtgtactacgccgtgctggaaagagcttgcagatccgtgctgctgcacgccccctccgaagcccctcagatcgtgagaggcgcctccgatgaagccagaaaacacacctacaacctgaccatcgcctggtacagaatgggcgataactgcgccatccccatcaccgtgatggaatacaccgaatgcccctacaacaaatccctgggcgtgtgccccatcagaacccagcccagatggtcctactacgattccttctccgccgtgtccgaagataacctgggcttcctgatgcacgcccccgccttcgaaaccgccggcacctacctgagactggtgaaaatcaacgattggaccgaaatcacccagttcatcctggaacacagagccagagcctcctgcaaatacgccctgcccctgagaatccctcccgccgcctgcctgacctccaaagcctaccagcagggcgtgaccgtggattccatcggcatgctgcccagattcatccccgaaaaccagagaaccgtggccctgtactccctgaaaatcgccggctggcacggccccaaacccccttacacctccaccctgctgccccctgaactgtccgataccaccaacgccacccagcccgaactggtgcccgaagatcccgaagattccgccctgctggaagatcccgccggcaccgtgtcctcccagatccctcccaactggcacatcccctccatccaggatgtggcccctcaccactga

SEQ ID NO: 234 - RNA, 1206 nt, synthetic construct
  source 1..1206: mol_type = other RNA
atgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 235 - RNA, 1206 nt, synthetic construct
  source 1..1206: mol_type = other RNA
atgagaatgcagctgctgctcctgatcgccctgtctctggccctggtcaccaatagcagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 236 - RNA, 1206 nt, synthetic construct
  source 1..1206: mol_type = other RNA
atgcgaatgcagcttctgctgctcattgccttgtcccttgccttggtgaccaactcacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 237 - RNA, 1206 nt, synthetic construct
  source 1..1206: mol_type = other RNA
atgagaatgcagctgctgctgctgatcgccctgtccctggccctggtgaccaactccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 238 - RNA, 1233 nt, synthetic construct
  source 1..1233: mol_type = other RNA
atgggccgcctgacctccggcgtgggcaccgccgccctgctggtggtggccgtgggcctgcgcgtggtgtgcgccaagtacgcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 239 - RNA, 1233 nt, synthetic construct
  source 1..1233: mol_type = other RNA
atgggcagactgacatctggcgtgggaacagctgctctgctggtggttgctgtgggcctgagagtcgtgtgtgccaaatacgccagaaccagctggaaaagagtgaccagcggcgaggatgtggtgctgcttcctgctcctgctggccccgaggaaagaacaagagcccacaaactgctgtgggccgctgagcctcttgatgcctgtggacctctcagacctagctgggttgcactgtggccacctcggagagtgctggaaacagtggtggatgccgcctgcatgagagcccctgaacctctggccattgcctactctccaccatttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtcctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgtgccgagatgagaatctatgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgcctcagagacagcccgagcctgtggaacctacaagacctcatgttcgggcctga

SEQ ID NO: 240 - RNA, 1233 nt, synthetic construct
  source 1..1233: mol_type = other RNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 241 - RNA, 1233 nt, synthetic construct
  source 1..1233: mol_type = other RNA
atggggagactcacatcaggcgtaggaaccgctgccctgttggtcgtggccgttggtctgagagttgtgtgtgccaaatatgctcgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 242 - RNA, 1233 nt, synthetic construct
  source 1..1233: mol_type = other RNA
atgggcagactgacctccggcgtgggcaccgccgccctgctggtggtggccgtgggcctgagagtggtgtgcgccaaatacgccagaacctcctggaaaagagtgacctccggcgaagatgtggtgctgctgcccgcccccgccggccccgaagaaagaaccagagcccacaaactgctgtgggccgccgaacccctggatgcctgcggccccctcagaccctcctgggtggccctgtggccccctagaagggtgctggaaaccgtggtggatgccgcctgcatgagagcccccgaacccctggccatcgcctactcccctcccttccccgccggcgatgaaggcctgtactccgaactggcctggagagatagagtggccgtggtgaacgaatccctggtgatctacggcgccctggaaaccgattccggcctgtacaccctgtccgtggtgggcctgtccgatgaagccagacaggtggcctccgtggtgctggtggtggaacccgcccccgtgcccacccccacccccgatgattacgatgaagaagatgatgccggcgtgtccgaaagaacccccgtgtccgtgccccctcccacccctccccgcagaccccctgtggcccctcccacccaccccagagtgatccccgaagtgtcccacgtgagaggcgtgaccgtgcacatggaaacccccgaagccatcctgttcgcccccggcgaaaccttcggcaccaacgtgtccatccacgccatcgcccacgatgatggcccctacgccatggatgtggtgtggatgagattcgatgtgccctcctcctgcgccgaaatgagaatctacgaagcctgcctgtaccacccccagctgcccgaatgcctgtcccccgccgatgccccctgcgccgtgtcctcctgggcctacagactggccgtgagatcctacgccggctgctccagaaccacccctccccctagatgcttcgccgaagccagaatggaacccgtgcccggcctggcctggctggcctccaccgtgaacctggaattccagcacgccagcccccagcacgccggcctgtacctgtgcgtggtgtacgtggatgatcacatccacgcctggggccacatgaccatctccaccgccgcccagtacagaaacgccgtggtggaacagcacctgccccagagacagcccgaacccgtggaacccaccagaccccacgtgagagcctga

SEQ ID NO: 243 - RNA, 1218 nt, synthetic construct
  source 1..1218: mol_type = other RNA
atggcacggggagccggattggtgttctttgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 244 - RNA, 1218 nt, synthetic construct
  source 1..1218: mol_type = other RNA
atggcacggggcgcaggtttggtctttttcgtgggcgtgtgggtggtgagctgcttggcagccgcaccacgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 245 - RNA, 1218 nt, synthetic construct
  source 1..1218: mol_type = other RNA
atggcgagaggagccggactcgtgttctttgtgggagtctgggttgtgagctgcctggcagcagctccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 246 - RNA, 1218 nt, synthetic construct
  source 1..1218: mol_type = other RNA
atggcgagaggagccgggctcgtgttctttgtgggcgtatgggtcgtttcctgcctggctgccgcaccccgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 247 - RNA, 1218 nt, synthetic construct
  source 1..1218: mol_type = other RNA
atggcgagaggagccggactcgtgttctttgtgggagtctgggttgtgagctgcctggtagtagttccacgcacctcttggaaacgcgttacttccggggaggacgttgtcctccttccagcacccgcaggacctgaagagaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaagaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 248 - RNA, 1152 nt, synthetic construct
  source 1..1152: mol_type = other RNA
atgcgcacctcttggaaacgcgttacttccggggaggacgttgtcctccttccagcacccgcaggacctgaggaaaggactagggcccacaagctgctgtgggccgctgaacctctggatgcctgtggtcctctgagacctagctgggtcgccctttggccacctagacgcgttctggagacggtcgtggatgccgcgtgcatgcgtgcacccgaacctctggccatcgcctatagtcccccttttcccgctggcgacgaggggctttactccgaactggcctggcgggatagggtggcggtggtgaacgagagcctcgtcatctacggtgctctggaaaccgactcaggactgtatacgctcagcgttgttggcctctccgatgaggctcgacaggttgcctccgtagtgctggtcgtagaaccagcccccgtaccaacacccacacccgacgactacgacgaggaggacgacgctggagttagcgaaagaacaccggtgagtgtgccacctcccacaccgccaaggagacccccagtagcacctccaacccatccgagagtgattcccgaggtcagccatgtgcgcggcgtaactgtgcacatggagacgcccgaagcgatactgtttgcccctggagagacattcggcaccaatgtgtccatacacgcaattgcgcacgatgatggcccatacgctatggacgtcgtctggatgaggttcgatgtgccttcttcttgcgccgagatgaggatctacgaggcatgcctgtatcacccccaattgccggagtgtctgtctcccgcagatgcaccgtgtgcagtgagtagctgggcttatcggttggctgtccggagttatgctgggtgttcacggaccaccccacctccacgttgctttgctgaagccagaatggaacccgtgcctggtctggcttggctggcatcaactgtcaacctggagttccagcatgcctctccacagcacgcaggcctgtatctctgcgtggtgtacgttgacgatcacatccatgcgtgggggcatatgaccatcagcacagctgcccagtaccgcaatgccgtcgtggagcagcacctcccccaacggcagccagaaccagtggagcccactcggcctcatgtgcgagcctga

SEQ ID NO: 249 - RNA, 1233 nt, synthetic construct
  source 1..1233: mol_type = other RNA
atgggcggagctgctgctagactgggagccgtgatcctgttcgtggttatcgtgggactgcatggcgtgcggggcaagtatagaacctcttggaagcgcgtgacaagcggcgaggatgtggttctgcttcctgctcctgccggacctgaggaaagaacaagagcccacaagctgctgtgggccgctgaacctttggatgcctgtggacctctgaggccttcttgggttgcactgtggccacctcggagagtgctggaaacagtggtggatgccgcctgcatgagagcccctgaacctctggccattgcctactctccaccttttccagccggcgacgagggcctgtattctgagcttgcttggagagacagagtggccgtggtcaacgagagcctggttatctatggcgccctggaaaccgacagcggcctgtacacactgtctgtcgtgggcctgtctgacgaggctagacaggtggcatctgtggtgctggtggtggaacctgctccagtgcctacacctacacctgacgactacgacgaggaagatgacgctggcgtcagcgagagaacccctgtttctgtgcctcctcctacgcctcctcgtagacctcctgttgctcctccaacacaccccagagtgatccctgaagtgtctcacgtgcggggcgtgaccgtgcacatggaaacacctgaggccatcctgttcgcccctggcgagacatttggcaccaacgtgtccatccacgctatcgcccacgacgatggcccttacgccatggatgtcgtgtggatgagattcgacgtgcccagcagctgcgccgagatgagaatctacgaggcctgcctgtatcaccctcagctgcccgaatgtctgagccctgctgatgccccttgtgccgttagcagctgggcctatagactggccgtgcggtcttatgccggctgctctagaacaacccctcctcctcggtgtttcgccgaggccagaatggaacctgttcctggactggcctggctggcctccacagtgaacctggaatttcagcacgcctctccacagcacgccggcctgtatctgtgtgtggtgtacgtggacgatcacatccacgcctggggccacatgaccatctctacagccgctcagtaccggaacgccgtggttgaacagcatctgccccagagacagcccgagcctgtggaacctacaagacctcatgttcgggcctgataa

SEQ ID NO: 250 - RNA, 1230 nt, synthetic construct
  source 1..1230: mol_type = other RNA
atgggcggagctgcggctcgcctcggagccgtcattctgttcgtggtgatcgtgggtctgcatggggtcagaggaaagtaccggaccagctggaaaagggtgacatctggggaagatgtcgtgctccttcctgcgcctgctggcccagaagaacgcactagggcccacaagcttctgtgggccgcggagccactggatgcgtgtggtcccctgcgaccctcttgggtggcattgtggccaccccgacgcgtactcgaaacggtcgttgacgccgcctgtatgagagcgccagagcccctcgccattgcctacagcccgccttttcccgccggtgacgaaggactgtacagtgagctggcctggcgcgatagggtcgccgtagttaacgagtccctggtgatatacggcgctctggaaaccgacagtggcttgtacaccctgtccgttgtaggcctgtccgatgaagcacggcaagtggcttccgtggtactggtcgtagagcccgcaccagttcccacacccaccccggatgactacgatgaggaggacgatgccggtgtgagtgagcgtacacctgttagcgtacctccaccaactcctccacgccgccctcctgttgcaccgcctacacatccccgtgtgattcctgaagtgtcacacgttagaggggtgacagtccacatggagactcccgaagccatcctctttgcaccaggcgagacttttgggaccaatgtgagcattcacgccatagctcacgatgacgggccctatgccatggacgtggtgtggatgaggttcgatgtgccctcatcatgcgctgagatgcggatctacgaagcttgcctgtatcacccacagcttcccgagtgcttgtctcccgctgacgccccttgtgctgttagctcttgggcttatcggcttgccgtcaggagctatgctggatgctccagaaccacacctccaccgaggtgtttcgccgaggccagaatggagcctgtgccaggactggcctggctggcaagtactgtgaacctggaattccagcacgcatcacctcagcatgcaggcctgtacctctgcgttgtctatgtcgacgaccatatccacgcatggggccatatgaccatcagcacggcagcacagtatcggaatgctgtggtcgagcagcacttgccccagcgacaacccgaaccagtggagccaaccagaccgcatgtgcgggcctga

SEQ ID NO: 251 - RNA, 1233 nt, synthetic construct
  source 1..1233: mol_type = other RNA
atgggaggagctgctgctagattgggagctgtgattctgtttgtggtgattgtgggactgcatggagtgagaggaaaatacagaacctcttggaaaagagtgacctctggagaagatgtggtgctgctgccagctccagctggaccagaagaaagaaccagagcacacaaactgctgtgggctgctgaacctctggatgcttgtggacctctgagaccttcttgggtggctctgtggccaccaagaagggtgctggaaacagtggtggatgctgcttgcatgagagcacctgaacctctggcaattgcatactctcctccttttccagctggagatgaaggactgtattctgaactggcttggagagacagagtggctgtggtgaatgaatctctggtgatctatggagcactggaaacagattctggactgtacaccctgtctgtggtgggactgtctgatgaagcaagacaggtggcatctgtggtgctggtggtggaaccagctccagtgccaaccccaaccccagatgattatgatgaagaagatgatgctggagtgtctgaaagaaccccagtgtctgtgccaccaccaaccccaccaagaagaccaccagtggctccaccaacccacccaagagtgatcccagaagtgtctcatgtgagaggagtgacagtgcacatggaaacccctgaagcaatcctgtttgcacctggagaaacctttggaaccaatgtgtccatccatgcaattgcacatgatgatggaccttatgcaatggatgtggtgtggatgagatttgatgtgccttcttcttgtgctgaaatgagaatctatgaagcttgcctgtaccaccctcagctgcctgaatgcctgtctcctgctgatgctccttgtgctgtgtcttcttgggcttacagactggctgtgagatcttatgctggatgctccagaaccaccccaccaccaagatgctttgctgaagcaagaatggaacctgtgcctggactggcatggctggcatccacagtgaatctggaatttcagcatgcttctcctcagcatgctggactgtacctgtgtgtggtgtatgtggacgatcacatccatgcttggggacacatgaccatcagcacagctgctcagtacagaaatgctgtggtggaacagcacctgccacagagacagccagaaccagtggaaccaaccagaccacatgtgagagcttgataa

SEQ ID NO: 252 - RNA, 1230 nt, synthetic construct; identical to SEQ ID NO: 250
  source 1..1230: mol_type = other RNA

(19) United States
(12) Patent Application Publication
(10) Pub. No.: US 2024/0016922 A1
(43) Pub. Date: Jan. 18, 2024

(54) RNA VACCINES ENCODING HERPES SIMPLEX VIRUS GLYCOPROTEINS AND USES THEREOF

(71) Applicant: THE TRUSTEES OF THE UNIVERSITY OF PENNSYLVANIA, Philadelphia, PA (US)

(72) Inventors: Harvey FRIEDMAN, Merion, PA (US); Sita AWASTHI, Bala Cynwyd, PA (US); Gary Cohen, Havertown, PA (US); Drew WEISSMAN, Wynnewood, PA (US)

(73) Assignee: THE TRUSTEES OF THE UNIVERSITY OF PENNSYLVANIA, Philadelphia, PA (US)

(21) Appl. No.: 18/365,166

(22) Filed: Aug. 3, 2023

Related U.S. Application Data
(63) Continuation-in-part of application No. 17/349,929, filed on Jun. 17, 2021, now Pat. No. 11,793,872.
Application No. 17/349,929 is a continuation-in-part of application No. 16/640,008, filed on Feb. 18, 2020, now Pat. No. 11,141,478.
(60) Provisional application No. 62/546,648, filed on Aug. 17, 2017.
(60) Provisional application No. 62/701,019, filed on Jul. 20, 2018.

(51) Int. Cl. A61K 39/245; C07K 14/005; A61P 31/22

(52) U.S. Cl. CPC A61K 39/245; C07K 14/005; A61P 31/22; A61K 2039/53

(57) ABSTRACT

The present disclosure provides compositions for the prevention and treatment of genital herpes, comprising nucleoside-modified RNAs that encode herpes simplex virus (HSV) glycoproteins, including those involved in virus entry and immune evasion, and methods of use thereof.

### STATEMENT REGARDING FEDERALLY SPONSORED RESEARCH OR DEVELOPMENT

Certain inventions described herein were made with U.S. government support under AI139618 awarded by The National Institutes of Health. The government has certain rights in the invention.

### SEQUENCE LISTING STATEMENT

The instant application contains a Sequence Listing that has been submitted electronically in XML format and is hereby incorporated by reference in its entirety. The XML copy, created on Aug. 3, 2023, is named P-572137-US2-SEQLIST.xml and is 367 kilobytes in size.

### TECHNICAL FIELD

The present disclosure provides compositions for the prevention and treatment of genital herpes, comprising nucleoside-modified RNAs that encode herpes simplex virus (HSV) glycoproteins, including those involved in virus entry and immune evasion, and methods of use thereof.

### BACKGROUND

A half-billion people worldwide are infected with herpes simplex virus type 2 (HSV-2). Many of these individuals are unaware they are infected, yet they are at risk of transmitting infection to intimate partners. About 20% of infected people have frequent, painful recurrent genital lesions. Lifelong daily suppressive therapy with acyclovir or valacyclovir reduces the frequency of recurrences and lowers risk for transmission, but not all people respond or are willing to take daily therapy. Anxiety about transmission to intimate partners is perhaps the greatest concern of people with genital herpes.

One of the most dreaded complications of genital herpes is neonatal herpes. This infection is uncommon (1:3,000 births in the U.S.) but devastating with high morbidity and mortality in newborns. Neonates acquire HSV-1 or HSV-2 infection from mothers who have reactivation infection at the time of labor and delivery, or the infection in the pregnant woman can be a first-time infection late in pregnancy.

Prophylactic vaccines that are under development are intended to prevent first-time HSV infections, and those vaccines may not be effective in preventing recurrences in people already infected. From a public health perspective, the biggest impact of an effective genital herpes vaccine will be on HIV infection. Genital herpes increases the risk of acquiring or transmitting HIV by 3-4-fold.

### SUMMARY

The present disclosure provides pharmaceutical compositions (e.g., immunogenic compositions, e.g., vaccines) for delivering a particular herpes simplex virus (HSV) glycoprotein or immunogenic fragment thereof (e.g., HSV gE, HSV gC, or HSV gD) to a subject (e.g., a patient) and related technologies (e.g., methods).

In some embodiments, the present disclosure provides a nucleoside-modified RNA encoding the ectodomain of HSV glycoprotein E (gE) or immunogenic fragment thereof.

In another embodiment, the present disclosure provides a composition comprising a nucleoside-modified RNA encoding the ectodomain of HSV glycoprotein E (gE) or immunogenic fragment thereof.

In another embodiment, the present disclosure provides a method of treating a Herpes Simplex Virus (HSV) infection or suppressing, inhibiting, or reducing the incidence of an HSV infection in a subject, comprising the step of administering a nucleoside-modified RNA encoding the ectodomain of HSV glycoprotein E (gE) or immunogenic fragment thereof, or a composition comprising a nucleoside-modified RNA encoding the ectodomain of HSV glycoprotein E (gE) or an immunogenic fragment thereof.

In another embodiment, the present disclosure provides a method of inducing an immune response in a subject, comprising the step of administering a nucleoside-modified RNA encoding the ectodomain of HSV glycoprotein E (gE) or an immunogenic fragment thereof, or a composition comprising a nucleoside-modified RNA encoding the ectodomain of HSV glycoprotein E (gE) or an immunogenic fragment thereof.

Other features and advantages of the present disclosure will become apparent from the following detailed description examples and figures. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1C. Characterization of exemplary translational products of an immunogenic fragment of the ectodomain from HSV-2 gC (gC2)-, HSV-2 gD (gD2)- and HSV-2 gE (gE2)-modified RNA in Vero cells.

FIG. 1A. Western blot showing expression of an immunogenic fragment of HSV-2 gC from an exemplary modified RNA.

FIG. 1B. Western blot showing expression of an immunogenic fragment of HSV-2 gD from an exemplary modified RNA.

FIG. 1C. Western blot showing expression of an immunogenic fragment of HSV-2 gE from an exemplary modified RNA.

FIGS. 2A-2B. CD4⁺ T cell responses to exemplary RNA encoding immunongenic fragments of HSV-2 gC, HSV-2 gD or HSV-2 gE each administered at a different intradermal site. Splenocytes were stimulated with exemplary immunogenic fragment subunit antigen glycoproteins (FIG. 2A) or 15 amino acid peptides with 11 overlapping amino acids to stimulate HSV-2 specific T cell responses (FIG. 2B). * indicates p<0.05 (t test) comparing gC, gD or gE with PBS stimulated CD4⁺ T cells or DMSO stimulated CD4⁺ T cells. Error bars represent SD.

FIGS. 3A-3B. CD8⁺ T cell responses to exemplary RNA encoding immunogenic fragments HSV-2 gC, HSV-2 gD, or HSV-2 gE, each administered at a different intradermal site. Splenocytes were stimulated with subunit antigen glycoproteins (FIG. 3A) or 15 amino acid peptides with 11 overlapping amino acids to stimulate HSV-2 specific T cell responses (FIG. 3B). * indicates p<0.05 comparing gE pool 2 with DMSO control. Error bars represent SD.

FIGS. 4A-4B shows cumulative recurrent genital lesion days per group. Previously infected guinea pigs were immunized on days 35 and 65 post-infection with nucleoside-modified mRNARNA encapsulated in lipid nanoparticle 315 and expressing gE2 (30 ug), 10 ug each of exemplary gC2, gD2, gE2 immunogenic fragments (gC2/gD2/gE2), or PBS (control). Animals were scored daily Monday to Friday for recurrent genital lesions from 1 day after the first immunization until the end of the study on day 116. From the time of the second immunization, differences appeared comparing exemplary gC2/gD2/gE2 immunogenic fragments or exemplary gE2 immunogenic fragment with the PBS group.

FIGS. 5A-5B show days with recurrent genital lesions for each animal starting 1 day after the second immunization. The same animals as in FIGS. 4A-4B are shown here. P values were calculated by the two-tailed Mann Whitney test and demonstrate highly significant differences comparing the group administered with exemplary gC2/gD2/gE2 immunogenic fragments with PBS (**, P<0.01; ns, P value not significant).

FIG. 6A shows CD4⁺ T cell responses to immunization with gE2 mRNA. Mice were immunized twice with 10 μg is gE2 mRNA-LNP. Splenocytes from these mice were stimulated with a gE2 overlapping peptide pool. CD4⁺ and CD8⁺ T-cells producing cytokines were analyzed by flow cytometry.

FIG. 6B shows CD8⁺ T cell responses to immunization with gE2 mRNA. Mice were immunized twice with 10 μg is gE2 mRNA-LNP. Splenocytes from these mice were stimulated with a gE2 overlapping peptide pool. CD4⁺ and CD8⁺ T-cells producing cytokines were analyzed by flow cytometry.

### CERTAIN DEFINITIONS

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

So that the present disclosure may be more readily understood, certain terms are first defined below. Additional definitions for the following terms and other terms are set forth throughout the specification.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amino acid: In its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody agent: As used herein, the term “antibody agent” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs 1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antigen: Those skilled in the art, reading the present specification, will appreciate that the term “antigen” refers to a molecule that is recognized by the immune system, e.g., in particular embodiments the adaptive immune system, such that it elicits an antigen-specific immune response. In some embodiments, an antigen-specific immune response may be or comprise generation of antibodies and/or antigen-specific T cells. In some embodiments, an antigen is a peptide or polypeptide that comprises at least one epitope against which an immune response can be generated. In some embodiments, an antigen is presented by cells of the immune system such as antigen presenting cells like dendritic cells or macrophages. In some embodiments, an antigen or a processed product thereof such as a T-cell epitope is bound by a T- or B-cell receptor, or by an immunoglobulin molecule such as an antibody. Accordingly, an antigen or a processed product thereof may react specifically with antibodies or T lymphocytes (T cells). In some embodiments, an antigen is a parasitic antigen. In accordance with the present disclosure, in some embodiments, an antigen may be delivered by RNA molecules as described herein. In some embodiments, a peptide or polypeptide antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide or polypeptide antigen can be greater than 50 amino acids. In some embodiments, a peptide or polypeptide antigen can be greater than 100 amino acids. In some embodiments, an antigen is recognized by an immune effector cell. In some embodiments, an antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present disclosure, in some embodiments, an antigen can be presented or present on the surface of a cell, e.g., an antigen presenting cell. In some embodiments, an antigen is presented by a diseased cell such as a virus-infected cell. In some embodiments, an antigen receptor is a TCR which binds to an epitope of an antigen presented in the context of MHC. In some embodiments, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells preferably release cytotoxic factors, e.g. perforins and granzymes.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: Those skilled in the art, reading the present specification, will appreciate that the term “binding” typically refers to a non-covalent association between or among entities or moieties. In some embodiments, binding data are expressed in terms of “IC50”. As is understood in the art, IC50 is the concentration of an assessed agent in a binding assay at which 50% inhibition of binding of reference agent known to bind the relevant binding partner is observed. In some embodiments, assays are run under conditions in which the assays are run (e.g., limiting binding target and reference concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

Cap: As used herein, the term “cap” refers to a structure comprising or essentially consisting of a nucleoside-5 ‘-triphosphate that is typically joined to a 5’-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a 7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a single-stranded DNA template in the presence of a dinucleotide or trinucleotide cap analog.

Cell-mediated immunity: “Cell-mediated immunity,” “cellular immunity,” “cellular immune response,” or similar terms are meant to include a cellular response directed to cells characterized by expression of an antigen, in particular characterized by presentation of an antigen with class I or class II MHC. A cellular response relates to immune effector cells, in particular to T cells or T lymphocytes which act as either “helpers” or “killers.” The helper T cells (also termed CD4⁺ T cells or CD4 T cells) play a central role by regulating the immune response and the killer cells (also termed cytotoxic T cells, cytolytic T cells, CD8⁺ T cells, CD8 T cells, or CTLs) kill diseased cells such as virus-infected cells, preventing the production of more diseased cells.

Co-administration: As used herein, the term “co-administration” refers to use of a pharmaceutical composition (e.g., immunogenic composition, e.g., vaccine) described herein and an additional therapeutic agent. The combined use of a pharmaceutical composition (e.g., immunogenic composition, e.g., vaccine) described herein and an additional therapeutic agent may be performed concurrently or separately (e.g., sequentially in any order). In some embodiments, a pharmaceutical composition (e.g., immunogenic composition, e.g., vaccine) described herein and an additional therapeutic agent may be combined in one pharmaceutically-acceptable carrier, or they may be placed in separate carriers and delivered to a target cell or administered to a subject at different times. Each of these situations is contemplated as falling within the meaning of “co-administration” or “combination,” provided that a pharmaceutical composition (e.g., immunogenic composition, e.g., vaccine) described herein and an additional therapeutic agent are delivered or administered sufficiently close in time that there is at least some temporal overlap in biological effect(s) generated by each on a target cell or a subject being treated.

Codon-optimized: As used herein, the term “codon-optimized” refers to alteration of codons in a coding region of a nucleic acid molecule to reflect the typical codon usage of a host organism without preferably altering the amino acid sequence encoded by the nucleic acid molecule. Within the context of the present disclosure, in some embodiments coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190 th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Derived: In the context of an amino acid sequence (peptide or polypeptide) “derived from” a designated amino acid sequence (peptide or polypeptide), it refers to a structural analogue of a designated amino acid sequence. In some embodiments, an amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

Designed: As used herein, the term “designed” refers to an agent (i) whose structure is or was selected by the hand of man; (ii) that is produced by a process requiring the hand of man; and/or (iii) that is distinct from natural substances and other known agents.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

Epitope: As used herein, the term “epitope” refers to a moiety that is specifically recognized by an immunoglobulin (e.g., antibody or receptor) binding component. For example, an epitope may be recognized by a T cell, a B cell, or an antibody. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Accordingly, in some embodiments, an epitope of an antigen may include a continuous or discontinuous portion of the antigen. In some embodiments, an epitope is or comprises a T cell epitope. In some embodiments, an epitope may have a length of about 5 to about 30 amino acids, or about 10 to about 25 amino acids, or about 5 to about 15 amino acids, or about 5 to 12 amino acids, or about 6 to about 9 amino acids.

Expression: As used herein, the term “expression” of a nucleic acid sequence refers to the generation of a gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Five prime untranslated region: As used herein, the terms “five prime untranslated region” or “5′ UTR” refer to a sequence of an mRNA molecule between a transcription start site and a start codon of a coding region of an RNA. In some embodiments, “5′ UTR” refers to a sequence of an mRNA molecule that begins at a transcription start site and ends one nucleotide (nt) before a start codon (usually AUG) of a coding region of an RNA molecule, e.g., in its natural context.

Humoral immunity: As used herein, the term “humoral immunity” or “humoral immune response” refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Increased, Induced, or Reduced: As used herein, these terms or grammatically comparable comparative terms, indicate values that are relative to a comparable reference measurement. For example, in some embodiments, an assessed value achieved with a provided pharmaceutical composition (e.g., immunogenic composition, e.g., vaccine) may be “increased” relative to that obtained with a comparable reference pharmaceutical composition (e.g., immunogenic composition, e.g., vaccine). Alternatively or additionally, in some embodiments, an assessed value achieved in a subject may be “increased” relative to that obtained in the same subject under different conditions (e.g., prior to or after an event; or presence or absence of an event such as administration of a pharmaceutical composition (e.g., immunogenic composition, e.g., vaccine) as described herein, or in a different, comparable subject (e.g., in a comparable subject that differs from the subject of interest in prior exposure to a condition, e.g., absence of administration of a pharmaceutical composition (e.g., immunogenic composition, e.g., vaccine) as described herein). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance. In some embodiments, the term “reduced” or equivalent terms refers to a reduction in the level of an assessed value by at least 5%, at least 10%, at least 20%, at least 50%, at least 75% or higher, as compared to a comparable reference. In some embodiments, the term “reduced” or equivalent terms refers to a complete or essentially complete inhibition, i.e., a reduction to zero or essentially to zero. In some embodiments, the term “increased” or “induced” refers to an increase in the level of an assessed value by at least 10%, at least 20%, at least 30%, at least 40%, at least 50%, at least 80%, at least 100%, at least 200%, at least 500%, or higher, as compared to a comparable reference.

Ionizable: The term “ionizable” refers to a compound or group or atom that is charged at a certain pH. In the context of an ionizable amino lipid, such a lipid or a function group or atom thereof bears a positive charge at a certain pH. In some embodiments, an ionizable amino lipid is positively charged at an acidic pH. In some embodiments, an ionizable amino lipid is predominately neutral at physiological pH values, e.g., in some embodiments about 7.0-7.4, but becomes positively charged at lower pH values. In some embodiments, an ionizable amino lipid may have a pKa within a range of about 5 to about 7.

Isolated: The term “isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

RNA lipid nanoparticle: As used herein, the term “RNA lipid nanoparticle” refers to a nanoparticle comprising at least one lipid and RNA molecule(s). In some embodiments, an RNA lipid nanoparticle comprises at least one ionizable amino lipid. In some embodiments, an RNA lipid nanoparticle comprises at least one ionizable amino lipid, at least one helper lipid, and at least one polymer-conjugated lipid (e.g., PEG-conjugated lipid). In various embodiments, RNA lipid nanoparticles as described herein can have an average size (e.g., Z-average) of about 100 nm to 1000 nm, or about 200 nm to 900 nm, or about 200 nm to 800 nm, or about 250 nm to about 700 nm. In some embodiments of the present disclosure, RNA lipid nanoparticles can have a particle size (e.g., Z-average) of about 30 nm to about 200 nm, or about 30 nm to about 150 nm, about 40 nm to about 150 nm, about 50 nm to about 150 nm, about 60 nm to about 130 nm, about 70 nm to about 110 nm, about 70 nm to about 100 nm, about 80 nm to about 100 nm, about 90 nm to about 100 nm, about 70 to about 90 nm, about 80 nm to about 90 nm, or about 70 nm to about 80 nm. In some embodiments, an average size of lipid nanoparticles is determined by measuring the particle diameter. In some embodiments, RNA lipid nanoparticles may be prepared by mixing lipids with RNA molecules described herein.

Lipidoid: As used herein, a “lipidoid” refers to a lipid-like molecule. In some embodiments, a lipoid is an amphiphilic molecule with one or more lipid-like physical properties. In the context of the present disclosure, the term lipid is considered to encompass lipidoids.

Nanoparticle: As used herein, the term “nanoparticle” refers to a particle having an average size suitable for parenteral administration. In some embodiments, a nanoparticle has a longest dimension (e.g., a diameter) of less than 1,000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 μm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

Naturally occurring: The term “naturally occurring” as used herein refers to an entity that can be found in nature. For example, a peptide or nucleic acid that is present in an organism (including viruses) and can be isolated from a source in nature and which has not been intentionally modified by man in the laboratory is naturally occurring.

Neutralization: As used herein, the term “neutralization” refers to an event in which binding agents such as antibodies bind to a biological active site of a virus such as a receptor binding protein, thereby inhibiting the parasitic infection of cells. In some embodiments, the term “neutralization” refers to an event in which binding agents eliminate or significantly reduce ability of infecting cells.

Nucleic acid particle: A “nucleic acid particle” can be used to deliver nucleic acid to a target site of interest (e.g., cell, tissue, organ, and the like). A nucleic acid particle may comprise at least one cationic or cationically ionizable lipid or lipid-like material, at least one cationic polymer such as protamine, or a mixture thereof and nucleic acid. In some embodiments, a nucleic acid particle is a lipid nanoparticle. In some embodiments, a nucleic acid particle is a lipoplex particle.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes a HSV infection. In some embodiments, a patient is receiving or has received certain therapy to diagnose and/or to treat a disease, disorder, or condition. In some embodiments, a patient is a patient suffering from or susceptible to a HSV infection.

PEG-conjugated lipid: The term “PEG-conjugated lipid” refers to a molecule comprising a lipid portion and a polyethylene glycol portion.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for parenteral administration, for example, by subcutaneous, intramuscular, or intravenous injection as, for example, a sterile solution or suspension formulation.

Pharmaceutically effective amount: The term “pharmaceutically effective amount” or “therapeutically effective amount” refers to the amount which achieves a desired reaction or a desired effect alone or together with further doses. In the case of the treatment of a particular disease, a desired reaction in some embodiments relates to inhibition of the course of the disease. In some embodiments, such inhibition may comprise slowing down the progress of a disease and/or interrupting or reversing the progress of the disease. In some embodiments, a desired reaction in a treatment of a disease may be or comprise delay or prevention of the onset of a disease or a condition. An effective amount of pharmaceutical compositions (e.g., immunogenic compositions, e.g., vaccines) described herein will depend, for example, on a disease or condition to be treated, the severity of such a disease or condition, individual parameters of the patient, including, e.g., age, physiological condition, size and weight, the duration of treatment, the type of an accompanying therapy (if present), the specific route of administration and similar factors. Accordingly, doses of pharmaceutical compositions (e.g., immunogenic compositions, e.g., vaccines) described herein may depend on various of such parameters. In the case that a reaction in a patient is insufficient with an initial dose, higher doses (or effectively higher doses achieved by a different, more localized route of administration) may be used.

Poly(A) sequence: As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3 ‘-UTR in the RNAs described herein. An uninterrupted poly(A) sequence is characterized by consecutive adenylate residues. In nature, an uninterrupted poly(A) sequence is typical. RNAs disclosed herein can have a poly(A) sequence attached to the free 3’-end of the RNA by a template-independent RNA polymerase after transcription or a poly(A) sequence encoded by DNA and transcribed by a template-dependent RNA polymerase.

Polypeptide: As used herein, the term “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications comprise acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide.

Prevent: As used herein, the term “prevent” or “prevention” when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: The term “recombinant” in the context of the present disclosure means “made through genetic engineering”. In some embodiments, a “recombinant” entity such as a recombinant nucleic acid in the context of the present disclosure is not naturally occurring.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Risk: As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

RNA lipoplex particle: As used herein, the term “RNA lipoplex particle” refers to a complex comprising liposomes, in particular cationic liposomes, and RNA molecules. Without wishing to bound by a particular theory, electrostatic interactions between positively charged liposomes and negatively charged RNA results in complexation and spontaneous formation of RNA lipoplex particles. In some embodiments, positively charged liposomes may comprise a cationic lipid, such as in some embodiments DOTMA, and additional lipids, such as in some embodiments DOPE. In some embodiments, a RNA lipoplex particle is a nanoparticle.

Selective or specific: The term “selective” or “specific”, when used herein in reference to an agent having an activity, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Stable: As used herein, the term “stable” in the context of the present disclosure refers to a pharmaceutical composition (e.g., immunogenic composition, e.g., vaccine) as a whole and/or components thereof meeting or exceeding pre-determined acceptance criteria. For example, in some embodiments, a stable pharmaceutical composition (e.g., immunogenic composition, e.g., vaccine) exhibits no unacceptable levels of microbial growth, and substantially no or no breakdown or degradation of the active biological molecule component(s). In some embodiments, a stable pharmaceutical composition (e.g., immunogenic composition, e.g., vaccine) refers to the integrity of RNA molecules being maintained at least above 90% or more. In some embodiments, a stable pharmaceutical composition (e.g., immunogenic composition, e.g., vaccine) refers to at least 90% or more (including, e.g., at least 95%, at least 96%, at least 97%, or more) of RNA molecules being maintained to be encapsulated within lipid nanoparticles. In some embodiments, a stable pharmaceutical composition (e.g., immunogenic composition, e.g., vaccine) refers to a formulation that remains capable of eliciting a desired immunologic response when administered to a subject. In some embodiments, a pharmaceutical composition (e.g., immunogenic composition, e.g., vaccine) remains stable for a specified period of time under certain conditions.

Subject: As used herein, the term “subject” refers to an organism to be administered with a composition described herein, e.g., for experimental, diagnostic, prophylactic, and/or therapeutic purposes. Typical subjects include animals (e.g., mammals such as mice, rats, rabbits, non-human primates, domestic pets, etc.) and humans. In some embodiments, a subject is a human subject. In some embodiments, a subject is suffering from a disease, disorder, or condition (e.g., a HSV infection). In some embodiments, a subject is susceptible to a disease, disorder, or condition (e.g., a HSV infection). In some embodiments, a subject displays one or more symptoms or characteristics of a disease, disorder, or condition (e.g., a HSV infection). In some embodiments, a subject displays one or more non-specific symptoms of a disease, disorder, or condition (e.g., a HSV infection). In some embodiments, a subject does not display any symptom or characteristic of a disease, disorder, or condition (e.g., a HSV infection). In some embodiments, a subject is someone with one or more features characteristic of susceptibility to or risk of a disease, disorder, or condition (e.g., a HSV infection). In some embodiments, a subject is a patient. In some embodiments, a subject is an individual to whom diagnosis and/or therapy is and/or has been administered.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Therapy: The term “therapy” refers to an administration or delivery of an agent or intervention that has a therapeutic effect and/or elicits a desired biological and/or pharmacological effect (e.g., has been demonstrated to be statistically likely to have such effect when administered to a relevant population). In some embodiments, a therapeutic agent or therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapeutic agent or therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition.

Three prime untranslated region: As used herein, the terms “three prime untranslated region” or “3′ UTR” refer to a sequence of an RNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g. a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition.

Vaccination: As used herein, the term “vaccination” refers to the administration of a composition intended to generate an immune response, for example to a disease-associated (e.g., disease-causing) agent. In some embodiments, vaccination can be administered before, during, and/or after exposure to a disease-associated agent, and in certain embodiments, before, during, and/or shortly after exposure to the agent. In some embodiments, vaccination includes multiple administrations, appropriately spaced in time, of a vaccine composition. In some embodiments, vaccination generates an immune response to an infectious agent.

Vaccine: As used herein, the term “vaccine” refers to a composition that induces an immune response upon administration to a subject. In some embodiments, an induced immune response provides protective immunity.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In some embodiments, known techniques may be used, for example, for generation or manipulation of recombinant DNA, for oligonucleotide synthesis, and for tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (4th ed., Cold Spring Harbor Laboratory Press, Cold Spring Harbor, N.Y. (2012)), which is incorporated herein by reference for any purpose.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

### DETAILED DESCRIPTION

### Compositions

In some embodiments, the present disclosure provides compositions comprising one or more RNAs (also referred to herein as “polyribonucleotides.”) In some embodiments, one or more RNAs encode a Herpes Simplex Virus (HSV) glycoprotein or immunogenic fragment thereof. In some embodiments, an RNA is a modified RNA as described herein below.

In some embodiments, an immunogenic fragment of an HSV glycoprotein comprises the ectodomain of the glycoprotein or a portion thereof. In another embodiment, an immunogenic fragment consists of the ectodomain of the glycoprotein or a portion thereof.

In some embodiments, the present disclosure provides a composition comprising one or more nucleoside-modified RNAs, wherein each of said modified RNAs encodes a Herpes Simplex Virus (HSV) glycoprotein or immunogenic fragment thereof, and wherein said modified RNA comprises one or more pseudouridine or pseudouridine family residues.

In some embodiments, an HSV glycoprotein comprises glycoprotein D (gD), glycoprotein C (gC), glycoprotein E (gE), glycoprotein B (gB), glycoprotein H (gH), glycoprotein L (gL) glycoprotein I (gI), or a combination thereof.

Thus, in some embodiments, the present disclosure provides a composition comprising one or more modified RNAs encoding HSV gD, gC, gE, gB, gH, gL, gI, or immunogenic fragments thereof. In some embodiments, the modified RNAs comprise pseudouridine-modified RNAs.

In some embodiments, the present disclosure provides compositions comprising an RNA encoding HSV gD or an immunogenic fragment thereof. In another embodiment, the present disclosure provides compositions comprising an RNA encoding HSV gC or an immunogenic fragment thereof. In another embodiment, the present disclosure provides compositions comprising an RNA encoding HSV gE or an immunogenic fragment thereof. In another embodiment, the present disclosure provides compositions comprising an RNA encoding HSV gB or an immunogenic fragment thereof. In another embodiment, the present disclosure provides compositions comprising an RNA encoding HSV gH or an immunogenic fragment thereof. In another embodiment, the present disclosure provides compositions comprising an RNA encoding HSV gL or an immunogenic fragment thereof. In another embodiment, the present disclosure provides compositions comprising an RNA encoding HSV gI or an immunogenic fragment thereof.

In some embodiments, the present disclosure provides a composition comprising: (a) an RNA encoding HSV gD or an immunogenic fragment thereof; and (b) an RNA encoding HSV gC or an immunogenic fragment thereof.

In another embodiment, the present disclosure provides a composition comprising: (a) an RNA encoding HSV gD or an immunogenic fragment thereof; and (b) an RNA encoding HSV gE or an immunogenic fragment thereof.

In another embodiment, the present disclosure provides a composition comprising: (a) an RNA encoding HSV gC or an immunogenic fragment thereof; and (b) an RNA encoding HSV gE or an immunogenic fragment thereof.

In another embodiment, the present disclosure provides a composition comprising: (a) an RNA encoding HSV gD or an immunogenic fragment thereof; (b) an RNA encoding HSV gC or an immunogenic fragment thereof, and (c) an RNA encoding HSV gE or an immunogenic fragment thereof.

In another embodiment, the present disclosure provides a composition comprising: (a) an RNA encoding HSV gD or an immunogenic fragment thereof; (b) an RNA encoding HSV gC or an immunogenic fragment thereof, (c) an RNA encoding HSV gE or an immunogenic fragment thereof; and (d) an RNA encoding HSV gB or an immunogenic fragment thereof.

In some embodiments, the HSV glycoproteins are HSV-2 glycoproteins or immunogenic fragments thereof. In another embodiment, the HSV glycoproteins are HSV-1 glycoproteins or immunogenic fragments thereof. In some embodiments, the HSV glycoproteins comprise both HSV-2 glycoproteins or immunogenic fragments thereof and HSV-1 glycoproteins or immunogenic fragments thereof. In another embodiment, the HSV glycoproteins comprise a mixture of HSV-2 glycoproteins, or immunogenic fragments thereof, and HSV-1 glycoproteins or immunogenic fragments thereof.

In some embodiments, the present disclosure provides compositions comprising an RNA encoding HSV-2 gD or an immunogenic fragment thereof. In another embodiment, the present disclosure provides compositions comprising an RNA encoding HSV-2 gC or an immunogenic fragment thereof. In another embodiment, the present disclosure provides compositions comprising an RNA encoding HSV-2 gE or an immunogenic fragment thereof. In another embodiment, the present disclosure provides compositions comprising an RNA encoding HSV-2 gE or an immunogenic fragment thereof. In another embodiment, the present disclosure provides compositions comprising an RNA encoding HSV-2 gB or an immunogenic fragment thereof. In another embodiment, the present disclosure provides compositions comprising an RNA encoding HSV-2 gH or an immunogenic fragment thereof. In another embodiment, the present disclosure provides compositions comprising an RNA encoding HSV-2 gL or an immunogenic fragment thereof. In another embodiment, the present disclosure provides compositions comprising an RNA encoding HSV-2 gI or an immunogenic fragment thereof.

In some embodiments, the present disclosure provides a composition comprising: (a) an RNA encoding HSV-2 gD or an immunogenic fragment thereof; and (b) an RNA encoding HSV-2 gC or an immunogenic fragment thereof.

In another embodiment, the present disclosure provides a composition comprising: (a) an RNA encoding HSV-2 gD or an immunogenic fragment thereof; and (b) an RNA encoding HSV-2 gE or an immunogenic fragment thereof.

In another embodiment, the present disclosure provides a composition comprising: (a) an RNA encoding HSV-2 gC or an immunogenic fragment thereof; and (b) an RNA encoding HSV-2 gE or an immunogenic fragment thereof.

In another embodiment, the present disclosure provides a composition comprising: (a) an RNA encoding HSV-2 gD or an immunogenic fragment thereof; (b) an RNA encoding HSV-2 gC or an immunogenic fragment thereof, and (c) an RNA encoding HSV-2 gE or an immunogenic fragment thereof.

In some embodiments, the present disclosure provides compositions comprising an RNA encoding HSV-1 gD or an immunogenic fragment thereof. In another embodiment, the present disclosure provides compositions comprising an RNA encoding HSV-1 gC or an immunogenic fragment thereof. In another embodiment, the present disclosure provides compositions comprising an RNA encoding HSV-1 gE or an immunogenic fragment thereof. In another embodiment, the present disclosure provides compositions comprising an RNA encoding HSV-1 gE or an immunogenic fragment thereof. In another embodiment, the present disclosure provides compositions comprising an RNA encoding HSV-1 gB or an immunogenic fragment thereof. In another embodiment, the present disclosure provides compositions comprising an RNA encoding HSV-1 gH or an immunogenic fragment thereof. In another embodiment, the present disclosure provides compositions comprising an RNA encoding HSV-1 gL or an immunogenic fragment thereof. In another embodiment, the present disclosure provides compositions comprising an RNA encoding HSV-1 gI or an immunogenic fragment thereof.

In some embodiments, the present disclosure provides a composition comprising: (a) an RNA encoding HSV-1 gD or fragment thereof; and (b) an RNA encoding HSV-1 gC or fragment thereof.

In another embodiment, the present disclosure provides a composition comprising: (a) an RNA encoding HSV-1 gD or an immunogenic fragment thereof; and (b) an RNA encoding HSV-1 gE or an immunogenic fragment thereof.

In another embodiment, the present disclosure provides a composition comprising: (a) an RNA encoding HSV-1 gC or an immunogenic fragment thereof; and (b) an RNA encoding HSV-1 gE or an immunogenic fragment thereof.

In another embodiment, the present disclosure provides a composition comprising: (a) an RNA encoding HSV-1 gD or an immunogenic fragment thereof; (b) an RNA encoding HSV-1 gC or an immunogenic fragment thereof, and (c) an RNA encoding HSV-1 gE or an immunogenic fragment thereof.

In some embodiments, any of the compositions as described herein consists essentially of one or more RNAs, wherein each of said RNAs encodes an HSV glycoprotein or immunogenic fragment thereof. In another embodiment, any of the compositions as described herein consists of one or more RNAs, wherein each of said RNAs encodes an HSV glycoprotein or immunogenic fragment thereof.

In another embodiment, the present disclosure provides compositions comprising an RNA encoding an HSV gD protein or an immunogenic fragment thereof, an RNA encoding an HSV gC protein or an immunogenic fragment thereof, an RNA encoding an HSV gE protein or an immunogenic fragment thereof and RNAs encoding one or more additional HSV glycoproteins or an immunogenic fragment thereof. In some embodiments, said additional HSV glycoproteins comprise gB or an immunogenic fragment thereof, gH or an immunogenic fragment thereof, gL or an immunogenic fragment thereof, gI or an immunogenic fragment thereof, or any combination thereof. In some embodiments, said additional HSV glycoproteins comprise glycoprotein M (gM), glycoprotein N (gN), glycoprotein K (gK), glycoprotein G (gG), glycoprotein J (gJ), or an immunogenic fragment(s) thereof.

In some embodiments, compositions of the present disclosure and for use in the methods of the present disclosure comprise both HSV-2 glycoproteins or immunogenic glycoprotein fragments and HSV-1 glycoproteins or immunogenic glycoprotein fragments. In another embodiment, compositions of the present disclosure and for use in the methods of the present disclosure comprise a mixture of HSV-2 glycoproteins or immunogenic glycoprotein fragments and HSV-1 glycoproteins or immunogenic glycoprotein fragments. For example, in some embodiments, a composition of the present disclosure comprises HSV-2 gC, HSV-1 gD, and HSV-2 gE, or immunogenic fragments thereof. In another embodiment, a composition of the present disclosure comprises HSV-1 gC, HSV-2 gD, and HSV-2 gE, or immunogenic fragments thereof. In another embodiment, a composition of the present disclosure comprises HSV-2 gC, HSV-2 gD, and HSV-1 gE, or immunogenic fragments thereof. In another embodiment, a composition of the present disclosure comprises HSV-1 gC, HSV-1 gD, and HSV-2 gE, or immunogenic fragments thereof. In another embodiment, a composition of the present disclosure comprises HSV-1 gC, HSV-2 gD, and HSV-1 gE, or immunogenic fragments thereof. In another embodiment, a composition of the present disclosure comprises HSV-2 gC, HSV-1 gD, and HSV-1 gE, or immunogenic fragments thereof.

In another embodiment, the compositions of the present disclosure comprise one or more additional HSV-1 glycoproteins or immunogenic fragments thereof, or HSV-2 glycoproteins or both HSV-1 and HSV-2 glycoproteins, as described herein. For example, in some embodiments, a composition of the present disclosure comprising HSV-2 gC or an immunogenic fragment thereof, HSV-1 gD or an immunogenic fragment thereof and HSV-2 gE or an immunogenic fragment thereof may further comprise HSV-1 gI or an immunogenic fragment thereof. In another embodiment, a composition of the present disclosure comprising HSV-2 gC or an immunogenic fragment thereof, HSV-2 gD or an immunogenic fragment thereof, and HSV-2 gE or an immunogenic fragment thereof may further comprise HSV-1 gB or an immunogenic fragment thereof. Each of the possible combinations of HSV-1 and HSV-2 glycoproteins, or immunogenic fragments thereof, represents a separate embodiment of the disclosure.

In some embodiments, the present disclosure provides an RNA construct comprising one or more coding sequences, a 5′UTR, a 3′UTR, a polyA tail, a cap, or a combination thereof. In some embodiments, the 5′UTR is from tobacco etch virus. In some embodiments, the 3′UTR is from Xenopus beta globin.

As used herein, “encoding” refers to an RNA molecule that contains a gene that encodes a protein of interest, or a fragment thereof. In another embodiment, an RNA molecule comprises a protein coding sequence that encodes a protein of interest, or a fragment thereof. In another embodiment, one or more other proteins, or a fragments thereof is also encoded. In another embodiment, the protein of interest, or a fragment thereof, is the only protein encoded. Each possibility represents a separate embodiment of the present disclosure.

“Immunogenic fragment” refers, in another embodiment, to a portion of a protein that is immunogenic and elicits a protective immune response when administered to a subject.

In some embodiments, “immunogenicity” or “immunogenic” is used herein to refer to the innate ability of a protein, peptide, protein fragment, nucleic acid, antigen or organism to elicit an immune response in an animal when the protein, peptide, protein fragment, nucleic acid, antigen or organism is administered to the animal. Thus, “enhancing the immunogenicity” in some embodiments, refers to increasing the ability of a protein, peptide, nucleic acid, antigen or organism to elicit an immune response in an animal when the protein, peptide, protein fragment, nucleic acid, antigen or organism is administered to an animal. The increased ability of a protein, peptide, protein fragment, nucleic acid, antigen or organism to elicit an immune response can be measured by, in some embodiments, a greater number of antibodies to a protein, peptide, protein fragment, nucleic acid, antigen or organism, a greater diversity of antibodies to an antigen or organism, a greater number of T-cells specific for a protein, peptide, protein fragment, nucleic acid, antigen or organism, a greater cytotoxic or helper T-cell response to a protein, peptide, nucleic acid, antigen or organism, and the like.

In some embodiments, a protein, peptide, protein fragment, nucleic acid or organism can be antigenic. “Antigenic” refers, in another embodiment, to a protein, peptide, protein fragment, nucleic acid, or organism capable of specifically interacting with an antigen recognition molecule of the immune system, e.g., an immunoglobulin (antibody) or T cell antigen receptor. An antigenic protein, peptide, or protein fragment contains, in another embodiment, an epitope of at least about 8 amino acids (AAs). An antigenic portion of a a protein, peptide, protein fragment, nucleic acid, or organism, also called herein an epitope, can be a portion that is immunodominant for antibody or T cell receptor recognition, or it can be a portion used to generate an antibody to the molecule by conjugating an antigenic portion to a carrier polypeptide for immunization. A molecule that is antigenic need not itself be immunogenic, i.e., capable of eliciting an immune response without a carrier.

In some embodiments, “functional” is used herein to refer to the innate ability of a protein, peptide, nucleic acid, fragment or a variant thereof to exhibit a biological activity or function. In some embodiments, such a biological function is its binding property to an interaction partner, e.g., a membrane-associated receptor, and in another embodiment, its trimerization property. In the case of functional fragments and the functional variants of the disclosure, these biological functions may in fact be changed, e.g., with respect to their specificity or selectivity, but with retention of the basic biological function.

In some embodiments, the term “fragment” is used herein to refer to a protein or polypeptide that is shorter or comprises fewer amino acids than the full-length protein or polypeptide. In another embodiment, fragment refers to a nucleic acid encoding the protein fragment that is shorter or comprises fewer nucleotides than the full-length nucleic acid. In another embodiment, the fragment is an N-terminal fragment. In another embodiment, the fragment is a C-terminal fragment. In some embodiments, the fragment is an intrasequential section of the protein, peptide, or nucleic acid. In another embodiment, the fragment is an immunogenic intrasequential section of the protein, peptide or nucleic acid. In another embodiment, the fragment is a functional intrasequential section within the protein, peptide or nucleic acid. In another embodiment, the fragment is an N-terminal immunogenic fragment. In some embodiments, the fragment is a C-terminal immunogenic fragment. In another embodiment, the fragment is an N-terminal functional fragment. In another embodiment, the fragment is a C-terminal functional fragment. In another embodiment, the fragment contains pieces of the protein linked together or pieces of multiple proteins linked together. In some embodiments, the fragment of the HSV protein is the ectodomain of the protein. In another embodiment, the fragment is 1, 2, 3, 4, 5, 6, 7, 8, 9, or 10 amino acids shorter than the full-length protein. In another embodiment, the fragment is 50-100, 100-150, 150-300, or 300-600 amino acids shorter than the full-length protein. In another embodiment, the fragment comprises 60%, 65%, 70%, 75%, 80%, 85%, 90%, 95% of the full-length protein. In another embodiment, the fragment comprises approximately 42%, 83%, 78%, or 66% of the full-length protein (excluding the signal sequence), as is described herein, in some embodiments, for HSV-2 gE, gC, gD, and gI. In some embodiments, a fragment is a domain (e.g., an ectodomain).

Thus, in some embodiments, an “immunogenic fragment” of a protein as described in the present disclosure refers to a portion of the protein that is immunogenic, in some embodiments and in another embodiment, elicits a protective immune response when administered to a subject.

In another aspect, the present disclosure provides compositions comprising RNAs, wherein each of said RNAs encodes a) HSV glycoprotein D (gD) or an immunogenic fragment thereof, b) HSV glycoprotein C (gC) or an immunogenic fragment thereof, c) HSV glycoprotein E (gE) or an immunogenic fragment thereof, or any combination thereof.

In some embodiments, the present disclosure provides a composition comprising an RNA encoding an HSV gD or an immunogenic fragment thereof, an RNA encoding an HSV gC or an immunogenic fragment thereof, and an RNA encoding an HSV gE or an immunogenic fragment thereof.

In another embodiment, inclusion of an RNA encoding gC or an immunogenic fragment thereof, and/or an RNA encoding gE or an immunogenic fragment thereof, in the composition of the present disclosure increases the efficaciousness of anti-gD antibodies elicited by the composition.

In another embodiment, inclusion of an RNA encoding gC or an immunogenic fragment thereof, and/or an RNA encoding gE or an immunogenic fragment thereof in the composition of the present disclosure enhances the effectiveness of an innate immune response. In another embodiment, the innate immune response is an antibody-mediated immune response. In another embodiment, the innate immune response is a non-antibody-mediated immune response. In another embodiment, the innate immune response is an NK (natural killer) cell response. In another embodiment, the innate immune response is any other innate immune response known in the art.

In another embodiment, inclusion of an RNA encoding gC or an immunogenic fragment thereof, and/or an RNA encoding gE or an immunogenic fragment thereof, in the composition of the present disclosure increases the efficaciousness of antibodies elicited by the composition against one of the glycoproteins described herein. In another embodiment, inclusion of an RNA encoding gC or an immunogenic fragment thereof, and/or an RNA encoding gE or an immunogenic fragment thereof, in the composition of the present disclosure decreases the dose of one of the above glycoproteins required to elicit antibodies that inhibit binding of the glycoprotein to a cellular receptor thereof, when a dose of one of the glycoproteins is administered separately from one of the other glycoproteins.

In some embodiments, a composition comprises one or more RNAs encoding HSV glycoproteins or immunogenic fragments thereof and lipid nanoparticles, polyplexes (PLX), lipidated polyplexes (LPLX), or liposomes.

### Glycoprotein E

In some embodiments, the present disclosure provides an RNA encoding HSV glycoprotein E (gE) or an immunogenic fragment thereof. In another embodiment, the present disclosure provides a composition comprising an RNA encoding HSV gE or an immunogenic fragment thereof.

### HSV-1 gE

In another embodiment, an RNA encoding HSV gE as described herein comprises RNA encoding HSV-1 gE. In another embodiment, an RNA encoding HSV gE as described herein comprises RNA encoding a fragment of an HSV-1 gE protein (e.g., an immunogenic fragment).

In some embodiments, a nucleotide sequence of the RNA encoding an HSV-1 gE fragment comprises:

(SEQ ID NO: 62)
GGAAUAAAAGUCUCAACACAACAUAUACAAAACAAACGAAUCUCAA
GCAAUCAAGCAUUCUACUUCUAUUGCAGCAAUUUAAAUCAUUUCUU
UUAAAGCAAAAGCAAUUUUCUGAAAAUUUUCACCAUUUACGAACGA
UAGC
AUGCGCAUGCAGCUGCUGCUGCUGAUCGCCC
UGUCCCUGGCCCUGGUGACCAACUCC
AAGACCUCCUGGCGCCGCGUGUCCGUGGGCGAGGACGUGUCCCUGC
UGCCCGCCCCCGGCCCCACCGGCCGCGGCCCCACCCAGAAGCUGCU
GUGGGCCGUGGAGCCCCUGGACGGCUGCGGCCCCCUGCACCCCUCC
UGGGUGUCCCUGAUGCCCCCCAAGCAGGUGCCCGAGACCGUGGUGG
ACGCCGCCUGCAUGCGCGCCCCCGUGCCCCUGGCCAUGGCCUACGC
CCCCCCCGCCCCCUCCGCCACCGGCGGCCUGCGCACCGACUUCGUG
UGGCAGGAGCGCGCCGCCGUGGUGAACCGCUCCCUGGUGAUCUACG
GCGUGCGCGAGACCGACUCCGGCCUGUACACCCUGUCCGUGGGCGA
CAUCAAGGACCCCGCCCGCCAGGUGGCCUCCGUGGUGCUGGUGGUG
CAGCCCGCCCCCGUGCCCACCCCCCCCCCCACCCCCGCCGACUACG
ACGAGGACGACAACGACGAGGGCGAGGGCGAGGACGAGUCCCUGGC
CGGCACCCCCGCCUCCGGCACCCCCCGCCUGCCCCCCUCCCCCGCC
CCCCCCCGCUCCUGGCCCUCCGCCCCCGAGGUGUCCCACGUGCGCG
GCGUGACCGUGCGCAUGGAGACCCCCGAGGCCAUCCUGUUCUCCCC
CGGCGAGGCCUUCUCCACCAACGUGUCCAUCCACGCCAUCGCCCAC
GACGACCAGACCUACACCAUGGACGUGGUGUGGCUGCGCUUCGACG
UGCCCACCUCCUGCGCCGAGAUGCGCAUCUACGAGUCCUGCCUGUA
CCACCCCCAGCUGCCCGAGUGCCUGUCCCCCGCCGACGCCCCCUGC
GCCGCCUCCACCUGGACCUCCCGCCUGGCCGUGCGCUCCUACGCCG
GCUGCUCCCGCACCAACCCCCCCCCCCGCUGCUCCGCCGAGGCCCA
CAUGGAGCCCUUCCCCGGCCUGGCCUGGCAGGCCGCCUCCGUGAAC
CUGGAGUUCCGCGACGCCUCCCCCCAGCACUCCGGCCUGUACCUGU
GCGUGGUGUACGUGAACGACCACAUCCACGCCUGGGGCCACAUCAC
CAUCAACACCGCCGCCCAGUACCGCAACGCCGUGGUGGAGCAGCCC
CUGCCCCAGCGCGGCGCCGACCUGGCCGAGCCCACCCACCCCCACG
UGGGCGCCUAACUAGUAGUGACUGACUAGGAUCUGGUUACCACUAA
ACCAGCCUCAAGAACACCCGAAUGGAGUCUCUAAGCUACAUAAUAC
CAACUUACACUUACAAAAUGUUGUCCCCCAAAAUGUAGCCAUUCGU
AUCUGCUCCUAAUAAAAAGAAAGUUUCUUCACAUUCUAAAAAAAAA
AAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAA
C.

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 150). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-1 gE fragment lacks the 5′ untranslated sequences, the signal sequence, the 3′ untranslated sequences, the poly adenylation tail, or a combination thereof. In some embodiments, the sequence of the HSV-1 gE fragment is as set forth in SEQ ID NO: 22.

In some embodiments, an HSV-1 gE fragment encoded by RNA utilized in the methods and compositions of the present disclosure comprises amino acids 24-409 of gE from HSV-1 (e.g., NS strain), as set forth in the following amino acid sequence:

(SEQ ID NO: 1)
KTSWRRVSVGEDVSLLPAPGPTGRGPTQKLLWAVEPLDGCGPLHPSWVSL
MPPKQVPETVVDAACMRAPVPLAMAYAPPAPSATGGLRTDFVWQERAAVV
NRSLVIYGVRETDSGLYTLSVGDIKDPARQVASVVLVVQPAPVPTPPPTP
ADYDEDDNDEGEGEDESLAGTPASGTPRLPPSPAPPRSWPSAPEVSHVRG
VTVRMETPEAILFSPGEAFSTNVSIHAIAHDDQTYTMDVVWLRFDVPTSC
AEMRIYESCLYHPQLPECLSPADAPCAASTWTSRLAVRSYAGCSRTNPPP
RCSAEAHMEPFPGLAWQAASVNLEFRDASPQHSGLYLCVVYVNDHIHAWG
HITINTAAQYRNAVVEQPLPQRGADLAEPTHPHVGA.

In some embodiments, an HSV-1 gE fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 1. In some embodiments, an HSV-1 gE fragment has an amino acid sequence that is identical to the amino acid sequence SEQ ID NO: 1.

In some embodiments, the gE fragment encoded by RNA utilized in the methods and compositions of the present disclosure comprises amino acids 24-409 of gE from an HSV-1 strain (e.g., SEQ ID NO: 1).

In some embodiments, the HSV-1 gE fragment encoded by RNA utilized in the methods and compositions of the present disclosure comprises amino acids 21-409 of gE from HSV-1 (e.g., NS strain or US8), as set forth in the following amino acid sequence:

(SEQ ID NO: 2)
GTPKTSWRRVSVGEDVSLLPAPGPTGRGPTQKLLWAVEPLDGCGPLHPSW
VSLMPPKQVPETVVDAACMRAPVPLAMAYAPPAPSATGGLRTDFVWQERA
AVVNRSLVIYGVRETDSGLYTLSVGDIKDPARQVASVVLVVQPAPVPTPP
PTPADYDEDDNDEGEGEDESLAGTPASGTPRLPPSPAPPRSWPSAPEVSH
VRGVTVRMETPEAILFSPGEAFSTNVSIHAIAHDDQTYTMDVVWLRFDVP
TSCAEMRIYESCLYHPQLPECLSPADAPCAASTWTSRLAVRSYAGCSRTN
PPPRCSAEAHMEPFPGLAWQAASVNLEFRDASPQHSGLYLCVVYVNDHIH
AWGHITINTAAQYRNAVVEQPLPQRGADLAEPTHPHVGA.

In some embodiments, an HSV-1 gE fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 2. In some embodiments, an HSV-1 gE fragment has an amino acid sequence that is identical to the amino acid sequence SEQ ID NO: 2.

In some embodiments, the gE fragment encoded by RNA utilized in the methods and compositions of the present disclosure comprises amino acids 21-409 of gE from an HSV-1 strain (e.g., SEQ ID NO: 2).

In some embodiments, full-length HSV-1 gE encoded by RNA utilized in the methods and compositions of the present disclosure comprises the following amino acid sequence:

(SEQ ID NO: 3)
MDRGAVVGFLLGVCVVSCLAGTPKTSWRRVSVGEDVSLLPAPGPTGRGPT
QKLLWAVEPLDGCGPLHPSWVSLMPPKQVPETVVDAACMRAPVPLAMAYA
PPAPSATGGLRTDFVWQERAAVVNRSLVIYGVRETDSGLYTLSVGDIKDP
ARQVASVVLVVQPAPVPTPPPTPADYDEDDNDEGEGEDESLAGTPASGTP
RLPPSPAPPRSWPSAPEVSHVRGVTVRMETPEAILFSPGEAFSTNVSIHA
IAHDDQTYTMDVVWLRFDVPTSCAEMRIYESCLYHPQLPECLSPADAPCA
ASTWTSRLAVRSYAGCSRTNPPPRCSAEAHMEPFPGLAWQAASVNLEFRD
ASPQHSGLYLCVVYVNDHIHAWGHITINTAAQYRNAVVEQPLPQRGADLA
EPTHPHVGAPPHAPPTHGALRLGAVMGAALLLSALGLSVWACMTCWRRRA
WRAVKSRASGKGPTYIRVADSELYADWSSDSEGERDQVPWLAPPERPDSP
STNGSGFEILSPTAPSVYPRSDGHQSRRQLTTFGSGRPDRRYSQASDSSV
FW.

In some embodiments, an HSV-1 gE comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 3. In some embodiments, an HSV-1 gE has an amino acid sequence that is identical to the amino acid sequence SEQ ID NO: 3.

In another embodiment, the HSV-1 gE or an immunogenic fragment thereof, encoded by RNA utilized in the methods and compositions of the present disclosure comprises the amino acid sequences as set forth in any of the following GenBank Accession Numbers: AAA45779.1, AAA96680.1, ABI63526.1, ACM62297.1, ADD60055.1, ADD60132.1, ADM22391.1, ADM22468.1, ADM22544.1, ADM22621.1, ADM22698.1, ADM22775.1, ADM22851.1, ADM22928.1, ADM23005.1, ADM23081.1, ADM23157.1, ADM23233.1, ADM23311.1, ADM23385.1, ADM23459.1, ADM23533.1, ADM23607.1, ADM23682.1, ADM23757.1, ADM23833.1, ADN34689.1, ADN34692.1, ADN34695.1, AEQ77099.1, AER37649.1, AER37717.1, AER37788.1, AER37859.1, AER37931.1, AER38002.1, AER38072.1, AFA36179.1, AFA36180.1, AFA36181.1, AFA36182.1, AFA36183.1, AFA36184.1, AFA36185.1, AFA36186.1, AFA36187.1, AFA36188.1, AFA36189.1, AFA36190.1, AFA36191.1, AFA36192.1, AFA36193.1, AFA36194.1, AFA36195.1, AFA36196.1, AFA36197.1, AFA36198.1, AFA36199.1, AFA36200.1, AFA36201.1, AFA36202.1, AFA36203.1, AFE62896.1, AFI23659.1, AFK50417.1, AFP86432.1, AGZ01930.1, AIR95859.1, AJE60011.1, AJE60082.1, AJE60153.1, AJE60224.1, AJE60295.1, AKE48647.1, AKE98373.1, AKE98374.1, AKE98375.1, AKE98376.1, AKE98377.1, AKE98378.1, AKE98379.1, AKE98380.1, AKE98381.1, AKE98382.1, AKE98383.1, AKE98384.1, AKE98385.1, AKE98386.1, AKE98387.1, AKE98388.1, AKE98389.1, AKE98390.1, AKE98391.1, AKE98392.1, AKE98393.1, AKG59248.1, AKG59320.1, AKG59393.1, AKG59464.1, AKG59538.1, AKG59611.1, AKG59684.1, AKG59757.1, AKG59828.1, AKG59900.1, AKG59974.1, AKG60048.1, AKG60120.1, AKG60191.1, AKG60263.1, AKG60336.1, AKG60406.1, AKG60476.1, AKG60548.1, AKG60622.1, AKG60694.1, AKG60765.1, AKG60837.1, AKG60908.1, AKG60980.1, AKG61052.1, AKG61125.1, AKG61196.1, AKG61269.1, AKG61341.1, AKG61413.1, AKG61486.1, AKG61558.1, AKG61631.1, AKG61705.1, AKG61776.1, AKG61849.1, AKG61922.1, AKG61995.1, AKH80465.1, AKH80538.1, ALM22637.1, ALM22711.1, ALM22785.1, ALM22859.1, ALO18664.1, ALO18740.1, AMB65664.1, AMB65737.1, AMB65811.1, AMB65887.1, AMB65958.1, AMN09834.1, ANN83966.1, ANN84043.1, ANN84119.1, ANN84196.1, ANN84273.1, ANN84350.1, ANN84426.1, ANN84502.1, ANN84579.1, ANN84655.1, ANN84732.1, ANN84808.1, ANN84885.1, ANN84961.1, ANN85038.1, ANN85114.1, ANN85189.1, ANN85266.1, ANN85343.1, ANN85418.1, ANN85496.1, ANN85573.1, ANN85650.1, ANN85726.1, ANN85803.1, AOY34085.1, AOY36687.1, ARB08959.1, ARO38073.1, ARO38074.1, ARO38075.1, ARO38076.1, ARO38077.1, ARO38078.1, ARO38079.1, ARO38080.1, ASM47642.1, ASM47666.1, ASM47743.1, ASM47820.1, ASM47895.1, BAM73421.1, CAA26062.1, CAA32272.1, CAF24756.1, CAF24757.1, CAF24758.1, CAF24759.1, CAF24760.1, CAF24761.1, CAF24762.1, CAF24763.1, CAF24764.1, CAF24765.1, CAF24766.1, CAF24767.1, CAF24768.1, CAF24769.1, CAF24770.1, CAF24771.1, CAF24772.1, CAF24773.1, CAF24774.1, CAF24775.1, CAF24776.1, CAF24777.1, CAF24778.1, CAF24779.1, CAF24780.1, CAF24781.1, CAF24782.1, CAF24783.1, CAF24784.1, CAF24785.1, P04290.1, P04488.1, P28986.1, Q703F0.1, SB007910.1, SBS69571.1, SBS69576.1, SBS69595.1, SBS69636.1, SBS69693.1, SBS69701.1, SBS69722.1, SBS69732.1, SBS69813.1, SBT69397.1, or YP 009137143.1.

### HSV-2 gE

In another embodiment, an RNA encoding HSV gE as described herein comprises RNA encoding HSV-2 gE. In another embodiment, an RNA encoding HSV gE as described herein comprises RNA encoding a fragment of an HSV-2 gE protein (e.g., an immunogenic fragment).

In some embodiments, a nucleotide sequence of the RNA encoding an HSV-2 gE fragment comprises:

(SEQ ID NO: 63)
GGAAUAAAAGUCUCAACACAACAUAUACAAAACAAACGAAUCUCAAGCAA
UCAAGCAUUCUACUUCUAUUGCAGCAAUUUAAAUCAUUUCUUUUAAAGCA
AAAGCAAUUUUCUGAAAAUUUUCACCAUUUACGAACGAUAGC
AUGCGCAUGCAGCUGCUGCUGCUGAUCGCCCUGU
CCCUGGCCCUGGUGACCAACUCC
CGCACCUCCUGGAAGCGCGUGACCUCCGGCGAGGACGUGGUGCUGCUGCC
CGCCCCCGCCGGCCCCGAGGAGCGCACCCGCGCCCACAAGCUGCUGUGGG
CCGCCGAGCCCCUGGACGCCUGCGGCCCCCUGCGCCCCUCCUGGGUGGCC
CUGUGGCCCCCCCGCCGCGUGCUGGAGACCGUGGUGGACGCCGCCUGCAU
GCGCGCCCCCGAGCCCCUGGCCAUCGCCUACUCCCCCCCCUUCCCCGCCG
GCGACGAGGGCCUGUACUCCGAGCUGGCCUGGCGCGACCGCGUGGCCGUG
GUGAACGAGUCCCUGGUGAUCUACGGCGCCCUGGAGACCGACUCCGGCCU
GUACACCCUGUCCGUGGUGGGCCUGUCCGACGAGGCCCGCCAGGUGGCCU
CCGUGGUGCUGGUGGUGGAGCCCGCCCCCGUGCCCACCCCCACCCCCGAC
GACUACGACGAGGAGGACGACGCCGGCGUGUCCGAGCGCACCCCCGUGUC
CGUGCCCCCCCCCACCCCCCCCCGCCGCCCCCCCGUGGCCCCCCCCACCC
ACCCCCGCGUGAUCCCCGAGGUGUCCCACGUGCGCGGCGUGACCGUGCAC
AUGGAGACCCCCGAGGCCAUCCUGUUCGCCCCCGGCGAGACCUUCGGCAC
CAACGUGUCCAUCCACGCCAUCGCCCACGACGACGGCCCCUACGCCAUGG
ACGUGGUGUGGAUGCGCUUCGACGUGCCCUCCUCCUGCGCCGAGAUGCGC
AUCUACGAGGCCUGCCUGUACCACCCCCAGCUGCCCGAGUGCCUGUCCCC
CGCCGACGCCCCCUGCGCCGUGUCCUCCUGGGCCUACCGCCUGGCCGUGC
GCUCCUACGCCGGCUGCUCCCGCACCACCCCCCCCCCCCGCUGCUUCGCC
GAGGCCCGCAUGGAGCCCGUGCCCGGCCUGGCCUGGCUGGCCUCCACCGU
GAACCUGGAGUUCCAGCACGCCUCCCCCCAGCACGCCGGCCUGUACCUGU
GCGUGGUGUACGUGGACGACCACAUCCACGCCUGGGGCCACAUGACCAUC
UCCACCGCCGCCCAGUACCGCAACGCCGUGGUGGAGCAGCACCUGCCCCA
GCGCCAGCCCGAGCCCGUGGAGCCCACCCGCCCCCACGUGCGCGCCUAA
CUAGUAGUGACUGACUAGGAUCUGGUUACCACUAAACCAGCCUC
AAGAACACCCGAAUGGAGUCUCUAAGCUACAUAAUACCAACUUA
CACUUACAAAAUGUUGUCCCCCAAAAUGUAGCCAUUCGUAUCUG
CUCCUAAUAAAAAGAAAGUUUCUUCACAUUCUAAAAAAAAAAAA
AAAAAAAAAAAAAAAAAAAAAAAAAAAAAAAAAAAAAAAAAAAA
AAAAAAAAAAAAAAAAAAAAAAAAAAAAAAAAAAAAAAAAAAAA
AC.

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 150). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-2 gE fragment lacks the 5′ untranslated sequences, the signal sequence, the 3′ untranslated sequences, the poly adenylation tail, or a combination thereof. In some embodiments, the sequence of the HSV-2 gE fragment is as set forth in SEQ ID NO: 23.

In some embodiments, an HSV-2 gE fragment encoded by RNA utilized in the methods and compositions of the present disclosure comprises amino acids 24-405 of gE from HSV-2 (e.g., strain 2.12 or US8) as set forth in the following amino acid sequence:

(SEQ ID NO: 4)
RTSWKRVTSGEDVVLLPAPAGPEERTRAHKLLWAAEPLDACGPLRPSWVA
LWPPRRVLETVVDAACMRAPEPLAIAYSPPFPAGDEGLYSELAWRDRVAV
VNESLVIYGALETDSGLYTLSVVGLSDEARQVASVVLVVEPAPVPTPTPD
DYDEEDDAGVSERTPVSVPPPTPPRRPPVAPPTHPRVIPEVSHVRGVTVH
METPEAILFAPGETFGTNVSIHAIAHDDGPYAMDVVWMRFDVPSSCAEMR
IYEACLYHPQLPECLSPADAPCAVSSWAYRLAVRSYAGCSRTTPPPRCFA
EARMEPVPGLAWLASTVNLEFQHASPQHAGLYLCVVYVDDHIHAWGHMTI
STAAQYRNAVVEQHLPQRQPEPVEPTRPHVRA.

In some embodiments, an HSV-2 gE fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 4. In some embodiments, an HSV-2 gE fragment has an amino acid sequence that is identical to the amino acid sequence SEQ ID NO: 4.

In some embodiments, the full-length HSV-2 gE encoded by RNA utilized in the methods and compositions of the present disclosure comprises the following amino acid sequence:

(SEQ ID NO: 5)
MARGAGLVFFVGVWVVSCLAAAPRTSWKRVTSGEDVVLLPAPAERTRAHK
LLWAAEPLDACGPLRPSWVALWPPRRVLETVVDAACMRAPEPLAIAYSPP
FPAGDEGLYSELAWRDRVAVVNESLVIYGALETDSGLYTLSVVGLSDEAR
QVASVVLVVEPAPVPTPTPDDYDEEDDAGVTNARRSAFPPQPPPRRPPVA
PPTHPRVIPEVSHVRGVTVHMETLEAILFAPGETFGTNVSIHAIAHDDGP
YAMDVVWMRFDVPSSCADMRIYEACLYHPQLPECLSPADAPCAVSSWAYR
LAVRSYAGCSRTTPPPRCFAEARMEPVPGLAWLASTVNLEFQHASPQHAG
LYLCVVYVDDHIHAWGHMTISTAAQYRNAVVEQHLPQRQPEPVEPTRPHV
RAPHPAPSARGPLRLGAVLGAALLLAALGLSAWACMTCWRRRSWRAVKSR
ASATGPTYIRVADSELYADWSSDSEGERDGSLWQDPPERPDSPSTNGSGF
EILSPTAPSVYPHSEGRKSRRPLTTFGSGSPGRRHSQASYPSVLW.

In some embodiments, an HSV-2 gE comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 5. In some embodiments, an HSV-2 gE has an amino acid sequence that is identical to the amino acid sequence SEQ ID NO: 5.

In another embodiment, the HSV-2 gE or an immunogenic fragment thereof, encoded by RNA utilized in the methods and compositions of the present disclosure comprises the amino acid sequences as set forth in any of the following GenBank Accession Numbers: ABU45436.1, ABU45437.1, ABU45438.1, ABU45439.1, ABW83306.1, ABW83308.1, ABW83310.1, ABW83312.1, ABW83314.1, ABW83316.1, ABW83318.1, ABW83320.1, ABW83322.1, ABW83324.1, ABW83326.1, ABW83328.1, ABW83330.1, ABW83332.1, ABW83334.1, ABW83336.1, ABW83338.1, ABW83340.1, ABW83342.1, ABW83344.1, ABW83346.1, ABW83348.1, ABW83350.1, ABW83352.1, ABW83354.1, ABW83356.1, ABW83358.1, ABW83360.1, ABW83362.1, ABW83364.1, ABW83366.1, ABW83368.1, ABW83370.1, ABW83372.1, ABW83374.1, ABW83376.1, ABW83378.1, ABW83380.1, ABW83382.1, ABW83384.1, ABW83386.1, ABW83388.1, ABW83390.1, ABW83392.1, ABW83394.1, ABW83396.1, ABW83398.1, ABW83400.1, ABZ04069.1, AEV91407.1, AHG54732.1, AKC42830.1, AKC59307.1, AKC59378.1, AKC59449.1, AKC59520.1, AKC59591.1, AMB66104.1, AMB66173.1, AMB66246.1, AMB66465.1, AQZ55756.1, AQZ55827.1, AQZ55898.1, AQZ55969.2, AQZ56040.2, AQZ56111.2, AQZ56182.1, AQZ56253.2, AQZ56324.1, AQZ56395.1, AQZ56466.2, AQZ56537.1, AQZ56608.1, AQZ56679.1, AQZ56750.1, AQZ56821.2, AQZ56892.1, AQZ56963.2, AQZ57034.2, AQZ57105.1, AQZ57176.1, AQZ57247.2, AQZ57318.2, AQZ57389.2, AQZ57460.2, AQZ57531.2, AQZ57602.2, AQZ57673.1, AQZ57744.2, AQZ57815.1, AQZ57886.1, AQZ57957.2, AQZ58028.2, AQZ58099.1, AQZ58170.2, AQZ58241.2, AQZ58312.2, AQZ58383.2, AQZ58454.2, AQZ58525.2, AQZ58596.1, AQZ58667.1, AQZ58738.2, AQZ58809.2, AQZ58880.2, AQZ58951.2, AQZ59022.2, AQZ59093.1, AQZ59164.1, ARO38081.1, ARO38082.1, ARO38083.1, ARO38084.1, ARO38085.1, ARO38086.1, CAB06715.1, P89436.1, P89475.1, or YP 009137220.1.

In another embodiment, a gE fragment encoded by RNA utilized in the methods and compositions of the present disclosure comprises an IgG Fc-binding domain of the gE protein. In another embodiment, the gE domain encoded by RNA utilized in the methods and compositions of the present disclosure is any other gE domain known in the art to mediate binding to IgG Fc.

In another embodiment, a gE protein encoded by RNA utilized in the methods and compositions of the present disclosure comprises a gE domain involved in cell-to-cell spread.

In another embodiment, a gE fragment encoded by RNA fragment utilized in the methods and compositions of the present disclosure comprises an immune evasion domain. In another embodiment, a gE fragment encoded by RNA fragment utilized in the methods and compositions of the present disclosure comprises a portion of an immune evasion domain.

Each RNA encoding HSV-1 gE or HSV-2 gE protein or fragment thereof represents a separate embodiment of the present disclosure.

In another embodiment, a gE protein fragment encoded by RNA utilized in the methods and compositions of the present disclosure is an immunogenic fragment. In another embodiment, a gE immunoprotective antigen need not be the entire protein. The protective immune response generally involves, in another embodiment, an antibody response. In another embodiment, mutants, sequence conservative variants, and functional conservative variants of gE are useful in methods and compositions of the present disclosure, provided that all such variants retain the required immuno-protective effect. In another embodiment, the immunogenic fragment can comprise an immuno-protective gE antigen from any strain of HSV. In another embodiment, the immunogenic fragment can comprise sequence variants of HSV, as found in infected individuals.

In some embodiments, the present disclosure provides a nucleoside-modified RNA encoding the ectodomain of HSV gE which comprises a sequence that is at least 95% identical to any one of SEQ ID NOs: 22-31.

### Glycoprotein C

In some embodiments, the present disclosure provides an RNA encoding HSV glycoprotein C (gC) or an immunogenic fragment thereof. In another embodiment, the present disclosure provides a composition comprising an RNA encoding HSV gC or an immunogenic fragment thereof. In some embodiments, the ectodomain comprises a sequence that is at least 95% identical to SEQ ID NO: 4. In some embodiments, the nucleoside-modified RNA encoding the ectodomain of HSV gE comprises a sequence that is at least 95% identical to SEQ ID NO: 28. In some embodiments, the nucleoside-modified RNA comprises a sequence that is at least 95% identical to SEQ ID NO: 240.

### HSV-1 gC

In another embodiment, RNA encoding HSV gC as described herein comprises an RNA encoding HSV-1 gC or an immunogenic fragment thereof. In another embodiment, an RNA encoding HSV gC as described herein comprises an RNA encoding a fragment of an HSV-1 gC protein (e.g., an immunogenic fragment).

In some embodiments, a nucleotide sequence of the RNA encoding an HSV-1 gC fragment comprises:

(SEQ ID NO: 64)
GGAAUAAAAGUCUCAACACAACAUAUACAAAACAAACGAAUCUCAAGC
AAUCAAGCAUUCUACUUCUAUUGCAGCAAUUUAAAUCAUUUCUUUUAA
AGCAAAAGCAAUUUUCUGAAAAUUUUCACCAUUUACGAACGAUAGC
AUGGCCAUCUCCGGCGUGCCCGUGCUGGGCUU
CUUCAUCAUCGCCGUGCUGAUGUCCGCCCAGG
AGUCCUGGGCCGAGACCGCCUCCACCGGCCCCACCAUCACCGCCGG
CGCCGUGACCAACGCCUCCGAGGCCCCCACCUCCGGCUCCCCCGGCUC
CGCCGCCUCCCCCGAGGUGACCCCCACCUCCACCCCCAACCCCAACAA
CGUGACCCAGAACAAGACCACCCCCACCGAGCCCGCCUCCCCCCCCAC
CACCCCCAAGCCCACCUCCACCCCCAAGUCCCCCCCCACCUCCACCCC
CGACCCCAAGCCCAAGAACAACACCACCCCCGCCAAGUCCGGCCGCCC
CACCAAGCCCCCCGGCCCCGUGUGGUGCGACCGCCGCGACCCCCUGGC
CCGCUACGGCUCCCGCGUGCAGAUCCGCUGCCGCUUCCGCAACUCCAC
CCGCAUGGAGUUCCGCCUGCAGAUCUGGCGCUACUCCAUGGGCCCCUC
CCCCCCCAUCGCCCCCGCCCCCGACCUGGAGGAGGUGCUGACCAACAU
CACCGCCCCCCCCGGCGGCCUGCUGGUGUACGACUCCGCCCCCAACCU
GACCGACCCCCACGUGCUGUGGGCCGAGGGCGCCGGCCCCGGCGCCGA
CCCCCCCCUGUACUCCGUGACCGGCCCCCUGCCCACCCAGCGCCUGAU
CAUCGGCGAGGUGACCCCCGCCACCCAGGGCAUGUACUACCUGGCCUG
GGGCCGCAUGGACUCCCCCCACGAGUACGGCACCUGGGUGCGCGUGCG
CAUGUUCCGCCCCCCCUCCCUGACCCUGCAGCCCCACGCCGUGAUGGA
GGGCCAGCCCUUCAAGGCCACCUGCACCGCCGCCGCCUACUACCCCCG
CAACCCCGUGGAGUUCGACUGGUUCGAGGACGACCGCCAGGUGUUCAA
CCCCGGCCAGAUCGACACCCAGACCCACGAGCACCCCGACGGCUUCAC
CACCGUGUCCACCGUGACCUCCGAGGCCGUGGGCGGCCAGGUGCCCCC
CCGCACCUUCACCUGCCAGAUGACCUGGCACCGCGACUCCGUGACCUU
CUCCCGCCGCAACGCCACCGGCCUGGCCCUGGUGCUGCCCCGCCCCAC
CAUCACCAUGGAGUUCGGCGUGCGCCACGUGGUGUGCACCGCCGGCUG
CGUGCCCGAGGGCGUGACCUUCGCCUGGUUCCUGGGCGACGACCCCUC
CCCCGCCGCCAAGUCCGCCGUGACCGCCCAGGAGUCCUGCGACCACCC
CGGCCUGGCCACCGUGCGCUCCACCCUGCCCAUCUCCUACGACUACUC
CGAGUACAUCUGCCGCCUGACCGGCUACCCCGCCGGCAUCCCCGUGCU
GGAGCACCACUAA
CUAGUAGUGACUGACUAGGAUCUGGUUACCACUAA
ACCAGCCUCAAGAACACCCGAAUGGAGUCUCUAAG
CUACAUAAUACCAACUUACACUUACAAAAUGUUGU
CCCCCAAAAUGUAGCCAUUCGUAUCUGCUCCUAAU
AAAAAGAAAGUUUCUUCACAUUCUAAAAAAAAAAA
AAAAAAAAAAAAAAAAAAAAAAAAAAAAAAAAAAA
AAAAAAAAAAAAAAAAAAAAAAAAAAAAAAAAAAA
AAAAAAAAAAAAAAAAAAAAC

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 155). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-1 gC fragment lacks the 5′ untranslated sequences, the signal sequence, the 3′ untranslated sequences, the poly adenylation tail, or a combination thereof. In some embodiments, the sequence of the HSV-1 gC fragment is as set forth in SEQ ID NO: 32.

In some embodiments, an HSV-1 gC fragment encoded by RNA utilized in the methods and compositions of the present disclosure comprises amino acids 27-457 of gC from HSV-1 (e.g., KOS strain), as set forth in the following amino acid sequence:

(SEQ ID NO: 6)
ETASTGPTITAGAVTNASEAPTSGSPGSAASPEVTPTSTPNPNNVTQNKT
TPTEPASPPTTPKPTSTPKSPPTSTPDPKPKNNTTPAKSGRPTKPPGPVW
CDRRDPLARYGSRVQIRCRFRNSTRMEFRLQIWRYSMGPSPPIAPAPDLE
EVLTNITAPPGGLLVYDSAPNLTDPHVLWAEGAGPGADPPLYSVTGPLPT
QRLIIGEVTPATQGMYYLAWGRMDSPHEYGTWVRVRMFRPPSLTLQPHAV
MEGQPFKATCTAAAYYPRNPVEFDWFEDDRQVFNPGQIDTQTHEHPDGFT
TVSTVTSEAVGGQVPPRTFTCQMTWHRDSVTFSRRNATGLALVLPRPTIT
MEFGVRHVVCTAGCVPEGVTFAWFLGDDPSPAAKSAVTAQESCDHPGLAT
VRSTLPISYDYSEYICRLTGYPAGIPVLEHH.

In some embodiments, an HSV-1 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 6. In some embodiments, an HSV-1 gC fragment has an amino acid sequence that is identical to the amino acid sequence SEQ ID NO: 6.

In some embodiments, a gC fragment encoded by RNA utilized in the methods and compositions of the present disclosure comprises amino acids 27-457 of gC from an HSV-1 strain.

In some embodiments, an HSV-1 gC fragment encoded by RNA utilized in the methods and compositions of the present disclosure comprises amino acids 25-457 of gC from HSV-1 (e.g., KOS strain), as set forth in the following amino acid sequence:

(SEQ ID NO: 7)
GSETASTGPTITAGAVTNASEAPTSGSPGSAASPEVTPTSTPNPNNVTQN
KTTPTEPASPPTTPKPTSTPKSPPTSTPDPKPKNNTTPAKSGRPTKPPGP
VWCDRRDPLARYGSRVQIRCRFRNSTRMEFRLQIWRYSMGPSPPIAPAPD
LEEVLTNITAPPGGLLVYDSAPNLTDPHVLWAEGAGPGADPPLYSVTGPL
PTQRLIIGEVTPATQGMYYLAWGRMDSPHEYGTWVRVRMFRPPSLTLQPH
AVMEGQPFKATCTAAAYYPRNPVEFDWFEDDRQVFNPGQIDTQTHEHPDG
FTTVSTVTSEAVGGQVPPRTFTCQMTWHRDSVTFSRRNATGLALVLPRPT
ITMEFGVRHVVCTAGCVPEGVTFAWFLGDDPSPAAKSAVTAQESCDHPGL
ATVRSTLPISYDYSEYICRLTGYPAGIPVLEHH.

In some embodiments, an HSV-1 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 7. In some embodiments, an HSV-1 gC fragment has an amino acid sequence that is identical to the amino acid sequence SEQ ID NO: 7.

In some embodiments, full-length HSV-1 gC encoded by RNA utilized in the methods and compositions of the present disclosure comprises the following amino acid sequence:

(SEQ ID NO: 8)
MAPGRVGLAVVLWGLLWLGAGVAGGSETASTGPTITAGAVTNASEAPTSG
SPGSAASPEVTPTSTPNPNNVTQNKTTPTEPASPPTTPKPTSTPKSPPTS
TPDPKPKNNTTPAKSGRPTKPPGPVWCDRRDPLARYGSRVQIRCRFRNST
RMEFRLQIWRYSMGPSPPIAPAPDLEEVLTNITAPPGGLLVYDSAPNLTD
PHVLWAEGAGPGADPPLYSVTGPLPTQRLIIGEVTPATQGMYYLAWGRMD
GSPHEYTWVRVRMFRPPSLTLQPHAVMEGQPFKATCTAAAYYPRNPVEFD
WFEDDRQVFNPGQIDTQTHEHPDGFTTVSTVTSEAVGGQVPPRTFTCQMT
WHRDSVTFSRRNATGLALVLPRPTITMEFGVRHVVCTAGCVPEGVTFAWF
LGDDPSPAAKSAVTAQESCDHPGLATVRSTLPISYDYSEYICRLTGYPAG
IPVLEHHGSHQPPPRDPTERQVIEAIEWVGIGIGVLAAGVLVVTAIVYVV
RTSQSRQRHRR.

In some embodiments, an HSV-1 gC comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 8. In some embodiments, an HSV-1 gC has an amino acid sequence that is identical to the amino acid sequence SEQ ID NO: 8.

In another embodiment, the HSV-1 gC or an immunogenic fragment thereof, encoded by RNA utilized in the methods and compositions of the present disclosure comprises the amino acid sequences as set forth in any of the following GenBank Accession Numbers: AAA45779.1, AAA96680.1, ABI63505.1, ABM52973.1, ABM52976.1, ABM52977.1, ACM62267.1, ADD60042.1, ADD60119.1, ADM22367.1, ADM22444.1, ADM22520.1, ADM22597.1, ADM22674.1, ADM22751.1, ADM22827.1, ADM22904.1, ADM22981.1, ADM23057.1, ADM23133.1, ADM23210.1, ADM23287.1, ADM23361.1, ADM23435.1, ADM23509.1, ADM23583.1, ADM23658.1, ADM23733.1, ADM23809.1, AEQ77075.1, AEQ77099.1, AER37628.1, AER37697.1, AER37767.1, AER37838.1, AER37910.1, AER37981.1, AER38051.2, AFA36179.1, AFA36180.1, AFA36181.1, AFA36182.1, AFA36183.1, AFA36184.1, AFA36185.1, AFA36186.1, AFA36187.1, AFA36188.1, AFA36189.1, AFA36190.1, AFA36191.1, AFA36192.1, AFA36193.1, AFA36194.1, AFA36195.1, AFA36196.1, AFA36197.1, AFA36198.1, AFA36199.1, AFA36200.1, AFA36201.1, AFA36202.1, AFA36203.1, AFE62872.1, AFH78104.1, AFI23635.1, AFK50391.1, AFP86408.1, AGZ01906.1, AIR95840.1, AJE59989.1, AJE60060.1, AJE60131.1, AJE60202.1, AKE48623.1, AKE98415.1, AKE98416.1, AKE98417.1, AKE98418.1, AKE98419.1, AKE98420.1, AKE98421.1, AKE98422.1, AKE98423.1, AKE98424.1, AKE98425.1, AKE98426.1, AKE98427.1, AKE98428.1, AKE98429.1, AKE98430.1, AKE98431.1, AKE98432.1, AKE98433.1, AKE98434.1, AKE98435.1, AKG59227.1, AKG59299.1, AKG59372.1, AKG59444.1, AKG59516.1, AKG59591.1, AKG59663.1, AKG59736.1, AKG59807.1, AKG59879.1, AKG59953.1, AKG60027.1, AKG60099.1, AKG60170.1, AKG60243.1, AKG60316.1, AKG60386.1, AKG60456.1, AKG60528.1, AKG60601.1, AKG60674.1, AKG60745.1, AKG60817.1, AKG60887.1, AKG60959.1, AKG61032.1, AKG61104.1, AKG61175.1, AKG61248.1, AKG61321.1, AKG61392.1, AKG61464.1, AKG61537.1, AKG61611.1, AKG61684.1, AKG61756.1, AKG61828.1, AKG61902.1, AKG61974.1, AKH80444.1, AKH80517.1, AKM76368.1, ALM22613.1, ALM22687.1, ALM22761.1, ALM22835.1, ALO18641.1, ALO18717.1, AMB65642.1, AMB65715.1, AMB65862.1, AMN09813.1, ANN83942.1, ANN84019.1, ANN84095.1, ANN84172.1, ANN84249.1, ANN84326.1, ANN84403.1, ANN84478.1, ANN84555.1, ANN84632.1, ANN84708.1, ANN84785.1, ANN84861.1, ANN84938.1, ANN85014.1, ANN85091.1, ANN85167.1, ANN85242.1, ANN85319.1, ANN85396.1, ANN85472.1, ANN85549.1, ANN85626.1, ANN85703.1, ANN85779.1, AOY34308.1, AOY36663.1, AOY36687.1, ARB08935.1, ARO38059.1, ARO38060.1, ARO38061.1, ARO38062.1, ARO38063.1, ARO38064.1, ARO38065.1, ARO38066.1, ASM47642.1, ASM47719.1, ASM47796.1, ASM47871.1, BAM73394.1, CAA32294.1, CAB40083.1, CAD13356.1, CAD13357.1, CAD13358.1, CAD13359.1, CAD13360.1, CAD13361.1, CAD13362.1, CAD13363.1, CAD13364.1, CAD13365.1, CAD13366.1, CAD13367.1, CAD13368.1, CAD13369.1, CAD13370.1, CAD13371.1, CAD13372.1, CAD13373.1, CAD13374.1, CAD13375.1, CAD13376.1, CAD13377.1, CAD13378.1, P04290.1, P04488.1, P09855.1, P10228.1, P28986.1, SB007729.1, SB007793.1, SB007798.1, SB007812.1, SB007880.1, SBS69375.1, SBS69379.1, SBS69440.1, SBS69448.1, SBS69560.1, SBS69599.1, SBS69602.1, SBS69637.1, SBS69790.1, SBT69374.1, SCL76887.1, YP 009137119.1, or YP 009137143.1.

### HSV-2 gC

In another embodiment, an RNA encoding HSV gC as described herein comprises an RNA encoding HSV-2 gC. In another embodiment, an RNA encoding HSV gC as described herein comprises RNA encoding a fragment of an HSV-2 gC protein (e.g., an immunogenic fragment).

In some embodiments, a nucleotide sequence of the RNA encoding an HSV-2 gC fragment comprises:

(SEQ ID NO: 65)
GGAAUAAAAGUCUCAACACAACAUAUACAAAACAAACGAAUCUC
AAGCAAUCAAGCAUUCUACUUCUAUUGCAGCAAUUUAAAUCAUU
UCUUUUAAAGCAAAAGCAAUUUUCUGAAAAUUUUCACCAUUUAC
GAACGAUAGC
AUGCGCAUGCAGCUGCUGCUGCUGAUCGC
CCUGUCCCUGGCCCUGGUGACCAACUCC
GCCUCCCCCGGCCGCACCAUCACCGUGGGCCCCCGCGGCAACGC
CUCCAACGCCGCCCCCUCCGCCUCCCCCCGCAACGCCUCCGCCC
CCCGCACCACCCCCACCCCCCCCCAGCCCCGCAAGGCCACCAAG
UCCAAGGCCUCCACCGCCAAGCCCGCCCCCCCCCCCAAGACCGG
CCCCCCCAAGACCUCCUCCGAGCCCGUGCGCUGCAACCGCCACG
ACCCCCUGGCCCGCUACGGCUCCCGCGUGCAGAUCCGCUGCCGC
UUCCCCAACUCCACCCGCACCGAGUUCCGCCUGCAGAUCUGGCG
CUACGCCACCGCCACCGACGCCGAGAUCGGCACCGCCCCCUCCC
UGGAGGAGGUGAUGGUGAACGUGUCCGCCCCCCCCGGCGGCCAG
CUGGUGUACGACUCCGCCCCCAACCGCACCGACCCCCACGUGAU
CUGGGCCGAGGGCGCCGGCCCCGGCGCCUCCCCCCGCCUGUACU
CCGUGGUGGGCCCCCUGGGCCGCCAGCGCCUGAUCAUCGAGGAG
CUGACCCUGGAGACCCAGGGCAUGUACUACUGGGUGUGGGGCCG
CACCGACCGCCCCUCCGCCUACGGCACCUGGGUGCGCGUGCGCG
UGUUCCGCCCCCCCUCCCUGACCAUCCACCCCCACGCCGUGCUG
GAGGGCCAGCCCUUCAAGGCCACCUGCACCGCCGCCACCUACUA
CCCCGGCAACCGCGCCGAGUUCGUGUGGUUCGAGGACGGCCGCC
GCGUGUUCGACCCCGCCCAGAUCCACACCCAGACCCAGGAGAAC
CCCGACGGCUUCUCCACCGUGUCCACCGUGACCUCCGCCGCCGU
GGGCGGCCAGGGCCCCCCCCGCACCUUCACCUGCCAGCUGACCU
GGCACCGCGACUCCGUGUCCUUCUCCCGCCGCAACGCCUCCGGC
ACCGCCUCCGUGCUGCCCCGCCCCACCAUCACCAUGGAGUUCAC
CGGCGACCACGCCGUGUGCACCGCCGGCUGCGUGCCCGAGGGCG
UGACCUUCGCCUGGUUCCUGGGCGACGACUCCUCCCCCGCCGAG
AAGGUGGCCGUGGCCUCCCAGACCUCCUGCGGCCGCCCCGGCAC
CGCCACCAUCCGCUCCACCCUGCCCGUGUCCUACGAGCAGACCG
AGUACAUCUGCCGCCUGGCCGGCUACCCCGACGGCAUCCCCGUG
CUGGAGCACCACUAACUAGUAGUGACUGACUAGGAUCUGGUUAC
CACUAAACCAGCCUCAAGAACACCCGAAUGGAGUCUCUAAGCUA
CAUAAUACCAACUUACACUUACAAAAUGUUGUCCCCCAAAAUGU
AGCCAUUCGUAUCUGCUCCUAAUAAAAAGAAAGUUUCUUCACAU
UCUAAAAAAAAAAAAAAAAAAAAAAAAAAAAAAAAAAAAAAAAA
AAAAAAAAAAAAAAAAAAAAAAAAAAAAAAAAAAAAAAAAAAAA
AAAAAAAAAAAAAAAAC.

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 150). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-2 gC fragment lacks the 5′ untranslated sequences, the signal sequence, the 3′ untranslated sequences, the poly adenylation tail, or a combination thereof. In some embodiments, the sequence of the HSV-2 gC fragment is as set forth in SEQ ID NO: 33.

In some embodiments, an HSV-2 gC fragment encoded by RNA utilized in the methods and compositions of the present disclosure comprises amino acids 27-426 of gC from HSV-2 (e.g., strain 333 or UL44), as set forth in the following amino acid sequence:

(SEQ ID NO: 9)
ASPGRTITVGPRGNASNAAPSASPRNASAPRTTPTPPQPRKATKSKASTA
KPAPPPKTGPPKTSSEPVRCNRHDPLARYGSRVQIRCRFPNSTRTESRLQ
IWRYATATDAEIGTAPSLEEVMVNVSAPPGGQLVYDSAPNRTDPHVIWAE
GAGPGASPRLYSVVGPLGRQRLIIEELTLETQGMYYWVWGRTDRPSAYGT
WVRVRVFRPPSLTIHPHAVLEGQPFKATCTAATYYPGNRAEFVWFEDGRR
VFDPAQIHTQTQENPDGFSTVSTVTSAAVGGQGPPRTFTCQLTWHRDSVS
FSRRNASGTASVLPRPTITMEFTGDHAVCTAGCVPEGVTFAWFLGDDSSP
AEKVAVASQTSCGRPGTATIRSTLPVSYEQTEYICRLAGYPDGIPVLEH
H.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 9. In some embodiments, an HSV-2 gC fragment has an amino acid sequence that is identical to the amino acid sequence SEQ ID NO: 9.

In some embodiments, an HSV-2 gC fragment comprises the following amino acid sequence:

(SEQ ID NO: 10)
ASPGRTITVGPRGNASNAAPSASPRNASAPRTTPTPPQPRKATKSKASTA
KPAPPPKTGPPKTSSEPVRCNRHDPLARYGSRVQIRCRFPNSTRTEFRLQ
IWRYATATDAEIGTAPSLEEVMVNVSAPPGGQLVYDSAPNRTDPHVIWAE
GAGPGASPRLYSVVGPLGRQRLIIEELTLETQGMYYWVWGRTDRPSAYGT
WVRVRVFRPPSLTIHPHAVLEGQPFKATCTAATYYPGNRAEFVWFEDGRR
VFDPAQIHTQTQENPDGFSTVSTVTSAAVGGQGPPRTFTCQLTWHRDSVS
FSRRNASGTASVLPRPTITMEFTGDHAVCTAGCVPEGVTFAWFLGDDSSP
AEKVAVASQTSCGRPGTATIRSTLPVSYEQTEYICRLAGYPDGIPVLEH
H.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 10. In some embodiments, an HSV-2 gC fragment has an amino acid sequence that is identical to the amino acid sequence SEQ ID NO: 10.

In some embodiments, the HSV-2 gC fragment encoded by RNA utilized in the methods and compositions of the present disclosure comprises amino acids 28-426 of gC from HSV-2 (e.g., strain 333 or UL44), as set forth in the following amino acid sequence:

(SEQ ID NO: 11)
SPGRTITVGPRGNASNAAPSASPRNASAPRTTPTPPQPRKATKSKAST
AKPAPPPKTGPPKTSSEPVRCNRHDPLARYGSRVQIRCRFPNSTRTES
RLQIWRYATATDAEIGTAPSLEEVMVNVSAPPGGQLVYDSAPNRTDPH
VIWAEGAGPGASPRLYSVVGPLGRQRLIIEELTLETQGMYYWVWGRTD
RPSAYGTWVRVRVFRPPSLTIHPHAVLEGQPFKATCTAATYYPGNRAE
FVWFEDGRRVFDPAQIHTQTQENPDGFSTVSTVTSAAVGGQGPPRTFT
CQLTWHRDSVSFSRRNASGTASVLPRPTITMEFTGDHAVCTAGCVPEG
VTFAWFLGDDSSPAEKVAVASQTSCGRPGTATIRSTLPVSYEQTEYIC
RLAGYPDGIPVLEHH.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 11. In some embodiments, an HSV-2 gC fragment has an amino acid sequence that is identical to the amino acid sequence SEQ ID NO: 11.

In some embodiments, an HSV-2 gC fragment comprises the following amino acid sequence:

(SEQ ID NO: 12)
SPGRTITVGPRGNASNAAPSASPRNASAPRTTPTPPQPRKATKSKAST
AKPAPPPKTGPPKTSSEPVRCNRHDPLARYGSRVQIRCRFPNSTRTEF
RLQIWRYATATDAEIGTAPSLEEVMVNVSAPPGGQLVYDSAPNRTDPH
VIWAEGAGPGASPRLYSVVGPLGRQRLIIEELTLETQGMYYWVWGRTD
RPSAYGTWVRVRVFRPPSLTIHPHAVLEGQPFKATCTAATYYPGNRAE
FVWFEDGRRVFDPAQIHTQTQENPDGFSTVSTVTSAAVGGQGPPRTFT
CQLTWHRDSVSFSRRNASGTASVLPRPTITMEFTGDHAVCTAGCVPEG
VTFAWFLGDDSSPAEKVAVASQTSCGRPGTATIRSTLPVSYEQTEYIC
RLAGYPDGIPVLEHH.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 12. In some embodiments, an HSV-2 gC fragment has an amino acid sequence that is identical to the amino acid sequence SEQ ID NO:12.

In some embodiments, the HSV-2 gC fragment encoded by RNA utilized in the methods and compositions of the present disclosure comprises amino acids 26-426 of gC from HSV-2 (e.g., strain 333 or UL44), as set forth in the following amino acid sequence:

(SEQ ID NO: 13)
SASPGRTITVGPRGNASNAAPSASPRNASAPRTTPTPPQPRKATKSKA
STAKPAPPPKTGPPKTSSEPVRCNRHDPLARYGSRVQIRCRFPNSTRT
ESRLQIWRYATATDAEIGTAPSLEEVMVNVSAPPGGQLVYDSAPNRTD
PHVIWAEGAGPGASPRLYSVVGPLGRQRLIIEELTLETQGMYYWVWGR
TDRPSAYGTWVRVRVFRPPSLTIHPHAVLEGQPFKATCTAATYYPGNR
AEFVWFEDGRRVFDPAQIHTQTQENPDGFSTVSTVTSAAVGGQGPPRT
FTCQLTWHRDSVSFSRRNASGTASVLPRPTITMEFTGDHAVCTAGCVP
EGVTFAWFLGDDSSPAEKVAVASQTSCGRPGTATIRSTLPVSYEQTEY
ICRLAGYPDGIPVLEHH.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 13. In some embodiments, an HSV-2 gC fragment has an amino acid sequence that is identical to the amino acid sequence SEQ ID NO: 13.

In some embodiments, an HSV-2 gC fragment comprises the following amino acid sequence:

(SEQ ID NO: 14)
SASPGRTITVGPRGNASNAAPSASPRNASAPRTTPTPPQPRKATKSKA
STAKPAPPPKTGPPKTSSEPVRCNRHDPLARYGSRVQIRCRFPNSTRT
EFRLQIWRYATATDAEIGTAPSLEEVMVNVSAPPGGQLVYDSAPNRTD
PHVIWAEGAGPGASPRLYSVVGPLGRQRLIIEELTLETQGMYYWVWGR
TDRPSAYGTWVRVRVFRPPSLTIHPHAVLEGQPFKATCTAATYYPGNR
AEFVWFEDGRRVFDPAQIHTQTQENPDGFSTVSTVTSAAVGGQGPPRT
FTCQLTWHRDSVSFSRRNASGTASVLPRPTITMEFTGDHAVCTAGCVP
EGVTFAWFLGDDSSPAEKVAVASQTSCGRPGTATIRSTLPVSYEQTEY
ICRLAGYPDGIPVLEHH.

In some embodiments, an HSV-2 gC fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 14. In some embodiments, an HSV-2 gC fragment has an amino acid sequence that is identical to the amino acid sequence SEQ ID NO: 14.

In some embodiments, full-length HSV-2 gC encoded by RNA utilized in the methods and compositions of the present disclosure comprises the following amino acid sequence:

(SEQ ID NO: 15)
MALGRVGLAVGLWGLLWVGVVVVLANASPGRTITVGPRGNASNAAPSA
SPRNASAPRTTPTPPQPRKATKSKASTAKPAPPPKTGPPKTSSEPVRC
NRHDPLARYGSRVQIRCRFPNSTRTEFRLQIWRYATATDAEIGTAPSL
EEVMVNVSAPPGGQLVYDSAPNRTDPHVIWAEGAGPGASPRLYSVVGP
LGRQRLIIEELTLETQGMYYWVWGRTDRPSAYGTWVRVRVFRPPSLTI
HPHAVLEGQPFKATCTAATYYPGNRAEFVWFEDGRRVFDPAQIHTQTQ
ENPDGFSTVSTVTSAAVGGQGPPRTFTCQLTWHRDSVSFSRRNASGTA
SVLPRPTITMEFTGDHAVCTAGCVPEGVTFAWFLGDDSSPAEKVAVAS
QTSCGRPGTATIRSTLPVSYEQTEYICRLAGYPDGIPVLEHHGSHQPP
PRDPTERQVIRAVEGAGIGVAVLVAVVLAGTAVVYLTHASSVRYRRL
R.

In some embodiments, an HSV-2 gC comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 15. In some embodiments, an HSV-2 gC has an amino acid sequence that is identical to the amino acid sequence SEQ ID NO: 15.

In another embodiment, the HSV-2 gC or an immunogenic fragment thereof, encoded by RNA utilized in the methods and compositions of the present disclosure comprises the amino acid sequences as set forth in any of the following GenBank Accession Numbers: AAA20532.1, AAA66442.1, AAB60549.1, AAB60550.1, AAB60551.1, AAB72101.1, ABU45429.1, ABU45430.1, ABU45431.1, ABU45432.1, ABU45459.1, ABU45460.1, AEV91348.1, AEV91383.1, AEV91407.1, AFM93864.1, AHG54708.1, AKC42808.1, AKC59285.1, AKC59357.1, AKC59428.1, AKC59499.1, AKC59570.1, AMB66008.1, AMB66079.1, AMB66151.1, AMB66224.1, AMB66252.1, AMB66253.1, AMB66368.1, AMB66441.1, AQZ55735.2, AQZ55806.1, AQZ55877.1, AQZ55948.1, AQZ56019.1, AQZ56090.1, AQZ56161.2, AQZ56232.2, AQZ56303.2, AQZ56374.2, AQZ56445.1, AQZ56516.1, AQZ56587.1, AQZ56658.1, AQZ56729.2, AQZ56800.1, AQZ56871.1, AQZ56942.2, AQZ57013.1, AQZ57084.2, AQZ57155.1, AQZ57226.1, AQZ57297.1, AQZ57368.1, AQZ57439.1, AQZ57510.1, AQZ57581.1, AQZ57652.1, AQZ57723.1, AQZ57794.2, AQZ57865.2, AQZ57936.1, AQZ58007.2, AQZ58078.1, AQZ58149.2, AQZ58220.1, AQZ58291.1, AQZ58362.1, AQZ58433.1, AQZ58504.1, AQZ58575.1, AQZ58646.1, AQZ58717.2, AQZ58788.2, AQZ58859.2, AQZ58930.1, AQZ59001.2, AQZ59072.1, AQZ59143.1, ARO38067.1, ARO38068.1, ARO38069.1, ARO38070.1, ARO38071.1, ARO38072.1, CAA25687.1, CAA26025.1, CAB06730.1, CAB06734.1, CAB96544.1, P03173.1, P06475.1, P89475.1, Q89730.1, YP_009137161.1, YP_009137196.1, or YP_009137220.1.

In another embodiment, a gC protein fragment encoded by RNA utilized in the methods and compositions of the present disclosure comprises a properdin interfering domain. “Properdin-interfering domain” refers, in some embodiments, to a domain that blocks or inhibits binding of a host C3b molecule with a host properdin molecule. In another embodiment, the term refers to a domain that blocks or inhibits an interaction of a host C3b molecule with a host properdin molecule.

In another embodiment, a gC protein fragment encoded by RNA utilized in the methods and compositions of the present disclosure is a C5 interfering domain. In another embodiment, the gC protein fragment is a portion of a C5 interfering domain. “C5-interfering domain” refers, in another embodiment, to a domain that interferes with binding of a host C3b molecule with a host C5 molecule. In another embodiment, the term refers to a domain that interferes with the interaction of a host C3b molecule with a host C5 molecule.

Each RNA encoding HSV-1 gC or HSV-2 gC protein or fragment thereof represents a separate embodiment of the present disclosure.

In another embodiment, a gC protein fragment encoded by RNA utilized in the methods and compositions of the present disclosure is an immunogenic fragment. In another embodiment, a gC immunoprotective antigen need not be the entire protein. The protective immune response generally involves, in another embodiment, an antibody response. In another embodiment, mutants, sequence conservative variants, and functional conservative variants of gC are useful in methods and compositions of the present disclosure, provided that all such variants retain the required immuno-protective effect. In another embodiment, the immunogenic fragment can comprise an immuno-protective gC antigen from any strain of HSV. In another embodiment, the immunogenic fragment can comprise sequence variants of HSV, as found in infected individuals.

### Glycoprotein D

In some embodiments, the present disclosure provides an RNA encoding HSV glycoprotein D (gD) or an immunogenic fragment thereof. In another embodiment, the present disclosure provides a composition comprising an RNA encoding HSV gD or an immunogenic fragment thereof.

### HSV-1 gD

In another embodiment, RNA encoding HSV gD as described herein comprises an RNA encoding HSV-1 gD. In another embodiment, an RNA encoding HSV gD as described herein comprises an RNA encoding a fragment of an HSV-1 gD protein (e.g., immunogenic fragment).

In some embodiments, a nucleotide sequence of the RNA encoding an HSV-1 gD fragment comprises:

(SEQ ID NO: 16)
GGAAUAAAAGUCUCAACACAACAUAUACAAAACAAACGAAUCUCAAGC
AAUCAAGCAUUCUACUUCUAUUGCAGCAAUUUAAAUCAUUUCUUUUAA
AGCAAAAGCAAUUUUCUGAAAAUUUUCACCAUUUACGAACGAUAGCAU
GCGCAUGCAGCUGCUGCUGCUGAUCGCCCUGUCCCUGGCCCUGGUGAC
CAACUCCCGCACCUCCUGGAAGCGCGUGACCUCCGGCGAGGACGUGGU
GCUGCUGCCCGCCCCCGCCGGCCCCGAGGAGCGCACCCGCGCCCACAA
GCUGCUGUGGGCCGCCGAGCCCCUGGACGCCUGCGGCCCCCUGCGCCC
CUCCUGGGUGGCCCUGUGGCCCCCCCGCCGCGUGCUGGAGACCGUGGU
GGACGCCGCCUGCAUGCGCGCCCCCGAGCCCCUGGCCAUCGCCUACUC
CCCCCCCUUCCCCGCCGGCGACGAGGGCCUGUACUCCGAGCUGGCCUG
GCGCGACCGCGUGGCCGUGGUGAACGAGUCCCUGGUGAUCUACGGCGC
CCUGGAGACCGACUCCGGCCUGUACACCCUGUCCGUGGUGGGCCUGUC
CGACGAGGCCCGCCAGGUGGCCUCCGUGGUGCUGGUGGUGGAGCCCGC
CCCCGUGCCCACCCCCACCCCCGACGACUACGACGAGGAGGACGACGC
CGGCGUGUCCGAGCGCACCCCCGUGUCCGUGCCCCCCCCCACCCCCCC
CCGCCGCCCCCCCGUGGCCCCCCCCACCCACCCCCGCGUGAUCCCCGA
GGUGUCCCACGUGCGCGGCGUGACCGUGCACAUGGAGACCCCCGAGGC
CAUCCUGUUCGCCCCCGGCGAGACCUUCGGCACCAACGUGUCCAUCCA
CGCCAUCGCCCACGACGACGGCCCCUACGCCAUGGACGUGGUGUGGAU
GCGCUUCGACGUGCCCUCCUCCUGCGCCGAGAUGCGCAUCUACGAGGC
CUGCCUGUACCACCCCCAGCUGCCCGAGUGCCUGUCCCCCGCCGACGC
CCCCUGCGCCGUGUCCUCCUGGGCCUACCGCCUGGCCGUGCGCUCCUA
CGCCGGCUGCUCCCGCACCACCCCCCCCCCCCGCUGCUUCGCCGAGGC
CCGCAUGGAGCCCGUGCCCGGCCUGGCCUGGCUGGCCUCCACCGUGAA
CCUGGAGUUCCAGCACGCCUCCCCCCAGCACGCCGGCCUGUACCUGUG
CGUGGUGUACGUGGACGACCACAUCCACGCCUGGGGCCACAUGACCAU
CUCCACCGCCGCCCAGUACCGCAACGCCGUGGUGGAGCAGCACCUGCC
CCAGCGCCAGCCCGAGCCCGUGGAGCCCACCCGCCCCCACGUGCGCGC
CUAACUAGUAGUGACUGACUAGGAUCUGGUUACCACUAAACCAGCCUC
AAGAACACCCGAAUGGAGUCUCUAAGCUACAUAAUACCAACUUACACU
UACAAAAUGUUGUCCCCCAAAAUGUAGCCAUUCGUAUCUGCUCCUAAU
AAAAAGAAAGUUUCUUCACAUUCUAAAAAAAAAAAAAAAAAAAAAAAA
AAAAAAAAAAAAAAAAAAAAAAAAAAAAAAAAAAAAAAAAAAAAAAAA
AAAAAAAAAAAAAAAAAAAAAAAAAAAAAC

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 150). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-1 gD fragment lacks the 5′ untranslated sequences, the signal sequence, the 3′ untranslated sequences, the poly adenylation tail, or a combination thereof. In some embodiments, the polynucleotide sequence of the HSV-1 gD fragment is as set forth in SEQ ID NO: 54.

In some embodiments, an HSV-1 gD fragment encoded by RNA utilized in the methods and compositions of the present disclosure comprises amino acids 26-331 of gD (e.g., from HSV-1 Patton strain), as set forth in the following amino acid sequence:

(SEQ ID NO: 16)
KYALADASLKMADPNRFRGKDLPVLDQLTDPPGVRRVYHIQAGLPDPF
QPPSLPITVYYAVLERACRSVLLNAPSEAPQIVRGASEDVRKQPYNLT
IAWFRMGGNCAIPITVMEYTECSYNKSLGACPIRTQPRWNYYDSFSAV
SEDNLGFLMHAPAFETAGTYLRLVKINDWTEITQFILEHRAKGSCKYA
LPLRIPPSACLSPQAYQQGVTVDSIGMLPRFIPENQRTVAVYSLKIAG
WHGPKAPYTSTLLPPELSETPNATQPELAPEDPEDSALLEDPVGTVAP
QIPPNWHIPSIQDAATPY

In some embodiments, an HSV-1 gD fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 16. In some embodiments, an HSV-1 gD fragment has an amino acid sequence that is identical to the amino acid sequence SEQ ID NO: 16.

In some embodiments, a full-length HSV-1 gD encoded by RNA utilized in the methods and compositions of the present disclosure comprises the following amino acid sequence:

(SEQ ID NO: 17)
MGGAAARLGAVILFVVIVGLHGVRGKYALADASLKLADPNRFRRKDLP
VLDQLTDPPGVRRVYHIQAGLPDPFQPPSLPITVYYAVLERACRSVLL
NAPSEAPQIVRGASEDVRKQPYNLTIAWFRMGGNCAIPITVMEYTECS
YNKSLGACPIRTQPRWNYYDSFSAVSEDNLGFLMHAPAFETAGTYLRL
VKINDWTEITQFILEHRAKGSCKYALPLRIPPSACLSPQAYQQGVTVD
SIGMLPRFIPENQRTVAVYSLKIAGWHGPKAPYTSTLLPPELSETPNA
TQPELAPEAPEDSALLEDPVGTVAPQIPPNWHIPSIQDAATPYHPPAT
PNNMGLIAGAVGGSLLAALVICGIVYWMRRRTQKAPKRIRLPHIREDD
QPSSHQPLFY

In some embodiments, an HSV-1 gD comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 17. In some embodiments, an HSV-1 gD has an amino acid sequence that is identical to the amino acid sequence SEQ ID NO: 17.

In another embodiment, an HSV-1 gD or an immunogenic fragment thereof, encoded by RNA utilized in the methods and compositions of the present disclosure comprises the amino acid sequences as set forth in any one of the following GenBank Accession Numbers: AAL90884.1 (KHS2 strain), AAL90883.1 (KHS1 strain), AAK93950.1 (F strain), AAB59754.1 (F strain), AAA19631.1 (mutant strain not identified), AAA19630.1 (mutant strain not identified), or AAA19629.1 (strain not identified).

In another embodiment, an HSV-1 gD or an immunogenic fragment thereof, encoded by RNA utilized in the methods and compositions of the present disclosure comprises the amino acid sequences as set forth in any of the following GenBank Accession Numbers: A1Z0Q5.2, AAA45780.1, AAA45785.1, AAA45786.1, AAA96682.1, AAK19597.1, AAN74642.1, ABI63524.1, ABM52978.1, ABM52979.1, ABM52980.1, ABM52981.1, ABM66847.1, ABM66848.1, ACM62295.1, ADD60053.1, ADD60130.1, ADM22389.1, ADM22466.1, ADM22542.1, ADM22619.1, ADM22696.1, ADM22773.1, ADM22849.1, ADM22926.1, ADM23003.1, ADM23079.1, ADM23155.1, ADM23231.1, ADM23309.1, ADM23383.1, ADM23457.1, ADM23531.1, ADM23605.1, ADM23680.1, ADM23755.1, ADM23831.1, AEQ77097.1, AER37647.1, AER37715.1, AER37786.1, AER37857.1, AER37929.1, AER38000.1, AER38070.1, AFE62894.1, AFH41180.1, AFI23657.1, AFK50415.1, AFP86430.1, AGZ01928.1, AIR95858.1, AJE60009.1, AJE60080.1, AJE60151.1, AJE60222.1, AJE60293.1, AJE60439.1, AKE48645.1, AKG59246.1, AKG59318.1, AKG59391.1, AKG59462.1, AKG59536.1, AKG59609.1, AKG59682.1, AKG59755.1, AKG59826.1, AKG59898.1, AKG59972.1, AKG60046.1, AKG60118.1, AKG60189.1, AKG60261.1, AKG60334.1, AKG60404.1, AKG60474.1, AKG60546.1, AKG60620.1, AKG60692.1, AKG60763.1, AKG60835.1, AKG60906.1, AKG60978.1, AKG61050.1, AKG61123.1, AKG61194.1, AKG61267.1, AKG61339.1, AKG61411.1, AKG61484.1, AKG61556.1, AKG61629.1, AKG61703.1, AKG61774.1, AKG61847.1, AKG61920.1, AKG61993.1, AKH80463.1, AKH80536.1, ALM22635.1, ALM22709.1, ALM22783.1, ALM22857.1, ALO18662.1, ALO18738.1, AMB65662.1, AMB65735.1, AMB65809.1, AMB65885.1, AMB65956.1, AMN09832.1, ANN83964.1, ANN84041.1, ANN84117.1, ANN84194.1, ANN84271.1, ANN84348.1, ANN84424.1, ANN84500.1, ANN84577.1, ANN84653.1, ANN84730.1, ANN84806.1, ANN84883.1, ANN84959.1, ANN85036.1, ANN85112.1, ANN85187.1, ANN85264.1, ANN85341.1, ANN85416.1, ANN85494.1, ANN85571.1, ANN85648.1, ANN85724.1, ANN85801.1, AOY34093.1, AOY34141.1, AOY34243.1, AOY34271.1, AOY34337.1, AOY36685.1, ARB08957.1, ARO37961.1, ARO37962.1, ARO37963.1, ARO37964.1, ARO37965.1, ARO37966.1, ARO37967.1, ARO37968.1, ARO37969.1, ARO37970.1, ARO37971.1, ARO37972.1, ARO37973.1, ARO37974.1, ARO37975.1, ARO37976.1, ARO37977.1, ARO37978.1, ARO37979.1, ARO37980.1, ARO37981.1, ARO37982.1, ARO37983.1, ARO37984.1, ARO37985.1, ARO37986.1, ARO37987.1, ARO37988.1, ARO37989.1, ARO37990.1, ARO37991.1, ARO37992.1, ARO37993.1, ARO37994.1, ARO37995.1, ARO37996.1, ARO37997.1, ARO37998.1, ARO37999.1, ASM47664.1, ASM47741.1, ASM47818.1, ASM47893.1, BAM73419.1, CAA26060.1, CAA32283.1, CAA32284.1, CAA32289.1, CAA38245.1, CAT05431.1, P06476.1, P36318.1, P57083.1, P68331.1, Q05059.1, Q69091.1, SB007792.1, SB007819.1, SB007855.1, SB007869.1, SB007887.1, SB007908.1, SBS69553.1, SBS69561.1, SBS69579.1, SBS69625.1, SBS69688.1, SBS69694.1, SBS69717.1, SBS69727.1, SBS69811.1, SBT69395.1, SCL76902.1, VGBEDZ, or YP_009137141.1.

### HSV-2 gD

In another embodiment, an RNA encoding HSV gD as described herein comprises an RNA encoding HSV-2 gD. In another embodiment, RNA encoding HSV gD as described herein comprises RNA encoding a fragment of an HSV-2 gD protein (e.g., immunogenic fragment).

In some embodiments, a nucleotide sequence of the RNA encoding an HSV-2 gD fragment comprises:

(SEQ ID NO: 67)
GGAAUAAAAGUCUCAACACAACAUAUACAAAACAAACGAAUCUCAAGC
AAUCAAGCAUUCUACUUCUAUUGCAGCAAUUUAAAUCAUUUCUUUUAA
AGCAAAAGCAAUUUUCUGAAAAUUUUCACCAUUUACGAACGAUAGCAU
GACCCGCCUGACCGUGCUGGCCCUGCUGGCCGGCCUGCUGGCCUCCUC
CCGCGCCAAGUACGCCCUGGCCGACCCCUCCCUGAAGAUGGCCGACCC
CAACCGCUUCCGCGGCAAGAACCUGCCCGUGCUGGACCAGCUGACCGA
CCCCCCCGGCGUGAAGCGCGUGUACCACAUCCAGCCCUCCCUGGAGGA
CCCCUUCCAGCCCCCCUCCAUCCCCAUCACCGUGUACUACGCCGUGCU
GGAGCGCGCCUGCCGCUCCGUGCUGCUGCACGCCCCCUCCGAGGCCCC
CCAGAUCGUGCGCGGCGCCUCCGACGAGGCCCGCAAGCACACCUACAA
CCUGACCAUCGCCUGGUACCGCAUGGGCGACAACUGCGCCAUCCCCAU
CACCGUGAUGGAGUACACCGAGUGCCCCUACAACAAGUCCCUGGGCGU
GUGCCCCAUCCGCACCCAGCCCCGCUGGUCCUACUACGACUCCUUCUC
CGCCGUGUCCGAGGACAACCUGGGCUUCCUGAUGCACGCCCCCGCCUU
CGAGACCGCCGGCACCUACCUGCGCCUGGUGAAGAUCAACGACUGGAC
CGAGAUCACCCAGUUCAUCCUGGAGCACCGCGCCCGCGCCUCCUGCAA
GUACGCCCUGCCCCUGCGCAUCCCCCCCGCCGCCUGCCUGACCUCCAA
GGCCUACCAGCAGGGCGUGACCGUGGACUCCAUCGGCAUGCUGCCCCG
CUUCAUCCCCGAGAACCAGCGCACCGUGGCCCUGUACUCCCUGAAGAU
CGCCGGCUGGCACGGCCCCAAGCCCCCCUACACCUCCACCCUGCUGCC
CCCCGAGCUGUCCGACACCACCAACGCCACCCAGCCCGAGCUGGUGCC
CGAGGACCCCGAGGACUCCGCCCUGCUGGAGGACCCCGCCGGCACCGU
GUCCUCCCAGAUCCCCCCCAACUGGCACAUCCCCUCCAUCCAGGACGU
GGCCCCCCACCACUAACUAGUAGUGACUGACUAGGAUCUGGUUACCAC
UAAACCAGCCUCAAGAACACCCGAAUGGAGUCUCUAAGCUACAUAAUA
CCAACUUACACUUACAAAAUGUUGUCCCCCAAAAUGUAGCCAUUCGUA
UCUGCUCCUAAUAAAAAGAAAGUUUCUUCACAUUCUAAAAAAAAAAAA
AAAAAAAAAAAAAAAAAAAAAAAAAAAAAAAAAAAAAAAAAAAAAAAA
AAAAAAAAAAAAAAAAAAAAAAAAAAAAAAAAAAAAAAAAAC

In some embodiments, all uridine residues are 1-methyl-pseudouridine. In some embodiments, underlined residues represent 5′ untranslated sequences (SEQ ID NO: 253). In some embodiments, bold residues represent a signal sequence (leader sequence) (SEQ ID NO: 154). In some embodiments, italicized residues represent 3′ untranslated sequences (SEQ ID NO: 254) and poly adenylation tail (SEQ ID NO: 255).

In another embodiment, a nucleotide sequence of the RNA encoding an HSV-2 gD fragment lacks the 5′ untranslated sequences, the signal sequence, the 3′ untranslated sequences, the poly adenylation tail, or a combination thereof. In some embodiments, the sequence of the HSV-2 gD fragment is as set forth in SEQ ID NO: 55.

In some embodiments, an HSV-2 gD fragment encoded by RNA utilized in the methods and compositions of the present disclosure comprises amino acids 26-331 of gD (e.g., from HSV-2 strain 333 or US6), as set forth in the following amino acid sequence:

(SEQ ID NO: 18)
KYALADPSLKMADPNRFRGKNLPVLDQLTDPPGVKRVYHIQPSLEDPF
QPPSIPITVYYAVLERACRSVLLHAPSEAPQIVRGASDEARKHTYNLT
IAWYRMGDNCAIPITVMEYTECPYNKSLGVCPIRTQPRWSYYDSFSAV
SEDNLGFLMHAPAFETAGTYLRLVKINDWTEITQFILEHRARASCKYA
LPLRIPPAACLTSKAYQQGVTVDSIGMLPRFIPENQRTVALYSLKIAG
WHGPKPPYTSTLLPPELSDTTNATQPELVPEDPEDSALLEDPAGTVSS
QIPPNWHIPSIQDVAPHH.

In some embodiments, an HSV-2 gD fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 18. In some embodiments, an HSV-2 gD fragment has an amino acid sequence that is identical to the amino acid sequence SEQ ID NO: 18.

In some embodiments, an HSV-2 gD fragment encoded by RNA utilized in the methods and compositions of the present disclosure comprises amino acids 30-331 of gD from HSV-2(e.g., strain 333 or US6), as set forth in the following amino acid sequence:

(SEQ ID NO: 19)
ADPSLKMADPNRFRGKNLPVLDQLTDPPGVKRVYHIQPSLEDPFQPPS
IPITVYYAVLERACRSVLLHAPSEAPQIVRGASDEARKHTYNLTIAWY
RMGDNCAIPITVMEYTECPYNKSLGVCPIRTQPRWSYYDSFSAVSEDN
LGFLMHAPAFETAGTYLRLVKINDWTEITQFILEHRARASCKYALPLR
IPPAACLTSKAYQQGVTVDSIGMLPRFIPENQRTVALYSLKIAGWHGP
KPPYTSTLLPPELSDTTNATQPELVPEDPEDSALLEDPAGTVSSQIPP
NWHIPSIQDVAPHH.

In some embodiments, an HSV-2 gD fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 19. In some embodiments, an HSV-2 gD fragment has an amino acid sequence that is identical to the amino acid sequence SEQ ID NO: 19.

In some embodiments, an HSV-2 gD fragment encoded by RNA utilized in the methods and compositions of the present disclosure comprises amino acids 31-331 of gD from HSV-2 (e.g., strain 333 or US6), as set forth in the following amino acid sequence:

(SEQ ID NO: 20)
DPSLKMADPNRFRGKNLPVLDQLTDPPGVKRVYHIQPSLEDPFQPPSI
PITVYYAVLERACRSVLLHAPSEAPQIVRGASDEARKHTYNLTIAWYR
MGDNCAIPITVMEYTECPYNKSLGVCPIRTQPRWSYYDSFSAVSEDNL
GFLMHAPAFETAGTYLRLVKINDWTEITQFILEHRARASCKYALPLRI
PPAACLTSKAYQQGVTVDSIGMLPRFIPENQRTVALYSLKIAGWHGPK
PPYTSTLLPPELSDTTNATQPELVPEDPEDSALLEDPAGTVSSQIPPN
WHIPSIQDVAPHH.

In some embodiments, an HSV-2 gD fragment comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 20. In some embodiments, an HSV-2 gD fragment has an amino acid sequence that is identical to the amino acid sequence SEQ ID NO: 20.

In some embodiments, the full-length HSV-2 gD encoded by RNA utilized in the methods and compositions of the present disclosure comprises the following amino acid sequence:

(SEQ ID NO: 21)
MGRLTSGVGTAALLVVAVGLRVVCAKYALADPSLKMADPNRFRGKNLP
VLDQLTDPPGVKRVYHIQPSLEDPFQPPSIPITVYYAVLERACRSVLL
HAPSEAPQIVRGASDEARKHTYNLTIAWYRMGDNCAIPITVMEYTECP
YNKSLGVCPIRTQPRWSYYDSFSAVSEDNLGFLMHAPAFETAGTYLRL
VKINDWTEITQFILEHRARASCKYALPLRIPPAACLTSKAYQQGVTVD
SIGMLPRFIPENQRTVALYSLKIAGWHGPKPPYTSTLLPPELSDTTNA
TQPELVPEDPEDSALLEDPAGTVSSQIPPNWHIPSIQDVAPHHAPAAP
SNPGLIIGALAGSTLAVLVIGGIAFWVRRRAQMAPKRLRLPHIRDDDA
PPSHQPLFY.

In some embodiments, an HSV-2 gD comprises an amino acid sequence that is at least 85%, at least 90%, at least 91%, at least 92%, at least 93%, at least 94%, at least 95%, at least 96%, at least 97%, at least 98%, at least 99%, or 100% identical to the amino acid sequence SEQ ID NO: 21. In some embodiments, an HSV-2 gD has an amino acid sequence that is identical to the amino acid sequence SEQ ID NO: 21. In another embodiment, the HSV-2 gD or immunogenic fragment thereof, encoded by RNA utilized in the methods and compositions of the present disclosure comprises the amino acid sequences as set forth in GenBank Accession Numbers: 1003204A, AAA45841.1, AAA45842.1, AAB60552.1, AAB60553.1, AAB60554.1, AAB60555.1, AAB72102.1, AAS01730.1, AAW23130.1, AAW23131.1, AAW23132.1, AAW23133.1, AAW23134.1, ABS84899.1, ABU45433.1, ABU45434.1, ABU45435.1, ABU45461.1, ABU45462.1, ACA28831.1, AEV91405.1, AFM93876.1, AFS18198.1, AFS18199.1, AFS18200.1, AFS18201.1, AFS18202.1, AFS18203.1, AFS18204.1, AFS18205.1, AFS18206.1, AFS18207.1, AFS18208.1, AFS18209.1, AFS18210.1, AFS18211.1, AFS18212.1, AFS18213.1, AFS18214.1, AFS18215.1, AFS18216.1, AFS18217.1, AFS18218.1, AFS18219.1, AFS18220.1, AFS18221.1, AHG54730.1, AIL27720.1, AIL27721.1, AIL27722.1, AIL27723.1, AIL27724.1, AIL27725.1, AIL27726.1, AIL27727.1, AIL27728.1, AIL27729.1, AIL27730.1, AIL27731.1, AIL28069.1, AIL28070.1, AKC42828.1, AKC59305.1, AKC59376.1, AKC59447.1, AKC59518.1, AKC59589.1, AMB66102.1, AMB66171.1, AMB66244.1, AMB66321.1, AMB66394.1, AMB66463.1, AQZ55754.1, AQZ55825.1, AQZ55896.1, AQZ55967.1, AQZ56038.1, AQZ56109.1, AQZ56180.1, AQZ56251.1, AQZ56322.1, AQZ56393.1, AQZ56464.1, AQZ56535.1, AQZ56606.1, AQZ56677.1, AQZ56748.1, AQZ56819.1, AQZ56890.1, AQZ56961.1, AQZ57032.1, AQZ57103.1, AQZ57174.1, AQZ57245.1, AQZ57316.1, AQZ57387.1, AQZ57458.1, AQZ57529.1, AQZ57600.1, AQZ57671.1, AQZ57742.1, AQZ57813.1, AQZ57884.1, AQZ57955.1, AQZ58026.1, AQZ58097.1, AQZ58168.1, AQZ58239.1, AQZ58310.1, AQZ58381.1, AQZ58452.1, AQZ58523.1, AQZ58594.1, AQZ58665.1, AQZ58736.1, AQZ58807.1, AQZ58878.1, AQZ58949.1, AQZ59020.1, AQZ59091.1, AQZ59162.1, ARO38000.1, ARO38001.1, ARO38002.1, ARO38003.1, ARO38004.1, ARO38005.1, ARO38006.1, ARO38007.1, ARO38008.1, ARO38009.1, ARO38010.1, ARO38011.1, ARO38012.1, ARO38013.1, ARO38014.1, ARO38015.1, ARO38016.1, ARO38017.1, ARO38018.1, ARO38019.1, ARO38020.1, ARO38021.1, ARO38022.1, ARO38023.1, ARO38024.1, ARO38025.1, ARO38026.1, ARO38027.1, ARO38028.1, ARO38029.1, ARO38030.1, ARO38031.1, ARO38032.1, ARO38033.1, ARO38034.1, ARO38035.1, ARO38036.1, ARO38037.1, ARO38038.1, ARO38039.1, ARO38040.1, ARO38041.1, ARO38042.1, ARO38043.1, ARO38044.1, CAA26025.1, CAB06713.1, CAC33573.1, CAT05432.1, P03172.2, Q69467.1, or YP_009137218.1.

In another embodiment, the gD protein or fragment (e.g., immunogenic fragment) includes Y63. In another embodiment, the gD protein or fragment (e.g., immunogenic fragment) includes R159. In another embodiment, the gD protein or fragment (e.g., immunogenic fragment) includes D240. In another embodiment, the gD protein or fragment (e.g., immunogenic fragment) includes P246. In another embodiment, the gD protein or fragment (e.g., immunogenic fragment) includes a residue selected from Y63, R159, D240, and P246. In another embodiment, inclusion of one of these residues elicits antibodies that inhibit binding to nectin-1.

The nomenclature used herein for gD amino acid residues includes the residues of the signal peptide encoded by the signal sequence. Thus, residue one of the mature protein is referred to as “26.”

Each RNA encoding HSV-1 gD and HSV-2 gD protein or fragment thereof represents a separate embodiment of the present disclosure.

In another embodiment, the HSV gD, gC, and gE proteins, and fragments thereof, encoded by the modified RNA as disclosed herein are described in US Patent Publication No. 2013-0028925-A1, which is incorporated by reference herein in its entirety.

In another embodiment, a gD protein fragment encoded by RNA utilized in the methods and compositions of the present disclosure is an immunogenic fragment. In another embodiment, a gD immunoprotective antigen need not be the entire protein. The protective immune response generally involves, in another embodiment, an antibody response. In another embodiment, mutants, sequence conservative variants, and functional conservative variants of gD are useful in methods and compositions of the present disclosure, provided that all such variants retain the required immuno-protective effect. In another embodiment, the immunogenic fragment can comprise an immuno-protective gD antigen from any strain of HSV. In another embodiment, the immunogenic fragment can comprise sequence variants of HSV, as found in infected individuals.

In some embodiments, an RNA of the present disclosure encodes an HSV polypeptide, or fragment thereof, at least 80%, at least 85%, at least 90%, at least 91%, at least 92%, at least 93%, at least 94%, at least 95%, at least 96%, at least 97%, at least 98%, at least 99%, or 100% identical to a sequence listed in Table 1.

In some embodiments, methods of the present disclosure comprise administering to a subject an HSV polypeptide, or fragment thereof, at least 80%, at least 85%, at least 90%, at least 91%, at least 92%, at least 93%, at least 94%, at least 95%, at least 96%, at least 97%, at least 98%, at least 99%, or 100% identical to a sequence listed in Table 1.

TABLE 1
Exemplary amino acid sequences of HSV immunogens

| Sequence | | |
|---|---|---|
| Name | Amino Acid Sequence | SEQ ID NO: |
| HSV-1 gE | KTSWRRVSVGEDVSLLPAPGPTGRGPTQKLLWAVEPL | 1 |
| (24-409) | DGCGPLHPSWVSLMPPKQVPETVVDAACMRAPVPLA | |
| | MAYAPPAPSATGGLRTDFVWQERAAVVNRSLVIYGVR | |
| | ETDSGLYTLSVGDIKDPARQVASVVLVVQPAPVPTPPP | |
| | TPADYDEDDNDEGEGEDESLAGTPASGTPRLPPSPAPP | |
| | RSWPSAPEVSHVRGVTVRMETPEAILFSPGEAFSTNVSI | |
| | HAIAHDDQTYTMDVVWLRFDVPTSCAEMRIYESCLYH | |
| | PQLPECLSPADAPCAASTWTSRLAVRSYAGCSRTNPPP | |
| | RCSAEAHMEPFPGLAWQAASVNLEFRDASPQHSGLYL | |
| | CVVYVNDHIHAWGHITINTAAQYRNAVVEQPLPQRGA | |
| | DLAEPTHPHVGA | |
| HSV-1 gE | GTPKTSWRRVSVGEDVSLLPAPGPTGRGPTQKLLWAV | 2 |
| (21-409) | EPLDGCGPLHPSWVSLMPPKQVPETVVDAACMRAPVP | |
| | LAMAYAPPAPSATGGLRTDFVWQERAAVVNRSLVIYG | |
| | VRETDSGLYTLSVGDIKDPARQVASVVLVVQPAPVPTP | |
| | PPTPADYDEDDNDEGEGEDESLAGTPASGTPRLPPSPAP | |
| | PRSWPSAPEVSHVRGVTVRMETPEAILFSPGEAFSTNVS | |
| | IHAIAHDDQTYTMDVVWLRFDVPTSCAEMRIYESCLY | |
| | HPQLPECLSPADAPCAASTWTSRLAVRSYAGCSRTNPP | |
| | PRCSAEAHMEPFPGLAWQAASVNLEFRDASPQHSGLY | |
| | LCVVYVNDHIHAWGHITINTAAQYRNAVVEQPLPQRG | |
| | ADLAEPTHPHVGA | |
| HSV-1 gE | MDRGAVVGFLLGVCVVSCLAGTPKTSWRRVSVGEDV | 3 |
| Full- | SLLPAPGPTGRGPTQKLLWAVEPLDGCGPLHPSWVSL | |
| length | MPPKQVPETVVDAACMRAPVPLAMAYAPPAPSATGGL | |
| | RTDFVWQERAAVVNRSLVIYGVRETDSGLYTLSVGDI | |
| | KDPARQVASVVLVVQPAPVPTPPPTPADYDEDDNDEG | |
| | EGEDESLAGTPASGTPRLPPSPAPPRSWPSAPEVSHVRG | |
| | VTVRMETPEAILFSPGEAFSTNVSIHAIAHDDQTYTMD | |
| | VVWLRFDVPTSCAEMRIYESCLYHPQLPECLSPADAPC | |
| | AASTWTSRLAVRSYAGCSRTNPPPRCSAEAHMEPFPGL | |
| | AWQAASVNLEFRDASPQHSGLYLCVVYVNDHIHAWG | |
| | HITINTAAQYRNAVVEQPLPQRGADLAEPTHPHVGAPP | |
| | HAPPTHGALRLGAVMGAALLLSALGLSVWACMTCWR | |
| | RRAWRAVKSRASGKGPTYIRVADSELYADWSSDSEGE | |
| | RDQVPWLAPPERPDSPSTNGSGFEILSPTAPSVYPRSDG | |
| | HQSRRQLTTFGSGRPDRRYSQASDSSVFW | |
| HSV-2 gE | RTSWKRVTSGEDVVLLPAPAGPEERTRAHKLLWAAEP | 4 |
| (24-405) | LDACGPLRPSWVALWPPRRVLETVVDAACMRAPEPLA | |
| | IAYSPPFPAGDEGLYSELAWRDRVAVVNESLVIYGALE | |
| | TDSGLYTLSVVGLSDEARQVASVVLVVEPAPVPTPTPD | |
| | DYDEEDDAGVSERTPVSVPPPTPPRRPPVAPPTHPRVIP | |
| | EVSHVRGVTVHMETPEAILFAPGETFGTNVSIHAIAHD | |
| | DGPYAMDVVWMRFDVPSSCAEMRIYEACLYHPQLPEC | |
| | LSPADAPCAVSSWAYRLAVRSYAGCSRTTPPPRCFAEA | |
| | RMEPVPGLAWLASTVNLEFQHASPQHAGLYLCVVYV | |
| | DDHIHAWGHMTISTAAQYRNAVVEQHLPQRQPEPVEP | |
| | TRPHVRA | |
| HSV-2 gE | MARGAGLVFFVGVWVVSCLAAAPRTSWKRVTSGEDV | 5 |
| Full- | VLLPAPAERTRAHKLLWAAEPLDACGPLRPSWVALWP | |
| length | PRRVLETVVDAACMRAPEPLAIAYSPPFPAGDEGLYSE | |
| | LAWRDRVAVVNESLVIYGALETDSGLYTLSVVGLSDE | |
| | ARQVASVVLVVEPAPVPTPTPDDYDEEDDAGVTNARR | |
| | SAFPPQPPPRRPPVAPPTHPRVIPEVSHVRGVTVHMETL | |
| | EAILFAPGETFGTNVSIHAIAHDDGPYAMDVVWMRFD | |
| | VPSSCADMRIYEACLYHPQLPECLSPADAPCAVSSWAY | |
| | RLAVRSYAGCSRTTPPPRCFAEARMEPVPGLAWLASTV | |
| | NLEFQHASPQHAGLYLCVVYVDDHIHAWGHMTISTAA | |
| | QYRNAVVEQHLPQRQPEPVEPTRPHVRAPHPAPSARGP | |
| | LRLGAVLGAALLLAALGLSAWACMTCWRRRSWRAV | |
| | KSRASATGPTYIRVADSELYADWSSDSEGERDGSLWQ | |
| | DPPERPDSPSTNGSGFEILSPTAPSVYPHSEGRKSRRPLT | |
| | TFGSGSPGRRHSQASYPSVLW | |
| HSV-1 gC | ETASTGPTITAGAVTNASEAPTSGSPGSAASPEVTPTSTP | 6 |
| (27-457) | NPNNVTQNKTTPTEPASPPTTPKPTSTPKSPPTSTPDPKP | |
| | KNNTTPAKSGRPTKPPGPVWCDRRDPLARYGSRVQIRC | |
| | RFRNSTRMEFRLQIWRYSMGPSPPIAPAPDLEEVLTNIT | |
| | APPGGLLVYDSAPNLTDPHVLWAEGAGPGADPPLYSV | |
| | TGPLPTQRLIIGEVTPATQGMYYLAWGRMDSPHEYGT | |
| | WVRVRMFRPPSLTLQPHAVMEGQPFKATCTAAAYYPR | |
| | NPVEFDWFEDDRQVFNPGQIDTQTHEHPDGFTTVSTVT | |
| | SEAVGGQVPPRTFTCQMTWHRDSVTFSRRNATGLALV | |
| | LPRPTITMEFGVRHVVCTAGCVPEGVTFAWFLGDDPSP | |
| | AAKSAVTAQESCDHPGLATVRSTLPISYDYSEYICRLTG | |
| | YPAGIPVLEHH | |
| HSV-1 gC | GSETASTGPTITAGAVTNASEAPTSGSPGSAASPEVTPT | 7 |
| (25-457) | STPNPNNVTQNKTTPTEPASPPTTPKPTSTPKSPPTSTPD | |
| | PKPKNNTTPAKSGRPTKPPGPVWCDRRDPLARYGSRV | |
| | QIRCRFRNSTRMEFRLQIWRYSMGPSPPIAPAPDLEEVL | |
| | TNITAPPGGLLVYDSAPNLTDPHVLWAEGAGPGADPPL | |
| | YSVTGPLPTQRLIIGEVTPATQGMYYLAWGRMDSPHE | |
| | YGTWVRVRMFRPPSLTLQPHAVMEGQPFKATCTAAA | |
| | YYPRNPVEFDWFEDDRQVFNPGQIDTQTHEHPDGFTTV | |
| | STVTSEAVGGQVPPRTFTCQMTWHRDSVTFSRRNATG | |
| | LALVLPRPTITMEFGVRHVVCTAGCVPEGVTFAWFLG | |
| | DDPSPAAKSAVTAQESCDHPGLATVRSTLPISYDYSEYI | |
| | CRLTGYPAGIPVLEHH | |
| HSV-1 gC | MAPGRVGLAVVLWGLLWLGAGVAGGSETASTGPTIT | 8 |
| Full- | AGAVTNASEAPTSGSPGSAASPEVTPTSTPNPNNVTQN | |
| length | KTTPTEPASPPTTPKPTSTPKSPPTSTPDPKPKNNTTPAK | |
| | SGRPTKPPGPVWCDRRDPLARYGSRVQIRCRFRNSTRM | |
| | EFRLQIWRYSMGPSPPIAPAPDLEEVLTNITAPPGGLLV | |
| | YDSAPNLTDPHVLWAEGAGPGADPPLYSVTGPLPTQR | |
| | LIIGEVTPATQGMYYLAWGRMDSPHEYGTWVRVRMF | |
| | RPPSLTLQPHAVMEGQPFKATCTAAAYYPRNPVEFDW | |
| | FEDDRQVFNPGQIDTQTHEHPDGFTTVSTVTSEAVGGQ | |
| | VPPRTFTCQMTWHRDSVTFSRRNATGLALVLPRPTITM | |
| | EFGVRHVVCTAGCVPEGVTFAWFLGDDPSPAAKSAVT | |
| | AQESCDHPGLATVRSTLPISYDYSEYICRLTGYPAGIPV | |
| | LEHHGSHQPPPRDPTERQVIEAIEWVGIGIGVLAAGVLV | |
| | VTAIVYVVRTSQSRQRHRR | |
| HSV-2 gC | ASPGRTITVGPRGNASNAAPSASPRNASAPRTTPTPPQP | 9 |
| (27-426) | RKATKSKASTAKPAPPPKTGPPKTSSEPVRCNRHDPLA | |
| | RYGSRVQIRCRFPNSTRTESRLQIWRYATATDAEIGTAP | |
| | SLEEVMVNVSAPPGGQLVYDSAPNRTDPHVIWAEGAG | |
| | PGASPRLYSVVGPLGRQRLIIEELTLETQGMYYWVWG | |
| | RTDRPSAYGTWVRVRVFRPPSLTIHPHAVLEGQPFKAT | |
| | CTAATYYPGNRAEFVWFEDGRRVFDPAQIHTQTQENP | |
| | DGFSTVSTVTSAAVGGQGPPRTFTCQLTWHRDSVSFSR | |
| | RNASGTASVLPRPTITMEFTGDHAVCTAGCVPEGVTFA | |
| | WFLGDDSSPAEKVAVASQTSCGRPGTATIRSTLPVSYE | |
| | QTEYICRLAGYPDGIPVLEHH | |
| HSV-2 gC | ASPGRTITVGPRGNASNAAPSASPRNASAPRTTPTPPQP | 10 |
| S123F | RKATKSKASTAKPAPPPKTGPPKTSSEPVRCNRHDPLA | |
| Variant | RYGSRVQIRCRFPNSTRTEFRLQIWRYATATDAEIGTAP | |
| (27-426) | SLEEVMVNVSAPPGGQLVYDSAPNRTDPHVIWAEGAG | |
| | PGASPRLYSVVGPLGRQRLIIEELTLETQGMYYWVWG | |
| | RTDRPSAYGTWVRVRVFRPPSLTIHPHAVLEGQPFKAT | |
| | CTAATYYPGNRAEFVWFEDGRRVFDPAQIHTQTQENP | |
| | DGFSTVSTVTSAAVGGQGPPRTFTCQLTWHRDSVSFSR | |
| | RNASGTASVLPRPTITMEFTGDHAVCTAGCVPEGVTFA | |
| | WFLGDDSSPAEKVAVASQTSCGRPGTATIRSTLPVSYE | |
| | QTEYICRLAGYPDGIPVLEHH | |
| HSV-2 gC | SPGRTITVGPRGNASNAAPSASPRNASAPRTTPTPPQPR | 11 |
| (28-426) | KATKSKASTAKPAPPPKTGPPKTSSEPVRCNRHDPLAR | |
| | YGSRVQIRCRFPNSTRTESRLQIWRYATATDAEIGTAPS | |
| | LEEVMVNVSAPPGGQLVYDSAPNRTDPHVIWAEGAGP | |
| | GASPRLYSVVGPLGRQRLIIEELTLETQGMYYWVWGR | |
| | TDRPSAYGTWVRVRVFRPPSLTIHPHAVLEGQPFKATC | |
| | TAATYYPGNRAEFVWFEDGRRVFDPAQIHTQTQENPD | |
| | GFSTVSTVTSAAVGGQGPPRTFTCQLTWHRDSVSFSRR | |
| | NASGTASVLPRPTITMEFTGDHAVCTAGCVPEGVTFA | |
| | WFLGDDSSPAEKVAVASQTSCGRPGTATIRSTLPVSYE | |
| | QTEYICRLAGYPDGIPVLEHH | |
| HSV-2 gC | SPGRTITVGPRGNASNAAPSASPRNASAPRTTPTPPQPR | 12 |
| S123F | KATKSKASTAKPAPPPKTGPPKTSSEPVRCNRHDPLAR | |
| Variant | YGSRVQIRCRFPNSTRTEFRLQIWRYATATDAEIGTAPS | |
| (28-426) | LEEVMVNVSAPPGGQLVYDSAPNRTDPHVIWAEGAGP | |
| | GASPRLYSVVGPLGRQRLIIEELTLETQGMYYWVWGR | |
| | TDRPSAYGTWVRVRVFRPPSLTIHPHAVLEGQPFKATC | |
| | TAATYYPGNRAEFVWFEDGRRVFDPAQIHTQTQENPD | |
| | GFSTVSTVTSAAVGGQGPPRTFTCQLTWHRDSVSFSRR | |
| | NASGTASVLPRPTITMEFTGDHAVCTAGCVPEGVTFA | |
| | WFLGDDSSPAEKVAVASQTSCGRPGTATIRSTLPVSYE | |
| | QTEYICRLAGYPDGIPVLEHH | |
| HSV-2 gC | SASPGRTITVGPRGNASNAAPSASPRNASAPRTTPTPPQ | 13 |
| (26-426) | PRKATKSKASTAKPAPPPKTGPPKTSSEPVRCNRHDPL | |
| | ARYGSRVQIRCRFPNSTRTESRLQIWRYATATDAEIGTA | |
| | PSLEEVMVNVSAPPGGQLVYDSAPNRTDPHVIWAEGA | |
| | GPGASPRLYSVVGPLGRQRLIIEELTLETQGMYYWVW | |
| | GRTDRPSAYGTWVRVRVFRPPSLTIHPHAVLEGQPFKA | |
| | TCTAATYYPGNRAEFVWFEDGRRVFDPAQIHTQTQEN | |
| | PDGFSTVSTVTSAAVGGQGPPRTFTCQLTWHRDSVSFS | |
| | RRNASGTASVLPRPTITMEFTGDHAVCTAGCVPEGVTF | |
| | AWFLGDDSSPAEKVAVASQTSCGRPGTATIRSTLPVSY | |
| | EQTEYICRLAGYPDGIPVLEHH | |
| HSV-2 gC | SASPGRTITVGPRGNASNAAPSASPRNASAPRTTPTPPQ | 14 |
| S123F | PRKATKSKASTAKPAPPPKTGPPKTSSEPVRCNRHDPL | |
| Variant | ARYGSRVQIRCRFPNSTRTEFRLQIWRYATATDAEIGTA | |
| (26-426) | PSLEEVMVNVSAPPGGQLVYDSAPNRTDPHVIWAEGA | |
| | GPGASPRLYSVVGPLGRQRLIIEELTLETQGMYYWVW | |
| | GRTDRPSAYGTWVRVRVFRPPSLTIHPHAVLEGQPFKA | |
| | TCTAATYYPGNRAEFVWFEDGRRVFDPAQIHTQTQEN | |
| | PDGFSTVSTVTSAAVGGQGPPRTFTCQLTWHRDSVSFS | |
| | RRNASGTASVLPRPTITMEFTGDHAVCTAGCVPEGVTF | |
| | AWFLGDDSSPAEKVAVASQTSCGRPGTATIRSTLPVSY | |
| | EQTEYICRLAGYPDGIPVLEHH | |
| HSV-2 gC | MALGRVGLAVGLWGLLWVGVVVVLANASPGRTITVG | 15 |
| Full- | PRGNASNAAPSASPRNASAPRTTPTPPQPRKATKSKAST | |
| length | AKPAPPPKTGPPKTSSEPVRCNRHDPLARYGSRVQIRCR | |
| | FPNSTRTEFRLQIWRYATATDAEIGTAPSLEEVMVNVS | |
| | APPGGQLVYDSAPNRTDPHVIWAEGAGPGASPRLYSV | |
| | VGPLGRQRLIIEELTLETQGMYYWVWGRTDRPSAYGT | |
| | WVRVRVFRPPSLTIHPHAVLEGQPFKATCTAATYYPGN | |
| | RAEFVWFEDGRRVFDPAQIHTQTQENPDGFSTVSTVTS | |
| | AAVGGQGPPRTFTCQLTWHRDSVSFSRRNASGTASVLP | |
| | RPTITMEFTGDHAVCTAGCVPEGVTFAWFLGDDSSPAE | |
| | KVAVASQTSCGRPGTATIRSTLPVSYEQTEYICRLAGYP | |
| | DGIPVLEHHGSHQPPPRDPTERQVIRAVEGAGIGVAVL | |
| | VAVVLAGTAVVYLTHASSVRYRRLR | |
| HSV-1 | KYALADASLKMADPNRFRGKDLPVLDQLTDPPGVRRV | 16 |
| gD (26- | YHIQAGLPDPFQPPSLPITVYYAVLERACRSVLLNAPSE | |
| 331) | APQIVRGASEDVRKQPYNLTIAWFRMGGNCAIPITVME | |
| | YTECSYNKSLGACPIRTQPRWNYYDSFSAVSEDNLGFL | |
| | MHAPAFETAGTYLRLVKINDWTEITQFILEHRAKGSCK | |
| | YALPLRIPPSACLSPQAYQQGVTVDSIGMLPRFIPENQR | |
| | TVAVYSLKIAGWHGPKAPYTSTLLPPELSETPNATQPEL | |
| | APEDPEDSALLEDPVGTVAPQIPPNWHIPSIQDAATPY | |
| HSV-1 | MGGAAARLGAVILFVVIVGLHGVRGKYALADASLKLA | 17 |
| gD Full- | DPNRFRRKDLPVLDQLTDPPGVRRVYHIQAGLPDPFQP | |
| length | PSLPITVYYAVLERACRSVLLNAPSEAPQIVRGASEDVR | |
| | KQPYNLTIAWFRMGGNCAIPITVMEYTECSYNKSLGAC | |
| | PIRTQPRWNYYDSFSAVSEDNLGFLMHAPAFETAGTYL | |
| | RLVKINDWTEITQFILEHRAKGSCKYALPLRIPPSACLSP | |
| | QAYQQGVTVDSIGMLPRFIPENQRTVAVYSLKIAGWH | |
| | GPKAPYTSTLLPPELSETPNATQPELAPEAPEDSALLED | |
| | PVGTVAPQIPPNWHIPSIQDAATPYHPPATPNNMGLIAG | |
| | AVGGSLLAALVICGIVYWMRRRTQKAPKRIRLPHIRED | |
| | DQPSSHQPLFY | |
| HSV-2 | KYALADPSLKMADPNRFRGKNLPVLDQLTDPPGVKRV | 18 |
| gD (26- | YHIQPSLEDPFQPPSIPITVYYAVLERACRSVLLHAPSEA | |
| 331) | PQIVRGASDEARKHTYNLTIAWYRMGDNCAIPITVMEY | |
| | TECPYNKSLGVCPIRTQPRWSYYDSFSAVSEDNLGFLM | |
| | HAPAFETAGTYLRLVKINDWTEITQFILEHRARASCKY | |
| | ALPLRIPPAACLTSKAYQQGVTVDSIGMLPRFIPENQRT | |
| | VALYSLKIAGWHGPKPPYTSTLLPPELSDTTNATQPELV | |
| | PEDPEDSALLEDPAGTVSSQIPPNWHIPSIQDVAPHH | |
| HSV-2 | ADPSLKMADPNRFRGKNLPVLDQLTDPPGVKRVYHIQ | 19 |
| gD | PSLEDPFQPPSIPITVYYAVLERACRSVLLHAPSEAPQIV | |
| (30-331) | RGASDEARKHTYNLTIAWYRMGDNCAIPITVMEYTEC | |
| | PYNKSLGVCPIRTQPRWSYYDSFSAVSEDNLGFLMHAP | |
| | AFETAGTYLRLVKINDWTEITQFILEHRARASCKYALPL | |
| | RIPPAACLTSKAYQQGVTVDSIGMLPRFIPENQRTVALY | |
| | SLKIAGWHGPKPPYTSTLLPPELSDTTNATQPELVPEDP | |
| | EDSALLEDPAGTVSSQIPPNWHIPSIQDVAPHH | |
| HSV-2 | DPSLKMADPNRFRGKNLPVLDQLTDPPGVKRVYHIQPS | 20 |
| gD | LEDPFQPPSIPITVYYAVLERACRSVLLHAPSEAPQIVRG | |
| (31-331) | ASDEARKHTYNLTIAWYRMGDNCAIPITVMEYTECPY | |
| | NKSLGVCPIRTQPRWSYYDSFSAVSEDNLGFLMHAPAF | |
| | ETAGTYLRLVKINDWTEITQFILEHRARASCKYALPLRI | |
| | PPAACLTSKAYQQGVTVDSIGMLPRFIPENQRTVALYS | |
| | LKIAGWHGPKPPYTSTLLPPELSDTTNATQPELVPEDPE | |
| | DSALLEDPAGTVSSQIPPNWHIPSIQDVAPHH | |
| HSV-2 | MGRLTSGVGTAALLVVAVGLRVVCAKYALADPSLKM | 21 |
| gD Full- | ADPNRFRGKNLPVLDQLTDPPGVKRVYHIQPSLEDPFQ | |
| length | PPSIPITVYYAVLERACRSVLLHAPSEAPQIVRGASDEA | |
| | RKHTYNLTIAWYRMGDNCAIPITVMEYTECPYNKSLG | |
| | VCPIRTQPRWSYYDSFSAVSEDNLGFLMHAPAFETAGT | |
| | YLRLVKINDWTEITQFILEHRARASCKYALPLRIPPAAC | |
| | LTSKAYQQGVTVDSIGMLPRFIPENQRTVALYSLKIAG | |
| | WHGPKPPYTSTLLPPELSDTTNATQPELVPEDPEDSALL | |
| | EDPAGTVSSQIPPNWHIPSIQDVAPHHAPAAPSNPGLIIG | |
| | ALAGSTLAVL VIGGIAFWVRRRAQMAPKRLRLPHIRDD | |
| | DAPPSHQPLFY | |

In some embodiments, an RNA of the present disclosure comprises a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 2.

In some embodiments, methods of the present disclosure comprise administering to a subject an RNA comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 2.

TABLE 2
Exemplary nucleic acid sequences of HSV immunogens

| Sequence | | |
|---|---|---|
| Name | Nucleic Acid Sequence | SEQ ID NO: |
| HSV-1 gE | AAGACCUCCUGGCGCCGCGUGUCCGUGGGCGAGGA | 22 |
| (24-409) | CGUGUCCCUGCUGCCCGCCCCCGGCCCCACCGGCCG | |
| | CGGCCCCACCCAGAAGCUGCUGUGGGCCGUGGAGC | |
| | CCCUGGACGGCUGCGGCCCCCUGCACCCCUCCUGGG | |
| | UGUCCCUGAUGCCCCCCAAGCAGGUGCCCGAGACC | |
| | GUGGUGGACGCCGCCUGCAUGCGCGCCCCCGUGCC | |
| | CCUGGCCAUGGCCUACGCCCCCCCCGCCCCCUCCGC | |
| | CACCGGCGGCCUGCGCACCGACUUCGUGUGGCAGG | |
| | AGCGCGCCGCCGUGGUGAACCGCUCCCUGGUGAUC | |
| | UACGGCGUGCGCGAGACCGACUCCGGCCUGUACAC | |
| | CCUGUCCGUGGGCGACAUCAAGGACCCCGCCCGCC | |
| | AGGUGGCCUCCGUGGUGCUGGUGGUGCAGCCCGCC | |
| | CCCGUGCCCACCCCCCCCCCCACCCCCGCCGACUAC | |
| | GACGAGGACGACAACGACGAGGGCGAGGGCGAGGA | |
| | CGAGUCCCUGGCCGGCACCCCCGCCUCCGGCACCCC | |
| | CCGCCUGCCCCCCUCCCCCGCCCCCCCCCGCUCCUG | |
| | GCCCUCCGCCCCCGAGGUGUCCCACGUGCGCGGCGU | |
| | GACCGUGCGCAUGGAGACCCCCGAGGCCAUCCUGU | |
| | UCUCCCCCGGCGAGGCCUUCUCCACCAACGUGUCCA | |
| | UCCACGCCAUCGCCCACGACGACCAGACCUACACCA | |
| | UGGACGUGGUGUGGCUGCGCUUCGACGUGCCCACC | |
| | UCCUGCGCCGAGAUGCGCAUCUACGAGUCCUGCCU | |
| | GUACCACCCCCAGCUGCCCGAGUGCCUGUCCCCCGC | |
| | CGACGCCCCCUGCGCCGCCUCCACCUGGACCUCCCG | |
| | CCUGGCCGUGCGCUCCUACGCCGGCUGCUCCCGCAC | |
| | CAACCCCCCCCCCCGCUGCUCCGCCGAGGCCCACAU | |
| | GGAGCCCUUCCCCGGCCUGGCCUGGCAGGCCGCCUC | |
| | CGUGAACCUGGAGUUCCGCGACGCCUCCCCCCAGC | |
| | ACUCCGGCCUGUACCUGUGCGUGGUGUACGUGAAC | |
| | GACCACAUCCACGCCUGGGGCCACAUCACCAUCAA | |
| | CACCGCCGCCCAGUACCGCAACGCCGUGGUGGAGC | |
| | AGCCCCUGCCCCAGCGCGGCGCCGACCUGGCCGAGC | |
| | CCACCCACCCCCACGUGGGCGCCUAA | |
| HSV-2 gE | CGCACCUCCUGGAAGCGCGUGACCUCCGGCGAGGA | 23 |
| [US8] | CGUGGUGCUGCUGCCCGCCCCCGCCGGCCCCGAGGA | |
| (24-405) | GCGCACCCGCGCCCACAAGCUGCUGUGGGCCGCCG | |
| WT | AGCCCCUGGACGCCUGCGGCCCCCUGCGCCCCUCCU | |
| | GGGUGGCCCUGUGGCCCCCCCGCCGCGUGCUGGAG | |
| | ACCGUGGUGGACGCCGCCUGCAUGCGCGCCCCCGA | |
| | GCCCCUGGCCAUCGCCUACUCCCCCCCCUUCCCCGC | |
| | CGGCGACGAGGGCCUGUACUCCGAGCUGGCCUGGC | |
| | GCGACCGCGUGGCCGUGGUGAACGAGUCCCUGGUG | |
| | AUCUACGGCGCCCUGGAGACCGACUCCGGCCUGUA | |
| | CACCCUGUCCGUGGUGGGCCUGUCCGACGAGGCCC | |
| | GCCAGGUGGCCUCCGUGGUGCUGGUGGUGGAGCCC | |
| | GCCCCCGUGCCCACCCCCACCCCCGACGACUACGAC | |
| | GAGGAGGACGACGCCGGCGUGUCCGAGCGCACCCC | |
| | CGUGUCCGUGCCCCCCCCCACCCCCCCCCGCCGCCC | |
| | CCCCGUGGCCCCCCCCACCCACCCCCGCGUGAUCCC | |
| | CGAGGUGUCCCACGUGCGCGGCGUGACCGUGCACA | |
| | UGGAGACCCCCGAGGCCAUCCUGUUCGCCCCCGGC | |
| | GAGACCUUCGGCACCAACGUGUCCAUCCACGCCAU | |
| | CGCCCACGACGACGGCCCCUACGCCAUGGACGUGG | |
| | UGUGGAUGCGCUUCGACGUGCCCUCCUCCUGCGCC | |
| | GAGAUGCGCAUCUACGAGGCCUGCCUGUACCACCC | |
| | CCAGCUGCCCGAGUGCCUGUCCCCCGCCGACGCCCC | |
| | CUGCGCCGUGUCCUCCUGGGCCUACCGCCUGGCCG | |
| | UGCGCUCCUACGCCGGCUGCUCCCGCACCACCCCCC | |
| | CCCCCCGCUGCUUCGCCGAGGCCCGCAUGGAGCCCG | |
| | UGCCCGGCCUGGCCUGGCUGGCCUCCACCGUGAAC | |
| | CUGGAGUUCCAGCACGCCUCCCCCCAGCACGCCGGC | |
| | CUGUACCUGUGCGUGGUGUACGUGGACGACCACAU | |
| | CCACGCCUGGGGCCACAUGACCAUCUCCACCGCCGC | |
| | CCAGUACCGCAACGCCGUGGUGGAGCAGCACCUGC | |
| | CCCAGCGCCAGCCCGAGCCCGUGGAGCCCACCCGCC | |
| | CCCACGUGCGCGCCUAA | |
| HSV-2 gE | AGAACCAGCUGGAAAAGAGUGACCAGCGGCGAGGA | 24 |
| [US8] | UGUGGUGCUGCUUCCUGCUCCUGCUGGCCCCGAGG | |
| (24-405) | AAAGAACAAGAGCCCACAAACUGCUGUGGGCCGCU | |
| Version 1 | GAGCCUCUUGAUGCCUGUGGACCUCUCAGACCUAG | |
| | CUGGGUUGCACUGUGGCCACCUCGGAGAGUGCUGG | |
| | AAACAGUGGUGGAUGCCGCCUGCAUGAGAGCCCCU | |
| | GAACCUCUGGCCAUUGCCUACUCUCCACCAUUUCC | |
| | AGCCGGCGACGAGGGCCUGUAUUCUGAGCUUGCUU | |
| | GGAGAGACAGAGUGGCCGUGGUCAACGAGAGCCUG | |
| | GUUAUCUAUGGCGCCCUGGAAACCGACAGCGGCCU | |
| | GUACACACUGUCUGUCGUGGGCCUGUCUGACGAGG | |
| | CUAGACAGGUGGCAUCUGUGGUCCUGGUGGUGGAA | |
| | CCUGCUCCAGUGCCUACACCUACACCUGACGACUA | |
| | CGACGAGGAAGAUGACGCUGGCGUCAGCGAGAGAA | |
| | CCCCUGUUUCUGUGCCUCCUCCUACGCCUCCUCGUA | |
| | GACCUCCUGUUGCUCCUCCAACACACCCCAGAGUG | |
| | AUCCCUGAAGUGUCUCACGUGCGGGGCGUGACCGU | |
| | GCACAUGGAAACACCUGAGGCCAUCCUGUUCGCCC | |
| | CUGGCGAGACAUUUGGCACCAACGUGUCCAUCCAC | |
| | GCUAUCGCCCACGACGAUGGCCCUUACGCCAUGGA | |
| | UGUCGUGUGGAUGAGAUUCGACGUGCCCAGCAGCU | |
| | GUGCCGAGAUGAGAAUCUAUGAGGCCUGCCUGUAU | |
| | CACCCUCAGCUGCCCGAAUGUCUGAGCCCUGCUGA | |
| | UGCCCCUUGUGCCGUUAGCAGCUGGGCCUAUAGAC | |
| | UGGCCGUGCGGUCUUAUGCCGGCUGCUCUAGAACA | |
| | ACCCCUCCUCCUCGGUGUUUCGCCGAGGCCAGAAU | |
| | GGAACCUGUUCCUGGACUGGCCUGGCUGGCCUCCA | |
| | CAGUGAACCUGGAAUUUCAGCACGCCUCUCCACAG | |
| | CACGCCGGCCUGUAUCUGUGUGUGGUGUACGUGGA | |
| | CGAUCACAUCCACGCCUGGGGCCACAUGACCAUCU | |
| | CUACAGCCGCUCAGUACCGGAACGCCGUGGUUGAA | |
| | CAGCAUCUGCCUCAGAGACAGCCCGAGCCUGUGGA | |
| | ACCUACAAGACCUCAUGUUCGGGCCUGA | |
| HSV-2 gE | AGAACCUCUUGGAAGCGCGUGACAAGCGGCGAGGA | 25 |
| [US8] | UGUGGUUCUGCUUCCUGCUCCUGCCGGACCUGAGG | |
| (24-405) | AAAGAACAAGAGCCCACAAGCUGCUGUGGGCCGCU | |
| Version | GAACCUUUGGAUGCCUGUGGACCUCUGAGGCCUUC | |
| 1.1 | UUGGGUUGCACUGUGGCCACCUCGGAGAGUGCUGG | |
| | AAACAGUGGUGGAUGCCGCCUGCAUGAGAGCCCCU | |
| | GAACCUCUGGCCAUUGCCUACUCUCCACCUUUUCC | |
| | AGCCGGCGACGAGGGCCUGUAUUCUGAGCUUGCUU | |
| | GGAGAGACAGAGUGGCCGUGGUCAACGAGAGCCUG | |
| | GUUAUCUAUGGCGCCCUGGAAACCGACAGCGGCCU | |
| | GUACACACUGUCUGUCGUGGGCCUGUCUGACGAGG | |
| | CUAGACAGGUGGCAUCUGUGGUGCUGGUGGUGGAA | |
| | CCUGCUCCAGUGCCUACACCUACACCUGACGACUA | |
| | CGACGAGGAAGAUGACGCUGGCGUCAGCGAGAGAA | |
| | CCCCUGUUUCUGUGCCUCCUCCUACGCCUCCUCGUA | |
| | GACCUCCUGUUGCUCCUCCAACACACCCCAGAGUG | |
| | AUCCCUGAAGUGUCUCACGUGCGGGGCGUGACCGU | |
| | GCACAUGGAAACACCUGAGGCCAUCCUGUUCGCCC | |
| | CUGGCGAGACAUUUGGCACCAACGUGUCCAUCCAC | |
| | GCUAUCGCCCACGACGAUGGCCCUUACGCCAUGGA | |
| | UGUCGUGUGGAUGAGAUUCGACGUGCCCAGCAGCU | |
| | GCGCCGAGAUGAGAAUCUACGAGGCCUGCCUGUAU | |
| | CACCCUCAGCUGCCCGAAUGUCUGAGCCCUGCUGA | |
| | UGCCCCUUGUGCCGUUAGCAGCUGGGCCUAUAGAC | |
| | UGGCCGUGCGGUCUUAUGCCGGCUGCUCUAGAACA | |
| | ACCCCUCCUCCUCGGUGUUUCGCCGAGGCCAGAAU | |
| | GGAACCUGUUCCUGGACUGGCCUGGCUGGCCUCCA | |
| | CAGUGAACCUGGAAUUUCAGCACGCCUCUCCACAG | |
| | CACGCCGGCCUGUAUCUGUGUGUGGUGUACGUGGA | |
| | CGAUCACAUCCACGCCUGGGGCCACAUGACCAUCU | |
| | CUACAGCCGCUCAGUACCGGAACGCCGUGGUUGAA | |
| | CAGCAUCUGCCCCAGAGACAGCCCGAGCCUGUGGA | |
| | ACCUACAAGACCUCAUGUUCGGGCCUGAUAA | |
| HSV-2 gE | CGCACCUCUUGGAAACGCGUUACUUCCGGGGAGGA | 26 |
| [US8] | CGUUGUCCUCCUUCCAGCACCCGCAGGACCUGAGG | |
| (24-405) | AAAGGACUAGGGCCCACAAGCUGCUGUGGGCCGCU | |
| Version 2 | GAACCUCUGGAUGCCUGUGGUCCUCUGAGACCUAG | |
| | CUGGGUCGCCCUUUGGCCACCUAGACGCGUUCUGG | |
| | AGACGGUCGUGGAUGCCGCGUGCAUGCGUGCACCC | |
| | GAACCUCUGGCCAUCGCCUAUAGUCCCCCUUUUCC | |
| | CGCUGGCGACGAGGGGCUUUACUCCGAACUGGCCU | |
| | GGCGGGAUAGGGUGGCGGUGGUGAACGAGAGCCUC | |
| | GUCAUCUACGGUGCUCUGGAAACCGACUCAGGACU | |
| | GUAUACGCUCAGCGUUGUUGGCCUCUCCGAUGAGG | |
| | CUCGACAGGUUGCCUCCGUAGUGCUGGUCGUAGAA | |
| | CCAGCCCCCGUACCAACACCCACACCCGACGACUAC | |
| | GACGAGGAGGACGACGCUGGAGUUAGCGAAAGAAC | |
| | ACCGGUGAGUGUGCCACCUCCCACACCGCCAAGGA | |
| | GACCCCCAGUAGCACCUCCAACCCAUCCGAGAGUG | |
| | AUUCCCGAGGUCAGCCAUGUGCGCGGCGUAACUGU | |
| | GCACAUGGAGACGCCCGAAGCGAUACUGUUUGCCC | |
| | CUGGAGAGACAUUCGGCACCAAUGUGUCCAUACAC | |
| | GCAAUUGCGCACGAUGAUGGCCCAUACGCUAUGGA | |
| | CGUCGUCUGGAUGAGGUUCGAUGUGCCUUCUUCUU | |
| | GCGCCGAGAUGAGGAUCUACGAGGCAUGCCUGUAU | |
| | CACCCCCAAUUGCCGGAGUGUCUGUCUCCCGCAGA | |
| | UGCACCGUGUGCAGUGAGUAGCUGGGCUUAUCGGU | |
| | UGGCUGUCCGGAGUUAUGCUGGGUGUUCACGGACC | |
| | ACCCCACCUCCACGUUGCUUUGCUGAAGCCAGAAU | |
| | GGAACCCGUGCCUGGUCUGGCUUGGCUGGCAUCAA | |
| | CUGUCAACCUGGAGUUCCAGCAUGCCUCUCCACAG | |
| | CACGCAGGCCUGUAUCUCUGCGUGGUGUACGUUGA | |
| | CGAUCACAUCCAUGCGUGGGGGCAUAUGACCAUCA | |
| | GCACAGCUGCCCAGUACCGCAAUGCCGUCGUGGAG | |
| | CAGCACCUCCCCCAACGGCAGCCAGAACCAGUGGA | |
| | GCCCACUCGGCCUCAUGUGCGAGCCUGA | |
| HSV-2 gE | CGCACCUCUUGGAAACGCGUUACUUCCGGGGAGGA | 27 |
| [US8] | CGUUGUCCUCCUUCCAGCACCCGCAGGACCUGAAG | |
| (24-405) | AGAGGACUAGGGCCCACAAGCUGCUGUGGGCCGCU | |
| Version 2 | GAACCUCUGGAUGCCUGUGGUCCUCUGAGACCUAG | |
| | CUGGGUCGCCCUUUGGCCACCUAGACGCGUUCUGG | |
| | AGACGGUCGUGGAUGCCGCGUGCAUGCGUGCACCC | |
| | GAACCUCUGGCCAUCGCCUAUAGUCCCCCUUUUCC | |
| | CGCUGGCGACGAGGGGCUUUACUCCGAACUGGCCU | |
| | GGCGGGAUAGGGUGGCGGUGGUGAACGAGAGCCUC | |
| | GUCAUCUACGGUGCUCUGGAAACCGACUCAGGACU | |
| | GUAUACGCUCAGCGUUGUUGGCCUCUCCGAUGAGG | |
| | CUCGACAGGUUGCCUCCGUAGUGCUGGUCGUAGAA | |
| | CCAGCCCCCGUACCAACACCCACACCCGACGACUAC | |
| | GACGAAGAGGACGACGCUGGAGUUAGCGAAAGAAC | |
| | ACCGGUGAGUGUGCCACCUCCCACACCGCCAAGGA | |
| | GACCCCCAGUAGCACCUCCAACCCAUCCGAGAGUG | |
| | AUUCCCGAGGUCAGCCAUGUGCGCGGCGUAACUGU | |
| | GCACAUGGAGACGCCCGAAGCGAUACUGUUUGCCC | |
| | CUGGAGAGACAUUCGGCACCAAUGUGUCCAUACAC | |
| | GCAAUUGCGCACGAUGAUGGCCCAUACGCUAUGGA | |
| | CGUCGUCUGGAUGAGGUUCGAUGUGCCUUCUUCUU | |
| | GCGCCGAGAUGAGGAUCUACGAGGCAUGCCUGUAU | |
| | CACCCCCAAUUGCCGGAGUGUCUGUCUCCCGCAGA | |
| | UGCACCGUGUGCAGUGAGUAGCUGGGCUUAUCGGU | |
| | UGGCUGUCCGGAGUUAUGCUGGGUGUUCACGGACC | |
| | ACCCCACCUCCACGUUGCUUUGCUGAAGCCAGAAU | |
| | GGAACCCGUGCCUGGUCUGGCUUGGCUGGCAUCAA | |
| | CUGUCAACCUGGAGUUCCAGCAUGCCUCUCCACAG | |
| | CACGCAGGCCUGUAUCUCUGCGUGGUGUACGUUGA | |
| | CGAUCACAUCCAUGCGUGGGGGCAUAUGACCAUCA | |
| | GCACAGCUGCCCAGUACCGCAAUGCCGUCGUGGAG | |
| | CAGCACCUCCCCCAACGGCAGCCAGAACCAGUGGA | |
| | GCCCACUCGGCCUCAUGUGCGAGCCUGA | |
| HSV-2 gE | CGCACCUCUUGGAAACGCGUUACUUCCGGGGAGGA | 28 |
| [US8] | CGUUGUCCUCCUUCCAGCACCCGCAGGACCUGAGG | |
| (24-405) | AAAGGACUAGGGCCCACAAGCUGCUGUGGGCCGCU | |
| Version | GAACCUCUGGAUGCCUGUGGUCCUCUGAGACCUAG | |
| 2.2 | CUGGGUCGCCCUUUGGCCACCUAGACGCGUUCUGG | |
| | AGACGGUCGUGGAUGCCGCGUGCAUGCGUGCACCC | |
| | GAACCUCUGGCCAUCGCCUAUAGUCCCCCUUUUCC | |
| | CGCUGGCGACGAGGGGCUUUACUCCGAACUGGCCU | |
| | GGCGGGAUAGGGUGGCGGUGGUGAACGAGAGCCUC | |
| | GUCAUCUACGGUGCUCUGGAAACCGACUCAGGACU | |
| | GUAUACGCUCAGCGUUGUUGGCCUCUCCGAUGAGG | |
| | CUCGACAGGUUGCCUCCGUAGUGCUGGUCGUAGAA | |
| | CCAGCCCCCGUACCAACACCCACACCCGACGACUAC | |
| | GACGAGGAGGACGACGCUGGAGUUAGCGAAAGAAC | |
| | ACCGGUGAGUGUGCCACCUCCCACACCGCCAAGGA | |
| | GACCCCCAGUAGCACCUCCAACCCAUCCGAGAGUG | |
| | AUUCCCGAGGUCAGCCAUGUGCGCGGCGUAACUGU | |
| | GCACAUGGAGACGCCCGAAGCGAUACUGUUUGCCC | |
| | CUGGAGAGACAUUCGGCACCAAUGUGUCCAUACAC | |
| | GCAAUUGCGCACGAUGAUGGCCCAUACGCUAUGGA | |
| | CGUCGUCUGGAUGAGGUUCGAUGUGCCUUCUUCUU | |
| | GCGCCGAGAUGAGGAUCUACGAGGCAUGCCUGUAU | |
| | CACCCCCAAUUGCCGGAGUGUCUGUCUCCCGCAGA | |
| | UGCACCGUGUGCAGUGAGUAGCUGGGCUUAUCGGU | |
| | UGGCUGUCCGGAGUUAUGCUGGGUGUUCACGGACC | |
| | ACCCCACCUCCACGUUGCUUUGCUGAAGCCAGAAU | |
| | GGAACCCGUGCCUGGUCUGGCUUGGCUGGCAUCAA | |
| | CUGUCAACCUGGAGUUCCAGCAUGCCUCUCCACAG | |
| | CACGCAGGCCUGUAUCUCUGCGUGGUGUACGUUGA | |
| | CGAUCACAUCCAUGCGUGGGGGCAUAUGACCAUCA | |
| | GCACAGCUGCCCAGUACCGCAAUGCCGUCGUGGAG | |
| | CAGCACCUCCCCCAACGGCAGCCAGAACCAGUGGA | |
| | GCCCACUCGGCCUCAUGUGCGAGCCUGAUAA | |
| HSV-2 gE | AGAACCUCCUGGAAAAGAGUGACCUCCGGCGAAGA | 29 |
| [US8] | UGUGGUGCUGCUGCCCGCCCCCGCCGGCCCCGAAG | |
| (24-405) | AAAGAACCAGAGCCCACAAACUGCUGUGGGCCGCC | |
| Version 3 | GAACCCCUGGAUGCCUGCGGCCCCCUCAGACCCUCC | |
| | UGGGUGGCCCUGUGGCCCCCUAGAAGGGUGCUGGA | |
| | AACCGUGGUGGAUGCCGCCUGCAUGAGAGCCCCCG | |
| | AACCCCUGGCCAUCGCCUACUCCCCUCCCUUCCCCG | |
| | CCGGCGAUGAAGGCCUGUACUCCGAACUGGCCUGG | |
| | AGAGAUAGAGUGGCCGUGGUGAACGAAUCCCUGGU | |
| | GAUCUACGGCGCCCUGGAAACCGAUUCCGGCCUGU | |
| | ACACCCUGUCCGUGGUGGGCCUGUCCGAUGAAGCC | |
| | AGACAGGUGGCCUCCGUGGUGCUGGUGGUGGAACC | |
| | CGCCCCCGUGCCCACCCCCACCCCCGAUGAUUACGA | |
| | UGAAGAAGAUGAUGCCGGCGUGUCCGAAAGAACCC | |
| | CCGUGUCCGUGCCCCCUCCCACCCCUCCCCGCAGAC | |
| | CCCCUGUGGCCCCUCCCACCCACCCCAGAGUGAUCC | |
| | CCGAAGUGUCCCACGUGAGAGGCGUGACCGUGCAC | |
| | AUGGAAACCCCCGAAGCCAUCCUGUUCGCCCCCGG | |
| | CGAAACCUUCGGCACCAACGUGUCCAUCCACGCCA | |
| | UCGCCCACGAUGAUGGCCCCUACGCCAUGGAUGUG | |
| | GUGUGGAUGAGAUUCGAUGUGCCCUCCUCCUGCGC | |
| | CGAAAUGAGAAUCUACGAAGCCUGCCUGUACCACC | |
| | CCCAGCUGCCCGAAUGCCUGUCCCCCGCCGAUGCCC | |
| | CCUGCGCCGUGUCCUCCUGGGCCUACAGACUGGCC | |
| | GUGAGAUCCUACGCCGGCUGCUCCAGAACCACCCC | |
| | UCCCCCUAGAUGCUUCGCCGAAGCCAGAAUGGAAC | |
| | CCGUGCCCGGCCUGGCCUGGCUGGCCUCCACCGUG | |
| | AACCUGGAAUUCCAGCACGCCAGCCCCCAGCACGCC | |
| | GGCCUGUACCUGUGCGUGGUGUACGUGGAUGAUCA | |
| | CAUCCACGCCUGGGGCCACAUGACCAUCUCCACCGC | |
| | CGCCCAGUACAGAAACGCCGUGGUGGAACAGCACC | |
| | UGCCCCAGAGACAGCCCGAACCCGUGGAACCCACC | |
| | AGACCCCACGUGAGAGCCUGA | |
| HSV-2 gE | AGAACCUCUUGGAAAAGAGUGACCUCUGGAGAAGA | 30 |
| [US8] | UGUGGUGCUGCUGCCAGCUCCAGCUGGACCAGAAG | |
| (24-405) | AAAGAACCAGAGCACACAAACUGCUGUGGGCUGCU | |
| Version | GAACCUCUGGAUGCUUGUGGACCUCUGAGACCUUC | |
| 3.1 | UUGGGUGGCUCUGUGGCCACCAAGAAGGGUGCUGG | |
| | AAACAGUGGUGGAUGCUGCUUGCAUGAGAGCACCU | |
| | GAACCUCUGGCAAUUGCAUACUCUCCUCCUUUUCC | |
| | AGCUGGAGAUGAAGGACUGUAUUCUGAACUGGCUU | |
| | GGAGAGACAGAGUGGCUGUGGUGAAUGAAUCUCUG | |
| | GUGAUCUAUGGAGCACUGGAAACAGAUUCUGGACU | |
| | GUACACCCUGUCUGUGGUGGGACUGUCUGAUGAAG | |
| | CAAGACAGGUGGCAUCUGUGGUGCUGGUGGUGGAA | |
| | CCAGCUCCAGUGCCAACCCCAACCCCAGAUGAUUA | |
| | UGAUGAAGAAGAUGAUGCUGGAGUGUCUGAAAGA | |
| | ACCCCAGUGUCUGUGCCACCACCAACCCCACCAAGA | |
| | AGACCACCAGUGGCUCCACCAACCCACCCAAGAGU | |
| | GAUCCCAGAAGUGUCUCAUGUGAGAGGAGUGACAG | |
| | UGCACAUGGAAACCCCUGAAGCAAUCCUGUUUGCA | |
| | CCUGGAGAAACCUUUGGAACCAAUGUGUCCAUCCA | |
| | UGCAAUUGCACAUGAUGAUGGACCUUAUGCAAUGG | |
| | AUGUGGUGUGGAUGAGAUUUGAUGUGCCUUCUUCU | |
| | UGUGCUGAAAUGAGAAUCUAUGAAGCUUGCCUGUA | |
| | CCACCCUCAGCUGCCUGAAUGCCUGUCUCCUGCUG | |
| | AUGCUCCUUGUGCUGUGUCUUCUUGGGCUUACAGA | |
| | CUGGCUGUGAGAUCUUAUGCUGGAUGCUCCAGAAC | |
| | CACCCCACCACCAAGAUGCUUUGCUGAAGCAAGAA | |
| | UGGAACCUGUGCCUGGACUGGCAUGGCUGGCAUCC | |
| | ACAGUGAAUCUGGAAUUUCAGCAUGCUUCUCCUCA | |
| | GCAUGCUGGACUGUACCUGUGUGUGGUGUAUGUGG | |
| | ACGAUCACAUCCAUGCUUGGGGACACAUGACCAUC | |
| | AGCACAGCUGCUCAGUACAGAAAUGCUGUGGUGGA | |
| | ACAGCACCUGCCACAGAGACAGCCAGAACCAGUGG | |
| | AACCAACCAGACCACAUGUGAGAGCUUGAUAA | |
| HSV-2 gE | CGGACCAGCUGGAAAAGGGUGACAUCUGGGGAAGA | 31 |
| [US8] | UGUCGUGCUCCUUCCUGCGCCUGCUGGCCCAGAAG | |
| (24-405) | AACGCACUAGGGCCCACAAGCUUCUGUGGGCCGCG | |
| Version 4 | GAGCCACUGGAUGCGUGUGGUCCCCUGCGACCCUC | |
| | UUGGGUGGCAUUGUGGCCACCCCGACGCGUACUCG | |
| | AAACGGUCGUUGACGCCGCCUGUAUGAGAGCGCCA | |
| | GAGCCCCUCGCCAUUGCCUACAGCCCGCCUUUUCCC | |
| | GCCGGUGACGAAGGACUGUACAGUGAGCUGGCCUG | |
| | GCGCGAUAGGGUCGCCGUAGUUAACGAGUCCCUGG | |
| | UGAUAUACGGCGCUCUGGAAACCGACAGUGGCUUG | |
| | UACACCCUGUCCGUUGUAGGCCUGUCCGAUGAAGC | |
| | ACGGCAAGUGGCUUCCGUGGUACUGGUCGUAGAGC | |
| | CCGCACCAGUUCCCACACCCACCCCGGAUGACUACG | |
| | AUGAGGAGGACGAUGCCGGUGUGAGUGAGCGUACA | |
| | CCUGUUAGCGUACCUCCACCAACUCCUCCACGCCGC | |
| | CCUCCUGUUGCACCGCCUACACAUCCCCGUGUGAU | |
| | UCCUGAAGUGUCACACGUUAGAGGGGUGACAGUCC | |
| | ACAUGGAGACUCCCGAAGCCAUCCUCUUUGCACCA | |
| | GGCGAGACUUUUGGGACCAAUGUGAGCAUUCACGC | |
| | CAUAGCUCACGAUGACGGGCCCUAUGCCAUGGACG | |
| | UGGUGUGGAUGAGGUUCGAUGUGCCCUCAUCAUGC | |
| | GCUGAGAUGCGGAUCUACGAAGCUUGCCUGUAUCA | |
| | CCCACAGCUUCCCGAGUGCUUGUCUCCCGCUGACG | |
| | CCCCUUGUGCUGUUAGCUCUUGGGCUUAUCGGCUU | |
| | GCCGUCAGGAGCUAUGCUGGAUGCUCCAGAACCAC | |
| | ACCUCCACCGAGGUGUUUCGCCGAGGCCAGAAUGG | |
| | AGCCUGUGCCAGGACUGGCCUGGCUGGCAAGUACU | |
| | GUGAACCUGGAAUUCCAGCACGCAUCACCUCAGCA | |
| | UGCAGGCCUGUACCUCUGCGUUGUCUAUGUCGACG | |
| | ACCAUAUCCACGCAUGGGGCCAUAUGACCAUCAGC | |
| | ACGGCAGCACAGUAUCGGAAUGCUGUGGUCGAGCA | |
| | GCACUUGCCCCAGCGACAACCCGAACCAGUGGAGC | |
| | CAACCAGACCGCAUGUGCGGGCCUGA | |
| HSV-1 gC | GAGACCGCCUCCACCGGCCCCACCAUCACCGCCGGC | 32 |
| (27-457) | GCCGUGACCAACGCCUCCGAGGCCCCCACCUCCGGC | |
| WT | UCCCCCGGCUCCGCCGCCUCCCCCGAGGUGACCCCC | |
| | ACCUCCACCCCCAACCCCAACAACGUGACCCAGAAC | |
| | AAGACCACCCCCACCGAGCCCGCCUCCCCCCCCACC | |
| | ACCCCCAAGCCCACCUCCACCCCCAAGUCCCCCCCC | |
| | ACCUCCACCCCCGACCCCAAGCCCAAGAACAACACC | |
| | ACCCCCGCCAAGUCCGGCCGCCCCACCAAGCCCCCC | |
| | GGCCCCGUGUGGUGCGACCGCCGCGACCCCCUGGCC | |
| | CGCUACGGCUCCCGCGUGCAGAUCCGCUGCCGCUU | |
| | CCGCAACUCCACCCGCAUGGAGUUCCGCCUGCAGA | |
| | UCUGGCGCUACUCCAUGGGCCCCUCCCCCCCCAUCG | |
| | CCCCCGCCCCCGACCUGGAGGAGGUGCUGACCAAC | |
| | AUCACCGCCCCCCCCGGCGGCCUGCUGGUGUACGAC | |
| | UCCGCCCCCAACCUGACCGACCCCCACGUGCUGUGG | |
| | GCCGAGGGCGCCGGCCCCGGCGCCGACCCCCCCCUG | |
| | UACUCCGUGACCGGCCCCCUGCCCACCCAGCGCCUG | |
| | AUCAUCGGCGAGGUGACCCCCGCCACCCAGGGCAU | |
| | GUACUACCUGGCCUGGGGCCGCAUGGACUCCCCCC | |
| | ACGAGUACGGCACCUGGGUGCGCGUGCGCAUGUUC | |
| | CGCCCCCCCUCCCUGACCCUGCAGCCCCACGCCGUG | |
| | AUGGAGGGCCAGCCCUUCAAGGCCACCUGCACCGC | |
| | CGCCGCCUACUACCCCCGCAACCCCGUGGAGUUCGA | |
| | CUGGUUCGAGGACGACCGCCAGGUGUUCAACCCCG | |
| | GCCAGAUCGACACCCAGACCCACGAGCACCCCGACG | |
| | GCUUCACCACCGUGUCCACCGUGACCUCCGAGGCC | |
| | GUGGGCGGCCAGGUGCCCCCCCGCACCUUCACCUGC | |
| | CAGAUGACCUGGCACCGCGACUCCGUGACCUUCUC | |
| | CCGCCGCAACGCCACCGGCCUGGCCCUGGUGCUGCC | |
| | CCGCCCCACCAUCACCAUGGAGUUCGGCGUGCGCC | |
| | ACGUGGUGUGCACCGCCGGCUGCGUGCCCGAGGGC | |
| | GUGACCUUCGCCUGGUUCCUGGGCGACGACCCCUC | |
| | CCCCGCCGCCAAGUCCGCCGUGACCGCCCAGGAGUC | |
| | CUGCGACCACCCCGGCCUGGCCACCGUGCGCUCCAC | |
| | CCUGCCCAUCUCCUACGACUACUCCGAGUACAUCU | |
| | GCCGCCUGACCGGCUACCCCGCCGGCAUCCCCGUGC | |
| | UGGAGCACCACUAA | |
| HSV-2 gC | GCCUCCCCCGGCCGCACCAUCACCGUGGGCCCCCGC | 33 |
| [UL44] | GGCAACGCCUCCAACGCCGCCCCCUCCGCCUCCCCC | |
| (27-426) | CGCAACGCCUCCGCCCCCCGCACCACCCCCACCCCC | |
| WT | CCCCAGCCCCGCAAGGCCACCAAGUCCAAGGCCUCC | |
| | ACCGCCAAGCCCGCCCCCCCCCCCAAGACCGGCCCC | |
| | CCCAAGACCUCCUCCGAGCCCGUGCGCUGCAACCGC | |
| | CACGACCCCCUGGCCCGCUACGGCUCCCGCGUGCAG | |
| | AUCCGCUGCCGCUUCCCCAACUCCACCCGCACCGAG | |
| | UUCCGCCUGCAGAUCUGGCGCUACGCCACCGCCACC | |
| | GACGCCGAGAUCGGCACCGCCCCCUCCCUGGAGGA | |
| | GGUGAUGGUGAACGUGUCCGCCCCCCCCGGCGGCC | |
| | AGCUGGUGUACGACUCCGCCCCCAACCGCACCGACC | |
| | CCCACGUGAUCUGGGCCGAGGGCGCCGGCCCCGGC | |
| | GCCUCCCCCCGCCUGUACUCCGUGGUGGGCCCCCUG | |
| | GGCCGCCAGCGCCUGAUCAUCGAGGAGCUGACCCU | |
| | GGAGACCCAGGGCAUGUACUACUGGGUGUGGGGCC | |
| | GCACCGACCGCCCCUCCGCCUACGGCACCUGGGUGC | |
| | GCGUGCGCGUGUUCCGCCCCCCCUCCCUGACCAUCC | |
| | ACCCCCACGCCGUGCUGGAGGGCCAGCCCUUCAAG | |
| | GCCACCUGCACCGCCGCCACCUACUACCCCGGCAAC | |
| | CGCGCCGAGUUCGUGUGGUUCGAGGACGGCCGCCG | |
| | CGUGUUCGACCCCGCCCAGAUCCACACCCAGACCCA | |
| | GGAGAACCCCGACGGCUUCUCCACCGUGUCCACCG | |
| | UGACCUCCGCCGCCGUGGGCGGCCAGGGCCCCCCCC | |
| | GCACCUUCACCUGCCAGCUGACCUGGCACCGCGAC | |
| | UCCGUGUCCUUCUCCCGCCGCAACGCCUCCGGCACC | |
| | GCCUCCGUGCUGCCCCGCCCCACCAUCACCAUGGAG | |
| | UUCACCGGCGACCACGCCGUGUGCACCGCCGGCUG | |
| | CGUGCCCGAGGGCGUGACCUUCGCCUGGUUCCUGG | |
| | GCGACGACUCCUCCCCCGCCGAGAAGGUGGCCGUG | |
| | GCCUCCCAGACCUCCUGCGGCCGCCCCGGCACCGCC | |
| | ACCAUCCGCUCCACCCUGCCCGUGUCCUACGAGCAG | |
| | ACCGAGUACAUCUGCCGCCUGGCCGGCUACCCCGA | |
| | CGGCAUCCCCGUGCUGGAGCACCACUAA | |
| HSV-2 gC | UCCCCCGGCCGCACCAUCACCGUGGGCCCCCGCGGC | 34 |
| [UL44] | AACGCCUCCAACGCCGCCCCCUCCGCCUCCCCCCGC | |
| (28-426) | AACGCCUCCGCCCCCCGCACCACCCCCACCCCCCCC | |
| WT | CAGCCCCGCAAGGCCACCAAGUCCAAGGCCUCCACC | |
| | GCCAAGCCCGCCCCCCCCCCCAAGACCGGCCCCCCC | |
| | AAGACCUCCUCCGAGCCCGUGCGCUGCAACCGCCAC | |
| | GACCCCCUGGCCCGCUACGGCUCCCGCGUGCAGAUC | |
| | CGCUGCCGCUUCCCCAACUCCACCCGCACCGAGUUC | |
| | CGCCUGCAGAUCUGGCGCUACGCCACCGCCACCGAC | |
| | GCCGAGAUCGGCACCGCCCCCUCCCUGGAGGAGGU | |
| | GAUGGUGAACGUGUCCGCCCCCCCCGGCGGCCAGC | |
| | UGGUGUACGACUCCGCCCCCAACCGCACCGACCCCC | |
| | ACGUGAUCUGGGCCGAGGGCGCCGGCCCCGGCGCC | |
| | UCCCCCCGCCUGUACUCCGUGGUGGGCCCCCUGGGC | |
| | CGCCAGCGCCUGAUCAUCGAGGAGCUGACCCUGGA | |
| | GACCCAGGGCAUGUACUACUGGGUGUGGGGCCGCA | |
| | CCGACCGCCCCUCCGCCUACGGCACCUGGGUGCGCG | |
| | UGCGCGUGUUCCGCCCCCCCUCCCUGACCAUCCACC | |
| | CCCACGCCGUGCUGGAGGGCCAGCCCUUCAAGGCC | |
| | ACCUGCACCGCCGCCACCUACUACCCCGGCAACCGC | |
| | GCCGAGUUCGUGUGGUUCGAGGACGGCCGCCGCGU | |
| | GUUCGACCCCGCCCAGAUCCACACCCAGACCCAGGA | |
| | GAACCCCGACGGCUUCUCCACCGUGUCCACCGUGA | |
| | CCUCCGCCGCCGUGGGCGGCCAGGGCCCCCCCCGCA | |
| | CCUUCACCUGCCAGCUGACCUGGCACCGCGACUCCG | |
| | UGUCCUUCUCCCGCCGCAACGCCUCCGGCACCGCCU | |
| | CCGUGCUGCCCCGCCCCACCAUCACCAUGGAGUUCA | |
| | CCGGCGACCACGCCGUGUGCACCGCCGGCUGCGUG | |
| | CCCGAGGGCGUGACCUUCGCCUGGUUCCUGGGCGA | |
| | CGACUCCUCCCCCGCCGAGAAGGUGGCCGUGGCCU | |
| | CCCAGACCUCCUGCGGCCGCCCCGGCACCGCCACCA | |
| | UCCGCUCCACCCUGCCCGUGUCCUACGAGCAGACCG | |
| | AGUACAUCUGCCGCCUGGCCGGCUACCCCGACGGC | |
| | AUCCCCGUGCUGGAGCACCACUAA | |
| HSV-2 gC | AGCGCUUCUCCCGGCAGAACCAUCACAGUGGGCCC | 35 |
| [UL44] | UAGAGGCAACGCCUCUAAUGCCGCUCCUAGCGCCU | |
| (26-426) | CUCCUAGAAACGCCUCUGCUCCCAGAACCACACCU | |
| Version 1 | ACACCUCCACAGCCUAGAAAGGCCACCAAGAGCAA | |
| | GGCCAGCACAGCCAAACCUGCUCCUCCACCUAAGA | |
| | CAGGCCCUCCAAAGACAAGCUCUGAGCCCGUGCGG | |
| | UGCAACAGACACGAUCCACUGGCCAGAUACGGCAG | |
| | CCGGGUGCAGAUCAGAUGCAGAUUCCCCAACAGCA | |
| | CCCGGACCGAGUUCCGGCUCCAGAUUUGGAGAUAC | |
| | GCCACCGCCACAGAUGCCGAGAUUGGAACAGCCCC | |
| | UAGCCUGGAAGAAGUGAUGGUCAACGUUUCAGCCC | |
| | CUCCUGGCGGCCAGCUGGUGUAUGAUUCUGCCCCU | |
| | AACCGGACCGAUCCUCACGUGAUAUGGGCUGAAGG | |
| | UGCUGGCCCAGGCGCAAGCCCUAGACUGUAUUCUG | |
| | UUGUGGGCCCUCUGGGCAGACAGCGGCUGAUCAUU | |
| | GAGGAACUGACCCUGGAAACCCAGGGCAUGUACUA | |
| | CUGGGUCUGGGGCAGAACCGAUAGACCAAGCGCCU | |
| | AUGGCACCUGGGUUCGAGUGCGAGUGUUCAGACCU | |
| | CCUAGCCUGACCAUCCAUCCUCACGCCGUUCUGGA | |
| | AGGCCAGCCUUUCAAGGCCACAUGUACCGCCGCCA | |
| | CCUACUAUCCCGGAAACAGAGCCGAGUUCGUUUGG | |
| | UUCGAGGACGGCAGAAGGGUGUUCGACCCCGCUCA | |
| | GAUCCACACACAGACCCAAGAGAACCCCGACGGCU | |
| | UUAGCACCGUGUCCACAGUGACAUCUGCCGCCGUU | |
| | GGAGGACAGGGCCCUCCUAGAACCUUUACCUGCCA | |
| | GCUGACCUGGCACAGAGACAGCGUGUCCUUCAGCA | |
| | GAAGAAACGCCAGCGGCACAGCCAGCGUUCUGCCU | |
| | AGACCUACCAUCACCAUGGAAUUCACCGGCGACCA | |
| | CGCCGUGUGUACAGCUGGAUGUGUUCCUGAGGGCG | |
| | UGACCUUCGCUUGGUUUCUGGGCGACGAUAGCAGC | |
| | CCUGCCGAAAAAGUGGCUGUGGCCAGCCAGACAAG | |
| | CUGUGGCAGACCUGGAACCGCCACCAUCAGAAGCA | |
| | CACUGCCUGUCAGCUACGAGCAGACCGAGUACAUC | |
| | UGUCGGCUGGCCGGCUAUCCUGAUGGCAUCCCUGU | |
| | GCUGGAACACCACUGA | |
| HSV-2 gC | UCUCCCGGCAGAACCAUCACAGUGGGCCCUAGAGG | 36 |
| [UL44] | CAACGCCUCUAAUGCCGCUCCUAGCGCCUCUCCUA | |
| (28-426) | GAAACGCCUCUGCUCCCAGAACCACACCUACACCUC | |
| Version 1 | CACAGCCUAGAAAGGCCACCAAGAGCAAGGCCAGC | |
| | ACAGCCAAACCUGCUCCUCCACCUAAGACAGGCCC | |
| | UCCAAAGACAAGCUCUGAGCCCGUGCGGUGCAACA | |
| | GACACGAUCCACUGGCCAGAUACGGCAGCCGGGUG | |
| | CAGAUCAGAUGCAGAUUCCCCAACAGCACCCGGAC | |
| | CGAGUUCCGGCUCCAGAUUUGGAGAUACGCCACCG | |
| | CCACAGAUGCCGAGAUUGGAACAGCCCCUAGCCUG | |
| | GAAGAAGUGAUGGUCAACGUUUCAGCCCCUCCUGG | |
| | CGGCCAGCUGGUGUAUGAUUCUGCCCCUAACCGGA | |
| | CCGAUCCUCACGUGAUAUGGGCUGAAGGUGCUGGC | |
| | CCAGGCGCAAGCCCUAGACUGUAUUCUGUUGUGGG | |
| | CCCUCUGGGCAGACAGCGGCUGAUCAUUGAGGAAC | |
| | UGACCCUGGAAACCCAGGGCAUGUACUACUGGGUC | |
| | UGGGGCAGAACCGAUAGACCAAGCGCCUAUGGCAC | |
| | CUGGGUUCGAGUGCGAGUGUUCAGACCUCCUAGCC | |
| | UGACCAUCCAUCCUCACGCCGUUCUGGAAGGCCAG | |
| | CCUUUCAAGGCCACAUGUACCGCCGCCACCUACUA | |
| | UCCCGGAAACAGAGCCGAGUUCGUUUGGUUCGAGG | |
| | ACGGCAGAAGGGUGUUCGACCCCGCUCAGAUCCAC | |
| | ACACAGACCCAAGAGAACCCCGACGGCUUUAGCAC | |
| | CGUGUCCACAGUGACAUCUGCCGCCGUUGGAGGAC | |
| | AGGGCCCUCCUAGAACCUUUACCUGCCAGCUGACC | |
| | UGGCACAGAGACAGCGUGUCCUUCAGCAGAAGAAA | |
| | CGCCAGCGGCACAGCCAGCGUUCUGCCUAGACCUA | |
| | CCAUCACCAUGGAAUUCACCGGCGACCACGCCGUG | |
| | UGUACAGCUGGAUGUGUUCCUGAGGGCGUGACCUU | |
| | CGCUUGGUUUCUGGGCGACGAUAGCAGCCCUGCCG | |
| | AAAAAGUGGCUGUGGCCAGCCAGACAAGCUGUGGC | |
| | AGACCUGGAACCGCCACCAUCAGAAGCACACUGCC | |
| | UGUCAGCUACGAGCAGACCGAGUACAUCUGUCGGC | |
| | UGGCCGGCUAUCCUGAUGGCAUCCCUGUGCUGGAA | |
| | CACCACUGA | |
| HSV-2 gC | AGCCCUGGCAGAACCAUCACAGUGGGCCCUAGAGG | 37 |
| [UL44] | CAACGCCUCUAAUGCCGCUCCUAGCGCCUCUCCUA | |
| (28-426) | GAAACGCCUCUGCUCCCAGAACCACACCUACACCUC | |
| Version | CACAGCCUAGAAAGGCCACCAAGAGCAAGGCCAGC | |
| 1.1 | ACAGCCAAACCUGCUCCUCCACCUAAGACAGGCCC | |
| | UCCAAAGACAAGCUCUGAGCCCGUGCGGUGCAACA | |
| | GACACGAUCCACUGGCCAGAUACGGCAGCCGGGUG | |
| | CAGAUCAGAUGCAGAUUCCCCAACAGCACCCGGAC | |
| | CGAGUUCCGGCUCCAGAUUUGGAGAUACGCCACCG | |
| | CCACAGAUGCCGAGAUUGGAACAGCCCCUAGCCUG | |
| | GAAGAAGUGAUGGUCAACGUUUCAGCCCCUCCUGG | |
| | CGGCCAGCUGGUGUAUGAUUCUGCCCCUAACCGGA | |
| | CCGAUCCUCACGUGAUAUGGGCUGAAGGUGCUGGC | |
| | CCUGGCGCUUCCCCUAGACUGUAUUCUGUUGUGGG | |
| | CCCUCUGGGCAGACAGCGGCUGAUCAUUGAGGAAC | |
| | UGACCCUGGAAACCCAGGGCAUGUACUACUGGGUC | |
| | UGGGGCAGAACCGAUAGACCAAGCGCCUAUGGCAC | |
| | CUGGGUUCGAGUGCGAGUGUUCAGACCUCCUAGCC | |
| | UGACCAUCCAUCCUCACGCCGUUCUGGAAGGCCAG | |
| | CCUUUCAAGGCCACAUGUACCGCCGCCACCUACUA | |
| | UCCCGGAAACAGAGCCGAGUUCGUUUGGUUCGAGG | |
| | ACGGCAGAAGGGUGUUCGACCCCGCUCAGAUCCAC | |
| | ACACAGACCCAAGAGAACCCCGACGGCUUUAGCAC | |
| | CGUGUCCACAGUGACAUCUGCCGCCGUUGGAGGAC | |
| | AGGGCCCUCCUAGAACCUUUACCUGCCAGCUGACC | |
| | UGGCACAGAGACAGCGUGUCCUUCAGCAGAAGAAA | |
| | CGCCAGCGGCACAGCCAGCGUUCUGCCUAGACCUA | |
| | CCAUCACCAUGGAAUUCACCGGCGACCACGCCGUG | |
| | UGUACAGCUGGAUGUGUUCCUGAGGGCGUGACCUU | |
| | CGCUUGGUUUCUGGGCGACGAUAGCAGCCCUGCCG | |
| | AAAAAGUGGCUGUGGCCAGCCAGACAAGCUGUGGC | |
| | AGACCUGGAACCGCCACCAUCAGAAGCACACUGCC | |
| | UGUCAGCUACGAGCAGACCGAGUACAUCUGUCGGC | |
| | UGGCCGGCUAUCCUGAUGGCAUCCCUGUGCUGGAA | |
| | CACCACUGAUAA | |
| HSV-2 gC | GCAAGCCCCGGCAGAACCAUAACAGUAGGGCCACG | 38 |
| [UL44] | GGGGAAUGCUUCCAAUGCUGCACCUUCCGCUUCAC | |
| (27-426) | CGAGGAAUGCUUCUGCCCCAAGAACUACCCCCACU | |
| Version 2 | CCUCCUCAACCCAGGAAAGCGACAAAGUCCAAGGC | |
| | CAGCACCGCAAAACCCGCUCCUCCUCCAAAGACUG | |
| | GGCCCCCAAAGACAAGUAGCGAACCAGUUCGGUGC | |
| | AACAGGCAUGACCCACUUGCACGCUAUGGGUCAAG | |
| | AGUCCAGAUACGGUGUCGCUUCCCUAACAGUACAA | |
| | GGACUGAGUUUCGGCUGCAGAUCUGGCGUUAUGCC | |
| | ACAGCUACUGACGCAGAGAUUGGUACCGCCCCCAG | |
| | UUUGGAGGAAGUGAUGGUCAACGUGUCCGCACCCC | |
| | CAGGAGGACAGCUGGUCUAUGACUCAGCGCCCAAU | |
| | AGGACCGAUCCCCACGUGAUCUGGGCAGAAGGAGC | |
| | CGGUCCUGGGGCCUCUCCACGGCUGUACUCAGUUG | |
| | UUGGCCCGCUUGGACGACAGAGACUCAUCAUCGAG | |
| | GAACUGACACUGGAGACACAGGGGAUGUACUACUG | |
| | GGUGUGGGGCCGUACUGACCGCCCUUCCGCAUAUG | |
| | GCACUUGGGUGAGAGUUCGCGUCUUUCGGCCCCCU | |
| | UCUCUCACCAUCCAUCCUCAUGCCGUGCUCGAAGG | |
| | CCAGCCCUUUAAGGCCACAUGCACUGCUGCGACCU | |
| | ACUACCCUGGCAACAGAGCCGAGUUUGUCUGGUUU | |
| | GAGGAUGGUCGGCGAGUAUUCGAUCCAGCCCAGAU | |
| | UCACACACAAACGCAGGAAAAUCCGGACGGCUUCA | |
| | GCACAGUGUCCACGGUGACCUCUGCUGCAGUUGGU | |
| | GGACAAGGACCCCCUCGAACCUUCACCUGUCAGCU | |
| | GACCUGGCACAGAGACUCCGUAAGCUUCAGCCGUA | |
| | GAAACGCCUCUGGAACCGCCAGUGUGUUGCCGAGG | |
| | CCGACUAUCACGAUGGAAUUCACAGGCGAUCAUGC | |
| | CGUCUGUACUGCCGGCUGUGUGCCAGAAGGCGUAA | |
| | CCUUCGCUUGGUUUCUCGGGGAUGACUCAAGUCCU | |
| | GCAGAGAAAGUGGCUGUGGCCUCUCAGACGAGCUG | |
| | CGGUCGACCAGGAACAGCUACCAUUCGCAGCACUC | |
| | UGCCCGUGUCCUACGAGCAGACGGAGUACAUCUGC | |
| | AGGCUGGCCGGCUAUCCCGAUGGGAUUCCAGUCCU | |
| | GGAGCACCACUGA | |
| HSV-2 gC | AGCCCCGGCAGAACCAUAACAGUAGGGCCACGGGG | 39 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version 2 | CCUCAACCCAGGAAAGCGACAAAGUCCAAGGCCAG | |
| | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAGGAAGUGAUGGUCAACGUGUCCGCACCCCCAGG | |
| | AGGACAGCUGGUCUAUGACUCAGCGCCCAAUAGGA | |
| | CCGAUCCCCACGUGAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAGGAAC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGA | |
| HSV-2 gC | GCAAGCCCCGGCAGAACCAUAACAGUAGGGCCACG | 40 |
| [UL44] | GGGGAAUGCUUCCAAUGCUGCACCUUCCGCUUCAC | |
| (27-426) | CGAGGAAUGCUUCUGCCCCAAGAACUACCCCCACU | |
| Version | CCUCCUCAACCCAGGAAAGCGACAAAGUCCAAGGC | |
| 2.1 | CAGCACCGCAAAACCCGCUCCUCCUCCAAAGACUG | |
| | GGCCCCCAAAGACAAGUAGCGAACCAGUUCGGUGC | |
| | AACAGGCAUGACCCACUUGCACGCUAUGGGUCAAG | |
| | AGUCCAGAUACGGUGUCGCUUCCCUAACAGUACAA | |
| | GGACUGAGUUUCGGCUGCAGAUCUGGCGUUAUGCC | |
| | ACAGCUACUGACGCAGAGAUUGGUACCGCCCCCAG | |
| | UUUGGAAGAGGUGAUGGUCAACGUGUCCGCACCCC | |
| | CAGGAGGACAGCUGGUCUAUGACUCAGCGCCCAAU | |
| | AGGACCGAUCCCCACGUGAUCUGGGCAGAAGGAGC | |
| | CGGUCCUGGGGCCUCUCCACGGCUGUACUCAGUUG | |
| | UUGGCCCGCUUGGACGACAGAGACUCAUCAUCGAA | |
| | GAGCUGACACUGGAGACACAGGGGAUGUACUACUG | |
| | GGUGUGGGGCCGUACUGACCGCCCUUCCGCAUAUG | |
| | GCACUUGGGUGAGAGUUCGCGUCUUUCGGCCCCCU | |
| | UCUCUCACCAUCCAUCCUCAUGCCGUGCUCGAAGG | |
| | CCAGCCCUUUAAGGCCACAUGCACUGCUGCGACCU | |
| | ACUACCCUGGCAACAGAGCCGAGUUUGUCUGGUUU | |
| | GAGGAUGGUCGGCGAGUAUUCGAUCCAGCCCAGAU | |
| | UCACACACAAACGCAGGAAAAUCCGGACGGCUUCA | |
| | GCACAGUGUCCACGGUGACCUCUGCUGCAGUUGGU | |
| | GGACAAGGACCCCCUCGAACCUUCACCUGUCAGCU | |
| | GACCUGGCACAGAGACUCCGUAAGCUUCAGCCGUA | |
| | GAAACGCCUCUGGAACCGCCAGUGUGUUGCCGAGG | |
| | CCGACUAUCACGAUGGAAUUCACAGGCGAUCAUGC | |
| | CGUCUGUACUGCCGGCUGUGUGCCAGAAGGCGUAA | |
| | CCUUCGCUUGGUUUCUCGGGGAUGACUCAAGUCCU | |
| | GCAGAGAAAGUGGCUGUGGCCUCUCAGACGAGCUG | |
| | CGGUCGACCAGGAACAGCUACCAUUCGCAGCACUC | |
| | UGCCCGUGUCCUACGAGCAGACGGAGUACAUCUGC | |
| | AGGCUGGCCGGCUAUCCCGAUGGGAUUCCAGUCCU | |
| | GGAGCACCACUGA | |
| HSV-2 gC | AGCCCCGGCAGAACCAUAACAGUAGGGCCACGGGG | 41 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version | CCUCAACCCAGGAAAGCGACAAAGUCCAAGGCCAG | |
| 2.1 | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAAGAGGUGAUGGUCAACGUGUCCGCACCCCCAGG | |
| | AGGACAGCUGGUCUAUGACUCAGCGCCCAAUAGGA | |
| | CCGAUCCCCACGUGAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAAGAGC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGA | |
| HSV-2 gC | AGCCCCGGCAGAACCAUAACAGUAGGGCCACGGGG | 42 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version | CCUCAACCCAGGAAAGCGACAAAGUCCAAGGCCAG | |
| 2.2 | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAAGAGGUGAUGGUCAACGUGUCCGCACCCCCAGG | |
| | AGGACAGCUGGUCUAUGACUCAGCGCCCAAUAGGA | |
| | CCGAUCCCCACGUGAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAAGAGC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGAUAA | |
| HSV-2 gC | AGCCCCGGCAGAACCAUAACAGUAGGGCCACGGGG | 43 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version | CCUCAACCCAGGAAAGCGACAAAGUCCAAGGCCAG | |
| 2.3 | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAGGAAGUGAUGGUCAACGUGUCCGCACCCCCAGG | |
| | AGGACAGCUGGUCUAUGACUCAGCGCCCAAUAGGA | |
| | CAGACCCACAUGUUAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAGGAAC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGA | |
| HSV-2 gC | AGCCCCGGCAGAACCAUAACAGUAGGGCCACGGGG | 44 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version | CCUCAACCCAGGAAAGCGACAAAGUCCAAGGCCAG | |
| 2.4 | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAGGAAGUGAUGGUCAACGUGUCCGCACCCCCAGG | |
| | AGGACAGCUGGUCUAUGACUCAGCGCCCAAUAGGA | |
| | CCGAUCCCCACGUGAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAGGAAC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGA | |
| HSV-2 gC | AGCCCCGGCAGAACCAUAACAGUAGGGCCACGGGG | 45 |
| [UL44] | GAAUGCUUCCAAUGCUGCACCUUCCGCUUCACCGA | |
| (28-426) | GGAAUGCUUCUGCCCCAAGAACUACCCCCACUCCU | |
| Version | CCUCAACCCAGGAAAGCGACAAAGUCCAAGGCCAG | |
| 2.8 | CACCGCAAAACCCGCUCCUCCUCCAAAGACUGGGCC | |
| | CCCAAAGACAAGUAGCGAACCAGUUCGGUGCAACA | |
| | GGCAUGACCCACUUGCACGCUAUGGGUCAAGAGUC | |
| | CAGAUACGGUGUCGCUUCCCUAACAGUACAAGGAC | |
| | UGAGUUUCGGCUGCAGAUCUGGCGUUAUGCCACAG | |
| | CUACUGACGCAGAGAUUGGUACCGCCCCCAGUUUG | |
| | GAGGAAGUGAUGGUCAACGUGUCCGCACCCCCAGG | |
| | AGGACAGCUGGUCUAUGACUCAGCGCCCAAUAGGA | |
| | CCGAUCCCCACGUGAUCUGGGCAGAAGGAGCCGGU | |
| | CCUGGGGCCUCUCCACGGCUGUACUCAGUUGUUGG | |
| | CCCGCUUGGACGACAGAGACUCAUCAUCGAGGAAC | |
| | UGACACUGGAGACACAGGGGAUGUACUACUGGGUG | |
| | UGGGGCCGUACUGACCGCCCUUCCGCAUAUGGCAC | |
| | UUGGGUGAGAGUUCGCGUCUUUCGGCCCCCUUCUC | |
| | UCACCAUCCAUCCUCAUGCCGUGCUCGAAGGCCAG | |
| | CCCUUUAAGGCCACAUGCACUGCUGCGACCUACUA | |
| | CCCUGGCAACAGAGCCGAGUUUGUCUGGUUUGAGG | |
| | AUGGUCGGCGAGUAUUCGAUCCAGCCCAGAUUCAC | |
| | ACACAAACGCAGGAAAAUCCGGACGGCUUCAGCAC | |
| | AGUGUCCACGGUGACCUCUGCUGCAGUUGGUGGAC | |
| | AAGGACCCCCUCGAACCUUCACCUGUCAGCUGACC | |
| | UGGCACAGAGACUCCGUAAGCUUCAGCCGUAGAAA | |
| | CGCCUCUGGAACCGCCAGUGUGUUGCCGAGGCCGA | |
| | CUAUCACGAUGGAAUUCACAGGCGAUCAUGCCGUC | |
| | UGUACUGCCGGCUGUGUGCCAGAAGGCGUAACCUU | |
| | CGCUUGGUUUCUCGGGGAUGACUCAAGUCCUGCAG | |
| | AGAAAGUGGCUGUGGCCUCUCAGACGAGCUGCGGU | |
| | CGACCAGGAACAGCUACCAUUCGCAGCACUCUGCC | |
| | CGUGUCCUACGAGCAGACGGAGUACAUCUGCAGGC | |
| | UGGCCGGCUAUCCCGAUGGGAUUCCAGUCCUGGAG | |
| | CACCACUGAUAA | |
| HSV-2 gC | GCCUCCCCCGGCAGAACCAUCACCGUGGGCCCCAGA | 46 |
| [UL44] | GGCAACGCCUCCAACGCCGCCCCCUCCGCCUCCCCC | |
| (27-426) | AGAAACGCCUCCGCCCCCAGAACCACCCCCACCCCU | |
| Version 3 | CCCCAGCCCAGAAAAGCCACCAAAUCCAAAGCCUCC | |
| | ACCGCCAAACCCGCCCCUCCUCCCAAAACCGGCCCU | |
| | CCCAAAACCUCCUCCGAACCCGUGAGAUGCAACAG | |
| | ACACGAUCCCCUGGCCAGAUACGGCUCCAGAGUGC | |
| | AGAUCAGAUGCAGAUUCCCCAACUCCACCAGAACC | |
| | GAAUUCAGACUGCAGAUCUGGAGAUACGCCACCGC | |
| | CACCGAUGCCGAAAUCGGCACCGCCCCCUCCCUGGA | |
| | AGAAGUGAUGGUGAACGUGUCCGCCCCUCCUGGCG | |
| | GCCAGCUGGUGUACGAUUCCGCCCCCAACAGAACC | |
| | GAUCCCCACGUGAUCUGGGCCGAAGGCGCCGGCCC | |
| | CGGCGCCUCCCCCAGACUGUACUCCGUGGUGGGCC | |
| | CCCUGGGCAGACAGAGACUGAUCAUCGAAGAACUG | |
| | ACCCUGGAAACCCAGGGCAUGUACUACUGGGUGUG | |
| | GGGCAGAACCGAUAGACCCUCCGCCUACGGCACCU | |
| | GGGUGAGAGUGAGAGUGUUCAGACCCCCUUCCCUG | |
| | ACCAUCCACCCCCACGCCGUGCUGGAAGGCCAGCCC | |
| | UUCAAAGCCACCUGCACCGCCGCCACCUACUACCCC | |
| | GGCAACAGAGCCGAAUUCGUGUGGUUCGAAGAUGG | |
| | CAGAAGGGUGUUCGAUCCCGCCCAGAUCCACACCC | |
| | AGACCCAGGAAAACCCCGACGGCUUCUCCACCGUG | |
| | UCCACCGUGACCUCCGCCGCCGUGGGCGGCCAGGG | |
| | CCCUCCCAGAACCUUCACCUGCCAGCUGACCUGGCA | |
| | CAGAGACUCCGUGUCCUUCUCCAGAAGAAACGCCU | |
| | CCGGCACCGCCUCCGUGCUGCCCAGACCCACCAUCA | |
| | CCAUGGAAUUCACCGGCGAUCACGCCGUGUGCACC | |
| | GCCGGCUGCGUGCCCGAAGGCGUGACCUUCGCCUG | |
| | GUUCCUGGGCGAUGAUUCCUCCCCCGCCGAAAAAG | |
| | UGGCCGUGGCCUCCCAGACCUCCUGCGGCAGACCC | |
| | GGCACCGCCACCAUCAGAUCCACCCUGCCCGUGUCC | |
| | UACGAACAGACCGAAUACAUCUGCAGACUGGCCGG | |
| | CUACCCCGAUGGCAUCCCCGUGCUGGAACACCACU | |
| | GA | |
| HSV-2 gC | UCCCCCGGCAGAACCAUCACCGUGGGCCCCAGAGG | 47 |
| [UL44] | CAACGCCUCCAACGCCGCCCCCUCCGCCUCCCCCAG | |
| (28-426) | AAACGCCUCCGCCCCCAGAACCACCCCCACCCCUCC | |
| Version 3 | CCAGCCCAGAAAAGCCACCAAAUCCAAAGCCUCCA | |
| | CCGCCAAACCCGCCCCUCCUCCCAAAACCGGCCCUC | |
| | CCAAAACCUCCUCCGAACCCGUGAGAUGCAACAGA | |
| | CACGAUCCCCUGGCCAGAUACGGCUCCAGAGUGCA | |
| | GAUCAGAUGCAGAUUCCCCAACUCCACCAGAACCG | |
| | AAUUCAGACUGCAGAUCUGGAGAUACGCCACCGCC | |
| | ACCGAUGCCGAAAUCGGCACCGCCCCCUCCCUGGA | |
| | AGAAGUGAUGGUGAACGUGUCCGCCCCUCCUGGCG | |
| | GCCAGCUGGUGUACGAUUCCGCCCCCAACAGAACC | |
| | GAUCCCCACGUGAUCUGGGCCGAAGGCGCCGGCCC | |
| | CGGCGCCUCCCCCAGACUGUACUCCGUGGUGGGCC | |
| | CCCUGGGCAGACAGAGACUGAUCAUCGAAGAACUG | |
| | ACCCUGGAAACCCAGGGCAUGUACUACUGGGUGUG | |
| | GGGCAGAACCGAUAGACCCUCCGCCUACGGCACCU | |
| | GGGUGAGAGUGAGAGUGUUCAGACCCCCUUCCCUG | |
| | ACCAUCCACCCCCACGCCGUGCUGGAAGGCCAGCCC | |
| | UUCAAAGCCACCUGCACCGCCGCCACCUACUACCCC | |
| | GGCAACAGAGCCGAAUUCGUGUGGUUCGAAGAUGG | |
| | CAGAAGGGUGUUCGAUCCCGCCCAGAUCCACACCC | |
| | AGACCCAGGAAAACCCCGACGGCUUCUCCACCGUG | |
| | UCCACCGUGACCUCCGCCGCCGUGGGCGGCCAGGG | |
| | CCCUCCCAGAACCUUCACCUGCCAGCUGACCUGGCA | |
| | CAGAGACUCCGUGUCCUUCUCCAGAAGAAACGCCU | |
| | CCGGCACCGCCUCCGUGCUGCCCAGACCCACCAUCA | |
| | CCAUGGAAUUCACCGGCGAUCACGCCGUGUGCACC | |
| | GCCGGCUGCGUGCCCGAAGGCGUGACCUUCGCCUG | |
| | GUUCCUGGGCGAUGAUUCCUCCCCCGCCGAAAAAG | |
| | UGGCCGUGGCCUCCCAGACCUCCUGCGGCAGACCC | |
| | GGCACCGCCACCAUCAGAUCCACCCUGCCCGUGUCC | |
| | UACGAACAGACCGAAUACAUCUGCAGACUGGCCGG | |
| | CUACCCCGAUGGCAUCCCCGUGCUGGAACACCACU | |
| | GA | |
| HSV-2 gC | UCUCCUGGAAGAACCAUCACAGUGGGACCAAGAGG | 48 |
| [UL44] | AAAUGCAAGCAAUGCAGCACCUUCUGCUUCUCCAA | |
| (28-426) | GAAAUGCUUCUGCUCCAAGAACCACCCCAACCCCU | |
| Version | CCUCAGCCAAGAAAAGCAACCAAAUCCAAAGCAUC | |
| 3.1 | CACAGCAAAACCUGCACCUCCUCCAAAAACAGGAC | |
| | CUCCAAAAACCUCCUCUGAACCUGUGAGAUGCAAC | |
| | AGACAUGAUCCUCUGGCAAGAUAUGGAUCAAGAGU | |
| | GCAGAUCAGAUGCAGAUUUCCAAAUUCCACCAGAA | |
| | CAGAAUUCAGACUCCAGAUCUGGAGAUAUGCAACA | |
| | GCAACAGAUGCAGAAAUUGGAACAGCACCAUCUCU | |
| | GGAAGAAGUGAUGGUGAAUGUGUCUGCUCCUCCUG | |
| | GAGGACAGCUGGUGUAUGAUUCUGCUCCAAACAGA | |
| | ACAGAUCCUCAUGUGAUCUGGGCUGAAGGAGCUGG | |
| | ACCUGGAGCUUCUCCAAGACUGUACUCUGUGGUGG | |
| | GACCUCUGGGAAGACAGAGACUGAUCAUUGAAGAA | |
| | CUGACCCUGGAAACCCAGGGAAUGUACUACUGGGU | |
| | GUGGGGAAGAACAGACAGACCUUCUGCUUAUGGAA | |
| | CCUGGGUGAGAGUGAGAGUGUUCAGACCUCCUUCU | |
| | CUGACCAUCCACCCUCAUGCUGUGCUGGAAGGACA | |
| | GCCUUUCAAAGCAACCUGCACAGCAGCAACCUACU | |
| | ACCCUGGAAACAGAGCUGAAUUUGUGUGGUUUGAA | |
| | GAUGGAAGAAGGGUGUUUGAUCCUGCUCAGAUCCA | |
| | CACCCAGACCCAGGAAAAUCCUGAUGGAUUUUCCA | |
| | CAGUGUCCACAGUGACAUCUGCUGCUGUGGGAGGA | |
| | CAGGGACCUCCAAGAACCUUCACCUGCCAGCUGAC | |
| | CUGGCACAGAGAUUCUGUGUCUUUUUCAAGAAGAA | |
| | AUGCUUCUGGAACAGCUUCUGUGCUGCCAAGACCA | |
| | ACCAUCACCAUGGAAUUCACAGGAGAUCAUGCUGU | |
| | GUGCACAGCUGGAUGUGUGCCUGAAGGAGUGACCU | |
| | UUGCUUGGUUUCUGGGAGAUGAUUCUUCUCCAGCU | |
| | GAAAAAGUGGCUGUGGCUUCCCAGACCUCUUGUGG | |
| | AAGACCUGGAACAGCAACCAUCAGAUCCACCCUGC | |
| | CUGUGUCUUAUGAACAGACAGAAUACAUUUGCAGA | |
| | CUGGCUGGAUACCCUGAUGGAAUCCCUGUGCUGGA | |
| | ACACCACUGAUAA | |
| HSV-2 gC | UCUCCGGGACGGACUAUAACCGUAGGUCCAAGAGG | 49 |
| [UL44] | AAACGCCUCUAACGCAGCCCCGUCUGCCUCACCACG | |
| (28-426) | AAACGCCUCAGCUCCCAGAACCACUCCUACUCCACC | |
| Version 4 | CCAGCCUAGGAAGGCGACGAAAUCCAAGGCUUCCA | |
| | CGGCCAAACCCGCCCCUCCACCCAAAACCGGACCUC | |
| | CUAAGACCAGCUCUGAACCGGUGCGGUGUAAUAGG | |
| | CACGACCCAUUGGCGCGAUAUGGCAGUAGGGUCCA | |
| | GAUACGGUGCAGAUUCCCAAACAGCACAAGAACAG | |
| | AAUUCCGGCUGCAAAUCUGGCGAUAUGCAACGGCC | |
| | ACCGAUGCCGAAAUCGGAACAGCACCCAGUCUGGA | |
| | AGAAGUGAUGGUGAACGUCAGUGCUCCACCUGGCG | |
| | GACAACUGGUGUACGACUCUGCACCCAAUCGCACA | |
| | GAUCCCCACGUGAUUUGGGCCGAGGGUGCUGGACC | |
| | UGGGGCUUCACCCAGGCUGUAUAGCGUUGUAGGGC | |
| | CACUUGGGAGGCAGAGACUCAUCAUUGAGGAACUG | |
| | ACCCUGGAAACUCAGGGCAUGUACUACUGGGUAUG | |
| | GGGCCGCACAGAUCGCCCCAGCGCUUAUGGCACCU | |
| | GGGUGCGGGUGCGGGUGUUUCGCCCACCCUCCCUC | |
| | ACCAUUCACCCUCAUGCGGUUCUGGAGGGACAGCC | |
| | UUUCAAGGCAACUUGUACCGCAGCCACCUACUAUC | |
| | CCGGCAAUAGAGCGGAGUUCGUCUGGUUUGAGGAC | |
| | GGCCGUAGGGUGUUCGAUCCUGCCCAGAUUCACAC | |
| | CCAGACACAGGAGAAUCCCGACGGCUUUAGCACAG | |
| | UGAGCACUGUGACGUCUGCUGCCGUUGGUGGUCAA | |
| | GGGCCUCCUCGUACCUUCACAUGCCAAUUGACCUG | |
| | GCACCGCGACUCAGUUAGCUUUAGCCGCCGGAAUG | |
| | CCAGUGGGACCGCCAGUGUUCUCCCAAGGCCGACA | |
| | AUCACCAUGGAGUUCACUGGCGACCAUGCAGUGUG | |
| | CACAGCUGGGUGUGUCCCAGAAGGCGUGACUUUCG | |
| | CCUGGUUUCUGGGUGAUGACUCCUCACCCGCCGAG | |
| | AAAGUAGCUGUCGCUUCCCAGACUUCCUGUGGACG | |
| | UCCUGGAACUGCGACAAUCCGAAGCACACUGCCGG | |
| | UUUCCUACGAGCAGACGGAGUACAUAUGCCGCCUU | |
| | GCAGGCUACCCCGAUGGAAUUCCAGUCCUUGAGCA | |
| | CCAUUGA | |
| HSV-2 gC | AGUCCAGGAAGGACGAUUACGGUGGGACCCAGAGG | 50 |
| [UL44] | UAAUGCGUCCAAUGCUGCGCCAUCCGCUUCUCCAC | |
| (28-426) | GGAACGCAUCUGCACCCAGGACUACACCGACACCA | |
| Version | CCUCAGCCGCGCAAAGCCACCAAGAGCAAGGCCAG | |
| 4.1 | CACAGCCAAACCCGCUCCUCCACCUAAAACCGGACC | |
| | ACCUAAGACCAGCUCUGAACCCGUCAGAUGCAACA | |
| | GGCACGAUCCGUUGGCCAGAUAUGGCAGUCGCGUC | |
| | CAGAUCAGGUGUCGCUUCCCUAACAGCACACGGAC | |
| | CGAGUUCAGGCUGCAAAUUUGGCGCUACGCUACAG | |
| | CCACUGACGCAGAGAUUGGCACUGCUCCCAGUCUG | |
| | GAGGAGGUCAUGGUGAACGUGUCUGCUCCACCAGG | |
| | CGGUCAGCUGGUCUAUGACUCAGCCCCUAAUCGCA | |
| | CAGAUCCUCACGUGAUUUGGGCAGAAGGUGCGGGG | |
| | CCUGGGGCCUCCCCAAGGCUCUACUCAGUGGUUGG | |
| | ACCCCUUGGGAGACAGCGGCUGAUCAUCGAGGAAC | |
| | UGACUCUCGAAACCCAAGGUAUGUACUACUGGGUA | |
| | UGGGGCAGAACAGACAGACCUUCAGCUUAUGGCAC | |
| | CUGGGUGCGGGUGAGAGUGUUUAGGCCUCCCUCCC | |
| | UGACGAUCCAUCCCCAUGCUGUGCUGGAAGGACAG | |
| | CCGUUCAAGGCAACAUGCACAGCAGCCACUUACUA | |
| | UCCCGGAAACCGUGCUGAGUUUGUGUGGUUCGAGG | |
| | AUGGGCGACGUGUAUUCGACCCUGCCCAGAUUCAC | |
| | ACCCAGACACAGGAGAAUCCCGACGGGUUUUCCAC | |
| | UGUGAGCACCGUGACAUCAGCGGCAGUAGGAGGGC | |
| | AGGGCCCACCCCGAACGUUCACUUGCCAGCUUACU | |
| | UGGCAUCGGGACAGUGUUAGCUUUAGCCGCCGGAA | |
| | UGCCUCUGGCACCGCAUCCGUCCUUCCUCGCCCAAC | |
| | CAUCACCAUGGAAUUCACUGGCGAUCACGCCGUUU | |
| | GUACAGCCGGGUGUGUUCCCGAGGGAGUGACCUUU | |
| | GCUUGGUUUCUGGGCGAUGACUCAAGCCCAGCCGA | |
| | AAAGGUGGCCGUCGCCUCCCAAACGAGCUGUGGGC | |
| | GACCUGGCACCGCUACCAUACGUAGCACUCUGCCC | |
| | GUUUCCUACGAACAGACCGAGUAUAUCUGCCGAUU | |
| | GGCCGGUUACCCCGAUGGGAUACCAGUCCUGGAGC | |
| | ACCACUGA | |
| HSV-2 gC | UCCCCGGGUCGAACAAUCACUGUUGGGCCCAGGGG | 51 |
| [UL44] | AAAUGCCAGCAAUGCUGCACCUUCAGCAAGCCCAC | |
| (28-426) | GAAACGCUUCAGCACCCAGGACAACACCCACUCCA | |
| Version | CCUCAACCGCGGAAAGCCACCAAGAGCAAGGCAAG | |
| 4.2 | UACCGCCAAACCCGCUCCUCCUCCCAAGACAGGGCC | |
| | ACCCAAGACCUCUAGUGAGCCAGUGAGGUGUAACC | |
| | GCCAUGAUCCCCUUGCCAGAUACGGGAGCAGAGUG | |
| | CAGAUUAGGUGCCGGUUUCCAAACUCCACGAGAAC | |
| | CGAAUUUCGCCUCCAGAUUUGGCGGUAUGCGACUG | |
| | CCACAGACGCAGAGAUUGGUACCGCUCCCAGCCUG | |
| | GAGGAGGUCAUGGUGAACGUGUCAGCGCCUCCGGG | |
| | UGGCCAGCUGGUCUACGACUCUGCCCCAAAUCGAA | |
| | CCGACCCUCACGUCAUCUGGGCUGAAGGAGCGGGA | |
| | CCAGGAGCCUCUCCACGCUUGUAUAGCGUAGUUGG | |
| | CCCUCUGGGGAGACAGCGCCUGAUCAUUGAGGAAC | |
| | UGACCCUUGAGACACAGGGGAUGUACUACUGGGUG | |
| | UGGGGCAGGACUGACAGGCCCAGUGCCUAUGGAAC | |
| | UUGGGUUAGGGUCCGCGUCUUUCGGCCACCCAGUC | |
| | UGACCAUCCAUCCACAUGCCGUGCUGGAAGGCCAG | |
| | CCCUUCAAAGCGACUUGCACUGCCGCCACGUACUA | |
| | UCCAGGGAAUAGAGCCGAGUUCGUUUGGUUCGAGG | |
| | AUGGCCGGAGAGUAUUCGAUCCAGCUCAGAUCCAC | |
| | ACCCAGACGCAGGAAAACCCGGACGGCUUUAGCAC | |
| | GGUGAGUACCGUCACCUCUGCUGCCGUCGGAGGCC | |
| | AAGGACCUCCCCGUACCUUCACAUGCCAGCUUACA | |
| | UGGCACCGGGACUCAGUAAGCUUUUCACGUCGUAA | |
| | UGCAUCCGGUACUGCUUCUGUGCUGCCUCGACCCA | |
| | CCAUCACCAUGGAGUUCACAGGGGAUCACGCAGUG | |
| | UGUACGGCAGGCUGCGUGCCUGAAGGCGUGACAUU | |
| | CGCCUGGUUUCUCGGUGAUGACUCCUCUCCUGCUG | |
| | AAAAGGUGGCUGUAGCCUCCCAAACAAGCUGUGGU | |
| | CGGCCUGGAACUGCCACUAUACGCUCCACUCUCCC | |
| | GGUGUCCUACGAACAGACCGAGUACAUAUGCAGAC | |
| | UGGCUGGAUAUCCCGAUGGCAUUCCCGUGCUGGAG | |
| | CAUCACUGA | |
| HSV-2 gC | UCUCCAGGCAGAACUAUCACAGUGGGACCCAGAGG | 52 |
| [UL44] | GAAUGCCAGCAAUGCAGCCCCGAGUGCCAGCCCUC | |
| (28-426) | GUAACGCCAGCGCUCCCAGAACAACCCCAACUCCAC | |
| Version | CGCAGCCUAGAAAGGCGACCAAGUCCAAAGCAUCC | |
| 4.3 | ACUGCAAAACCAGCCCCACCUCCCAAAACGGGACC | |
| | UCCCAAGACCAGCUCCGAGCCUGUAAGGUGCAAUC | |
| | GGCAUGACCCCUUGGCCCGAUAUGGCAGUCGCGUG | |
| | CAGAUUCGAUGUCGGUUUCCCAACUCUACCCGGAC | |
| | UGAGUUCCGGUUGCAGAUCUGGAGGUAUGCGACCG | |
| | CCACUGACGCUGAGAUCGGCACAGCACCAAGCCUG | |
| | GAAGAAGUGAUGGUGAACGUUAGUGCUCCUCCGGG | |
| | CGGGCAACUCGUGUAUGACUCCGCACCCAACCGCA | |
| | CAGAUCCUCACGUGAUUUGGGCCGAAGGAGCCGGA | |
| | CCCGGUGCGUCACCUAGGCUCUACUCUGUCGUAGG | |
| | ACCACUGGGCCGUCAACGCCUGAUAAUCGAGGAGC | |
| | UGACUCUGGAGACACAGGGUAUGUACUACUGGGUC | |
| | UGGGGCAGAACCGACAGGCCAUCUGCUUACGGGAC | |
| | AUGGGUCCGCGUUCGAGUAUUUCGGCCACCCUCAC | |
| | UGACCAUACAUCCCCAUGCCGUUCUUGAAGGGCAG | |
| | CCUUUCAAGGCAACCUGUACUGCUGCCACAUACUA | |
| | UCCCGGGAAUAGGGCCGAGUUCGUCUGGUUUGAAG | |
| | AUGGCCGAAGGGUGUUUGACCCGGCUCAGAUCCAC | |
| | ACCCAGACACAGGAGAACCCCGAUGGCUUCAGUAC | |
| | GGUGUCUACCGUCACAAGCGCCGCUGUGGGUGGCC | |
| | AAGGUCCUCCCAGAACUUUCACCUGUCAGCUGACG | |
| | UGGCACAGGGAUUCCGUGAGCUUUUCCCGCCGCAA | |
| | UGCGUCAGGGACCGCCUCCGUGCUUCCUCGGCCAA | |
| | CCAUCACAAUGGAAUUCACGGGUGAUCACGCUGUC | |
| | UGCACAGCUGGCUGCGUUCCUGAGGGCGUGACAUU | |
| | CGCAUGGUUUCUUGGUGACGACUCAUCUCCCGCAG | |
| | AGAAGGUGGCUGUUGCCUCACAAACGAGUUGUGGG | |
| | CGUCCAGGCACUGCCACCAUUCGGUCCACCCUCCCC | |
| | GUAAGCUACGAACAGACUGAGUAUAUUUGCAGACU | |
| | GGCUGGAUACCCGGAUGGGAUUCCUGUCCUGGAAC | |
| | AUCACUGA | |
| HSV-2 gC | UCUCCCGGACGAACUAUCACUGUAGGCCCAAGGGG | 53 |
| [UL44] | CAACGCUAGUAAUGCCGCUCCCAGUGCUUCACCAC | |
| (28-426) | GCAAUGCGAGCGCACCCAGAACUACACCCACACCU | |
| Version | CCUCAGCCGAGGAAAGCCACCAAGUCCAAAGCCAG | |
| 4.4 | CACCGCCAAACCCGCUCCUCCACCUAAAACAGGGCC | |
| | UCCCAAGACCUCAAGCGAGCCCGUUAGAUGCAAUC | |
| | GGCAUGAUCCACUGGCUCGUUACGGUUCUCGGGUC | |
| | CAGAUACGCUGUAGGUUUCCUAACUCCACACGAAC | |
| | CGAGUUCAGAUUGCAGAUCUGGAGAUAUGCCACAG | |
| | CCACUGACGCUGAGAUUGGCACUGCACCUAGUCUG | |
| | GAGGAGGUGAUGGUGAACGUGAGCGCCCCUCCUGG | |
| | CGGUCAGCUUGUGUAUGACUCAGCACCGAAUCGCA | |
| | CAGAUCCGCACGUCAUAUGGGCCGAAGGUGCAGGG | |
| | CCCGGUGCAUCCCCUCGGCUGUAUUCCGUGGUCGG | |
| | ACCACUCGGGCGCCAGAGGCUUAUCAUUGAGGAAC | |
| | UGACCCUCGAAACCCAGGGUAUGUACUACUGGGUA | |
| | UGGGGCCGUACCGACCGGCCUAGCGCCUACGGAAC | |
| | UUGGGUGAGAGUUCGGGUGUUCAGACCGCCAAGUC | |
| | UUACAAUUCACCCUCAUGCCGUGCUCGAAGGUCAG | |
| | CCAUUUAAGGCCACGUGUACUGCGGCUACGUACUA | |
| | UCCCGGGAAUCGGGCUGAAUUCGUGUGGUUUGAGG | |
| | AUGGCAGGAGAGUGUUCGACCCAGCCCAAAUCCAC | |
| | ACCCAAACACAGGAGAACCCAGACGGGUUUUCCAC | |
| | CGUGUCAACGGUCACAUCUGCCGCCGUCGGAGGAC | |
| | AAGGGCCACCCAGAACCUUCACAUGCCAGCUGACC | |
| | UGGCAUAGGGAUAGCGUAAGCUUUAGCCGGCGAAA | |
| | CGCAUCUGGAACGGCGAGCGUUCUGCCUCGACCAA | |
| | CAAUCACCAUGGAGUUCACCGGCGAUCACGCAGUG | |
| | UGCACUGCUGGGUGUGUACCCGAAGGCGUGACAUU | |
| | UGCCUGGUUUCUGGGAGAUGACUCCUCACCCGCAG | |
| | AAAAGGUCGCCGUUGCAUCUCAGACCAGUUGUGGC | |
| | AGGCCCGGGACUGCUACCAUCCGCAGCACUCUGCC | |
| | GGUGUCUUACGAACAGACGGAGUACAUUUGCCGCU | |
| | UGGCGGGCUAUCCAGACGGCAUUCCAGUUCUGGAG | |
| | CAUCACUGA | |
| HSV-1 | AAGUACGCCCUGGCCGACGCCUCCCUGAAGAUGGC | 54 |
| gD | CGACCCCAACCGCUUCCGCGGCAAGGACCUGCCCGU | |
| (26-331) | GCUGGACCAGCUGACCGACCCCCCCGGCGUGCGCCG | |
| WT | CGUGUACCACAUCCAGGCCGGCCUGCCCGACCCCUU | |
| | CCAGCCCCCCUCCCUGCCCAUCACCGUGUACUACGC | |
| | CGUGCUGGAGCGCGCCUGCCGCUCCGUGCUGCUGA | |
| | ACGCCCCCUCCGAGGCCCCCCAGAUCGUGCGCGGCG | |
| | CCUCCGAGGACGUGCGCAAGCAGCCCUACAACCUG | |
| | ACCAUCGCCUGGUUCCGCAUGGGCGGCAACUGCGC | |
| | CAUCCCCAUCACCGUGAUGGAGUACACCGAGUGCU | |
| | CCUACAACAAGUCCCUGGGCGCCUGCCCCAUCCGCA | |
| | CCCAGCCCCGCUGGAACUACUACGACUCCUUCUCCG | |
| | CCGUGUCCGAGGACAACCUGGGCUUCCUGAUGCAC | |
| | GCCCCCGCCUUCGAGACCGCCGGCACCUACCUGCGC | |
| | CUGGUGAAGAUCAACGACUGGACCGAGAUCACCCA | |
| | GUUCAUCCUGGAGCACCGCGCCAAGGGCUCCUGCA | |
| | AGUACGCCCUGCCCCUGCGCAUCCCCCCCUCCGCCU | |
| | GCCUGUCCCCCCAGGCCUACCAGCAGGGCGUGACC | |
| | GUGGACUCCAUCGGCAUGCUGCCCCGCUUCAUCCC | |
| | CGAGAACCAGCGCACCGUGGCCGUGUACUCCCUGA | |
| | AGAUCGCCGGCUGGCACGGCCCCAAGGCCCCCUAC | |
| | ACCUCCACCCUGCUGCCCCCCGAGCUGUCCGAGACC | |
| | CCCAACGCCACCCAGCCCGAGCUGGCCCCCGAGGAC | |
| | CCCGAGGACUCCGCCCUGCUGGAGGACCCCGUGGG | |
| | CACCGUGGCCCCCCAGAUCCCCCCCAACUGGCACAU | |
| | CCCCUCCAUCCAGGACGCCGCCACCCCCUACUAA | |
| HSV-2 | AAGUACGCCCUGGCCGACCCCUCCCUGAAGAUGGC | 55 |
| gD | CGACCCCAACCGCUUCCGCGGCAAGAACCUGCCCGU | |
| (26-331) | GCUGGACCAGCUGACCGACCCCCCCGGCGUGAAGC | |
| WT | GCGUGUACCACAUCCAGCCCUCCCUGGAGGACCCC | |
| | UUCCAGCCCCCCUCCAUCCCCAUCACCGUGUACUAC | |
| | GCCGUGCUGGAGCGCGCCUGCCGCUCCGUGCUGCU | |
| | GCACGCCCCCUCCGAGGCCCCCCAGAUCGUGCGCGG | |
| | CGCCUCCGACGAGGCCCGCAAGCACACCUACAACCU | |
| | GACCAUCGCCUGGUACCGCAUGGGCGACAACUGCG | |
| | CCAUCCCCAUCACCGUGAUGGAGUACACCGAGUGC | |
| | CCCUACAACAAGUCCCUGGGCGUGUGCCCCAUCCG | |
| | CACCCAGCCCCGCUGGUCCUACUACGACUCCUUCUC | |
| | CGCCGUGUCCGAGGACAACCUGGGCUUCCUGAUGC | |
| | ACGCCCCCGCCUUCGAGACCGCCGGCACCUACCUGC | |
| | GCCUGGUGAAGAUCAACGACUGGACCGAGAUCACC | |
| | CAGUUCAUCCUGGAGCACCGCGCCCGCGCCUCCUGC | |
| | AAGUACGCCCUGCCCCUGCGCAUCCCCCCCGCCGCC | |
| | UGCCUGACCUCCAAGGCCUACCAGCAGGGCGUGAC | |
| | CGUGGACUCCAUCGGCAUGCUGCCCCGCUUCAUCC | |
| | CCGAGAACCAGCGCACCGUGGCCCUGUACUCCCUG | |
| | AAGAUCGCCGGCUGGCACGGCCCCAAGCCCCCCUAC | |
| | ACCUCCACCCUGCUGCCCCCCGAGCUGUCCGACACC | |
| | ACCAACGCCACCCAGCCCGAGCUGGUGCCCGAGGA | |
| | CCCCGAGGACUCCGCCCUGCUGGAGGACCCCGCCGG | |
| | CACCGUGUCCUCCCAGAUCCCCCCCAACUGGCACAU | |
| | CCCCUCCAUCCAGGACGUGGCCCCCCACCACUAA | |
| HSV-2 | AAAUACGCCCUGGCCGAUCCUAGCCUGAAGAUGGC | 56 |
| gD | UGACCCCAACCGGUUCCGGGGCAAGAAUCUGCCUG | |
| [US6] | UUCUGGACCAGCUGACCGAUCCUCCUGGCGUGAAA | |
| (26-331) | CGGGUGUACCACAUCCAGCCAAGCCUGGAAGAUCC | |
| Version 1 | CUUCCAGCCUCCUAGCAUCCCCAUCACCGUGUACU | |
| | ACGCCGUGCUGGAAAGGGCCUGUAGAAGCGUGCUG | |
| | CUGCACGCCCCAUCUGAAGCCCCUCAAAUCGUCAG | |
| | AGGCGCUUCCGACGAGGCCAGAAAGCACACCUACA | |
| | ACCUGACAAUCGCCUGGUACAGAAUGGGCGACAAC | |
| | UGCGCCAUUCCUAUCACCGUGAUGGAGUACACCGA | |
| | GUGUCCCUACAACAAGAGCCUGGGCGUGUGCCCCA | |
| | UCAGAACACAGCCUAGAUGGUCCUACUACGACAGC | |
| | UUCAGCGCCGUGUCCGAGGACAAUCUGGGCUUCCU | |
| | GAUGCAUGCCCCUGCCUUUGAGACAGCCGGCACCU | |
| | AUCUGCGGCUGGUCAAGAUCAACGACUGGACCGAG | |
| | AUCACCCAGUUCAUCCUGGAACACAGAGCCAGAGC | |
| | CAGCUGCAAAUACGCUCUGCCCCUGAGAAUUCCUC | |
| | CUGCCGCCUGUCUGACAAGCAAGGCCUAUCAGCAG | |
| | GGCGUGACCGUGGAUAGCAUCGGCAUGCUGCCCAG | |
| | AUUCAUCCCCGAGAACCAGAGAACAGUGGCCCUGU | |
| | ACUCCCUGAAGAUCGCCGGAUGGCACGGACCCAAG | |
| | CCUCCAUACACAAGCACACUGCUGCCUCCAGAGCU | |
| | GAGCGACACCACCAAUGCCACACAGCCUGAACUGG | |
| | UGCCUGAGGACCCAGAGGAUUCUGCCCUGCUUGAA | |
| | GAUCCUGCCGGCACCGUGUCUAGCCAGAUUCCUCC | |
| | UAACUGGCACAUCCCCAGCAUCCAGGAUGUGGCCC | |
| | CUCAUCAUUGA | |
| HSV-2 | AAAUAUGCUCUCGCUGAUCCGAGCCUCAAGAUGGC | 57 |
| gD | AGAUCCCAACCGAUUUCGGGGAAAGAAUCUGCCAG | |
| [US6] | UACUGGACCAGCUGACGGACCCACCUGGCGUCAAA | |
| (26-331) | CGCGUCUACCACAUACAGCCUAGUCUUGAGGACCC | |
| Version 2 | UUUUCAGCCACCGUCUAUCCCCAUUACCGUGUACU | |
| | AUGCCGUGCUGGAACGCGCGUGUAGGUCAGUUCUG | |
| | CUGCAUGCCCCAUCCGAAGCCCCCCAGAUCGUCAG | |
| | AGGAGCUUCUGAUGAAGCACGCAAACACACCUACA | |
| | ACCUCACAAUAGCGUGGUAUCGAAUGGGCGAUAAC | |
| | UGCGCAAUUCCCAUCACAGUCAUGGAGUACACGGA | |
| | GUGCCCCUACAACAAGAGCCUCGGUGUUUGCCCUA | |
| | UCAGGACACAACCCAGGUGGAGCUAUUACGACAGU | |
| | UUCAGCGCCGUGUCUGAGGACAAUCUGGGGUUUCU | |
| | GAUGCACGCACCCGCCUUCGAGACUGCCGGCACCU | |
| | ACUUGCGGCUGGUGAAGAUCAACGACUGGACUGAG | |
| | AUCACCCAGUUCAUCCUGGAACAUAGGGCCAGAGC | |
| | CAGCUGCAAGUAUGCUCUUCCCCUGCGGAUUCCGC | |
| | CUGCAGCAUGUCUGACCUCAAAAGCCUACCAGCAA | |
| | GGGGUGACUGUGGACAGCAUUGGCAUGCUGCCUCG | |
| | UUUCAUUCCCGAGAAUCAACGGACAGUGGCUCUGU | |
| | AUUCCCUGAAGAUCGCAGGAUGGCAUGGGCCCAAA | |
| | CCACCUUAUACCUCUACGUUGCUUCCACCAGAACU | |
| | CAGUGACACCACUAAUGCGACACAGCCAGAACUUG | |
| | UGCCUGAGGAUCCUGAAGAUAGCGCUCUGUUGGAG | |
| | GAUCCAGCCGGUACUGUGUCCUCCCAGAUACCACC | |
| | CAAUUGGCACAUUCCUUCCAUUCAGGACGUAGCUC | |
| | CGCAUCACUGA | |
| HSV-2 | AAAUAUGCUCUCGCUGAUCCGAGCCUCAAGAUGGC | 58 |
| gD | AGAUCCCAACCGAUUUCGGGGAAAGAAUCUGCCAG | |
| [US6] | UACUGGACCAGCUGACGGACCCACCUGGCGUCAAA | |
| (26-331) | CGCGUCUACCACAUACAGCCUAGUCUUGAGGACCC | |
| Version | UUUUCAGCCACCGUCUAUCCCCAUUACCGUGUACU | |
| 2.1 | AUGCCGUGCUGGAACGCGCGUGUAGGUCAGUUCUG | |
| | CUGCAUGCCCCAUCCGAAGCCCCCCAGAUCGUCAG | |
| | AGGAGCUUCUGAUGAAGCACGCAAACACACCUACA | |
| | ACCUCACAAUAGCGUGGUAUCGAAUGGGCGAUAAC | |
| | UGCGCAAUUCCCAUCACAGUCAUGGAGUACACGGA | |
| | GUGCCCCUACAACAAGAGCCUCGGUGUUUGCCCUA | |
| | UCAGGACACAACCCAGGUGGAGCUAUUACGACAGU | |
| | UUCAGCGCCGUGUCUGAGGACAAUCUGGGGUUUCU | |
| | GAUGCACGCACCCGCCUUCGAGACUGCCGGCACCU | |
| | ACUUGCGGCUGGUGAAGAUCAACGACUGGACUGAG | |
| | AUCACCCAGUUCAUCCUGGAACAUAGGGCCAGAGC | |
| | CAGCUGCAAGUAUGCCCUUCCCCUGCGGAUUCCGC | |
| | CUGCAGCAUGUCUGACCUCAAAAGCCUACCAGCAA | |
| | GGGGUGACUGUGGACAGCAUUGGCAUGCUGCCUCG | |
| | UUUCAUUCCCGAGAAUCAACGGACAGUGGCUCUGU | |
| | AUUCCCUGAAGAUCGCAGGAUGGCAUGGGCCCAAA | |
| | CCACCUUAUACCUCUACGUUGCUUCCACCAGAACU | |
| | CAGUGACACCACUAAUGCGACACAGCCAGAACUUG | |
| | UGCCUGAGGAUCCUGAAGAUAGCGCUCUGUUGGAG | |
| | GAUCCAGCCGGUACUGUGUCCUCCCAGAUACCACC | |
| | CAAUUGGCACAUUCCUUCCAUUCAGGACGUAGCUC | |
| | CGCAUCACUGA | |
| HSV-2 | AAAUAUGCUCUCGCUGAUCCGAGCCUCAAGAUGGC | 59 |
| gD | AGAUCCCAACCGAUUUCGGGGAAAGAAUCUGCCAG | |
| [US6] | UACUGGACCAGCUGACGGACCCACCUGGCGUCAAA | |
| (26-331) | CGCGUCUACCACAUACAGCCUAGUCUUGAGGACCC | |
| Version | UUUUCAGCCACCGUCUAUCCCCAUUACCGUGUACU | |
| 2.2 | AUGCCGUGCUGGAACGCGCGUGUAGGUCAGUUCUG | |
| | CUGCAUGCCCCAUCCGAAGCCCCCCAGAUCGUCAG | |
| | AGGAGCUUCUGAUGAAGCACGCAAACACACCUACA | |
| | ACCUCACAAUAGCGUGGUAUCGAAUGGGCGAUAAC | |
| | UGCGCAAUUCCCAUCACAGUCAUGGAGUACACGGA | |
| | GUGCCCCUACAACAAGAGCCUCGGUGUUUGCCCUA | |
| | UCAGGACACAACCCAGGUGGAGCUAUUACGACAGU | |
| | UUCAGCGCCGUGUCUGAGGACAAUCUGGGGUUUCU | |
| | GAUGCACGCACCCGCCUUCGAGACUGCCGGCACCU | |
| | ACUUGCGGCUGGUGAAGAUCAACGACUGGACUGAG | |
| | AUCACCCAGUUCAUCCUGGAACAUAGGGCCAGAGC | |
| | CAGCUGCAAGUAUGCCCUUCCCCUGCGGAUUCCGC | |
| | CUGCAGCAUGUCUGACCUCAAAAGCCUACCAGCAA | |
| | GGGGUGACUGUGGACAGCAUUGGCAUGCUGCCUCG | |
| | UUUCAUUCCCGAGAAUCAACGGACAGUGGCUCUGU | |
| | AUUCCCUGAAGAUCGCAGGAUGGCAUGGGCCCAAA | |
| | CCACCUUAUACCUCUACGUUGCUUCCACCAGAACU | |
| | CAGUGACACCACUAAUGCGACACAGCCAGAACUUG | |
| | UGCCUGAGGAUCCUGAAGAUAGCGCUCUGUUGGAG | |
| | GAUCCAGCCGGUACUGUGUCCUCCCAGAUACCACC | |
| | CAAUUGGCACAUUCCUUCCAUUCAGGACGUAGCUC | |
| | CGCAUCACUGAUAA | |
| HSV-2 | AAAUACGCCCUGGCCGAUCCCUCCCUGAAAAUGGC | 60 |
| gD | CGAUCCCAACAGGUUCAGAGGCAAAAACCUGCCCG | |
| [US6] | UGCUGGAUCAGCUGACCGAUCCCCCUGGCGUGAAA | |
| (26-331) | AGAGUGUACCACAUCCAGCCCUCCCUGGAAGAUCC | |
| Version 3 | CUUCCAGCCCCCUUCCAUCCCCAUCACCGUGUACUA | |
| | CGCCGUGCUGGAAAGAGCUUGCAGAUCCGUGCUGC | |
| | UGCACGCCCCCUCCGAAGCCCCUCAGAUCGUGAGA | |
| | GGCGCCUCCGAUGAAGCCAGAAAACACACCUACAA | |
| | CCUGACCAUCGCCUGGUACAGAAUGGGCGAUAACU | |
| | GCGCCAUCCCCAUCACCGUGAUGGAAUACACCGAA | |
| | UGCCCCUACAACAAAUCCCUGGGCGUGUGCCCCAU | |
| | CAGAACCCAGCCCAGAUGGUCCUACUACGAUUCCU | |
| | UCUCCGCCGUGUCCGAAGAUAACCUGGGCUUCCUG | |
| | AUGCACGCCCCCGCCUUCGAAACCGCCGGCACCUAC | |
| | CUGAGACUGGUGAAAAUCAACGAUUGGACCGAAAU | |
| | CACCCAGUUCAUCCUGGAACACAGAGCCAGAGCCU | |
| | CCUGCAAAUACGCCCUGCCCCUGAGAAUCCCUCCCG | |
| | CCGCCUGCCUGACCUCCAAAGCCUACCAGCAGGGC | |
| | GUGACCGUGGAUUCCAUCGGCAUGCUGCCCAGAUU | |
| | CAUCCCCGAAAACCAGAGAACCGUGGCCCUGUACU | |
| | CCCUGAAAAUCGCCGGCUGGCACGGCCCCAAACCCC | |
| | CUUACACCUCCACCCUGCUGCCCCCUGAACUGUCCG | |
| | AUACCACCAACGCCACCCAGCCCGAACUGGUGCCCG | |
| | AAGAUCCCGAAGAUUCCGCCCUGCUGGAAGAUCCC | |
| | GCCGGCACCGUGUCCUCCCAGAUCCCUCCCAACUGG | |
| | CACAUCCCCUCCAUCCAGGAUGUGGCCCCUCACCAC | |
| | UGA | |
| HSV-2 | GAUCCGAGCCUCAAGAUGGCAGAUCCCAACCGAUU | 61 |
| gD | UCGGGGAAAGAAUCUGCCAGUACUGGACCAGCUGA | |
| [US6] | CGGACCCACCUGGCGUCAAACGCGUCUACCACAUA | |
| (31-331) | CAGCCUAGUCUUGAGGACCCUUUUCAGCCACCGUC | |
| Version 2 | UAUCCCCAUUACCGUGUACUAUGCCGUGCUGGAAC | |
| EAM | GCGCGUGUAGGUCAGUUCUGCUGCAUGCCCCAUCC | |
| | GAAGCCCCCCAGAUCGUCAGAGGAGCUUCUGAUGA | |
| | AGCACGCAAACACACCUACAACCUCACAAUAGCGU | |
| | GGUAUCGAAUGGGCGAUAACUGCGCAAUUCCCAUC | |
| | ACAGUCAUGGAGUACACGGAGUGCCCCUACAACAA | |
| | GAGCCUCGGUGUUUGCCCUAUCAGGACACAACCCA | |
| | GGUGGAGCUAUUACGACAGUUUCAGCGCCGUGUCU | |
| | GAGGACAAUCUGGGGUUUCUGAUGCACGCACCCGC | |
| | CUUCGAGACUGCCGGCACCUACUUGCGGCUGGUGA | |
| | AGAUCAACGACUGGACUGAGAUCACCCAGUUCAUC | |
| | CUGGAACAUAGGGCCAGAGCCAGCUGCAAGUAUGC | |
| | CCUUCCCCUGCGGAUUCCGCCUGCAGCAUGUCUGA | |
| | CCUCAAAAGCCUACCAGCAAGGGGUGACUGUGGAC | |
| | AGCAUUGGCAUGCUGCCUCGUUUCAUUCCCGAGAA | |
| | UCAACGGACAGUGGCUCUGUAUUCCCUGAAGAUCG | |
| | CAGGAUGGCAUGGGCCCAAACCACCUUAUACCUCU | |
| | ACGUUGCUUCCACCAGAACUCAGUGACACCACUAA | |
| | UGCGACACAGCCAGAACUUGUGCCUGAGGAUCCUG | |
| | AAGAUAGCGCUCUGUUGGAGGAUCCAGCCGGUACU | |
| | GUGUCCUCCCAGAUACCACCCAAUUGGCACAUUCC | |
| | UUCCAUUCAGGACGUAGCUCCGCAUCACUGA | |
| Complete | GGAAUAAAAGUCUCAACACAACAUAUACAAAACAA | 62 |
| HSV-1 gE | ACGAAUCUCAAGCAAUCAAGCAUUCUACUUCUAUU | |
| | GCAGCAAUUUAAAUCAUUUCUUUUAAAGCAAAAGC | |
| | AAUUUUCUGAAAAUUUUCACCAUUUACGAACGAUA | |
| | GCAUGCGCAUGCAGCUGCUGCUGCUGAUCGCCCU | |
| | GUCCCUGGCCCUGGUGACCAACUCCAAGACCUCC | |
| | UGGCGCCGCGUGUCCGUGGGCGAGGACGUGUCCCU | |
| | GCUGCCCGCCCCCGGCCCCACCGGCCGCGGCCCCAC | |
| | CCAGAAGCUGCUGUGGGCCGUGGAGCCCCUGGACG | |
| | GCUGCGGCCCCCUGCACCCCUCCUGGGUGUCCCUGA | |
| | UGCCCCCCAAGCAGGUGCCCGAGACCGUGGUGGAC | |
| | GCCGCCUGCAUGCGCGCCCCCGUGCCCCUGGCCAUG | |
| | GCCUACGCCCCCCCCGCCCCCUCCGCCACCGGCGGC | |
| | CUGCGCACCGACUUCGUGUGGCAGGAGCGCGCCGC | |
| | CGUGGUGAACCGCUCCCUGGUGAUCUACGGCGUGC | |
| | GCGAGACCGACUCCGGCCUGUACACCCUGUCCGUG | |
| | GGCGACAUCAAGGACCCCGCCCGCCAGGUGGCCUC | |
| | CGUGGUGCUGGUGGUGCAGCCCGCCCCCGUGCCCA | |
| | CCCCCCCCCCCACCCCCGCCGACUACGACGAGGACG | |
| | ACAACGACGAGGGCGAGGGCGAGGACGAGUCCCUG | |
| | GCCGGCACCCCCGCCUCCGGCACCCCCCGCCUGCCC | |
| | CCCUCCCCCGCCCCCCCCCGCUCCUGGCCCUCCGCC | |
| | CCCGAGGUGUCCCACGUGCGCGGCGUGACCGUGCG | |
| | CAUGGAGACCCCCGAGGCCAUCCUGUUCUCCCCCG | |
| | GCGAGGCCUUCUCCACCAACGUGUCCAUCCACGCC | |
| | AUCGCCCACGACGACCAGACCUACACCAUGGACGU | |
| | GGUGUGGCUGCGCUUCGACGUGCCCACCUCCUGCG | |
| | CCGAGAUGCGCAUCUACGAGUCCUGCCUGUACCAC | |
| | CCCCAGCUGCCCGAGUGCCUGUCCCCCGCCGACGCC | |
| | CCCUGCGCCGCCUCCACCUGGACCUCCCGCCUGGCC | |
| | GUGCGCUCCUACGCCGGCUGCUCCCGCACCAACCCC | |
| | CCCCCCCGCUGCUCCGCCGAGGCCCACAUGGAGCCC | |
| | UUCCCCGGCCUGGCCUGGCAGGCCGCCUCCGUGAA | |
| | CCUGGAGUUCCGCGACGCCUCCCCCCAGCACUCCGG | |
| | CCUGUACCUGUGCGUGGUGUACGUGAACGACCACA | |
| | UCCACGCCUGGGGCCACAUCACCAUCAACACCGCCG | |
| | CCCAGUACCGCAACGCCGUGGUGGAGCAGCCCCUG | |
| | CCCCAGCGCGGCGCCGACCUGGCCGAGCCCACCCAC | |
| | CCCCACGUGGGCGCCUAACUAGUAGUGACUGACUAG | |
| | GAUCUGGUUACCACUAAACCAGCCUCAAGAACACCCG | |
| | AAUGGAGUCUCUAAGCUACAUAAUACCAACUUACACU | |
| | UACAAAAUGUUGUCCCCCAAAAUGUAGCCAUUCGUAU | |
| | CUGCUCCUAAUAAAAAGAAAGUUUCUUCACAUUCUAA | |
| | AAAAAAAAAAAAAAAAAAAAAAAAAAAAAAAAAAAAAAAAA | |
| | AAAAAAAAAAAAAAAAAAAAAAAAAAAAAAAAAAAAAAAAA | |
| | AAAAAAAAAAAAAAAAAC | |
| Complete | GGAAUAAAAGUCUCAACACAACAUAUACAAAACAA | 63 |
| HSV-2 gE | ACGAAUCUCAAGCAAUCAAGCAUUCUACUUCUAUU | |
| | GCAGCAAUUUAAAUCAUUUCUUUUAAAGCAAAAGC | |
| | AAUUUUCUGAAAAUUUUCACCAUUUACGAACGAUA | |
| | GCAUGCGCAUGCAGCUGCUGCUGCUGAUCGCCCU | |
| | GUCCCUGGCCCUGGUGACCAACUCCCGCACCUCC | |
| | UGGAAGCGCGUGACCUCCGGCGAGGACGUGGUGCU | |
| | GCUGCCCGCCCCCGCCGGCCCCGAGGAGCGCACCCG | |
| | CGCCCACAAGCUGCUGUGGGCCGCCGAGCCCCUGG | |
| | ACGCCUGCGGCCCCCUGCGCCCCUCCUGGGUGGCCC | |
| | UGUGGCCCCCCCGCCGCGUGCUGGAGACCGUGGUG | |
| | GACGCCGCCUGCAUGCGCGCCCCCGAGCCCCUGGCC | |
| | AUCGCCUACUCCCCCCCCUUCCCCGCCGGCGACGAG | |
| | GGCCUGUACUCCGAGCUGGCCUGGCGCGACCGCGU | |
| | GGCCGUGGUGAACGAGUCCCUGGUGAUCUACGGCG | |
| | CCCUGGAGACCGACUCCGGCCUGUACACCCUGUCC | |
| | GUGGUGGGCCUGUCCGACGAGGCCCGCCAGGUGGC | |
| | CUCCGUGGUGCUGGUGGUGGAGCCCGCCCCCGUGC | |
| | CCACCCCCACCCCCGACGACUACGACGAGGAGGACG | |
| | ACGCCGGCGUGUCCGAGCGCACCCCCGUGUCCGUG | |
| | CCCCCCCCCACCCCCCCCCGCCGCCCCCCCGUGGCCC | |
| | CCCCCACCCACCCCCGCGUGAUCCCCGAGGUGUCCC | |
| | ACGUGCGCGGCGUGACCGUGCACAUGGAGACCCCC | |
| | GAGGCCAUCCUGUUCGCCCCCGGCGAGACCUUCGG | |
| | CACCAACGUGUCCAUCCACGCCAUCGCCCACGACGA | |
| | CGGCCCCUACGCCAUGGACGUGGUGUGGAUGCGCU | |
| | UCGACGUGCCCUCCUCCUGCGCCGAGAUGCGCAUC | |
| | UACGAGGCCUGCCUGUACCACCCCCAGCUGCCCGA | |
| | GUGCCUGUCCCCCGCCGACGCCCCCUGCGCCGUGUC | |
| | CUCCUGGGCCUACCGCCUGGCCGUGCGCUCCUACGC | |
| | CGGCUGCUCCCGCACCACCCCCCCCCCCCGCUGCUU | |
| | CGCCGAGGCCCGCAUGGAGCCCGUGCCCGGCCUGG | |
| | CCUGGCUGGCCUCCACCGUGAACCUGGAGUUCCAG | |
| | CACGCCUCCCCCCAGCACGCCGGCCUGUACCUGUGC | |
| | GUGGUGUACGUGGACGACCACAUCCACGCCUGGGG | |
| | CCACAUGACCAUCUCCACCGCCGCCCAGUACCGCAA | |
| | CGCCGUGGUGGAGCAGCACCUGCCCCAGCGCCAGC | |
| | CCGAGCCCGUGGAGCCCACCCGCCCCCACGUGCGCG | |
| | CCUAACUAGUAGUGACUGACUAGGAUCUGGUUACCA | |
| | CUAAACCAGCCUCAAGAACACCCGAAUGGAGUCUCUA | |
| | AGCUACAUAAUACCAACUUACACUUACAAAAUGUUGU | |
| | CCCCCAAAAUGUAGCCAUUCGUAUCUGCUCCUAAUAA | |
| | AAAGAAAGUUUCUUCACAUUCUAAAAAAAAAAAAAAAA | |
| | AAAAAAAAAAAAAAAAAAAAAAAAAAAAAAAAAAAAAAAAA | |
| | AAAAAAAAAAAAAAAAAAAAAAAAAAAAAAAAAAAAAAAAA | |
| | AAAC | |
| Complete | GGAAUAAAAGUCUCAACACAACAUAUACAAAACAA | 64 |
| HSV-1 gC | ACGAAUCUCAAGCAAUCAAGCAUUCUACUUCUAUU | |
| | GCAGCAAUUUAAAUCAUUUCUUUUAAAGCAAAAGC | |
| | AAUUUUCUGAAAAUUUUCACCAUUUACGAACGAUA | |
| | GCAUGGCCAUCUCCGGCGUGCCCGUGCUGGGCU | |
| | UCUUCAUCAUCGCCGUGCUGAUGUCCGCCCAGGA | |
| | GUCCUGGGCCGAGACCGCCUCCACCGGCCCCACCA | |
| | UCACCGCCGGCGCCGUGACCAACGCCUCCGAGGCCC | |
| | CCACCUCCGGCUCCCCCGGCUCCGCCGCCUCCCCCG | |
| | AGGUGACCCCCACCUCCACCCCCAACCCCAACAACG | |
| | UGACCCAGAACAAGACCACCCCCACCGAGCCCGCCU | |
| | CCCCCCCCACCACCCCCAAGCCCACCUCCACCCCCA | |
| | AGUCCCCCCCCACCUCCACCCCCGACCCCAAGCCCA | |
| | AGAACAACACCACCCCCGCCAAGUCCGGCCGCCCCA | |
| | CCAAGCCCCCCGGCCCCGUGUGGUGCGACCGCCGCG | |
| | ACCCCCUGGCCCGCUACGGCUCCCGCGUGCAGAUCC | |
| | GCUGCCGCUUCCGCAACUCCACCCGCAUGGAGUUC | |
| | CGCCUGCAGAUCUGGCGCUACUCCAUGGGCCCCUC | |
| | CCCCCCCAUCGCCCCCGCCCCCGACCUGGAGGAGGU | |
| | GCUGACCAACAUCACCGCCCCCCCCGGCGGCCUGCU | |
| | GGUGUACGACUCCGCCCCCAACCUGACCGACCCCCA | |
| | CGUGCUGUGGGCCGAGGGCGCCGGCCCCGGCGCCG | |
| | ACCCCCCCCUGUACUCCGUGACCGGCCCCCUGCCCA | |
| | CCCAGCGCCUGAUCAUCGGCGAGGUGACCCCCGCC | |
| | ACCCAGGGCAUGUACUACCUGGCCUGGGGCCGCAU | |
| | GGACUCCCCCCACGAGUACGGCACCUGGGUGCGCG | |
| | UGCGCAUGUUCCGCCCCCCCUCCCUGACCCUGCAGC | |
| | CCCACGCCGUGAUGGAGGGCCAGCCCUUCAAGGCC | |
| | ACCUGCACCGCCGCCGCCUACUACCCCCGCAACCCC | |
| | GUGGAGUUCGACUGGUUCGAGGACGACCGCCAGGU | |
| | GUUCAACCCCGGCCAGAUCGACACCCAGACCCACG | |
| | AGCACCCCGACGGCUUCACCACCGUGUCCACCGUG | |
| | ACCUCCGAGGCCGUGGGGGCCAGGUGCCCCCCCGC | |
| | ACCUUCACCUGCCAGAUGACCUGGCACCGCGACUC | |
| | CGUGACCUUCUCCCGCCGCAACGCCACCGGCCUGGC | |
| | CCUGGUGCUGCCCCGCCCCACCAUCACCAUGGAGU | |
| | UCGGCGUGCGCCACGUGGUGUGCACCGCCGGCUGC | |
| | GUGCCCGAGGGCGUGACCUUCGCCUGGUUCCUGGG | |
| | CGACGACCCCUCCCCCGCCGCCAAGUCCGCCGUGAC | |
| | CGCCCAGGAGUCCUGCGACCACCCCGGCCUGGCCAC | |
| | CGUGCGCUCCACCCUGCCCAUCUCCUACGACUACUC | |
| | CGAGUACAUCUGCCGCCUGACCGGCUACCCCGCCG | |
| | GCAUCCCCGUGCUGGAGCACCACUAACUAGUAGUGA | |
| | CUGACUAGGAUCUGGUUACCACUAAACCAGCCUCAAG | |
| | AACACCCGAAUGGAGUCUCUAAGCUACAUAAUACCAA | |
| | CUUACACUUACAAAAUGUUGUCCCCCAAAAUGUAGCC | |
| | AUUCGUAUCUGCUCCUAAUAAAAAGAAAGUUUCUUCA | |
| | CAUUCUAAAAAAAAAAAAAAAAAAAAAAAAAAAAAAAAAA | |
| | AAAAAAAAAAAAAAAAAAAAAAAAAAAAAAAAAAAAAAAAA | |
| | AAAAAAAAAAAAAAAAAAAAAAAAAAC | |
| Complete | GGAAUAAAAGUCUCAACACAACAUAUACAAAACAA | 65 |
| HSV-2 gC | ACGAAUCUCAAGCAAUCAAGCAUUCUACUUCUAUU | |
| | GCAGCAAUUUAAAUCAUUUCUUUUAAAGCAAAAGC | |
| | AAUUUUCUGAAAAUUUUCACCAUUUACGAACGAUA | |
| | GCAUGCGCAUGCAGCUGCUGCUGCUGAUCGCCCU | |
| | GUCCCUGGCCCUGGUGACCAACUCCGCCUCCCCC | |
| | GGCCGCACCAUCACCGUGGGCCCCCGCGGCAACGCC | |
| | UCCAACGCCGCCCCCUCCGCCUCCCCCCGCAACGCC | |
| | UCCGCCCCCCGCACCACCCCCACCCCCCCCCAGCCC | |
| | CGCAAGGCCACCAAGUCCAAGGCCUCCACCGCCAA | |
| | GCCCGCCCCCCCCCCCAAGACCGGCCCCCCCAAGAC | |
| | CUCCUCCGAGCCCGUGCGCUGCAACCGCCACGACCC | |
| | CCUGGCCCGCUACGGCUCCCGCGUGCAGAUCCGCU | |
| | GCCGCUUCCCCAACUCCACCCGCACCGAGUUCCGCC | |
| | UGCAGAUCUGGCGCUACGCCACCGCCACCGACGCC | |
| | GAGAUCGGCACCGCCCCCUCCCUGGAGGAGGUGAU | |
| | GGUGAACGUGUCCGCCCCCCCCGGCGGCCAGCUGG | |
| | UGUACGACUCCGCCCCCAACCGCACCGACCCCCACG | |
| | UGAUCUGGGCCGAGGGCGCCGGCCCCGGCGCCUCC | |
| | CCCCGCCUGUACUCCGUGGUGGGCCCCCUGGGCCGC | |
| | CAGCGCCUGAUCAUCGAGGAGCUGACCCUGGAGAC | |
| | CCAGGGCAUGUACUACUGGGUGUGGGGCCGCACCG | |
| | ACCGCCCCUCCGCCUACGGCACCUGGGUGCGCGUGC | |
| | GCGUGUUCCGCCCCCCCUCCCUGACCAUCCACCCCC | |
| | ACGCCGUGCUGGAGGGCCAGCCCUUCAAGGCCACC | |
| | UGCACCGCCGCCACCUACUACCCCGGCAACCGCGCC | |
| | GAGUUCGUGUGGUUCGAGGACGGCCGCCGCGUGUU | |
| | CGACCCCGCCCAGAUCCACACCCAGACCCAGGAGAA | |
| | CCCCGACGGCUUCUCCACCGUGUCCACCGUGACCUC | |
| | CGCCGCCGUGGGCGGCCAGGGCCCCCCCCGCACCUU | |
| | CACCUGCCAGCUGACCUGGCACCGCGACUCCGUGU | |
| | CCUUCUCCCGCCGCAACGCCUCCGGCACCGCCUCCG | |
| | UGCUGCCCCGCCCCACCAUCACCAUGGAGUUCACCG | |
| | GCGACCACGCCGUGUGCACCGCCGGCUGCGUGCCC | |
| | GAGGGCGUGACCUUCGCCUGGUUCCUGGGCGACGA | |
| | CUCCUCCCCCGCCGAGAAGGUGGCCGUGGCCUCCCA | |
| | GACCUCCUGCGGCCGCCCCGGCACCGCCACCAUCCG | |
| | CUCCACCCUGCCCGUGUCCUACGAGCAGACCGAGU | |
| | ACAUCUGCCGCCUGGCCGGCUACCCCGACGGCAUCC | |
| | CCGUGCUGGAGCACCACUAACUAGUAGUGACUGACU | |
| | AGGAUCUGGUUACCACUAAACCAGCCUCAAGAACACC | |
| | CGAAUGGAGUCUCUAAGCUACAUAAUACCAACUUACA | |
| | CUUACAAAAUGUUGUCCCCCAAAAUGUAGCCAUUCGU | |
| | AUCUGCUCCUAAUAAAAAGAAAGUUUCUUCACAUUCU | |
| | AAAAAAAAAAAAAAAAAAAAAAAAAAAAAAAAAAAAAAAAA | |
| | AAAAAAAAAAAAAAAAAAAAAAAAAAAAAAAAAAAAAAAAA | |
| | AAAAAAAAAAAAAAAAAAAC | |
| Complete | GGAAUAAAAGUCUCAACACAACAUAUACAAAACAA | 66 |
| HSV-1 | ACGAAUCUCAAGCAAUCAAGCAUUCUACUUCUAUU | |
| gD | GCAGCAAUUUAAAUCAUUUCUUUUAAAGCAAAAGC | |
| | AAUUUUCUGAAAAUUUUCACCAUUUACGAACGAUA | |
| | GCAUGCGCAUGCAGCUGCUGCUGCUGAUCGCCCU | |
| | GUCCCUGGCCCUGGUGACCAACUCCAAGUACGCC | |
| | CUGGCCGACGCCUCCCUGAAGAUGGCCGACCCCAA | |
| | CCGCUUCCGCGGCAAGGACCUGCCCGUGCUGGACC | |
| | AGCUGACCGACCCCCCCGGCGUGCGCCGCGUGUACC | |
| | ACAUCCAGGCCGGCCUGCCCGACCCCUUCCAGCCCC | |
| | CCUCCCUGCCCAUCACCGUGUACUACGCCGUGCUG | |
| | GAGCGCGCCUGCCGCUCCGUGCUGCUGAACGCCCCC | |
| | UCCGAGGCCCCCCAGAUCGUGCGCGGCGCCUCCGA | |
| | GGACGUGCGCAAGCAGCCCUACAACCUGACCAUCG | |
| | CCUGGUUCCGCAUGGGCGGCAACUGCGCCAUCCCC | |
| | AUCACCGUGAUGGAGUACACCGAGUGCUCCUACAA | |
| | CAAGUCCCUGGGCGCCUGCCCCAUCCGCACCCAGCC | |
| | CCGCUGGAACUACUACGACUCCUUCUCCGCCGUGU | |
| | CCGAGGACAACCUGGGCUUCCUGAUGCACGCCCCC | |
| | GCCUUCGAGACCGCCGGCACCUACCUGCGCCUGGU | |
| | GAAGAUCAACGACUGGACCGAGAUCACCCAGUUCA | |
| | UCCUGGAGCACCGCGCCAAGGGCUCCUGCAAGUAC | |
| | GCCCUGCCCCUGCGCAUCCCCCCCUCCGCCUGCCUG | |
| | UCCCCCCAGGCCUACCAGCAGGGCGUGACCGUGGA | |
| | CUCCAUCGGCAUGCUGCCCCGCUUCAUCCCCGAGA | |
| | ACCAGCGCACCGUGGCCGUGUACUCCCUGAAGAUC | |
| | GCCGGCUGGCACGGCCCCAAGGCCCCCUACACCUCC | |
| | ACCCUGCUGCCCCCCGAGCUGUCCGAGACCCCCAAC | |
| | GCCACCCAGCCCGAGCUGGCCCCCGAGGACCCCGAG | |
| | GACUCCGCCCUGCUGGAGGACCCCGUGGGCACCGU | |
| | GGCCCCCCAGAUCCCCCCCAACUGGCACAUCCCCUC | |
| | CAUCCAGGACGCCGCCACCCCCUACUAACUAGUAGU | |
| | GACUGACUAGGAUCUGGUUACCACUAAACCAGCCUCA | |
| | AGAACACCCGAAUGGAGUCUCUAAGCUACAUAAUACC | |
| | AACUUACACUUACAAAAUGUUGUCCCCCAAAAUGUAG | |
| | CCAUUCGUAUCUGCUCCUAAUAAAAAGAAAGUUUCUU | |
| | CACAUUCUAAAAAAAAAAAAAAAAAAAAAAAAAAAAAAAA | |
| | AAAAAAAAAAAAAAAAAAAAAAAAAAAAAAAAAAAAAAAAA | |
| | AAAAAAAAAAAAAAAAAAAAAAAAAAAAC | |
| Complete | GGAAUAAAAGUCUCAACACAACAUAUACAAAACAA | 67 |
| HSV-2 | ACGAAUCUCAAGCAAUCAAGCAUUCUACUUCUAUU | |
| gD | GCAGCAAUUUAAAUCAUUUCUUUUAAAGCAAAAGC | |
| | AAUUUUCUGAAAAUUUUCACCAUUUACGAACGAUA | |
| | GCAUGACCCGCCUGACCGUGCUGGCCCUGCUGGC | |
| | CGGCCUGCUGGCCUCCUCCCGCGCCAAGUACGCC | |
| | CUGGCCGACCCCUCCCUGAAGAUGGCCGACCCCAAC | |
| | CGCUUCCGCGGCAAGAACCUGCCCGUGCUGGACCA | |
| | GCUGACCGACCCCCCCGGCGUGAAGCGCGUGUACC | |
| | ACAUCCAGCCCUCCCUGGAGGACCCCUUCCAGCCCC | |
| | CCUCCAUCCCCAUCACCGUGUACUACGCCGUGCUG | |
| | GAGCGCGCCUGCCGCUCCGUGCUGCUGCACGCCCCC | |
| | UCCGAGGCCCCCCAGAUCGUGCGCGGCGCCUCCGAC | |
| | GAGGCCCGCAAGCACACCUACAACCUGACCAUCGC | |
| | CUGGUACCGCAUGGGCGACAACUGCGCCAUCCCCA | |
| | UCACCGUGAUGGAGUACACCGAGUGCCCCUACAAC | |
| | AAGUCCCUGGGCGUGUGCCCCAUCCGCACCCAGCCC | |
| | CGCUGGUCCUACUACGACUCCUUCUCCGCCGUGUC | |
| | CGAGGACAACCUGGGCUUCCUGAUGCACGCCCCCG | |
| | CCUUCGAGACCGCCGGCACCUACCUGCGCCUGGUG | |
| | AAGAUCAACGACUGGACCGAGAUCACCCAGUUCAU | |
| | CCUGGAGCACCGCGCCCGCGCCUCCUGCAAGUACGC | |
| | CCUGCCCCUGCGCAUCCCCCCCGCCGCCUGCCUGAC | |
| | CUCCAAGGCCUACCAGCAGGGCGUGACCGUGGACU | |
| | CCAUCGGCAUGCUGCCCCGCUUCAUCCCCGAGAACC | |
| | AGCGCACCGUGGCCCUGUACUCCCUGAAGAUCGCC | |
| | GGCUGGCACGGCCCCAAGCCCCCCUACACCUCCACC | |
| | CUGCUGCCCCCCGAGCUGUCCGACACCACCAACGCC | |
| | ACCCAGCCCGAGCUGGUGCCCGAGGACCCCGAGGA | |
| | CUCCGCCCUGCUGGAGGACCCCGCCGGCACCGUGUC | |
| | CUCCCAGAUCCCCCCCAACUGGCACAUCCCCUCCAU | |
| | CCAGGACGUGGCCCCCCACCACUAACUAGUAGUGAC | |
| | UGACUAGGAUCUGGUUACCACUAAACCAGCCUCAAGA | |
| | ACACCCGAAUGGAGUCUCUAAGCUACAUAAUACCAAC | |
| | UUACACUUACAAAAUGUUGUCCCCCAAAAUGUAGCCA | |
| | UUCGUAUCUGCUCCUAAUAAAAAGAAAGUUUCUUCAC | |
| | AUUCUAAAAAAAAAAAAAAAAAAAAAAAAAAAAAAAAAAA | |
| | AAAAAAAAAAAAAAAAAAAAAAAAAAAAAAAAAAAAAAAA | |
| | AAAAAAAAAAAAAAAAAAC | |

In another embodiment, an HSV glycoprotein protein fragment encoded by RNA utilized in the methods and compositions of the present disclosure is an immunogenic fragment. In another embodiment, an HSV glycoprotein protein fragment encoded by RNA utilized in the methods and compositions of the present disclosure is an immunoprotective antigen. In some embodiments, an immunoprotective antigen need not be the entire protein. The protective immune response generally involves, in another embodiment, an antibody response. In another embodiment, mutants, sequence conservative variants, and functional conservative variants of glycoproteins described herein are useful in methods and compositions of the present disclosure, provided that all such variants retain the required immuno-protective effect. In another embodiment, the immunogenic fragment can comprise an immuno-protective antigen from any strain of HSV. In another embodiment, the immunogenic fragment can comprise sequence variants of HSV, as found in infected individuals.

In some embodiments, an HSV glycoprotein or immunogenic fragment thereof, encoded by RNA utilized in the methods and compositions of the present disclosure is a homologue of a sequence provided herein. In another embodiment, an HSV glycoprotein or immunogenic fragment thereof, encoded by RNA utilized in the methods and compositions of the present disclosure is an isoform of the sequence provided herein. In another embodiment, an HSV glycoprotein or immunogenic fragment thereof, encoded by RNA utilized in the methods and compositions of the present disclosure is a variant of the sequence provided herein. In another embodiment, an HSV glycoprotein or immunogenic fragment thereof, encoded by RNA utilized in the methods and compositions of the present disclosure is a fragment of the sequence provided herein.

In another embodiment, the glycoprotein fragment encoded by RNA of the methods and compositions of the present disclosure comprises the ectodomain of the glycoprotein or immunogenic fragment thereof. In another embodiment, the glycoprotein fragment encoded by RNA of the methods and compositions of the present disclosure consists of the ectodomain of the glycoprotein or immunogenic fragment thereof. In another embodiment, the glycoprotein fragment encoded by RNA of the methods and compositions of the present disclosure comprises a fragment of the ectodomain of the glycoprotein. In another embodiment, the glycoprotein fragment may be any glycoprotein fragment known in the art.

In another embodiment, the glycoprotein or immunogenic fragment encoded by an RNA utilized in the methods and compositions of the present disclosure may be from any strain of HSV. In another embodiment, the immunogenic fragment encoded by RNA utilized in the methods and compositions of the present disclosure may comprise sequence variants of HSV, as found in infected individuals.

In some embodiments, “variant” refers to an amino acid or nucleic acid sequence (or in other embodiments, an organism or tissue) that is different from the majority of the population but is still sufficiently similar to the common mode to be considered to be one of them, for example splice variants. In some embodiments, the variant may a sequence conservative variant, while in another embodiment, the variant may be a functional conservative variant. In some embodiments, a variant may comprise an addition, deletion or substitution of one or more amino acids.

“Immune evasion domain” refers, in some embodiments, to a domain that interferes with or reduces in vivo anti-HSV efficacy of anti-HSV antibodies (e.g. anti-gD antibodies). In another embodiment, the domain interferes or reduces in vivo anti-HSV efficacy of an anti-HSV immune response. In another embodiment, the domain reduces the immunogenicity of an HSV protein (e.g. gD) during subsequent infection. In another embodiment, the domain reduces the immunogenicity of an HSV protein during subsequent challenge. In another embodiment, the domain reduces the immunogenicity of HSV during subsequent challenge. In another embodiment, the domain reduces the immunogenicity of an HSV protein in the context of ongoing HSV infection. In another embodiment, the domain reduces the immunogenicity of HSV in the context of ongoing HSV infection. In another embodiment, the domain functions as an IgG Fc receptor. In another embodiment, the domain promotes antibody bipolar bridging, which in some embodiments, is a term that refers to an antibody molecule binding by its Fab domain to an HSV antigen and by its Fc domain to a separate HSV antigen, such as in some embodiments, gE, thereby blocking the ability of the Fc domain to activate complement.

The present disclosure also provides for modified RNA encoding analogs of HSV proteins or polypeptides, or fragments thereof. Analogs may differ from naturally occurring proteins or peptides by conservative amino acid sequence substitutions or by modifications which do not affect sequence, or by both.

In another embodiment, an HSV glycoprotein encoded by modified RNA of the present disclosure is homologous to a sequence set forth hereinabove, either expressly or by reference to a GenBank entry. The terms “homology,” “homologous,” etc, when in reference to any protein or peptide, refer, in some embodiments, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, “homology” refers to identity of a protein sequence encoded by an RNA to a sequence disclosed herein of greater than 70%. In another embodiment, the identity is greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In some embodiments, “isoform” refers to a version of a molecule, for example, a protein, with only slight differences to another isoform of the same protein. In some embodiments, isoforms may be produced from different but related genes, or in another embodiment, may arise from the same gene by alternative splicing. In another embodiment, isoforms are caused by single nucleotide polymorphisms.

In another embodiment, the RNA encoding a glycoprotein or glycoprotein fragment as described herein further encodes an antigenic tag. In some embodiments, the tag is a histidine (“His”) tag. In some embodiments, the His tag comprises 5 histidine residues. In another embodiment, the His tag comprises 6 histidine residues.

In another embodiment, methods and compositions of the present disclosure utilize a chimeric molecule, comprising a fusion of an RNA encoding an HSV protein or immunogenic fragment thereof, with an RNA encoding a tag polypeptide that provides an epitope to which an anti-tag antibody can selectively bind. The epitope tag is placed, in other embodiments, at the amino- or carboxyl-terminus of the protein or in an internal location therein. The presence of such epitope-tagged forms of the recombinant HSV protein or immunogenic fragment thereof, is detected, in another embodiment, using an antibody against the tag polypeptide. In another embodiment, inclusion of the epitope tag enables the recombinant HSV protein or immunogenic fragment thereof, to be readily purified by affinity purification using an anti-tag antibody or another type of affinity matrix that binds to the epitope tag. Various tag polypeptides and their respective antibodies are known in the art.

In some embodiments, the compositions of the present disclosure comprise an adjuvant, while in another embodiment, the compositions do not comprise an adjuvant. “Adjuvant” refers, in another embodiment, to compounds that, when administered to an individual or tested in vitro, increase the immune response to an antigen in the individual or test system to which the antigen is administered. In another embodiment, an immune adjuvant enhances an immune response to an antigen that is weakly immunogenic when administered alone, i.e., inducing no or weak antibody titers or cell-mediated immune response. In another embodiment, the adjuvant increases antibody titers to the antigen. In another embodiment, the adjuvant lowers the dose of the antigen effective to achieve an immune response in the individual. Multiple types of adjuvants are known in the art and described in detail in U. S. Patent Publication 2013/0028925 which is hereby incorporated by reference herein.

### Signal Sequences and Signal Peptides

In some embodiments, an RNA encoding a glycoprotein as described herein comprises a signal sequence encoding a signal peptide, e.g., that is functional in mammalian cells. In some embodiments, a signal sequence encodes a modified signal peptide (e.g., comprising amino acid substitutions or amino acid additions). In some embodiments, a signal sequence is a codon optimized signal sequence.

In some embodiments, a utilized signal sequence is a heterologous signal sequence. In some embodiments, a heterologous signal sequence comprises or consists of a non-human signal sequence. In some embodiments, a heterologous signal sequence comprises or consists of a viral signal sequence. In some embodiments, a viral signal sequence comprises or consists of an HSV signal sequence (e.g., an HSV-1 or HSV-2 signal sequence). In some embodiments, a signal sequence comprises or consists of an HSV-1 signal sequence. In some embodiments, a signal sequence comprises or consists of an HSV-2 signal sequence. In some embodiments, a signal sequence encodes a signal peptide characterized by a length of about 15 to 30 amino acids. In some embodiments, a signal sequence encodes a signal peptide that preferably allows transport of an HSV-1 glycoprotein, or immunogenic fragment thereof, an HSV-2 glycoprotein, or immunogenic fragment thereof, or both, with which it is associated into a defined cellular compartment, preferably a cell surface, endoplasmic reticulum (ER) or endosomal-lysosomal compartment.

In some embodiments, a signal sequence is the native signal sequence of the encoded glycoprotein. In some embodiments, a signal sequence is or comprises an HSV glycoprotein D (gD) signal sequence (e.g., an HSV-1 or HSV-2 gD signal sequence). In some embodiments, a signal peptide encoded by a signal sequence is or comprises an HSV-2 gD signal peptide (SEQ ID NO: 68). In another embodiment, an HSV-2 gD signal peptide comprises KY (SEQ ID NO: 69), KYA (SEQ ID NO: 70), KYAL (SEQ ID NO: 71), or KYALA (SEQ ID NO: 72) at the C terminus of the signal peptide. In some embodiments, a signal peptide encoded by a signal sequence is or comprises an HSV-1 gD signal peptide (SEQ ID NO: 73). In some embodiments, an HSV-1 gD signal peptide comprises KY at the C terminus of the signal peptide (SEQ ID NO: 74).

In some embodiments, a signal sequence is or comprises an HSV glycoprotein C (gC) signal sequence (e.g., an HSV-1 or HSV-2 gC signal sequence). In some embodiments, a signal peptide encoded by a signal sequence is or comprises an HSV-2 gC signal peptide (SEQ ID NO: 75). In another embodiment, a signal peptide encoded by a signal sequence is or comprises an HSV-1 gC signal peptide (SEQ ID NO:76).

In some embodiments, a signal sequence is or comprises an HSV glycoprotein E (gE) signal sequence (e.g., an HSV-1 or HSV-2 gE signal sequence). In some embodiments, a signal peptide encoded by a signal sequence is or comprises an HSV-2 gE signal peptide (SEQ ID NO: 77). In some embodiments, an HSV-2 gE signal peptide comprises RTS at the C terminus of the signal peptide (SEQ ID NO: 78). In some embodiments, an HSV-2 gE signal peptide comprises A20V, A21V, and A22V substitutions (SEQ ID NO: 79). In some embodiments, a signal peptide is or comprises an HSV-1 gE signal peptide (SEQ ID NO: 80).

In some embodiments, a signal sequence is or comprises an HSV gB signal sequence (e.g., an HSV-1 or HSV-2 gB signal sequence). In some embodiments, a signal peptide encoded by a signal sequence is or comprises an HSV-2 gB signal peptide (SEQ ID NO: 81). In some embodiments, a signal peptide encoded by a signal sequence is or comprises an HSV-1 gB signal peptide (SEQ ID NO: 82). In some embodiments, an HSV-1 gB signal peptide comprises AP at the C terminus of the signal peptide (SEQ ID NO: 83).

In some embodiments, a signal sequence is or comprises an HSV gI signal sequence (e.g., an HSV-1 or HSV-2 gI signal sequence). In some embodiments, a signal peptide encoded by a signal sequence is or comprises an HSV-2 gI signal peptide (SEQ ID NO: 84). In some embodiments, an HSV-2 gI signal peptide comprises an additional leucine residue at the C terminus of the signal peptide (SEQ ID NO: 85). In some embodiments, an HSV-2 gI signal peptide comprises amino acid residues 1-18 of the wild-type peptide (SEQ ID NO: 86). In some embodiments, a signal peptide is or comprises an HSV-1 gI signal peptide (SEQ ID NO: 87).

In other embodiments, a signal sequence is a heterologous signal sequence. In some embodiments, a heterologous signal peptide encoded by a heterologous signal sequence comprises an IL-2 signal peptide (SEQ ID NO: 88). In some embodiments, a heterologous signal peptide encoded by a heterologous signal sequence comprises or consists of an azurocidin signal peptide (SEQ ID NO: 89). In some embodiments, a heterologous signal peptide encoded by a heterologous signal sequence comprises or consists of an MHC Class II signal peptide (SEQ ID NO: 90). In some embodiments, a signal sequence comprises or consists of an Ebola virus signal sequence. In some embodiments, an Ebola virus signal peptide encoded by an Ebola virus signal sequence comprises or consists of an Ebola virus spike glycoprotein (SGP) signal peptide (SEQ ID NO: 91). In other embodiments, an RNA encoding a glycoprotein as described herein does not comprise a signal sequence. In some embodiments, an RNA as described herein only encodes an ectodomain without a signal peptide.

In some embodiments, a signal sequence encodes a signal peptide listed in Table 3, or a signal peptide having 1, 2, 3, 4, or 5 amino acid differences thereto. In some embodiments, a signal peptide is selected from those listed in Table 3 and functionally connected to the N-terminus of an HSV immunogen selected from those listed in Table 1.

TABLE 3
Exemplary signal peptides

| Sequence Name | Amino Acid Sequence | SEQ ID NO: |
|---|---|---|
| HSV-2 gD Signal Sequence | MGRLTSGVGTAALLVVAVGLRVVCA | 68 |
| HSV-2 gD Signal Sequence | MGRLTSGVGTAALLVVAVGLRVVCAK | 69 |
| KY | Y | |
| HSV-2 gD Signal Sequence | MGRLTSGVGTAALLVVAVGLRVVCAK | 70 |
| KYA | YA | |
| HSV-2 gD Signal Sequence | MGRLTSGVGTAALLVVAVGLRVVCAK | 71 |
| KYAL | YAL | |
| HSV-2 gD Signal Sequence | MGRLTSGVGTAALLVVAVGLRVVCAK | 72 |
| KYALA | YALA | |
| HSV-1 gD Signal Sequence | MGGAAARLGAVILFVVIVGLHGVRG | 73 |
| HSV-1 gD Signal Sequence | MGGAAARLGAVILFVVIVGLHGVRGK | 74 |
| KY | Y | |
| HSV-2 gC Signal Sequence | MALGRVGLAVGLWGLLWVGVVVVLA | 75 |
| | NA | |
| HSV-1 gC Signal Sequence | MDRGAVVGFLLGVCVVSCLA | 76 |
| HSV-2 gE Signal Sequence | MARGAGLVFFVGVWVVSCLAAAP | 77 |
| HSV-2 gE Signal Sequence | MARGAGLVFFVGVWVVSCLAAAPRTS | 78 |
| RTS | | |
| HSV-2 gE Signal Sequence | MARGAGLVFFVGVWVVSCLVVVP | 79 |
| LVVVP | | |
| HSV-1 gE Signal Sequence | MDRGAVVGFLLGVCVVSCLA | 80 |
| HSV-2 gB Signal Sequence | MRGGGLICALVVGALVAAVASA | 81 |
| HSV-1 gB Signal Sequence | MHQGAPSWGRRWFVVWALLGLTLGV | 82 |
| | LVASA | |
| HSV-1 gB Signal Sequence | MHQGAPSWGRRWFVVWALLGLTLGV | 83 |
| AP | LVASAAP | |
| HSV-2 gI Signal Sequence | MPGRSLQGLAILGLWVCATG | 84 |
| HSV-2 gI Signal Sequence | MPGRSLQGLAILGLWVCATGL | 85 |
| L | | |
| HSV-2 gI Signal Sequence | MPGRSLQGLAILGLWVCA | 86 |
| (1-18) | | |
| HSV-1 gI Signal Sequence | MPCRPLQGLVLVGLWVCATS | 87 |
| IL2 Signal Sequence | MRMQLLLLIALSLALVINS | 88 |
| Azurocidin Signal | MTRLTVLALLAGLLASSRA | 89 |
| Sequence | | |
| MHCII Signal Sequence | MAISGVPVLGFFIIAVLMSAQESWA | 90 |
| EBV SGP Signal Sequence | MGVTGILQLPRDRFKRTSFFLWVIILFQ | 91 |
| | RTFSIP | |

In some embodiments, a signal sequence comprises a sequence listed in Table 4, or a signal sequence at least 80%, 85%, 90%, 91%, 92%, 93%, 94%, 95%, 96%, 97%, 98%, or 99% identical to a sequence listed in Table 4. In some embodiments, a signal sequence is selected from those listed in Table 4 and functionally connected (i.e., in frame) to the 5′ end of an HSV immunogen nucleic acid sequence selected from those listed in Table 2.

TABLE 4
Exemplary signal sequences

| Sequence Name | Version | Amino Acid Sequence | SEQ ID NO: |
|---|---|---|---|
| HSV-2 gD | WT | AUGGGCCGCCUGACCUCCGGCGUGGGC | 92 |
| | | ACCGCCGCCCUGCUGGUGGUGGCCGUG | |
| | | GGCCUGCGCGUGGUGUGCGCC | |
| HSV-2 gD | Version 1 | AUGGGCAGACUGACAUCUGGCGUGGG | 93 |
| | | AACAGCUGCUCUGCUGGUGGUUGCUG | |
| | | UGGGCCUGAGAGUCGUGUGUGCC | |
| HSV-2 gD | Version 2 | AUGGGGAGACUCACAUCAGGCGUAGG | 94 |
| | | AACCGCUGCCCUGUUGGUCGUGGCCG | |
| | | UUGGUCUGAGAGUUGUGUGUGCC | |
| HSV-2 gD | Version 3 | AUGGGCAGACUGACCUCCGGCGUGGG | 95 |
| | | CACCGCCGCCCUGCUGGUGGUGGCCGU | |
| | | GGGCCUGAGAGUGGUGUGCGCC | |
| HSV-2 gD KYA | WT | AUGGGCCGCCUGACCUCCGGCGUGGGC | 96 |
| | | ACCGCCGCCCUGCUGGUGGUGGCCGUG | |
| | | GGCCUGCGCGUGGUGUGCGCCAAGUA | |
| | | CGCC | |
| HSV-2 gD KYA | Version 1 | AUGGGCAGACUGACAUCUGGCGUGGG | 97 |
| | | AACAGCUGCUCUGCUGGUGGUUGCUG | |
| | | UGGGCCUGAGAGUCGUGUGUGCCAAA | |
| | | UACGCC | |
| HSV-2 gD KYA | Version 2 | AUGGGGAGACUCACAUCAGGCGUAGG | 98 |
| | | AACCGCUGCCCUGUUGGUCGUGGCCG | |
| | | UUGGUCUGAGAGUUGUGUGUGCCAAA | |
| | | UAUGCU | |
| HSV-2 gD KYA | Version 3 | AUGGGCAGACUGACCUCCGGCGUGGG | 99 |
| | | CACCGCCGCCCUGCUGGUGGUGGCCGU | |
| | | GGGCCUGAGAGUGGUGUGCGCCAAAU | |
| | | ACGCC | |
| HSV-2 gD KYAL | Version 2 | AUGGGGAGACUCACAUCAGGCGUAGG | 100 |
| | | AACCGCUGCCCUGUUGGUCGUGGCCG | |
| | | UUGGUCUGAGAGUUGUGUGUGCCAAA | |
| | | UAUGCUCUG | |
| HSV-2 gD KYAL | Version 3 | AUGGGCAGACUGACCUCCGGCGUGGG | 101 |
| | | CACCGCCGCCCUGCUGGUGGUGGCCGU | |
| | | GGGCCUGAGAGUGGUGUGCGCCAAAU | |
| | | ACGCCCUG | |
| HSV-2 gD KYALA | WT | AUGGGCCGCCUGACCUCCGGCGUGGGC | 102 |
| | | ACCGCCGCCCUGCUGGUGGUGGCCGUG | |
| | | GGCCUGCGCGUGGUGUGCGCCAAGUA | |
| | | CGCCCUGGCC | |
| HSV-2 gD KYALA | Version 1 | AUGGGCAGACUGACAUCUGGCGUGGG | 103 |
| | | AACAGCUGCUCUGCUGGUGGUUGCUG | |
| | | UGGGCCUGAGAGUCGUGUGUGCCAAA | |
| | | UACGCCCUGGCC | |
| HSV-2 gD KYALA | Version 2 | AUGGGGAGACUCACAUCAGGCGUAGG | 104 |
| | | AACCGCUGCCCUGUUGGUCGUGGCCG | |
| | | UUGGUCUGAGAGUUGUGUGUGCCAAA | |
| | | UAUGCUCUGGCA | |
| HSV-2 gD KYALA | Version 3 | AUGGGCAGACUGACCUCCGGCGUGGG | 105 |
| | | CACCGCCGCCCUGCUGGUGGUGGCCGU | |
| | | GGGCCUGAGAGUGGUGUGCGCCAAAU | |
| | | ACGCCCUG GCC | |
| HSV-1 gD | Version 1 | AUGGGCGGAGCUGCUGCUAGACUGGG | 106 |
| | | AGCCGUGAUCCUGUUCGUGGUUAUCG | |
| | | UGGGACUGCAUGGCGUGCGGGGC | |
| HSV-1 gD | Version 2 | AUGGGAGGAGCAGCUGCCAGACUCGG | 107 |
| | | UGCCGUGAUCCUGUUCGUGGUCAUUG | |
| | | UUGGCCUGCACGGGGUAAGGGGC | |
| HSV-1 gD | Version 3 | AUGGGAGGAGCUGCUGCUAGAUUGGG | 108 |
| | | AGCUGUGAUUCUGUUUGUGGUGAUUG | |
| | | UGGGACUGCAUGGAGUGAGAGGA | |
| HSV-1 gD | Version 4 | AUGGGCGGCGCAGCCGCUAGACUGGG | 109 |
| | | UGCAGUCAUCCUCUUUGUGGUGAUCG | |
| | | UGGGGCUGCAUGGGGUCAGAGGG | |
| HSV-1 gD | Version 4.1 | AUGGGCGGAGCUGCGGCUCGCCUCGG | 110 |
| | | AGCCGUCAUUCUGUUCGUGGUGAUCG | |
| | | UGGGUCUGCAUGGGGUCAGAGGA | |
| HSV-1 gD KY | Version 1 | AUGGGCGGAGCUGCUGCUAGACUGGG | 111 |
| | | AGCCGUGAUCCUGUUCGUGGUUAUCG | |
| | | UGGGACUGCAUGGCGUGCGGGGCAAG | |
| | | UAU | |
| HSV-1 gD KY | Version 2 | AUGGGAGGAGCAGCUGCCAGACUCGG | 112 |
| | | UGCCGUGAUCCUGUUCGUGGUCAUUG | |
| | | UUGGCCUGCACGGGGUAAGGGGCAAG | |
| | | UAC | |
| HSV-1 gD KY | Version 2.1 | AUGGGGAGACUCACAUCCGGAGUGGG | 113 |
| | | CACUGCUGCCCUGUUGGUCGUGGCCG | |
| | | UUGGUCUGAGAGUUGUGUGUGCCAAA | |
| | | UAUGCUCUGGCA | |
| HSV-1 gD KY | Version 3 | AUGGGAGGAGCUGCUGCUAGAUUGGG | 114 |
| | | AGCUGUGAUUCUGUUUGUGGUGAUUG | |
| | | UGGGACUGCAUGGAGUGAGAGGAAAA | |
| | | UAC | |
| HSV-1 gD KY | Version 4 | AUGGGCGGCGCAGCCGCUAGACUGGG | 115 |
| | | UGCAGUCAUCCUCUUUGUGGUGAUCG | |
| | | UGGGGCUGCAUGGGGUCAGAGGGAAG | |
| | | UAU | |
| HSV-1 gD KY | Version 4.1 | AUGGGCGGAGCUGCGGCUCGCCUCGG | 116 |
| | | AGCCGUCAUUCUGUUCGUGGUGAUCG | |
| | | UGGGUCUGCAUGGGGUCAGAGGAAAG | |
| | | UAC | |
| HSV-2 gC | Version 2 | AUGGCCUUGGGGAGAGUGGGCCUUGC | 117 |
| | | AGUGGGUCUGUGGGGACUGCUCUGGG | |
| | | UUGGCGUAGUCGUCGUGCUGGCUAAC | |
| | | GCA | |
| HSV-2 gC | Version 2.1 | AUGGCACUAGGGAGAGUGGGAUUAGC | 118 |
| | | UGUGGGUCUGUGGGGACUGCUCUGGG | |
| | | UAGGAGUCGUCGUCGUGCUGGCUAAC | |
| | | GCA | |
| HSV-2 gC | Version 2.2 | AUGGCCUUGGGGAGAGUCGGCCUUGC | 119 |
| | | AGUGGGACUGUGGGGUCUGCUGUGGG | |
| | | UUGGCGUGGUAGUCGUGCUCGCUAAC | |
| | | GCC | |
| HSV-2 gC | Version 2.3 | AUGGCUCUGGGCAGAGUUGGACUGGC | 120 |
| | | UGUUGGACUGUGGGGACUGCUGUGGG | |
| | | UUGGAGUGGUGGUGGUGCUGGCUAAU | |
| | | GCU | |
| HSV-2 gE | Version 2 | AUGGCACGGGGAGCCGGAUUGGUGUU | 121 |
| | | CUUUGUGGGCGUGUGGGUGGUGAGCU | |
| | | GCUUGGCAGCCGCACCA | |
| HSV-2 gE | Version 2.1 | AUGGCACGGGGCGCAGGUUUGGUCUU | 122 |
| | | UUUCGUGGGCGUGUGGGUGGUGAGCU | |
| | | GCUUGGCAGCCGCACCA | |
| HSV-2 gE | Version 2.2 | AUGGCGAGAGGAGCCGGACUCGUGUU | 123 |
| | | CUUUGUGGGAGUCUGGGUUGUGAGCU | |
| | | GCCUGGCAGCAGCUCCA | |
| HSV-2 gE | Version 2.3 | AUGGCGAGAGGAGCCGGGCUCGUGUU | 124 |
| | | CUUUGUGGGCGUAUGGGUCGUUUCCU | |
| | | GCCUGGCUGCCGCACCC | |
| HSV-2 gE RTS | Version 1 | AUGGCUAGAGGUGCCGGCCUGGUGUU | 125 |
| | | CUUUGUUGGCGUGUGGGUCGUGUCCU | |
| | | GUCUGGCCGCUGCUCCUAGAACAUCU | |
| HSV-2 gE RTS | Version 2 | AUGGCGAGAGGAGCCGGGCUCGUGUU | 126 |
| | | CUUUGUGGGCGUAUGGGUCGUUUCCU | |
| | | GCCUGGCUGCCGCACCCAGGACCAGC | |
| HSV-2 gE RTS | Version 3 | AUGGCAAGAGGAGCAGGACUGGUGUU | 127 |
| | | UUUCGUGGGAGUUUGGGUGGUGUCUU | |
| | | GCCUGGCUGCUGCUCCAAGAACCUCU | |
| HSV-2 gE RTS | Version 4 | AUGGCGAGAGGAGCCGGACUCGUGUU | 128 |
| | | CUUUGUGGGAGUCUGGGUUGUGAGCU | |
| | | GCCUGGCAGCAGCUCCACGCACUAGC | |
| HSV-2 gE LVVVP | Version 2 | AUGGCGAGAGGAGCCGGACUCGUGUU | 129 |
| | | CUUUGUGGGAGUCUGGGUUGUGAGCU | |
| | | GCCUGGUAGUAGUUCCA | |
| HSV-1 gB | Version 1 | AUGCAUCAGGGCGCUCCAUCUUGGGG | 130 |
| | | UAGACGUUGGUUCGUUGUGUGGGCCC | |
| | | UGCUGGGACUGACACUGGGAGUUCUG | |
| | | GUUGCCUCUGCU | |
| HSV-1 gB | Version 2 | AUGCACCAGGGUGCCCCUUCCUGGGGC | 131 |
| | | AGAAGGUGGUUCGUGGUGUGGGCCCU | |
| | | UCUGGGGCUGACCCUCGGAGUCCUGG | |
| | | UUGCGAGCGCA | |
| HSV-1 gB | Version 3 | AUGCACCAGGGAGCACCUUCUUGGGG | 132 |
| | | AAGAAGAUGGUUUGUGGUGUGGGCUC | |
| | | UGCUGGGACUGACCCUGGGAGUGCUG | |
| | | GUGGCUUCUGCU | |
| HSV-1 gB | Version 4 | AUGCAUCAAGGCGCACCCAGUUGGGG | 133 |
| | | CAGACGGUGGUUCGUAGUGUGGGCCU | |
| | | UGCUGGGACUGACUCUCGGUGUCCUC | |
| | | GUUGCGUCUGCU | |
| HSV-1 gB AP | Version 1 | AUGCAUCAGGGCGCUCCAUCUUGGGG | 134 |
| | | UAGACGUUGGUUCGUUGUGUGGGCCC | |
| | | UGCUGGGACUGACACUGGGAGUUCUG | |
| | | GUUGCCUCUGCUGCUCCU | |
| HSV-1 gB AP | Version 2 | AUGCACCAGGGUGCCCCUUCCUGGGGC | 135 |
| | | AGAAGGUGGUUCGUGGUGUGGGCCCU | |
| | | UCUGGGGCUGACCCUCGGAGUCCUGG | |
| | | UUGCGAGCGCAGCUCCC | |
| HSV-1 gB AP | Version 3 | AUGCACCAGGGAGCACCUUCUUGGGG | 136 |
| | | AAGAAGAUGGUUUGUGGUGUGGGCUC | |
| | | UGCUGGGACUGACCCUGGGAGUGCUG | |
| | | GUGGCUUCUGCUGCUCCU | |
| HSV-1 gB AP | Version 4 | AUGCAUCAAGGCGCACCCAGUUGGGG | 137 |
| | | CAGACGGUGGUUCGUAGUGUGGGCCU | |
| | | UGCUGGGACUGACUCUCGGUGUCCUC | |
| | | GUUGCGUCUGCUGCACCG | |
| HSV-2 gI | Version 1 | AUGCCUGGCAGAUCUCUGCAAGGACU | 138 |
| | | GGCCAUCCUCGGACUGUGGGUUUGCG | |
| | | CAACAGGA | |
| HSV-2 gI | Version 2 | AUGCCCGGCAGAAGCCUCCAGGGACU | 139 |
| | | GGCUAUCCUGGGGCUGUGGGUGUGCG | |
| | | CCACCGGU | |
| HSV-2 gI | Version 3 | AUGCCUGGAAGAUCUCUCCAGGGACU | 140 |
| | | GGCAAUCCUGGGACUGUGGGUGUGUG | |
| | | CAACAGGA | |
| HSV-2 gI | Version 4 | AUGCCUGGGAGAAGCCUGCAAGGGCU | 141 |
| | | CGCAAUCUUGGGCCUGUGGGUUUGUG | |
| | | CCACAGGC | |
| HSV-2 gI L | Version 1 | AUGCCUGGCAGAUCUCUGCAAGGACU | 142 |
| | | GGCCAUCCUCGGACUGUGGGUUUGCG | |
| | | CAACAGGACUG | |
| HSV-2 gI L | Version 2 | AUGCCCGGCAGAAGCCUCCAGGGACU | 143 |
| | | GGCUAUCCUGGGGCUGUGGGUGUGCG | |
| | | CCACCGGUCUU | |
| HSV-2 gI L | Version 3 | AUGCCUGGAAGAUCUCUCCAGGGACU | 144 |
| | | GGCAAUCCUGGGACUGUGGGUGUGUG | |
| | | CAACAGGACUG | |
| HSV-2 gI L | Version 4 | AUGCCUGGGAGAAGCCUGCAAGGGCU | 145 |
| | | CGCAAUCUUGGGCCUGUGGGUUUGUG | |
| | | CCACAGGCUUG | |
| HSV-2 gI (1-18) | Version 1 | AUGCCUGGCAGAUCUCUGCAAGGACU | 146 |
| | | GGCCAUCCUCGGACUGUGGGUUUGCG | |
| | | CA | |
| HSV-2 gI (1-18) | Version 2 | AUGCCCGGCAGAAGCCUCCAGGGACU | 147 |
| | | GGCUAUCCUGGGGCUGUGGGUGUGCG | |
| | | CC | |
| HSV-2 gI (1-18) | Version 3 | AUGCCUGGAAGAUCUCUCCAGGGACU | 148 |
| | | GGCAAUCCUGGGACUGUGGGUGUGUG | |
| | | CA | |
| HSV-2 gI (1-18) | Version 4 | AUGCCUGGGAGAAGCCUGCAAGGGCU | 149 |
| | | CGCAAUCUUGGGCCUGUGGGUUUGUG | |
| | | CC | |
| IL2 | WT | AUGCGCAUGCAGCUGCUGCUGCUGAU | 150 |
| | | CGCCCUGUCCCUGGCCCUGGUGACCAA | |
| | | CUCC | |
| IL2 | Version 1 | AUGAGAAUGCAGCUGCUGCUCCUGAU | 151 |
| | | CGCCCUGUCUCUGGCCCUGGUCACCAA | |
| | | UAGC | |
| IL2 | Version 2 | AUGCGCAUGCAACUGCUCCUGCUGAU | 152 |
| | | UGCGUUGAGCCUUGCCCUGGUGACCA | |
| | | ACAGC | |
| IL2 | Version 3 | AUGAGAAUGCAGCUGCUGCUGCUGAU | 153 |
| | | CGCCCUGUCCCUGGCCCUGGUGACCAA | |
| | | CUCC | |
| Azurocidin | WT | AUGACCCGCCUGACCGUGCUGGCCCUG | 154 |
| | | CUGGCCGGCCUGCUGGCCUCCUCCCGC | |
| | | GCC | |
| MHCII | WT | AUGGCCAUCUCCGGCGUGCCCGUGCUG | 155 |
| | | GGCUUCUUCAUCAUCGCCGUGCUGAU | |
| | | GUCCGCCCAGGAGUCCUGGGCC | |
| EBV SGP | Version 1 | AUGGGAGUGACCGGCAUUCUCCAGCU | 156 |
| | | GCCUCGGGACAGAUUCAAGCGGACCA | |
| | | GCUUCUUCCUGUGGGUCAUCAUCCUG | |
| | | UUCCAGCGGACCUUCAGCAUCCCC | |
| EBV SGP | Version 2 | AUGGGAGUGACUGGCAUACUCCAGCU | 157 |
| | | UCCUAGAGACAGGUUUAAGCGCACAU | |
| | | CCUUCUUUCUGUGGGUCAUCAUCCUG | |
| | | UUCCAACGGACCUUCAGCAUUCCC | |
| EBV SGP | Version 3 | AUGGGAGUAACCGGAAUUCUCCAGCU | 158 |
| | | GCCAAGAGAUCGAUUCAAAAGAACAU | |
| | | CAUUUUUCCUUUGGGUAAUUAUUCUG | |
| | | UUUCAGAGAACAUUUUCCAUCCCU | |
| EBV SGP | Version 4 | AUGGGAGUUACCGGAAUUCUGCAAUU | 159 |
| | | GCCCAGGGAUCGGUUCAAGCGCACAU | |
| | | CCUUCUUCCUGUGGGUCAUCAUCCUCU | |
| | | UUCAGCGUACUUUCUCCAUACCC | |

In some embodiments, the signal sequence of the RNA as described herein encodes a signal peptide of any one of SEQ ID NOs: 68-91. In other embodiments, the signal sequence of the RNA as described herein comprises any one of SEQ ID NOs: 92-159. In other embodiments, the signal sequence of the RNA as described herein comprises

a)
(SEQ ID NO: 154)
AUGACCCGCCUGACCGUGCUGGCCCUGCUGGCCGGCCUGCUGGCCUCCU
CCCGCGCC,
b)
(SEQ ID NO: 150)
AUGCGCAUGCAGCUGCUGCUGCUGAUCGCCCUGUCCCUGGCCCUGGUGA
CCAACUCC,
c)
(SEQ ID NO: 155)
AUGGCCAUCUCCGGCGUGCCCGUGCUGGGCUUCUUCAUCAUCGCCGUGC
UGAUGUCCGCCCAGGAGUCCUGGGCC,
or
d)
(SEQ ID NO: 98)
AUGGGGAGACUCACAUCAGGCGUAGGAACCGCUGCCCUGUUGGUCGUGG
CCGUUGGUCUGAGAGUUGUGUGUGCCAAAUAUGCu.

### Exemplary Signal Peptide and HSV Immunogen Combinations

The present disclosure also provides RNA comprising a nucleotide sequence encoding a protein, wherein the protein comprises an HSV (e.g., HSV-1, HSV-2, or both) glycoprotein or immunogenic fragment thereof, and a signal peptide. In some embodiments, a nucleotide sequence encodes a protein, wherein the protein comprises an HSV-2 glycoprotein, and a signal peptide.

Exemplary proteins comprising signal peptides and HSV-2 immunogens are shown in Table 5. In some embodiments, a protein of the present disclosure comprises an amino acid sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 5.

TABLE 5
Exemplary signal peptide/HSV-2 immunogen combinations

| SEQ | Signal | HSV | |
|---|---|---|---|
| ID NO: | Peptide | Glycoprotein | Amino Acid Sequence |
| 160 | IL2 | HSV-2 gC (27- | MRMQLLLLIALSLALVTNSASPGRTITVGPRGN |
| | | 426) | ASNAAPSASPRNASAPRTTPTPPQPRKATKSKAST |
| | | | AKPAPPPKTGPPKTSSEPVRCNRHDPLARYGSRV |
| | | | QIRCRFPNSTRTEFRLQIWRYATATDAEIGTAPSLE |
| | | | EVMVNVSAPPGGQLVYDSAPNRTDPHVIWAEGA |
| | | | GPGASPRLYSVVGPLGRQRLIIEELTLETQGMYY |
| | | | WVWGRTDRPSAYGTWVRVRVFRPPSLTIHPHAV |
| | | | LEGQPFKATCTAATYYPGNRAEFVWFEDGRRVF |
| | | | DPAQIHTQTQENPDGFSTVSTVTSAAVGGQGPPRT |
| | | | FTCQLTWHRDSVSFSRRNASGTASVLPRPTITMEF |
| | | | TGDHAVCTAGCVPEGVTFAWFLGDDSSPAEKVA |
| | | | VASQTSCGRPGTATIRSTLPVSYEQTEYICRLAGY |
| | | | PDGIPVLEHH |
| 161 | IL2 | HSV-2 gC (28- | MRMQLLLLIALSLALVTNSSPGRTITVGPRGNAS |
| | | 426) | NAAPSASPRNASAPRTTPTPPQPRKATKSKASTAK |
| | | | PAPPPKTGPPKTSSEPVRCNRHDPLARYGSRVQIR |
| | | | CRFPNSTRTEFRLQIWRYATATDAEIGTAPSLEEV |
| | | | MVNVSAPPGGQLVYDSAPNRTDPHVIWAEGAGP |
| | | | GASPRLYSVVGPLGRQRLIIEELTLETQGMYYWV |
| | | | WGRTDRPSAYGTWVRVRVFRPPSLTIHPHAVLEG |
| | | | QPFKATCTAATYYPGNRAEFVWFEDGRRVFDPA |
| | | | QIHTQTQENPDGFSTVSTVTSAAVGGQGPPRTFTC |
| | | | QLTWHRDSVSFSRRNASGTASVLPRPTITMEFTGD |
| | | | HAVCTAGCVPEGVTFAWFLGDDSSPAEKVAVAS |
| | | | QTSCGRPGTATIRSTLPVSYEQTEYICRLAGYPDGI |
| | | | PVLEHH |
| 162 | HSV-2 | HSV-2 gC (27- | MGRLTSGVGTAALLVVAVGLRVVCAASPGRTI |
| | gD | 426) | TVGPRGNASNAAPSASPRNASAPRTTPTPPQPRKA |
| | | | TKSKASTAKPAPPPKTGPPKTSSEPVRCNRHDPLA |
| | | | RYGSRVQIRCRFPNSTRTEFRLQIWRYATATDAEI |
| | | | GTAPSLEEVMVNVSAPPGGQLVYDSAPNRTDPHV |
| | | | IWAEGAGPGASPRLYSVVGPLGRQRLIIEELTLET |
| | | | QGMYYWVWGRTDRPSAYGTWVRVRVFRPPSLTI |
| | | | HPHAVLEGQPFKATCTAATYYPGNRAEFVWFED |
| | | | GRRVFDPAQIHTQTQENPDGFSTVSTVTSAAVGG |
| | | | QGPPRTFTCQLTWHRDSVSFSRRNASGTASVLPRP |
| | | | TITMEFTGDHAVCTAGCVPEGVTFAWFLGDDSSP |
| | | | AEKVAVASQTSCGRPGTATIRSTLPVSYEQTEYIC |
| | | | RLAGYPDGIPVLEHH |
| 163 | HSV-2 | HSV-2 gC (28- | MGRLTSGVGTAALLVVAVGLRVVCASPGRTIT |
| | gD | 426) | VGPRGNASNAAPSASPRNASAPRTTPTPPQPRKAT |
| | | | KSKASTAKPAPPPKTGPPKTSSEPVRCNRHDPLAR |
| | | | YGSRVQIRCRFPNSTRTEFRLQIWRYATATDAEIG |
| | | | TAPSLEEVMVNVSAPPGGQLVYDSAPNRTDPHVI |
| | | | WAEGAGPGASPRLYSVVGPLGRQRLIIEELTLETQ |
| | | | GMYYWVWGRTDRPSAYGTWVRVRVFRPPSLTIH |
| | | | PHAVLEGQPFKATCTAATYYPGNRAEFVWFEDG |
| | | | RRVFDPAQIHTQTQENPDGFSTVSTVTSAAVGGQ |
| | | | GPPRTFTCQLTWHRDSVSFSRRNASGTASVLPRPT |
| | | | ITMEFTGDHAVCTAGCVPEGVTFAWFLGDDSSPA |
| | | | EKVAVASQTSCGRPGTATIRSTLPVSYEQTEYICR |
| | | | LAGYPDGIPVLEHH |
| 164 | HSV-2 | HSV-2 gC (27- | MGRLTSGVGTAALLVVAVGLRVVCAKYAASP |
| | gD- | 426) | GRTITVGPRGNASNAAPSASPRNASAPRTTPTPPQ |
| | KYA | | PRKATKSKASTAKPAPPPKTGPPKTSSEPVRCNRH |
| | | | DPLARYGSRVQIRCRFPNSTRTEFRLQIWRYATAT |
| | | | DAEIGTAPSLEEVMVNVSAPPGGQLVYDSAPNRT |
| | | | DPHVIWAEGAGPGASPRLYSVVGPLGRQRLIIEEL |
| | | | TLETQGMYYWVWGRTDRPSAYGTWVRVRVFRP |
| | | | PSLTIHPHAVLEGQPFKATCTAATYYPGNRAEFV |
| | | | WFEDGRRVFDPAQIHTQTQENPDGFSTVSTVTSA |
| | | | AVGGQGPPRTFTCQLTWHRDSVSFSRRNASGTAS |
| | | | VLPRPTITMEFTGDHAVCTAGCVPEGVTFAWFLG |
| | | | DDSSPAEKVAVASQTSCGRPGTATIRSTLPVSYEQ |
| | | | TEYICRLAGYPDGIPVLEHH |
| 165 | HSV-2 | HSV-2 gC (28- | MGRLTSGVGTAALLVVAVGLRVVCAKYASPG |
| | gD- | 426) | RTITVGPRGNASNAAPSASPRNASAPRTTPTPPQP |
| | KYA | | RKATKSKASTAKPAPPPKTGPPKTSSEPVRCNRHD |
| | | | PLARYGSRVQIRCRFPNSTRTEFRLQIWRYATATD |
| | | | AEIGTAPSLEEVMVNVSAPPGGQLVYDSAPNRTD |
| | | | PHVIWAEGAGPGASPRLYSVVGPLGRQRLIIEELT |
| | | | LETQGMYYWVWGRTDRPSAYGTWVRVRVFRPP |
| | | | SLTIHPHAVLEGQPFKATCTAATYYPGNRAEFVW |
| | | | FEDGRRVFDPAQIHTQTQENPDGFSTVSTVTSAAV |
| | | | GGQGPPRTFTCQLTWHRDSVSFSRRNASGTASVL |
| | | | PRPTITMEFTGDHAVCTAGCVPEGVTFAWFLGDD |
| | | | SSPAEKVAVASQTSCGRPGTATIRSTLPVSYEQTE |
| | | | YICRLAGYPDGIPVLEHH |
| 166 | HSV-2 | HSV-2 gC (28- | MGRLTSGVGTAALLVVAVGLRVVCAKYALAS |
| | gD- | 426) | PGRTITVGPRGNASNAAPSASPRNASAPRTTPTPP |
| | KYALA | | QPRKATKSKASTAKPAPPPKTGPPKTSSEPVRCNR |
| | | | HDPLARYGSRVQIRCRFPNSTRTEFRLQIWRYATA |
| | | | TDAEIGTAPSLEEVMVNVSAPPGGQLVYDSAPNR |
| | | | TDPHVIWAEGAGPGASPRLYSVVGPLGRQRLIIEE |
| | | | LTLETQGMYYWVWGRTDRPSAYGTWVRVRVFR |
| | | | PPSLTIHPHAVLEGQPFKATCTAATYYPGNRAEFV |
| | | | WFEDGRRVFDPAQIHTQTQENPDGFSTVSTVTSA |
| | | | AVGGQGPPRTFTCQLTWHRDSVSFSRRNASGTAS |
| | | | VLPRPTITMEFTGDHAVCTAGCVPEGVTFAWFLG |
| | | | DDSSPAEKVAVASQTSCGRPGTATIRSTLPVSYEQ |
| | | | TEYICRLAGYPDGIPVLEHH |
| 167 | HSV-2 | HSV-2 gC (27- | MGRLTSGVGTAALLVVAVGLRVVCAKYALAA |
| | gD- | 426) | SPGRTITVGPRGNASNAAPSASPRNASAPRTTPTPP |
| | KYALA | | QPRKATKSKASTAKPAPPPKTGPPKTSSEPVRCNR |
| | | | HDPLARYGSRVQIRCRFPNSTRTEFRLQIWRYATA |
| | | | TDAEIGTAPSLEEVMVNVSAPPGGQLVYDSAPNR |
| | | | TDPHVIWAEGAGPGASPRLYSVVGPLGRQRLIIEE |
| | | | LTLETQGMYYWVWGRTDRPSAYGTWVRVRVFR |
| | | | PPSLTIHPHAVLEGQPFKATCTAATYYPGNRAEFV |
| | | | WFEDGRRVFDPAQIHTQTQENPDGFSTVSTVTSA |
| | | | AVGGQGPPRTFTCQLTWHRDSVSFSRRNASGTAS |
| | | | VLPRPTITMEFTGDHAVCTAGCVPEGVTFAWFLG |
| | | | DDSSPAEKVAVASQTSCGRPGTATIRSTLPVSYEQ |
| | | | TEYICRLAGYPDGIPVLEHH |
| 168 | HSV-2 | HSV-2 gC (28- | MALGRVGLAVGLWGLLWVGVVVVLANASPGR |
| | gC | 426) | TITVGPRGNASNAAPSASPRNASAPRTTPTPPQPR |
| | | | KATKSKASTAKPAPPPKTGPPKTSSEPVRCNRHDP |
| | | | LARYGSRVQIRCRFPNSTRTEFRLQIWRYATATDA |
| | | | EIGTAPSLEEVMVNVSAPPGGQLVYDSAPNRTDP |
| | | | HVIWAEGAGPGASPRLYSVVGPLGRQRLIIEELTL |
| | | | ETQGMYYWVWGRTDRPSAYGTWVRVRVFRPPS |
| | | | LTIHPHAVLEGQPFKATCTAATYYPGNRAEFVWF |
| | | | EDGRRVFDPAQIHTQTQENPDGFSTVSTVTSAAV |
| | | | GGQGPPRTFTCQLTWHRDSVSFSRRNASGTASVL |
| | | | PRPTITMEFTGDHAVCTAGCVPEGVTFAWFLGDD |
| | | | SSPAEKVAVASQTSCGRPGTATIRSTLPVSYEQTE |
| | | | YICRLAGYPDGIPVLEHH |
| 169 | HSV-2 | HSV-2 gC (27- | MALGRVGLAVGLWGLLWVGVVVVLANAASP |
| | gC | 426) | GRTITVGPRGNASNAAPSASPRNASAPRTTPTPPQ |
| | | | PRKATKSKASTAKPAPPPKTGPPKTSSEPVRCNRH |
| | | | DPLARYGSRVQIRCRFPNSTRTEFRLQIWRYATAT |
| | | | DAEIGTAPSLEEVMVNVSAPPGGQLVYDSAPNRT |
| | | | DPHVIWAEGAGPGASPRLYSVVGPLGRQRLIIEEL |
| | | | TLETQGMYYWVWGRTDRPSAYGTWVRVRVFRP |
| | | | PSLTIHPHAVLEGQPFKATCTAATYYPGNRAEFV |
| | | | WFEDGRRVFDPAQIHTQTQENPDGFSTVSTVTSA |
| | | | AVGGQGPPRTFTCQLTWHRDSVSFSRRNASGTAS |
| | | | VLPRPTITMEFTGDHAVCTAGCVPEGVTFAWFLG |
| | | | DDSSPAEKVAVASQTSCGRPGTATIRSTLPVSYEQ |
| | | | TEYICRLAGYPDGIPVLEHH |
| 170 | HSV-1 | HSV-2 gC (28- | MGGAAARLGAVILFVVIVGLHGVRGKYSPGRTI |
| | gD KY | 426) | TVGPRGNASNAAPSASPRNASAPRTTPTPPQPRKA |
| | | | TKSKASTAKPAPPPKTGPPKTSSEPVRCNRHDPLA |
| | | | RYGSRVQIRCRFPNSTRTEFRLQIWRYATATDAEI |
| | | | GTAPSLEEVMVNVSAPPGGQLVYDSAPNRTDPHV |
| | | | IWAEGAGPGASPRLYSVVGPLGRQRLIIEELTLET |
| | | | QGMYYWVWGRTDRPSAYGTWVRVRVFRPPSLTI |
| | | | HPHAVLEGQPFKATCTAATYYPGNRAEFVWFED |
| | | | GRRVFDPAQIHTQTQENPDGFSTVSTVTSAAVGG |
| | | | QGPPRTFTCQLTWHRDSVSFSRRNASGTASVLPRP |
| | | | TITMEFTGDHAVCTAGCVPEGVTFAWFLGDDSSP |
| | | | AEKVAVASQTSCGRPGTATIRSTLPVSYEQTEYIC |
| | | | RLAGYPDGIPVLEHH |
| 171 | HSV-1 | HSV-2 gC (27- | MGGAAARLGAVILFVVIVGLHGVRGKYASPGR |
| | gD KY | 426) | TITVGPRGNASNAAPSASPRNASAPRTTPTPPQPR |
| | | | KATKSKASTAKPAPPPKTGPPKTSSEPVRCNRHDP |
| | | | LARYGSRVQIRCRFPNSTRTEFRLQIWRYATATDA |
| | | | EIGTAPSLEEVMVNVSAPPGGQLVYDSAPNRTDP |
| | | | HVIWAEGAGPGASPRLYSVVGPLGRQRLIIEELTL |
| | | | ETQGMYYWVWGRTDRPSAYGTWVRVRVFRPPS |
| | | | LTIHPHAVLEGQPFKATCTAATYYPGNRAEFVWF |
| | | | EDGRRVFDPAQIHTQTQENPDGFSTVSTVTSAAV |
| | | | GGQGPPRTFTCQLTWHRDSVSFSRRNASGTASVL |
| | | | PRPTITMEFTGDHAVCTAGCVPEGVTFAWFLGDD |
| | | | SSPAEKVAVASQTSCGRPGTATIRSTLPVSYEQTE |
| | | | YICRLAGYPDGIPVLEHH |
| 172 | HSV-1 | HSV-2 gC (28- | MHQGAPSWGRRWFVVWALLGLTLGVLVASA |
| | gB AP | 426) | APSPGRTITVGPRGNASNAAPSASPRNASAPRTTP |
| | | | TPPQPRKATKSKASTAKPAPPPKTGPPKTSSEPVR |
| | | | CNRHDPLARYGSRVQIRCRFPNSTRTEFRLQIWRY |
| | | | ATATDAEIGTAPSLEEVMVNVSAPPGGQLVYDSA |
| | | | PNRTDPHVIWAEGAGPGASPRLYSVVGPLGRQRL |
| | | | IIEELTLETQGMYYWVWGRTDRPSAYGTWVRVR |
| | | | VFRPPSLTIHPHAVLEGQPFKATCTAATYYPGNRA |
| | | | EFVWFEDGRRVFDPAQIHTQTQENPDGFSTVSTV |
| | | | TSAAVGGQGPPRTFTCQLTWHRDSVSFSRRNASG |
| | | | TASVLPRPTITMEFTGDHAVCTAGCVPEGVTFAW |
| | | | FLGDDSSPAEKVAVASQTSCGRPGTATIRSTLPVS |
| | | | YEQTEYICRLAGYPDGIPVLEHH |
| 173 | HSV-1 | HSV-2 gC (27- | MHQGAPSWGRRWFVVWALLGLTLGVLVASA |
| | gB AP | 426) | APASPGRTITVGPRGNASNAAPSASPRNASAPRTT |
| | | | PTPPQPRKATKSKASTAKPAPPPKTGPPKTSSEPV |
| | | | RCNRHDPLARYGSRVQIRCRFPNSTRTEFRLQIWR |
| | | | YATATDAEIGTAPSLEEVMVNVSAPPGGQLVYDS |
| | | | APNRTDPHVIWAEGAGPGASPRLYSVVGPLGRQR |
| | | | LIIEELTLETQGMYYWVWGRTDRPSAYGTWVRV |
| | | | RVFRPPSLTIHPHAVLEGQPFKATCTAATYYPGNR |
| | | | AEFVWFEDGRRVFDPAQIHTQTQENPDGFSTVST |
| | | | VTSAAVGGQGPPRTFTCQLTWHRDSVSFSRRNAS |
| | | | GTASVLPRPTITMEFTGDHAVCTAGCVPEGVTFA |
| | | | WFLGDDSSPAEKVAVASQTSCGRPGTATIRSTLPV |
| | | | SYEQTEYICRLAGYPDGIPVLEHH |
| 174 | HSV-2 gI- | HSV-2 gC (28- | MPGRSLQGLAILGLWVCATGLSPGRTITVGPRG |
| | L | 426) | NASNAAPSASPRNASAPRTTPTPPQPRKATKSKAS |
| | | | TAKPAPPPKTGPPKTSSEPVRCNRHDPLARYGSRV |
| | | | QIRCRFPNSTRTEFRLQIWRYATATDAEIGTAPSLE |
| | | | EVMVNVSAPPGGQLVYDSAPNRTDPHVIWAEGA |
| | | | GPGASPRLYSVVGPLGRQRLIIEELTLETQGMYY |
| | | | WVWGRTDRPSAYGTWVRVRVFRPPSLTIHPHAV |
| | | | LEGQPFKATCTAATYYPGNRAEFVWFEDGRRVF |
| | | | DPAQIHTQTQENPDGFSTVSTVTSAAVGGQGPPRT |
| | | | FTCQLTWHRDSVSFSRRNASGTASVLPRPTITMEF |
| | | | TGDHAVCTAGCVPEGVTFAWFLGDDSSPAEKVA |
| | | | VASQTSCGRPGTATIRSTLPVSYEQTEYICRLAGY |
| | | | PDGIPVLEHH |
| 175 | HSV-2 gI- | HSV-2 gC (27- | MPGRSLQGLAILGLWVCATGLASPGRTITVGPR |
| | L | 426) | GNASNAAPSASPRNASAPRTTPTPPQPRKATKSKA |
| | | | STAKPAPPPKTGPPKTSSEPVRCNRHDPLARYGSR |
| | | | VQIRCRFPNSTRTEFRLQIWRYATATDAEIGTAPSL |
| | | | EEVMVNVSAPPGGQLVYDSAPNRTDPHVIWAEG |
| | | | AGPGASPRLYSVVGPLGRQRLIIEELTLETQGMYY |
| | | | WVWGRTDRPSAYGTWVRVRVFRPPSLTIHPHAV |
| | | | LEGQPFKATCTAATYYPGNRAEFVWFEDGRRVF |
| | | | DPAQIHTQTQENPDGFSTVSTVTSAAVGGQGPPRT |
| | | | FTCQLTWHRDSVSFSRRNASGTASVLPRPTITMEF |
| | | | TGDHAVCTAGCVPEGVTFAWFLGDDSSPAEKVA |
| | | | VASQTSCGRPGTATIRSTLPVSYEQTEYICRLAGY |
| | | | PDGIPVLEHH |
| 176 | HSV-2 | HSV-2 gC (28- | MARGAGLVFFVGVWVVSCLAAAPRTSSPGRTIT |
| | gE RTS | 426) | VGPRGNASNAAPSASPRNASAPRTTPTPPQPRKAT |
| | | | KSKASTAKPAPPPKTGPPKTSSEPVRCNRHDPLAR |
| | | | YGSRVQIRCRFPNSTRTEFRLQIWRYATATDAEIG |
| | | | TAPSLEEVMVNVSAPPGGQLVYDSAPNRTDPHVI |
| | | | WAEGAGPGASPRLYSVVGPLGRQRLIIEELTLETQ |
| | | | GMYYWVWGRTDRPSAYGTWVRVRVFRPPSLTIH |
| | | | PHAVLEGQPFKATCTAATYYPGNRAEFVWFEDG |
| | | | RRVFDPAQIHTQTQENPDGFSTVSTVTSAAVGGQ |
| | | | GPPRTFTCQLTWHRDSVSFSRRNASGTASVLPRPT |
| | | | ITMEFTGDHAVCTAGCVPEGVTFAWFLGDDSSPA |
| | | | EKVAVASQTSCGRPGTATIRSTLPVSYEQTEYICR |
| | | | LAGYPDGIPVLEHH |
| 177 | HSV-2 | HSV-2 gC (27- | MARGAGLVFFVGVWVVSCLAAAPRTSASPGRT |
| | gE RTS | 426) | ITVGPRGNASNAAPSASPRNASAPRTTPTPPQPRK |
| | | | ATKSKASTAKPAPPPKTGPPKTSSEPVRCNRHDPL |
| | | | ARYGSRVQIRCRFPNSTRTEFRLQIWRYATATDAE |
| | | | IGTAPSLEEVMVNVSAPPGGQLVYDSAPNRTDPH |
| | | | VIWAEGAGPGASPRLYSVVGPLGRQRLIIEELTLE |
| | | | TQGMYYWVWGRTDRPSAYGTWVRVRVFRPPSL |
| | | | TIHPHAVLEGQPFKATCTAATYYPGNRAEFVWFE |
| | | | DGRRVFDPAQIHTQTQENPDGFSTVSTVTSAAVG |
| | | | GQGPPRTFTCQLTWHRDSVSFSRRNASGTASVLP |
| | | | RPTITMEFTGDHAVCTAGCVPEGVTFAWFLGDDS |
| | | | SPAEKVAVASQTSCGRPGTATIRSTLPVSYEQTEYI |
| | | | CRLAGYPDGIPVLEHH |
| 178 | EboZ | HSV-2 gC (28- | MGVTGILQLPRDRFKRTSFFLWVIILFQRTFSIP |
| | | 426) | SPGRTITVGPRGNASNAAPSASPRNASAPRTTPTPP |
| | | | QPRKATKSKASTAKPAPPPKTGPPKTSSEPVRCNR |
| | | | HDPLARYGSRVQIRCRFPNSTRTEFRLQIWRYATA |
| | | | TDAEIGTAPSLEEVMVNVSAPPGGQLVYDSAPNR |
| | | | TDPHVIWAEGAGPGASPRLYSVVGPLGRQRLIIEE |
| | | | LTLETQGMYYWVWGRTDRPSAYGTWVRVRVFR |
| | | | PPSLTIHPHAVLEGQPFKATCTAATYYPGNRAEFV |
| | | | WFEDGRRVFDPAQIHTQTQENPDGFSTVSTVTSA |
| | | | AVGGQGPPRTFTCQLTWHRDSVSFSRRNASGTAS |
| | | | VLPRPTITMEFTGDHAVCTAGCVPEGVTFAWFLG |
| | | | DDSSPAEKVAVASQTSCGRPGTATIRSTLPVSYEQ |
| | | | TEYICRLAGYPDGIPVLEHH |
| 179 | EboZ | HSV-2 gC (27- | MGVTGILQLPRDRFKRTSFFLWVIILFQRTFSIP |
| | | 426) | ASPGRTITVGPRGNASNAAPSASPRNASAPRTTPT |
| | | | PPQPRKATKSKASTAKPAPPPKTGPPKTSSEPVRC |
| | | | NRHDPLARYGSRVQIRCRFPNSTRTEFRLQIWRYA |
| | | | TATDAEIGTAPSLEEVMVNVSAPPGGQLVYDSAP |
| | | | NRTDPHVIWAEGAGPGASPRLYSVVGPLGRQRLII |
| | | | EELTLETQGMYYWVWGRTDRPSAYGTWVRVRV |
| | | | FRPPSLTIHPHAVLEGQPFKATCTAATYYPGNRAE |
| | | | FVWFEDGRRVFDPAQIHTQTQENPDGFSTVSTVTS |
| | | | AAVGGQGPPRTFTCQLTWHRDSVSFSRRNASGTA |
| | | | SVLPRPTITMEFTGDHAVCTAGCVPEGVTFAWFL |
| | | | GDDSSPAEKVAVASQTSCGRPGTATIRSTLPVSYE |
| | | | QTEYICRLAGYPDGIPVLEHH |
| 180 | IL2 | HSV-2 gD | MRMQLLLLIALSLALVTNSADPSLKMADPNRFR |
| | | (30-331) | GKNLPVLDQLTDPPGVKRVYHIQPSLEDPFQPPSIP |
| | | | ITVYYAVLERACRSVLLHAPSEAPQIVRGASDEAR |
| | | | KHTYNLTIAWYRMGDNCAIPITVMEYTECPYNKS |
| | | | LGVCPIRTQPRWSYYDSFSAVSEDNLGFLMHAPA |
| | | | FETAGTYLRLVKINDWTEITQFILEHRARASCKYA |
| | | | LPLRIPPAACLTSKAYQQGVTVDSIGMLPRFIPENQ |
| | | | RTVALYSLKIAGWHGPKPPYTSTLLPPELSDTTNA |
| | | | TQPELVPEDPEDSALLEDPAGTVSSQIPPNWHIPSI |
| | | | QDVAPHH |
| 181 | IL2 | HSV-2 gD | MRMQLLLLIALSLALVTNSKYALADPSLKMADP |
| | | (26-331) | NRFRGKNLPVLDQLTDPPGVKRVYHIQPSLEDPF |
| | | | QPPSIPITVYYAVLERACRSVLLHAPSEAPQIVRGA |
| | | | SDEARKHTYNLTIAWYRMGDNCAIPITVMEYTEC |
| | | | PYNKSLGVCPIRTQPRWSYYDSFSAVSEDNLGFL |
| | | | MHAPAFETAGTYLRLVKINDWTEITQFILEHRARA |
| | | | SCKYALPLRIPPAACLTSKAYQQGVTVDSIGMLPR |
| | | | FIPENQRTVALYSLKIAGWHGPKPPYTSTLLPPELS |
| | | | DTTNATQPELVPEDPEDSALLEDPAGTVSSQIPPN |
| | | | WHIPSIQDVAPHH |
| 182 | HSV-2 | HSV-2 gD (30- | MGRLTSGVGTAALLVVAVGLRVVCAADPSLK |
| | gD | 331) | MADPNRFRGKNLPVLDQLTDPPGVKRVYHIQPSL |
| | | | EDPFQPPSIPITVYYAVLERACRSVLLHAPSEAPQI |
| | | | VRGASDEARKHTYNLTIAWYRMGDNCAIPITVME |
| | | | YTECPYNKSLGVCPIRTQPRWSYYDSFSAVSEDN |
| | | | LGFLMHAPAFETAGTYLRLVKINDWTEITQFILEH |
| | | | RARASCKYALPLRIPPAACLTSKAYQQGVTVDSIG |
| | | | MLPRFIPENQRTVALYSLKIAGWHGPKPPYTSTLL |
| | | | PPELSDTTNATQPELVPEDPEDSALLEDPAGTVSS |
| | | | QIPPNWHIPSIQDVAPHH |
| 183 | HSV-2 | HSV-2 gD (26- | MGRLTSGVGTAALLVVAVGLRVVCAKYALAD |
| | gD | 331) | PSLKMADPNRFRGKNLPVLDQLTDPPGVKRVYHI |
| | | | QPSLEDPFQPPSIPITVYYAVLERACRSVLLHAPSE |
| | | | APQIVRGASDEARKHTYNLTIAWYRMGDNCAIPI |
| | | | TVMEYTECPYNKSLGVCPIRTQPRWSYYDSFSAV |
| | | | SEDNLGFLMHAPAFETAGTYLRLVKINDWTEITQ |
| | | | FILEHRARASCKYALPLRIPPAACLTSKAYQQGVT |
| | | | VDSIGMLPRFIPENQRTVALYSLKIAGWHGPKPPY |
| | | | TSTLLPPELSDTTNATQPELVPEDPEDSALLEDPAG |
| | | | TVSSQIPPNWHIPSIQDVAPHH |
| 184 | HSV-2 | HSV-2 gD | MGRLTSGVGTAALLVVAVGLRVVCAKYADPSL |
| | gD-KY | (30-331) | KMADPNRFRGKNLPVLDQLTDPPGVKRVYHIQPS |
| | | | LEDPFQPPSIPITVYYAVLERACRSVLLHAPSEAPQ |
| | | | IVRGASDEARKHTYNLTIAWYRMGDNCAIPITVM |
| | | | EYTECPYNKSLGVCPIRTQPRWSYYDSFSAVSED |
| | | | NLGFLMHAPAFETAGTYLRLVKINDWTEITQFILE |
| | | | HRARASCKYALPLRIPPAACLTSKAYQQGVTVDSI |
| | | | GMLPRFIPENQRTVALYSLKIAGWHGPKPPYTSTL |
| | | | LPPELSDTTNATQPELVPEDPEDSALLEDPAGTVS |
| | | | SQIPPNWHIPSIQDVAPHH |
| 185 | IL2 | HSV-2 gE | MRMQLLLLIALSLALVTNSRTSWKRVTSGEDVV |
| | | (24-405) | LLPAPAGPEERTRAHKLLWAAEPLDACGPLRPSW |
| | | | VALWPPRRVLETVVDAACMRAPEPLAIAYSPPFP |
| | | | AGDEGLYSELAWRDRVAVVNESLVIYGALETDS |
| | | | GLYTLSVVGLSDEARQVASVVLVVEPAPVPTPTP |
| | | | DDYDEEDDAGVSERTPVSVPPPTPPRRPPVAPPTH |
| | | | PRVIPEVSHVRGVTVHMETPEAILFAPGETFGTNV |
| | | | SIHAIAHDDGPYAMDVVWMRFDVPSSCAEMRIYE |
| | | | ACLYHPQLPECLSPADAPCAVSSWAYRLAVRSYA |
| | | | GCSRTTPPPRCFAEARMEPVPGLAWLASTVNLEF |
| | | | QHASPQHAGLYLCVVYVDDHIHAWGHMTISTAA |
| | | | QYRNAVVEQHLPQRQPEPVEPTRPHVRA |
| 186 | HSV-2 | HSV-2 gE | MGRLTSGVGTAALLVVAVGLRVVCARTSWKR |
| | gD | (24-405) | VTSGEDVVLLPAPAGPEERTRAHKLLWAAEPLDA |
| | | | CGPLRPSWVALWPPRRVLETVVDAACMRAPEPL |
| | | | AIAYSPPFPAGDEGLYSELAWRDRVAVVNESLVI |
| | | | YGALETDSGLYTLSVVGLSDEARQVASVVLVVEP |
| | | | APVPTPTPDDYDEEDDAGVSERTPVSVPPPTPPRR |
| | | | PPVAPPTHPRVIPEVSHVRGVTVHMETPEAILFAP |
| | | | GETFGTNVSIHAIAHDDGPYAMDVVWMRFDVPS |
| | | | SCAEMRIYEACLYHPQLPECLSPADAPCAVSSWA |
| | | | YRLAVRSYAGCSRTTPPPRCFAEARMEPVPGLAW |
| | | | LASTVNLEFQHASPQHAGLYLCVVYVDDHIHAW |
| | | | GHMTISTAAQYRNAVVEQHLPQRQPEPVEPTRPH |
| | | | VRA |
| 187 | HSV-2 | HSV-2 gE | MGRLTSGVGTAALLVVAVGLRVVCAKYARTS |
| | gD- | (24-405) | WKRVTSGEDVVLLPAPAGPEERTRAHKLLWAAE |
| | KYA | | PLDACGPLRPSWVALWPPRRVLETVVDAACMRA |
| | | | PEPLAIAYSPPFPAGDEGLYSELAWRDRVAVVNES |
| | | | LVIYGALETDSGLYTLSVVGLSDEARQVASVVLV |
| | | | VEPAPVPTPTPDDYDEEDDAGVSERTPVSVPPPTP |
| | | | PRRPPVAPPTHPRVIPEVSHVRGVTVHMETPEAIL |
| | | | FAPGETFGTNVSIHAIAHDDGPYAMDVVWMRFD |
| | | | VPSSCAEMRIYEACLYHPQLPECLSPADAPCAVSS |
| | | | WAYRLAVRSYAGCSRTTPPPRCFAEARMEPVPGL |
| | | | AWLASTVNLEFQHASPQHAGLYLCVVYVDDHIH |
| | | | AWGHMTISTAAQYRNAVVEQHLPQRQPEPVEPT |
| | | | RPHVRA |
| 188 | HSV-2 | HSV-2 gE | MGRLTSGVGTAALLVVAVGLRVVCAKYALAR |
| | gD- | (24-405) | TSWKRVTSGEDVVLLPAPAGPEERTRAHKLLWA |
| | KYALA | | AEPLDACGPLRPSWVALWPPRRVLETVVDAACM |
| | | | RAPEPLAIAYSPPFPAGDEGLYSELAWRDRVAVV |
| | | | NESLVIYGALETDSGLYTLSVVGLSDEARQVASV |
| | | | VLVVEPAPVPTPTPDDYDEEDDAGVSERTPVSVPP |
| | | | PTPPRRPPVAPPTHPRVIPEVSHVRGVTVHMETPE |
| | | | AILFAPGETFGTNVSIHAIAHDDGPYAMDVVWMR |
| | | | FDVPSSCAEMRIYEACLYHPQLPECLSPADAPCAV |
| | | | SSWAYRLAVRSYAGCSRTTPPPRCFAEARMEPVP |
| | | | GLAWLASTVNLEFQHASPQHAGLYLCVVYVDDH |
| | | | IHAWGHMTISTAAQYRNAVVEQHLPQRQPEPVEP |
| | | | TRPHVRA |
| 189 | HSV-2 | HSV-2 gE | MARGAGLVFFVGVWVVSCLAAAPRTSWKRVTS |
| | gE | (24-405) | GEDVVLLPAPAGPEERTRAHKLLWAAEPLDACGP |
| | | | LRPSWVALWPPRRVLETVVDAACMRAPEPLAIAY |
| | | | SPPFPAGDEGLYSELAWRDRVAVVNESLVIYGAL |
| | | | ETDSGLYTLSVVGLSDEARQVASVVLVVEPAPVP |
| | | | TPTPDDYDEEDDAGVSERTPVSVPPPTPPRRPPVA |
| | | | PPTHPRVIPEVSHVRGVTVHMETPEAILFAPGETF |
| | | | GTNVSIHAIAHDDGPYAMDVVWMRFDVPSSCAE |
| | | | MRIYEACLYHPQLPECLSPADAPCAVSSWAYRLA |
| | | | VRSYAGCSRTTPPPRCFAEARMEPVPGLAWLAST |
| | | | VNLEFQHASPQHAGLYLCVVYVDDHIHAWGHMT |
| | | | ISTAAQYRNAVVEQHLPQRQPEPVEPTRPHVRA |
| 190 | HSV-2 | HSV-2 gE | MARGAGLVFFVGVWVVSCLVVVPRTSWKRVTS |
| | gE- | (24-405) | GEDVVLLPAPAGPEERTRAHKLLWAAEPLDACGP |
| | LVVVP | | LRPSWVALWPPRRVLETVVDAACMRAPEPLAIAY |
| | | | SPPFPAGDEGLYSELAWRDRVAVVNESLVIYGAL |
| | | | ETDSGLYTLSVVGLSDEARQVASVVLVVEPAPVP |
| | | | TPTPDDYDEEDDAGVSERTPVSVPPPTPPRRPPVA |
| | | | PPTHPRVIPEVSHVRGVTVHMETPEAILFAPGETF |
| | | | GTNVSIHAIAHDDGPYAMDVVWMRFDVPSSCAE |
| | | | MRIYEACLYHPQLPECLSPADAPCAVSSWAYRLA |
| | | | VRSYAGCSRTTPPPRCFAEARMEPVPGLAWLAST |
| | | | VNLEFQHASPQHAGLYLCVVYVDDHIHAWGHMT |
| | | | ISTAAQYRNAVVEQHLPQRQPEPVEPTRPHVRA |
| 191 | None | HSV-2 gE | MRTSWKRVTSGEDVVLLPAPAGPEERTRAHKLL |
| | (N-term | (24-405) | WAAEPLDACGPLRPSWVALWPPRRVLETVVDAA |
| | Met) | | CMRAPEPLAIAYSPPFPAGDEGLYSELAWRDRVA |
| | | | VVNESLVIYGALETDSGLYTLSVVGLSDEARQVA |
| | | | SVVLVVEPAPVPTPTPDDYDEEDDAGVSERTPVS |
| | | | VPPPTPPRRPPVAPPTHPRVIPEVSHVRGVTVHME |
| | | | TPEAILFAPGETFGTNVSIHAIAHDDGPYAMDVV |
| | | | WMRFDVPSSCAEMRIYEACLYHPQLPECLSPADA |
| | | | PCAVSSWAYRLAVRSYAGCSRTTPPPRCFAEARM |
| | | | EPVPGLAWLASTVNLEFQHASPQHAGLYLCVVY |
| | | | VDDHIHAWGHMTISTAAQYRNAVVEQHLPQRQP |
| | | | EPVEPTRPHVRA |
| 192 | HSV-1 | HSV-2 gE | MGGAAARLGAVILFVVIVGLHGVRGKYRTSWK |
| | gD-KY | (24-405) | RVTSGEDVVLLPAPAGPEERTRAHKLLWAAEPLD |
| | | | ACGPLRPSWVALWPPRRVLETVVDAACMRAPEP |
| | | | LAIAYSPPFPAGDEGLYSELAWRDRVAVVNESLVI |
| | | | YGALETDSGLYTLSVVGLSDEARQVASVVLVVEP |
| | | | APVPTPTPDDYDEEDDAGVSERTPVSVPPPTPPRR |
| | | | PPVAPPTHPRVIPEVSHVRGVTVHMETPEAILFAP |
| | | | GETFGTNVSIHAIAHDDGPYAMDVVWMRFDVPS |
| | | | SCAEMRIYEACLYHPQLPECLSPADAPCAVSSWA |
| | | | YRLAVRSYAGCSRTTPPPRCFAEARMEPVPGLAW |
| | | | LASTVNLEFQHASPQHAGLYLCVVYVDDHIHAW |
| | | | GHMTISTAAQYRNAVVEQHLPQRQPEPVEPTRPH |
| | | | VRA |

### Exemplary Signal Sequence and HSV Immunogen Nucleotide Sequence Combinations

The present disclosure also provides RNAs comprising nucleotide sequences as provided herein. In some embodiments, an RNA provided herein comprises a nucleotide sequence that encodes an HSV-2 gC protein or immunogenic fragment thereof. In some embodiments, an RNA provided herein comprises a nucleotide sequence that encodes an HSV-2 gD protein or immunogenic fragment thereof. In some embodiments, an RNA provided herein comprises a nucleotide sequence that encodes an HSV-2 gE protein or immunogenic fragment thereof.

Exemplary RNA comprising a signal sequence (i.e., encoding a signal peptide) and a sequence encoding an HSV-2 glycoprotein are shown in Table 6 below. In some embodiments, an RNA of the present disclosure comprises a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

TABLE 6
Exemplary signal sequence/HSV-2 glycoprotein sequence combinations

| SEQ | | | | |
|---|---|---|---|---|
| ID | Signal | | | |
| NO: | Sequence | Immunogen | Version | Nucleotide Sequence |
| 193 | IL2 | HSV-2 gC | | AUGCGCAUGCAGCUGCUGCUGCUGAUC |
| | | (27-426) | | GCCCUGUCCCUGGCCCUGGUGACCAACU |
| | | | | CCGCCUCCCCCGGCCGCACCAUCACCGU |
| | | | | GGGCCCCCGCGGCAACGCCUCCAACGCC |
| | | | | GCCCCCUCCGCCUCCCCCCGCAACGCCU |
| | | | | CCGCCCCCCGCACCACCCCCACCCCCCCC |
| | | | | CAGCCCCGCAAGGCCACCAAGUCCAAGG |
| | | | | CCUCCACCGCCAAGCCCGCCCCCCCCCC |
| | | | | CAAGACCGGCCCCCCCAAGACCUCCUCC |
| | | | | GAGCCCGUGCGCUGCAACCGCCACGACC |
| | | | | CCCUGGCCCGCUACGGCUCCCGCGUGCA |
| | | | | GAUCCGCUGCCGCUUCCCCAACUCCACC |
| | | | | CGCACCGAGUUCCGCCUGCAGAUCUGGC |
| | | | | GCUACGCCACCGCCACCGACGCCGAGAU |
| | | | | CGGCACCGCCCCCUCCCUGGAGGAGGUG |
| | | | | AUGGUGAACGUGUCCGCCCCCCCCGGCG |
| | | | | GCCAGCUGGUGUACGACUCCGCCCCCAA |
| | | | | CCGCACCGACCCCCACGUGAUCUGGGCC |
| | | | | GAGGGCGCCGGCCCCGGCGCCUCCCCCC |
| | | | | GCCUGUACUCCGUGGUGGGCCCCCUGGG |
| | | | | CCGCCAGCGCCUGAUCAUCGAGGAGCUG |
| | | | | ACCCUGGAGACCCAGGGCAUGUACUACU |
| | | | | GGGUGUGGGGCCGCACCGACCGCCCCUC |
| | | | | CGCCUACGGCACCUGGGUGCGCGUGCGC |
| | | | | GUGUUCCGCCCCCCCUCCCUGACCAUCC |
| | | | | ACCCCCACGCCGUGCUGGAGGGCCAGCC |
| | | | | CUUCAAGGCCACCUGCACCGCCGCCACC |
| | | | | UACUACCCCGGCAACCGCGCCGAGUUCG |
| | | | | UGUGGUUCGAGGACGGCCGCCGCGUGU |
| | | | | UCGACCCCGCCCAGAUCCACACCCAGAC |
| | | | | CCAGGAGAACCCCGACGGCUUCUCCACC |
| | | | | GUGUCCACCGUGACCUCCGCCGCCGUGG |
| | | | | GCGGCCAGGGCCCCCCCCGCACCUUCAC |
| | | | | CUGCCAGCUGACCUGGCACCGCGACUCC |
| | | | | GUGUCCUUCUCCCGCCGCAACGCCUCCG |
| | | | | GCACCGCCUCCGUGCUGCCCCGCCCCAC |
| | | | | CAUCACCAUGGAGUUCACCGGCGACCAC |
| | | | | GCCGUGUGCACCGCCGGCUGCGUGCCCG |
| | | | | AGGGCGUGACCUUCGCCUGGUUCCUGG |
| | | | | GCGACGACUCCUCCCCCGCCGAGAAGGU |
| | | | | GGCCGUGGCCUCCCAGACCUCCUGCGGC |
| | | | | CGCCCCGGCACCGCCACCAUCCGCUCCA |
| | | | | CCCUGCCCGUGUCCUACGAGCAGACCGA |
| | | | | GUACAUCUGCCGCCUGGCCGGCUACCCC |
| | | | | GACGGCAUCCCCGUGCUGGAGCACCACU |
| | | | | AA |
| 194 | IL2 | HSV-2 gC | Version 1 | AUGAGAAUGCAGCUGCUGCUCCUGAUC |
| | | (27-426) | | GCCCUGUCUCUGGCCCUGGUCACCAAUA |
| | | | | GCGCUUCUCCCGGCAGAACCAUCACAGU |
| | | | | GGGCCCUAGAGGCAACGCCUCUAAUGCC |
| | | | | GCUCCUAGCGCCUCUCCUAGAAACGCCU |
| | | | | CUGCUCCCAGAACCACACCUACACCUCC |
| | | | | ACAGCCUAGAAAGGCCACCAAGAGCAA |
| | | | | GGCCAGCACAGCCAAACCUGCUCCUCCA |
| | | | | CCUAAGACAGGCCCUCCAAAGACAAGCU |
| | | | | CUGAGCCCGUGCGGUGCAACAGACACGA |
| | | | | UCCACUGGCCAGAUACGGCAGCCGGGUG |
| | | | | CAGAUCAGAUGCAGAUUCCCCAACAGCA |
| | | | | CCCGGACCGAGUUCCGGCUCCAGAUUUG |
| | | | | GAGAUACGCCACCGCCACAGAUGCCGAG |
| | | | | AUUGGAACAGCCCCUAGCCUGGAAGAA |
| | | | | GUGAUGGUCAACGUUUCAGCCCCUCCUG |
| | | | | GCGGCCAGCUGGUGUAUGAUUCUGCCCC |
| | | | | UAACCGGACCGAUCCUCACGUGAUAUG |
| | | | | GGCUGAAGGUGCUGGCCCAGGCGCAAG |
| | | | | CCCUAGACUGUAUUCUGUUGUGGGCCC |
| | | | | UCUGGGCAGACAGCGGCUGAUCAUUGA |
| | | | | GGAACUGACCCUGGAAACCCAGGGCAU |
| | | | | GUACUACUGGGUCUGGGGCAGAACCGA |
| | | | | UAGACCAAGCGCCUAUGGCACCUGGGU |
| | | | | UCGAGUGCGAGUGUUCAGACCUCCUAG |
| | | | | CCUGACCAUCCAUCCUCACGCCGUUCUG |
| | | | | GAAGGCCAGCCUUUCAAGGCCACAUGU |
| | | | | ACCGCCGCCACCUACUAUCCCGGAAACA |
| | | | | GAGCCGAGUUCGUUUGGUUCGAGGACG |
| | | | | GCAGAAGGGUGUUCGACCCCGCUCAGA |
| | | | | UCCACACACAGACCCAAGAGAACCCCGA |
| | | | | CGGCUUUAGCACCGUGUCCACAGUGACA |
| | | | | UCUGCCGCCGUUGGAGGACAGGGCCCUC |
| | | | | CUAGAACCUUUACCUGCCAGCUGACCUG |
| | | | | GCACAGAGACAGCGUGUCCUUCAGCAG |
| | | | | AAGAAACGCCAGCGGCACAGCCAGCGUU |
| | | | | CUGCCUAGACCUACCAUCACCAUGGAAU |
| | | | | UCACCGGCGACCACGCCGUGUGUACAGC |
| | | | | UGGAUGUGUUCCUGAGGGCGUGACCUU |
| | | | | CGCUUGGUUUCUGGGCGACGAUAGCAG |
| | | | | CCCUGCCGAAAAAGUGGCUGUGGCCAGC |
| | | | | CAGACAAGCUGUGGCAGACCUGGAACC |
| | | | | GCCACCAUCAGAAGCACACUGCCUGUCA |
| | | | | GCUACGAGCAGACCGAGUACAUCUGUC |
| | | | | GGCUGGCCGGCUAUCCUGAUGGCAUCCC |
| | | | | UGUGCUGGAACACCACUGA |
| 195 | IL2 | HSV-2 gC | Version 2 | AUGCGCAUGCAACUGCUCCUGCUGAUU |
| | | (27-426) | | GCGUUGAGCCUUGCCCUGGUGACCAACA |
| | | | | GCGCAAGCCCCGGCAGAACCAUAACAGU |
| | | | | AGGGCCACGGGGGAAUGCUUCCAAUGC |
| | | | | UGCACCUUCCGCUUCACCGAGGAAUGCU |
| | | | | UCUGCCCCAAGAACUACCCCCACUCCUC |
| | | | | CUCAACCCAGGAAAGCGACAAAGUCCAA |
| | | | | GGCCAGCACCGCAAAACCCGCUCCUCCU |
| | | | | CCAAAGACUGGGCCCCCAAAGACAAGUA |
| | | | | GCGAACCAGUUCGGUGCAACAGGCAUG |
| | | | | ACCCACUUGCACGCUAUGGGUCAAGAG |
| | | | | UCCAGAUACGGUGUCGCUUCCCUAACAG |
| | | | | UACAAGGACUGAGUUUCGGCUGCAGAU |
| | | | | CUGGCGUUAUGCCACAGCUACUGACGCA |
| | | | | GAGAUUGGUACCGCCCCCAGUUUGGAA |
| | | | | GAGGUGAUGGUCAACGUGUCCGCACCCC |
| | | | | CAGGAGGACAGCUGGUCUAUGACUCAG |
| | | | | CGCCCAAUAGGACCGAUCCCCACGUGAU |
| | | | | CUGGGCAGAAGGAGCCGGUCCUGGGGC |
| | | | | CUCUCCACGGCUGUACUCAGUUGUUGGC |
| | | | | CCGCUUGGACGACAGAGACUCAUCAUCG |
| | | | | AAGAGCUGACACUGGAGACACAGGGGA |
| | | | | UGUACUACUGGGUGUGGGGCCGUACUG |
| | | | | ACCGCCCUUCCGCAUAUGGCACUUGGGU |
| | | | | GAGAGUUCGCGUCUUUCGGCCCCCUUCU |
| | | | | CUCACCAUCCAUCCUCAUGCCGUGCUCG |
| | | | | AAGGCCAGCCCUUUAAGGCCACAUGCAC |
| | | | | UGCUGCGACCUACUACCCUGGCAACAGA |
| | | | | GCCGAGUUUGUCUGGUUUGAGGAUGGU |
| | | | | CGGCGAGUAUUCGAUCCAGCCCAGAUUC |
| | | | | ACACACAAACGCAGGAAAAUCCGGACG |
| | | | | GCUUCAGCACAGUGUCCACGGUGACCUC |
| | | | | UGCUGCAGUUGGUGGACAAGGACCCCC |
| | | | | UCGAACCUUCACCUGUCAGCUGACCUGG |
| | | | | CACAGAGACUCCGUAAGCUUCAGCCGUA |
| | | | | GAAACGCCUCUGGAACCGCCAGUGUGU |
| | | | | UGCCGAGGCCGACUAUCACGAUGGAAU |
| | | | | UCACAGGCGAUCAUGCCGUCUGUACUGC |
| | | | | CGGCUGUGUGCCAGAAGGCGUAACCUU |
| | | | | CGCUUGGUUUCUCGGGGAUGACUCAAG |
| | | | | UCCUGCAGAGAAAGUGGCUGUGGCCUC |
| | | | | UCAGACGAGCUGCGGUCGACCAGGAAC |
| | | | | AGCUACCAUUCGCAGCACUCUGCCCGUG |
| | | | | UCCUACGAGCAGACGGAGUACAUCUGC |
| | | | | AGGCUGGCCGGCUAUCCCGAUGGGAUU |
| | | | | CCAGUCCUGGAGCACCACUGA |
| 196 | IL2 | HSV-2 gC | Version 3 | AUGAGAAUGCAGCUGCUGCUGCUGAUC |
| | | (27-426) | | GCCCUGUCCCUGGCCCUGGUGACCAACU |
| | | | | CCGCCUCCCCCGGCAGAACCAUCACCGU |
| | | | | GGGCCCCAGAGGCAACGCCUCCAACGCC |
| | | | | GCCCCCUCCGCCUCCCCCAGAAACGCCU |
| | | | | CCGCCCCCAGAACCACCCCCACCCCUCC |
| | | | | CCAGCCCAGAAAAGCCACCAAAUCCAAA |
| | | | | GCCUCCACCGCCAAACCCGCCCCUCCUC |
| | | | | CCAAAACCGGCCCUCCCAAAACCUCCUC |
| | | | | CGAACCCGUGAGAUGCAACAGACACGA |
| | | | | UCCCCUGGCCAGAUACGGCUCCAGAGUG |
| | | | | CAGAUCAGAUGCAGAUUCCCCAACUCCA |
| | | | | CCAGAACCGAAUUCAGACUGCAGAUCU |
| | | | | GGAGAUACGCCACCGCCACCGAUGCCGA |
| | | | | AAUCGGCACCGCCCCCUCCCUGGAAGAA |
| | | | | GUGAUGGUGAACGUGUCCGCCCCUCCUG |
| | | | | GCGGCCAGCUGGUGUACGAUUCCGCCCC |
| | | | | CAACAGAACCGAUCCCCACGUGAUCUGG |
| | | | | GCCGAAGGCGCCGGCCCCGGCGCCUCCC |
| | | | | CCAGACUGUACUCCGUGGUGGGCCCCCU |
| | | | | GGGCAGACAGAGACUGAUCAUCGAAGA |
| | | | | ACUGACCCUGGAAACCCAGGGCAUGUAC |
| | | | | UACUGGGUGUGGGGCAGAACCGAUAGA |
| | | | | CCCUCCGCCUACGGCACCUGGGUGAGAG |
| | | | | UGAGAGUGUUCAGACCCCCUUCCCUGAC |
| | | | | CAUCCACCCCCACGCCGUGCUGGAAGGC |
| | | | | CAGCCCUUCAAAGCCACCUGCACCGCCG |
| | | | | CCACCUACUACCCCGGCAACAGAGCCGA |
| | | | | AUUCGUGUGGUUCGAAGAUGGCAGAAG |
| | | | | GGUGUUCGAUCCCGCCCAGAUCCACACC |
| | | | | CAGACCCAGGAAAACCCCGACGGCUUCU |
| | | | | CCACCGUGUCCACCGUGACCUCCGCCGC |
| | | | | CGUGGGCGGCCAGGGCCCUCCCAGAACC |
| | | | | UUCACCUGCCAGCUGACCUGGCACAGAG |
| | | | | ACUCCGUGUCCUUCUCCAGAAGAAACGC |
| | | | | CUCCGGCACCGCCUCCGUGCUGCCCAGA |
| | | | | CCCACCAUCACCAUGGAAUUCACCGGCG |
| | | | | AUCACGCCGUGUGCACCGCCGGCUGCGU |
| | | | | GCCCGAAGGCGUGACCUUCGCCUGGUUC |
| | | | | CUGGGCGAUGAUUCCUCCCCCGCCGAAA |
| | | | | AAGUGGCCGUGGCCUCCCAGACCUCCUG |
| | | | | CGGCAGACCCGGCACCGCCACCAUCAGA |
| | | | | UCCACCCUGCCCGUGUCCUACGAACAGA |
| | | | | CCGAAUACAUCUGCAGACUGGCCGGCUA |
| | | | | CCCCGAUGGCAUCCCCGUGCUGGAACAC |
| | | | | CACUGA |
| 197 | HSV-2 gD- | HSV-2 gC | | AUGGGCCGCCUGACCUCCGGCGUGGGCA |
| | KYA | (27-426) | | CCGCCGCCCUGCUGGUGGUGGCCGUGGG |
| | | | | CCUGCGCGUGGUGUGCGCCAAGUACGCC |
| | | | | GCCUCCCCCGGCCGCACCAUCACCGUGG |
| | | | | GCCCCCGCGGCAACGCCUCCAACGCCGC |
| | | | | CCCCUCCGCCUCCCCCCGCAACGCCUCC |
| | | | | GCCCCCCGCACCACCCCCACCCCCCCCCA |
| | | | | GCCCCGCAAGGCCACCAAGUCCAAGGCC |
| | | | | UCCACCGCCAAGCCCGCCCCCCCCCCCA |
| | | | | AGACCGGCCCCCCCAAGACCUCCUCCGA |
| | | | | GCCCGUGCGCUGCAACCGCCACGACCCC |
| | | | | CUGGCCCGCUACGGCUCCCGCGUGCAGA |
| | | | | UCCGCUGCCGCUUCCCCAACUCCACCCG |
| | | | | CACCGAGUUCCGCCUGCAGAUCUGGCGC |
| | | | | UACGCCACCGCCACCGACGCCGAGAUCG |
| | | | | GCACCGCCCCCUCCCUGGAGGAGGUGAU |
| | | | | GGUGAACGUGUCCGCCCCCCCCGGCGGC |
| | | | | CAGCUGGUGUACGACUCCGCCCCCAACC |
| | | | | GCACCGACCCCCACGUGAUCUGGGCCGA |
| | | | | GGGCGCCGGCCCCGGCGCCUCCCCCCGC |
| | | | | CUGUACUCCGUGGUGGGCCCCCUGGGCC |
| | | | | GCCAGCGCCUGAUCAUCGAGGAGCUGAC |
| | | | | CCUGGAGACCCAGGGCAUGUACUACUG |
| | | | | GGUGUGGGGCCGCACCGACCGCCCCUCC |
| | | | | GCCUACGGCACCUGGGUGCGCGUGCGCG |
| | | | | UGUUCCGCCCCCCCUCCCUGACCAUCCA |
| | | | | CCCCCACGCCGUGCUGGAGGGCCAGCCC |
| | | | | UUCAAGGCCACCUGCACCGCCGCCACCU |
| | | | | ACUACCCCGGCAACCGCGCCGAGUUCGU |
| | | | | GUGGUUCGAGGACGGCCGCCGCGUGUU |
| | | | | CGACCCCGCCCAGAUCCACACCCAGACC |
| | | | | CAGGAGAACCCCGACGGCUUCUCCACCG |
| | | | | UGUCCACCGUGACCUCCGCCGCCGUGGG |
| | | | | CGGCCAGGGCCCCCCCCGCACCUUCACC |
| | | | | UGCCAGCUGACCUGGCACCGCGACUCCG |
| | | | | UGUCCUUCUCCCGCCGCAACGCCUCCGG |
| | | | | CACCGCCUCCGUGCUGCCCCGCCCCACC |
| | | | | AUCACCAUGGAGUUCACCGGCGACCACG |
| | | | | CCGUGUGCACCGCCGGCUGCGUGCCCGA |
| | | | | GGGCGUGACCUUCGCCUGGUUCCUGGGC |
| | | | | GACGACUCCUCCCCCGCCGAGAAGGUGG |
| | | | | CCGUGGCCUCCCAGACCUCCUGCGGCCG |
| | | | | CCCCGGCACCGCCACCAUCCGCUCCACC |
| | | | | CUGCCCGUGUCCUACGAGCAGACCGAGU |
| | | | | ACAUCUGCCGCCUGGCCGGCUACCCCGA |
| | | | | CGGCAUCCCCGUGCUGGAGCACCACUAA |
| 198 | HSV-2 gD- | HSV-2 gC | Version 2 | AUGGGGAGACUCACAUCAGGCGUAGGA |
| | KYA | (27-426) | | ACCGCUGCCCUGUUGGUCGUGGCCGUUG |
| | | | | GUCUGAGAGUUGUGUGUGCCAAAUAUG |
| | | | | CUGCAAGCCCCGGCAGAACCAUAACAGU |
| | | | | AGGGCCACGGGGGAAUGCUUCCAAUGC |
| | | | | UGCACCUUCCGCUUCACCGAGGAAUGCU |
| | | | | UCUGCCCCAAGAACUACCCCCACUCCUC |
| | | | | CUCAACCCAGGAAAGCGACAAAGUCCAA |
| | | | | GGCCAGCACCGCAAAACCCGCUCCUCCU |
| | | | | CCAAAGACUGGGCCCCCAAAGACAAGUA |
| | | | | GCGAACCAGUUCGGUGCAACAGGCAUG |
| | | | | ACCCACUUGCACGCUAUGGGUCAAGAG |
| | | | | UCCAGAUACGGUGUCGCUUCCCUAACAG |
| | | | | UACAAGGACUGAGUUUCGGCUGCAGAU |
| | | | | CUGGCGUUAUGCCACAGCUACUGACGCA |
| | | | | GAGAUUGGUACCGCCCCCAGUUUGGAG |
| | | | | GAAGUGAUGGUCAACGUGUCCGCACCCC |
| | | | | CAGGAGGACAGCUGGUCUAUGACUCAG |
| | | | | CGCCCAAUAGGACCGAUCCCCACGUGAU |
| | | | | CUGGGCAGAAGGAGCCGGUCCUGGGGC |
| | | | | CUCUCCACGGCUGUACUCAGUUGUUGGC |
| | | | | CCGCUUGGACGACAGAGACUCAUCAUCG |
| | | | | AGGAACUGACACUGGAGACACAGGGGA |
| | | | | UGUACUACUGGGUGUGGGGCCGUACUG |
| | | | | ACCGCCCUUCCGCAUAUGGCACUUGGGU |
| | | | | GAGAGUUCGCGUCUUUCGGCCCCCUUCU |
| | | | | CUCACCAUCCAUCCUCAUGCCGUGCUCG |
| | | | | AAGGCCAGCCCUUUAAGGCCACAUGCAC |
| | | | | UGCUGCGACCUACUACCCUGGCAACAGA |
| | | | | GCCGAGUUUGUCUGGUUUGAGGAUGGU |
| | | | | CGGCGAGUAUUCGAUCCAGCCCAGAUUC |
| | | | | ACACACAAACGCAGGAAAAUCCGGACG |
| | | | | GCUUCAGCACAGUGUCCACGGUGACCUC |
| | | | | UGCUGCAGUUGGUGGACAAGGACCCCC |
| | | | | UCGAACCUUCACCUGUCAGCUGACCUGG |
| | | | | CACAGAGACUCCGUAAGCUUCAGCCGUA |
| | | | | GAAACGCCUCUGGAACCGCCAGUGUGU |
| | | | | UGCCGAGGCCGACUAUCACGAUGGAAU |
| | | | | UCACAGGCGAUCAUGCCGUCUGUACUGC |
| | | | | CGGCUGUGUGCCAGAAGGCGUAACCUU |
| | | | | CGCUUGGUUUCUCGGGGAUGACUCAAG |
| | | | | UCCUGCAGAGAAAGUGGCUGUGGCCUC |
| | | | | UCAGACGAGCUGCGGUCGACCAGGAAC |
| | | | | AGCUACCAUUCGCAGCACUCUGCCCGUG |
| | | | | UCCUACGAGCAGACGGAGUACAUCUGC |
| | | | | AGGCUGGCCGGCUAUCCCGAUGGGAUU |
| | | | | CCAGUCCUGGAGCACCACUGA |
| 199 | HSV-2 gD- | HSV-2 gC | Version | AUGGGGAGACUCACAUCAGGCGUAGGA |
| | KYA | (27-426) | 2.1 | ACCGCUGCCCUGUUGGUCGUGGCCGUUG |
| | | | | GUCUGAGAGUUGUGUGUGCCAAAUAUG |
| | | | | CUGCAAGCCCCGGCAGAACCAUAACAGU |
| | | | | AGGGCCACGGGGGAAUGCUUCCAAUGC |
| | | | | UGCACCUUCCGCUUCACCGAGGAAUGCU |
| | | | | UCUGCCCCAAGAACUACCCCCACUCCUC |
| | | | | CUCAACCCAGGAAAGCGACAAAGUCCAA |
| | | | | GGCCAGCACCGCAAAACCCGCUCCUCCU |
| | | | | CCAAAGACUGGGCCCCCAAAGACAAGUA |
| | | | | GCGAACCAGUUCGGUGCAACAGGCAUG |
| | | | | ACCCACUUGCACGCUAUGGGUCAAGAG |
| | | | | UCCAGAUACGGUGUCGCUUCCCUAACAG |
| | | | | UACAAGGACUGAGUUUCGGCUGCAGAU |
| | | | | CUGGCGUUAUGCCACAGCUACUGACGCA |
| | | | | GAGAUUGGUACCGCCCCCAGUUUGGAA |
| | | | | GAGGUGAUGGUCAACGUGUCCGCACCCC |
| | | | | CAGGAGGACAGCUGGUCUAUGACUCAG |
| | | | | CGCCCAAUAGGACCGAUCCCCACGUGAU |
| | | | | CUGGGCAGAAGGAGCCGGUCCUGGGGC |
| | | | | CUCUCCACGGCUGUACUCAGUUGUUGGC |
| | | | | CCGCUUGGACGACAGAGACUCAUCAUCG |
| | | | | AAGAGCUGACACUGGAGACACAGGGGA |
| | | | | UGUACUACUGGGUGUGGGGCCGUACUG |
| | | | | ACCGCCCUUCCGCAUAUGGCACUUGGGU |
| | | | | GAGAGUUCGCGUCUUUCGGCCCCCUUCU |
| | | | | CUCACCAUCCAUCCUCAUGCCGUGCUCG |
| | | | | AAGGCCAGCCCUUUAAGGCCACAUGCAC |
| | | | | UGCUGCGACCUACUACCCUGGCAACAGA |
| | | | | GCCGAGUUUGUCUGGUUUGAGGAUGGU |
| | | | | CGGCGAGUAUUCGAUCCAGCCCAGAUUC |
| | | | | ACACACAAACGCAGGAAAAUCCGGACG |
| | | | | GCUUCAGCACAGUGUCCACGGUGACCUC |
| | | | | UGCUGCAGUUGGUGGACAAGGACCCCC |
| | | | | UCGAACCUUCACCUGUCAGCUGACCUGG |
| | | | | CACAGAGACUCCGUAAGCUUCAGCCGUA |
| | | | | GAAACGCCUCUGGAACCGCCAGUGUGU |
| | | | | UGCCGAGGCCGACUAUCACGAUGGAAU |
| | | | | UCACAGGCGAUCAUGCCGUCUGUACUGC |
| | | | | CGGCUGUGUGCCAGAAGGCGUAACCUU |
| | | | | CGCUUGGUUUCUCGGGGAUGACUCAAG |
| | | | | UCCUGCAGAGAAAGUGGCUGUGGCCUC |
| | | | | UCAGACGAGCUGCGGUCGACCAGGAAC |
| | | | | AGCUACCAUUCGCAGCACUCUGCCCGUG |
| | | | | UCCUACGAGCAGACGGAGUACAUCUGC |
| | | | | AGGCUGGCCGGCUAUCCCGAUGGGAUU |
| | | | | CCAGUCCUGGAGCACCACUGA |
| 200 | HSV-2 gD- | HSV-2 gC | Version | AUGGGGAGACUCACAUCAGGCGUAGGA |
| | KYA | (27-426) | 2.2 | ACCGCUGCCCUGUUGGUCGUGGCCGUUG |
| | | | | GUCUGAGAGUUGUGUGUGCCAAAUAUG |
| | | | | CUGCAAGCCCCGGCAGAACCAUAACAGU |
| | | | | AGGGCCACGGGGGAAUGCUUCCAAUGC |
| | | | | UGCACCUUCCGCUUCACCGAGGAAUGCU |
| | | | | UCUGCCCCAAGAACUACCCCCACUCCUC |
| | | | | CUCAACCCAGGAAAGCGACAAAGUCCAA |
| | | | | GGCCAGCACCGCAAAACCCGCUCCUCCU |
| | | | | CCAAAGACUGGGCCCCCAAAGACAAGUA |
| | | | | GCGAACCAGUUCGGUGCAACAGGCAUG |
| | | | | ACCCACUUGCACGCUAUGGGUCAAGAG |
| | | | | UCCAGAUACGGUGUCGCUUCCCUAACAG |
| | | | | UACAAGGACUGAGUUUCGGCUGCAGAU |
| | | | | CUGGCGUUAUGCCACAGCUACUGACGCA |
| | | | | GAGAUUGGUACCGCCCCCAGUUUGGAA |
| | | | | GAGGUGAUGGUCAACGUGUCCGCACCCC |
| | | | | CAGGAGGACAGCUGGUCUAUGACUCAG |
| | | | | CGCCCAAUAGGACCGAUCCCCACGUGAU |
| | | | | CUGGGCAGAAGGAGCCGGUCCUGGGGC |
| | | | | CUCUCCACGGCUGUACUCAGUUGUUGGC |
| | | | | CCGCUUGGACGACAGAGACUCAUCAUCG |
| | | | | AAGAGCUGACACUGGAGACACAGGGGA |
| | | | | UGUACUACUGGGUGUGGGGCCGUACUG |
| | | | | ACCGCCCUUCCGCAUAUGGCACUUGGGU |
| | | | | GAGAGUUCGCGUCUUUCGGCCCCCUUCU |
| | | | | CUCACCAUCCAUCCUCAUGCCGUGCUCG |
| | | | | AAGGCCAGCCCUUUAAGGCCACAUGCAC |
| | | | | UGCUGCGACCUACUACCCUGGCAACAGA |
| | | | | GCCGAGUUUGUCUGGUUUGAGGAUGGU |
| | | | | CGGCGAGUAUUCGAUCCAGCCCAGAUUC |
| | | | | ACACACAAACGCAGGAAAAUCCGGACG |
| | | | | GCUUCAGCACAGUGUCCACGGUGACCUC |
| | | | | UGCUGCAGUUGGUGGACAAGGACCCCC |
| | | | | UCGAACCUUCACCUGUCAGCUGACCUGG |
| | | | | CACAGAGACUCCGUAAGCUUCAGCCGUA |
| | | | | GAAACGCCUCUGGAACCGCCAGUGUGU |
| | | | | UGCCGAGGCCGACUAUCACGAUGGAAU |
| | | | | UCACAGGCGAUCAUGCCGUCUGUACUGC |
| | | | | CGGCUGUGUGCCAGAAGGCGUAACCUU |
| | | | | CGCUUGGUUUCUCGGGGAUGACUCAAG |
| | | | | UCCUGCAGAGAAAGUGGCUGUGGCCUC |
| | | | | UCAGACGAGCUGCGGUCGACCAGGAAC |
| | | | | AGCUACCAUUCGCAGCACUCUGCCCGUG |
| | | | | UCCUACGAGCAGACGGAGUACAUCUGC |
| | | | | AGGCUGGCCGGCUAUCCCGAUGGGAUU |
| | | | | CCAGUCCUGGAGCACCACUGA |
| 201 | HSV-2 gD- | HSV-2 gC | Version 1 | AUGGGCAGACUGACAUCUGGCGUGGGA |
| | KYALA | (28-426) | | ACAGCUGCUCUGCUGGUGGUUGCUGUG |
| | | | | GGCCUGAGAGUCGUGUGUGCCAAAUAC |
| | | | | GCCCUGGCCUCUCCCGGCAGAACCAUCA |
| | | | | CAGUGGGCCCUAGAGGCAACGCCUCUAA |
| | | | | UGCCGCUCCUAGCGCCUCUCCUAGAAAC |
| | | | | GCCUCUGCUCCCAGAACCACACCUACAC |
| | | | | CUCCACAGCCUAGAAAGGCCACCAAGAG |
| | | | | CAAGGCCAGCACAGCCAAACCUGCUCCU |
| | | | | CCACCUAAGACAGGCCCUCCAAAGACAA |
| | | | | GCUCUGAGCCCGUGCGGUGCAACAGACA |
| | | | | CGAUCCACUGGCCAGAUACGGCAGCCGG |
| | | | | GUGCAGAUCAGAUGCAGAUUCCCCAAC |
| | | | | AGCACCCGGACCGAGUUCCGGCUCCAGA |
| | | | | UUUGGAGAUACGCCACCGCCACAGAUGC |
| | | | | CGAGAUUGGAACAGCCCCUAGCCUGGA |
| | | | | AGAAGUGAUGGUCAACGUUUCAGCCCC |
| | | | | UCCUGGCGGCCAGCUGGUGUAUGAUUC |
| | | | | UGCCCCUAACCGGACCGAUCCUCACGUG |
| | | | | AUAUGGGCUGAAGGUGCUGGCCCAGGC |
| | | | | GCAAGCCCUAGACUGUAUUCUGUUGUG |
| | | | | GGCCCUCUGGGCAGACAGCGGCUGAUCA |
| | | | | UUGAGGAACUGACCCUGGAAACCCAGG |
| | | | | GCAUGUACUACUGGGUCUGGGGCAGAA |
| | | | | CCGAUAGACCAAGCGCCUAUGGCACCUG |
| | | | | GGUUCGAGUGCGAGUGUUCAGACCUCC |
| | | | | UAGCCUGACCAUCCAUCCUCACGCCGUU |
| | | | | CUGGAAGGCCAGCCUUUCAAGGCCACAU |
| | | | | GUACCGCCGCCACCUACUAUCCCGGAAA |
| | | | | CAGAGCCGAGUUCGUUUGGUUCGAGGA |
| | | | | CGGCAGAAGGGUGUUCGACCCCGCUCAG |
| | | | | AUCCACACACAGACCCAAGAGAACCCCG |
| | | | | ACGGCUUUAGCACCGUGUCCACAGUGAC |
| | | | | AUCUGCCGCCGUUGGAGGACAGGGCCCU |
| | | | | CCUAGAACCUUUACCUGCCAGCUGACCU |
| | | | | GGCACAGAGACAGCGUGUCCUUCAGCA |
| | | | | GAAGAAACGCCAGCGGCACAGCCAGCGU |
| | | | | UCUGCCUAGACCUACCAUCACCAUGGAA |
| | | | | UUCACCGGCGACCACGCCGUGUGUACAG |
| | | | | CUGGAUGUGUUCCUGAGGGCGUGACCU |
| | | | | UCGCUUGGUUUCUGGGCGACGAUAGCA |
| | | | | GCCCUGCCGAAAAAGUGGCUGUGGCCA |
| | | | | GCCAGACAAGCUGUGGCAGACCUGGAA |
| | | | | CCGCCACCAUCAGAAGCACACUGCCUGU |
| | | | | CAGCUACGAGCAGACCGAGUACAUCUG |
| | | | | UCGGCUGGCCGGCUAUCCUGAUGGCAUC |
| | | | | CCUGUGCUGGAACACCACUGA |
| 202 | HSV-2 gD- | HSV-2 gC | Version 2 | AUGGGGAGACUCACAUCAGGCGUAGGA |
| | KYALA | (28-426) | | ACCGCUGCCCUGUUGGUCGUGGCCGUUG |
| | | | | GUCUGAGAGUUGUGUGUGCCAAAUAUG |
| | | | | CUCUGGCAAGCCCCGGCAGAACCAUAAC |
| | | | | AGUAGGGCCACGGGGGAAUGCUUCCAA |
| | | | | UGCUGCACCUUCCGCUUCACCGAGGAAU |
| | | | | GCUUCUGCCCCAAGAACUACCCCCACUC |
| | | | | CUCCUCAACCCAGGAAAGCGACAAAGUC |
| | | | | CAAGGCCAGCACCGCAAAACCCGCUCCU |
| | | | | CCUCCAAAGACUGGGCCCCCAAAGACAA |
| | | | | GUAGCGAACCAGUUCGGUGCAACAGGC |
| | | | | AUGACCCACUUGCACGCUAUGGGUCAA |
| | | | | GAGUCCAGAUACGGUGUCGCUUCCCUA |
| | | | | ACAGUACAAGGACUGAGUUUCGGCUGC |
| | | | | AGAUCUGGCGUUAUGCCACAGCUACUG |
| | | | | ACGCAGAGAUUGGUACCGCCCCCAGUUU |
| | | | | GGAGGAAGUGAUGGUCAACGUGUCCGC |
| | | | | ACCCCCAGGAGGACAGCUGGUCUAUGAC |
| | | | | UCAGCGCCCAAUAGGACCGAUCCCCACG |
| | | | | UGAUCUGGGCAGAAGGAGCCGGUCCUG |
| | | | | GGGCCUCUCCACGGCUGUACUCAGUUGU |
| | | | | UGGCCCGCUUGGACGACAGAGACUCAUC |
| | | | | AUCGAGGAACUGACACUGGAGACACAG |
| | | | | GGGAUGUACUACUGGGUGUGGGGCCGU |
| | | | | ACUGACCGCCCUUCCGCAUAUGGCACUU |
| | | | | GGGUGAGAGUUCGCGUCUUUCGGCCCCC |
| | | | | UUCUCUCACCAUCCAUCCUCAUGCCGUG |
| | | | | CUCGAAGGCCAGCCCUUUAAGGCCACAU |
| | | | | GCACUGCUGCGACCUACUACCCUGGCAA |
| | | | | CAGAGCCGAGUUUGUCUGGUUUGAGGA |
| | | | | UGGUCGGCGAGUAUUCGAUCCAGCCCA |
| | | | | GAUUCACACACAAACGCAGGAAAAUCC |
| | | | | GGACGGCUUCAGCACAGUGUCCACGGU |
| | | | | GACCUCUGCUGCAGUUGGUGGACAAGG |
| | | | | ACCCCCUCGAACCUUCACCUGUCAGCUG |
| | | | | ACCUGGCACAGAGACUCCGUAAGCUUCA |
| | | | | GCCGUAGAAACGCCUCUGGAACCGCCAG |
| | | | | UGUGUUGCCGAGGCCGACUAUCACGAU |
| | | | | GGAAUUCACAGGCGAUCAUGCCGUCUG |
| | | | | UACUGCCGGCUGUGUGCCAGAAGGCGU |
| | | | | AACCUUCGCUUGGUUUCUCGGGGAUGA |
| | | | | CUCAAGUCCUGCAGAGAAAGUGGCUGU |
| | | | | GGCCUCUCAGACGAGCUGCGGUCGACCA |
| | | | | GGAACAGCUACCAUUCGCAGCACUCUGC |
| | | | | CCGUGUCCUACGAGCAGACGGAGUACA |
| | | | | UCUGCAGGCUGGCCGGCUAUCCCGAUGG |
| | | | | GAUUCCAGUCCUGGAGCACCACUGA |
| 203 | HSV-2 gD- | HSV-2 gC | Version | AUGGGGAGACUCACAUCCGGAGUGGGC |
| | KYALA | (28-426) | 2.1 | ACUGCUGCCCUGUUGGUCGUGGCCGUU |
| | | | | GGUCUGAGAGUUGUGUGUGCCAAAUAU |
| | | | | GCUCUGGCAAGCCCCGGCAGAACCAUAA |
| | | | | CAGUAGGGCCACGGGGGAAUGCUUCCA |
| | | | | AUGCUGCACCUUCCGCUUCACCGAGGAA |
| | | | | UGCUUCUGCCCCAAGAACUACCCCCACU |
| | | | | CCUCCUCAACCCAGGAAAGCGACAAAGU |
| | | | | CCAAGGCCAGCACCGCAAAACCCGCUCC |
| | | | | UCCUCCAAAGACUGGGCCCCCAAAGACA |
| | | | | AGUAGCGAACCAGUUCGGUGCAACAGG |
| | | | | CAUGACCCACUUGCACGCUAUGGGUCAA |
| | | | | GAGUCCAGAUACGGUGUCGCUUCCCUA |
| | | | | ACAGUACAAGGACUGAGUUUCGGCUGC |
| | | | | AGAUCUGGCGUUAUGCCACAGCUACUG |
| | | | | ACGCAGAGAUUGGUACCGCCCCCAGUUU |
| | | | | GGAGGAAGUGAUGGUCAACGUGUCCGC |
| | | | | ACCCCCAGGAGGACAGCUGGUCUAUGAC |
| | | | | UCAGCGCCCAAUAGGACAGACCCACAUG |
| | | | | UUAUCUGGGCAGAAGGAGCCGGUCCUG |
| | | | | GGGCCUCUCCACGGCUGUACUCAGUUGU |
| | | | | UGGCCCGCUUGGACGACAGAGACUCAUC |
| | | | | AUCGAGGAACUGACACUGGAGACACAG |
| | | | | GGGAUGUACUACUGGGUGUGGGGCCGU |
| | | | | ACUGACCGCCCUUCCGCAUAUGGCACUU |
| | | | | GGGUGAGAGUUCGCGUCUUUCGGCCCCC |
| | | | | UUCUCUCACCAUCCAUCCUCAUGCCGUG |
| | | | | CUCGAAGGCCAGCCCUUUAAGGCCACAU |
| | | | | GCACUGCUGCGACCUACUACCCUGGCAA |
| | | | | CAGAGCCGAGUUUGUCUGGUUUGAGGA |
| | | | | UGGUCGGCGAGUAUUCGAUCCAGCCCA |
| | | | | GAUUCACACACAAACGCAGGAAAAUCC |
| | | | | GGACGGCUUCAGCACAGUGUCCACGGU |
| | | | | GACCUCUGCUGCAGUUGGUGGACAAGG |
| | | | | ACCCCCUCGAACCUUCACCUGUCAGCUG |
| | | | | ACCUGGCACAGAGACUCCGUAAGCUUCA |
| | | | | GCCGUAGAAACGCCUCUGGAACCGCCAG |
| | | | | UGUGUUGCCGAGGCCGACUAUCACGAU |
| | | | | GGAAUUCACAGGCGAUCAUGCCGUCUG |
| | | | | UACUGCCGGCUGUGUGCCAGAAGGCGU |
| | | | | AACCUUCGCUUGGUUUCUCGGGGAUGA |
| | | | | CUCAAGUCCUGCAGAGAAAGUGGCUGU |
| | | | | GGCCUCUCAGACGAGCUGCGGUCGACCA |
| | | | | GGAACAGCUACCAUUCGCAGCACUCUGC |
| | | | | CCGUGUCCUACGAGCAGACGGAGUACA |
| | | | | UCUGCAGGCUGGCCGGCUAUCCCGAUGG |
| | | | | GAUUCCAGUCCUGGAGCACCACUGA |
| 204 | HSV-2 gD- | HSV-2 gC | Version 3 | AUGGGCAGACUGACCUCCGGCGUGGGC |
| | KYALA | (28-426) | | ACCGCCGCCCUGCUGGUGGUGGCCGUGG |
| | | | | GCCUGAGAGUGGUGUGCGCCAAAUACG |
| | | | | CCCUGGCCUCCCCCGGCAGAACCAUCAC |
| | | | | CGUGGGCCCCAGAGGCAACGCCUCCAAC |
| | | | | GCCGCCCCCUCCGCCUCCCCCAGAAACG |
| | | | | CCUCCGCCCCCAGAACCACCCCCACCCC |
| | | | | UCCCCAGCCCAGAAAAGCCACCAAAUCC |
| | | | | AAAGCCUCCACCGCCAAACCCGCCCCUC |
| | | | | CUCCCAAAACCGGCCCUCCCAAAACCUC |
| | | | | CUCCGAACCCGUGAGAUGCAACAGACAC |
| | | | | GAUCCCCUGGCCAGAUACGGCUCCAGAG |
| | | | | UGCAGAUCAGAUGCAGAUUCCCCAACUC |
| | | | | CACCAGAACCGAAUUCAGACUGCAGAUC |
| | | | | UGGAGAUACGCCACCGCCACCGAUGCCG |
| | | | | AAAUCGGCACCGCCCCCUCCCUGGAAGA |
| | | | | AGUGAUGGUGAACGUGUCCGCCCCUCCU |
| | | | | GGCGGCCAGCUGGUGUACGAUUCCGCCC |
| | | | | CCAACAGAACCGAUCCCCACGUGAUCUG |
| | | | | GGCCGAAGGCGCCGGCCCCGGCGCCUCC |
| | | | | CCCAGACUGUACUCCGUGGUGGGCCCCC |
| | | | | UGGGCAGACAGAGACUGAUCAUCGAAG |
| | | | | AACUGACCCUGGAAACCCAGGGCAUGU |
| | | | | ACUACUGGGUGUGGGGCAGAACCGAUA |
| | | | | GACCCUCCGCCUACGGCACCUGGGUGAG |
| | | | | AGUGAGAGUGUUCAGACCCCCUUCCCUG |
| | | | | ACCAUCCACCCCCACGCCGUGCUGGAAG |
| | | | | GCCAGCCCUUCAAAGCCACCUGCACCGC |
| | | | | CGCCACCUACUACCCCGGCAACAGAGCC |
| | | | | GAAUUCGUGUGGUUCGAAGAUGGCAGA |
| | | | | AGGGUGUUCGAUCCCGCCCAGAUCCACA |
| | | | | CCCAGACCCAGGAAAACCCCGACGGCUU |
| | | | | CUCCACCGUGUCCACCGUGACCUCCGCC |
| | | | | GCCGUGGGCGGCCAGGGCCCUCCCAGAA |
| | | | | CCUUCACCUGCCAGCUGACCUGGCACAG |
| | | | | AGACUCCGUGUCCUUCUCCAGAAGAAAC |
| | | | | GCCUCCGGCACCGCCUCCGUGCUGCCCA |
| | | | | GACCCACCAUCACCAUGGAAUUCACCGG |
| | | | | CGAUCACGCCGUGUGCACCGCCGGCUGC |
| | | | | GUGCCCGAAGGCGUGACCUUCGCCUGGU |
| | | | | UCCUGGGCGAUGAUUCCUCCCCCGCCGA |
| | | | | AAAAGUGGCCGUGGCCUCCCAGACCUCC |
| | | | | UGCGGCAGACCCGGCACCGCCACCAUCA |
| | | | | GAUCCACCCUGCCCGUGUCCUACGAACA |
| | | | | GACCGAAUACAUCUGCAGACUGGCCGGC |
| | | | | UACCCCGAUGGCAUCCCCGUGCUGGAAC |
| | | | | ACCACUGA |
| 205 | HSV-2 gC | HSV-2 gC | Version 2 | AUGGCCUUGGGGAGAGUGGGCCUUGCA |
| | | (28-426) | | GUGGGUCUGUGGGGACUGCUCUGGGUU |
| | | | | GGCGUAGUCGUCGUGCUGGCUAACGCA |
| | | | | AGCCCCGGCAGAACCAUAACAGUAGGGC |
| | | | | CACGGGGGAAUGCUUCCAAUGCUGCACC |
| | | | | UUCCGCUUCACCGAGGAAUGCUUCUGCC |
| | | | | CCAAGAACUACCCCCACUCCUCCUCAAC |
| | | | | CCAGGAAAGCGACAAAGUCCAAGGCCA |
| | | | | GCACCGCAAAACCCGCUCCUCCUCCAAA |
| | | | | GACUGGGCCCCCAAAGACAAGUAGCGA |
| | | | | ACCAGUUCGGUGCAACAGGCAUGACCCA |
| | | | | CUUGCACGCUAUGGGUCAAGAGUCCAG |
| | | | | AUACGGUGUCGCUUCCCUAACAGUACA |
| | | | | AGGACUGAGUUUCGGCUGCAGAUCUGG |
| | | | | CGUUAUGCCACAGCUACUGACGCAGAG |
| | | | | AUUGGUACCGCCCCCAGUUUGGAGGAA |
| | | | | GUGAUGGUCAACGUGUCCGCACCCCCAG |
| | | | | GAGGACAGCUGGUCUAUGACUCAGCGC |
| | | | | CCAAUAGGACCGAUCCCCACGUGAUCUG |
| | | | | GGCAGAAGGAGCCGGUCCUGGGGCCUC |
| | | | | UCCACGGCUGUACUCAGUUGUUGGCCCG |
| | | | | CUUGGACGACAGAGACUCAUCAUCGAG |
| | | | | GAACUGACACUGGAGACACAGGGGAUG |
| | | | | UACUACUGGGUGUGGGGCCGUACUGAC |
| | | | | CGCCCUUCCGCAUAUGGCACUUGGGUGA |
| | | | | GAGUUCGCGUCUUUCGGCCCCCUUCUCU |
| | | | | CACCAUCCAUCCUCAUGCCGUGCUCGAA |
| | | | | GGCCAGCCCUUUAAGGCCACAUGCACUG |
| | | | | CUGCGACCUACUACCCUGGCAACAGAGC |
| | | | | CGAGUUUGUCUGGUUUGAGGAUGGUCG |
| | | | | GCGAGUAUUCGAUCCAGCCCAGAUUCAC |
| | | | | ACACAAACGCAGGAAAAUCCGGACGGC |
| | | | | UUCAGCACAGUGUCCACGGUGACCUCUG |
| | | | | CUGCAGUUGGUGGACAAGGACCCCCUCG |
| | | | | AACCUUCACCUGUCAGCUGACCUGGCAC |
| | | | | AGAGACUCCGUAAGCUUCAGCCGUAGA |
| | | | | AACGCCUCUGGAACCGCCAGUGUGUUGC |
| | | | | CGAGGCCGACUAUCACGAUGGAAUUCA |
| | | | | CAGGCGAUCAUGCCGUCUGUACUGCCGG |
| | | | | CUGUGUGCCAGAAGGCGUAACCUUCGC |
| | | | | UUGGUUUCUCGGGGAUGACUCAAGUCC |
| | | | | UGCAGAGAAAGUGGCUGUGGCCUCUCA |
| | | | | GACGAGCUGCGGUCGACCAGGAACAGC |
| | | | | UACCAUUCGCAGCACUCUGCCCGUGUCC |
| | | | | UACGAGCAGACGGAGUACAUCUGCAGG |
| | | | | CUGGCCGGCUAUCCCGAUGGGAUUCCAG |
| | | | | UCCUGGAGCACCACUGA |
| 206 | HSV-2 gC | HSV-2 gC | Version | AUGGCACUAGGGAGAGUGGGAUUAGCU |
| | | (28-426) | 2.1 | GUGGGUCUGUGGGGACUGCUCUGGGUA |
| | | | | GGAGUCGUCGUCGUGCUGGCUAACGCA |
| | | | | AGCCCCGGCAGAACCAUAACAGUAGGGC |
| | | | | CACGGGGGAAUGCUUCCAAUGCUGCACC |
| | | | | UUCCGCUUCACCGAGGAAUGCUUCUGCC |
| | | | | CCAAGAACUACCCCCACUCCUCCUCAAC |
| | | | | CCAGGAAAGCGACAAAGUCCAAGGCCA |
| | | | | GCACCGCAAAACCCGCUCCUCCUCCAAA |
| | | | | GACUGGGCCCCCAAAGACAAGUAGCGA |
| | | | | ACCAGUUCGGUGCAACAGGCAUGACCCA |
| | | | | CUUGCACGCUAUGGGUCAAGAGUCCAG |
| | | | | AUACGGUGUCGCUUCCCUAACAGUACA |
| | | | | AGGACUGAGUUUCGGCUGCAGAUCUGG |
| | | | | CGUUAUGCCACAGCUACUGACGCAGAG |
| | | | | AUUGGUACCGCCCCCAGUUUGGAGGAA |
| | | | | GUGAUGGUCAACGUGUCCGCACCCCCAG |
| | | | | GAGGACAGCUGGUCUAUGACUCAGCGC |
| | | | | CCAAUAGGACCGAUCCCCACGUGAUCUG |
| | | | | GGCAGAAGGAGCCGGUCCUGGGGCCUC |
| | | | | UCCACGGCUGUACUCAGUUGUUGGCCCG |
| | | | | CUUGGACGACAGAGACUCAUCAUCGAG |
| | | | | GAACUGACACUGGAGACACAGGGGAUG |
| | | | | UACUACUGGGUGUGGGGCCGUACUGAC |
| | | | | CGCCCUUCCGCAUAUGGCACUUGGGUGA |
| | | | | GAGUUCGCGUCUUUCGGCCCCCUUCUCU |
| | | | | CACCAUCCAUCCUCAUGCCGUGCUCGAA |
| | | | | GGCCAGCCCUUUAAGGCCACAUGCACUG |
| | | | | CUGCGACCUACUACCCUGGCAACAGAGC |
| | | | | CGAGUUUGUCUGGUUUGAGGAUGGUCG |
| | | | | GCGAGUAUUCGAUCCAGCCCAGAUUCAC |
| | | | | ACACAAACGCAGGAAAAUCCGGACGGC |
| | | | | UUCAGCACAGUGUCCACGGUGACCUCUG |
| | | | | CUGCAGUUGGUGGACAAGGACCCCCUCG |
| | | | | AACCUUCACCUGUCAGCUGACCUGGCAC |
| | | | | AGAGACUCCGUAAGCUUCAGCCGUAGA |
| | | | | AACGCCUCUGGAACCGCCAGUGUGUUGC |
| | | | | CGAGGCCGACUAUCACGAUGGAAUUCA |
| | | | | CAGGCGAUCAUGCCGUCUGUACUGCCGG |
| | | | | CUGUGUGCCAGAAGGCGUAACCUUCGC |
| | | | | UUGGUUUCUCGGGGAUGACUCAAGUCC |
| | | | | UGCAGAGAAAGUGGCUGUGGCCUCUCA |
| | | | | GACGAGCUGCGGUCGACCAGGAACAGC |
| | | | | UACCAUUCGCAGCACUCUGCCCGUGUCC |
| | | | | UACGAGCAGACGGAGUACAUCUGCAGG |
| | | | | CUGGCCGGCUAUCCCGAUGGGAUUCCAG |
| | | | | UCCUGGAGCACCACUGA |
| 207 | HSV-2 gC | HSV-2 gC | Version | AUGGCCUUGGGGAGAGUCGGCCUUGCA |
| | | (28-426) | 2.2 | GUGGGACUGUGGGGUCUGCUGUGGGUU |
| | | | | GGCGUGGUAGUCGUGCUCGCUAACGCC |
| | | | | AGCCCCGGCAGAACCAUAACAGUAGGGC |
| | | | | CACGGGGGAAUGCUUCCAAUGCUGCACC |
| | | | | UUCCGCUUCACCGAGGAAUGCUUCUGCC |
| | | | | CCAAGAACUACCCCCACUCCUCCUCAAC |
| | | | | CCAGGAAAGCGACAAAGUCCAAGGCCA |
| | | | | GCACCGCAAAACCCGCUCCUCCUCCAAA |
| | | | | GACUGGGCCCCCAAAGACAAGUAGCGA |
| | | | | ACCAGUUCGGUGCAACAGGCAUGACCCA |
| | | | | CUUGCACGCUAUGGGUCAAGAGUCCAG |
| | | | | AUACGGUGUCGCUUCCCUAACAGUACA |
| | | | | AGGACUGAGUUUCGGCUGCAGAUCUGG |
| | | | | CGUUAUGCCACAGCUACUGACGCAGAG |
| | | | | AUUGGUACCGCCCCCAGUUUGGAGGAA |
| | | | | GUGAUGGUCAACGUGUCCGCACCCCCAG |
| | | | | GAGGACAGCUGGUCUAUGACUCAGCGC |
| | | | | CCAAUAGGACCGAUCCCCACGUGAUCUG |
| | | | | GGCAGAAGGAGCCGGUCCUGGGGCCUC |
| | | | | UCCACGGCUGUACUCAGUUGUUGGCCCG |
| | | | | CUUGGACGACAGAGACUCAUCAUCGAG |
| | | | | GAACUGACACUGGAGACACAGGGGAUG |
| | | | | UACUACUGGGUGUGGGGCCGUACUGAC |
| | | | | CGCCCUUCCGCAUAUGGCACUUGGGUGA |
| | | | | GAGUUCGCGUCUUUCGGCCCCCUUCUCU |
| | | | | CACCAUCCAUCCUCAUGCCGUGCUCGAA |
| | | | | GGCCAGCCCUUUAAGGCCACAUGCACUG |
| | | | | CUGCGACCUACUACCCUGGCAACAGAGC |
| | | | | CGAGUUUGUCUGGUUUGAGGAUGGUCG |
| | | | | GCGAGUAUUCGAUCCAGCCCAGAUUCAC |
| | | | | ACACAAACGCAGGAAAAUCCGGACGGC |
| | | | | UUCAGCACAGUGUCCACGGUGACCUCUG |
| | | | | CUGCAGUUGGUGGACAAGGACCCCCUCG |
| | | | | AACCUUCACCUGUCAGCUGACCUGGCAC |
| | | | | AGAGACUCCGUAAGCUUCAGCCGUAGA |
| | | | | AACGCCUCUGGAACCGCCAGUGUGUUGC |
| | | | | CGAGGCCGACUAUCACGAUGGAAUUCA |
| | | | | CAGGCGAUCAUGCCGUCUGUACUGCCGG |
| | | | | CUGUGUGCCAGAAGGCGUAACCUUCGC |
| | | | | UUGGUUUCUCGGGGAUGACUCAAGUCC |
| | | | | UGCAGAGAAAGUGGCUGUGGCCUCUCA |
| | | | | GACGAGCUGCGGUCGACCAGGAACAGC |
| | | | | UACCAUUCGCAGCACUCUGCCCGUGUCC |
| | | | | UACGAGCAGACGGAGUACAUCUGCAGG |
| | | | | CUGGCCGGCUAUCCCGAUGGGAUUCCAG |
| | | | | UCCUGGAGCACCACUGA |
| 208 | HSV-2 gC | HSV-2 gC | Version | AUGGCUCUGGGCAGAGUUGGACUGGCU |
| | | (28-426) | 2.3 | GUUGGACUGUGGGGACUGCUGUGGGUU |
| | | | | GGAGUGGUGGUGGUGCUGGCUAAUGCU |
| | | | | AGCCCCGGCAGAACCAUAACAGUAGGGC |
| | | | | CACGGGGGAAUGCUUCCAAUGCUGCACC |
| | | | | UUCCGCUUCACCGAGGAAUGCUUCUGCC |
| | | | | CCAAGAACUACCCCCACUCCUCCUCAAC |
| | | | | CCAGGAAAGCGACAAAGUCCAAGGCCA |
| | | | | GCACCGCAAAACCCGCUCCUCCUCCAAA |
| | | | | GACUGGGCCCCCAAAGACAAGUAGCGA |
| | | | | ACCAGUUCGGUGCAACAGGCAUGACCCA |
| | | | | CUUGCACGCUAUGGGUCAAGAGUCCAG |
| | | | | AUACGGUGUCGCUUCCCUAACAGUACA |
| | | | | AGGACUGAGUUUCGGCUGCAGAUCUGG |
| | | | | CGUUAUGCCACAGCUACUGACGCAGAG |
| | | | | AUUGGUACCGCCCCCAGUUUGGAGGAA |
| | | | | GUGAUGGUCAACGUGUCCGCACCCCCAG |
| | | | | GAGGACAGCUGGUCUAUGACUCAGCGC |
| | | | | CCAAUAGGACCGAUCCCCACGUGAUCUG |
| | | | | GGCAGAAGGAGCCGGUCCUGGGGCCUC |
| | | | | UCCACGGCUGUACUCAGUUGUUGGCCCG |
| | | | | CUUGGACGACAGAGACUCAUCAUCGAG |
| | | | | GAACUGACACUGGAGACACAGGGGAUG |
| | | | | UACUACUGGGUGUGGGGCCGUACUGAC |
| | | | | CGCCCUUCCGCAUAUGGCACUUGGGUGA |
| | | | | GAGUUCGCGUCUUUCGGCCCCCUUCUCU |
| | | | | CACCAUCCAUCCUCAUGCCGUGCUCGAA |
| | | | | GGCCAGCCCUUUAAGGCCACAUGCACUG |
| | | | | CUGCGACCUACUACCCUGGCAACAGAGC |
| | | | | CGAGUUUGUCUGGUUUGAGGAUGGUCG |
| | | | | GCGAGUAUUCGAUCCAGCCCAGAUUCAC |
| | | | | ACACAAACGCAGGAAAAUCCGGACGGC |
| | | | | UUCAGCACAGUGUCCACGGUGACCUCUG |
| | | | | CUGCAGUUGGUGGACAAGGACCCCCUCG |
| | | | | AACCUUCACCUGUCAGCUGACCUGGCAC |
| | | | | AGAGACUCCGUAAGCUUCAGCCGUAGA |
| | | | | AACGCCUCUGGAACCGCCAGUGUGUUGC |
| | | | | CGAGGCCGACUAUCACGAUGGAAUUCA |
| | | | | CAGGCGAUCAUGCCGUCUGUACUGCCGG |
| | | | | CUGUGUGCCAGAAGGCGUAACCUUCGC |
| | | | | UUGGUUUCUCGGGGAUGACUCAAGUCC |
| | | | | UGCAGAGAAAGUGGCUGUGGCCUCUCA |
| | | | | GACGAGCUGCGGUCGACCAGGAACAGC |
| | | | | UACCAUUCGCAGCACUCUGCCCGUGUCC |
| | | | | UACGAGCAGACGGAGUACAUCUGCAGG |
| | | | | CUGGCCGGCUAUCCCGAUGGGAUUCCAG |
| | | | | UCCUGGAGCACCACUGA |
| 209 | HSV-1 gD- | HSV-2 gC | Version 1 | AUGGGCGGAGCUGCUGCUAGACUGGGA |
| | KY | (28-426) | | GCCGUGAUCCUGUUCGUGGUUAUCGUG |
| | | | | GGACUGCAUGGCGUGCGGGGCAAGUAU |
| | | | | AGCCCUGGCAGAACCAUCACAGUGGGCC |
| | | | | CUAGAGGCAACGCCUCUAAUGCCGCUCC |
| | | | | UAGCGCCUCUCCUAGAAACGCCUCUGCU |
| | | | | CCCAGAACCACACCUACACCUCCACAGC |
| | | | | CUAGAAAGGCCACCAAGAGCAAGGCCA |
| | | | | GCACAGCCAAACCUGCUCCUCCACCUAA |
| | | | | GACAGGCCCUCCAAAGACAAGCUCUGAG |
| | | | | CCCGUGCGGUGCAACAGACACGAUCCAC |
| | | | | UGGCCAGAUACGGCAGCCGGGUGCAGA |
| | | | | UCAGAUGCAGAUUCCCCAACAGCACCCG |
| | | | | GACCGAGUUCCGGCUCCAGAUUUGGAG |
| | | | | AUACGCCACCGCCACAGAUGCCGAGAUU |
| | | | | GGAACAGCCCCUAGCCUGGAAGAAGUG |
| | | | | AUGGUCAACGUUUCAGCCCCUCCUGGCG |
| | | | | GCCAGCUGGUGUAUGAUUCUGCCCCUA |
| | | | | ACCGGACCGAUCCUCACGUGAUAUGGGC |
| | | | | UGAAGGUGCUGGCCCUGGCGCUUCCCCU |
| | | | | AGACUGUAUUCUGUUGUGGGCCCUCUG |
| | | | | GGCAGACAGCGGCUGAUCAUUGAGGAA |
| | | | | CUGACCCUGGAAACCCAGGGCAUGUACU |
| | | | | ACUGGGUCUGGGGCAGAACCGAUAGAC |
| | | | | CAAGCGCCUAUGGCACCUGGGUUCGAG |
| | | | | UGCGAGUGUUCAGACCUCCUAGCCUGAC |
| | | | | CAUCCAUCCUCACGCCGUUCUGGAAGGC |
| | | | | CAGCCUUUCAAGGCCACAUGUACCGCCG |
| | | | | CCACCUACUAUCCCGGAAACAGAGCCGA |
| | | | | GUUCGUUUGGUUCGAGGACGGCAGAAG |
| | | | | GGUGUUCGACCCCGCUCAGAUCCACACA |
| | | | | CAGACCCAAGAGAACCCCGACGGCUUUA |
| | | | | GCACCGUGUCCACAGUGACAUCUGCCGC |
| | | | | CGUUGGAGGACAGGGCCCUCCUAGAACC |
| | | | | UUUACCUGCCAGCUGACCUGGCACAGAG |
| | | | | ACAGCGUGUCCUUCAGCAGAAGAAACG |
| | | | | CCAGCGGCACAGCCAGCGUUCUGCCUAG |
| | | | | ACCUACCAUCACCAUGGAAUUCACCGGC |
| | | | | GACCACGCCGUGUGUACAGCUGGAUGU |
| | | | | GUUCCUGAGGGCGUGACCUUCGCUUGG |
| | | | | UUUCUGGGCGACGAUAGCAGCCCUGCCG |
| | | | | AAAAAGUGGCUGUGGCCAGCCAGACAA |
| | | | | GCUGUGGCAGACCUGGAACCGCCACCAU |
| | | | | CAGAAGCACACUGCCUGUCAGCUACGAG |
| | | | | CAGACCGAGUACAUCUGUCGGCUGGCCG |
| | | | | GCUAUCCUGAUGGCAUCCCUGUGCUGG |
| | | | | AACACCACUGAUAA |
| 210 | HSV-1 gD- | HSV-2 gC | Version 2 | AUGGGAGGAGCAGCUGCCAGACUCGGU |
| | KY | (28-426) | | GCCGUGAUCCUGUUCGUGGUCAUUGUU |
| | | | | GGCCUGCACGGGGUAAGGGGCAAGUAC |
| | | | | AGCCCCGGCAGAACCAUAACAGUAGGGC |
| | | | | CACGGGGGAAUGCUUCCAAUGCUGCACC |
| | | | | UUCCGCUUCACCGAGGAAUGCUUCUGCC |
| | | | | CCAAGAACUACCCCCACUCCUCCUCAAC |
| | | | | CCAGGAAAGCGACAAAGUCCAAGGCCA |
| | | | | GCACCGCAAAACCCGCUCCUCCUCCAAA |
| | | | | GACUGGGCCCCCAAAGACAAGUAGCGA |
| | | | | ACCAGUUCGGUGCAACAGGCAUGACCCA |
| | | | | CUUGCACGCUAUGGGUCAAGAGUCCAG |
| | | | | AUACGGUGUCGCUUCCCUAACAGUACA |
| | | | | AGGACUGAGUUUCGGCUGCAGAUCUGG |
| | | | | CGUUAUGCCACAGCUACUGACGCAGAG |
| | | | | AUUGGUACCGCCCCCAGUUUGGAAGAG |
| | | | | GUGAUGGUCAACGUGUCCGCACCCCCAG |
| | | | | GAGGACAGCUGGUCUAUGACUCAGCGC |
| | | | | CCAAUAGGACCGAUCCCCACGUGAUCUG |
| | | | | GGCAGAAGGAGCCGGUCCUGGGGCCUC |
| | | | | UCCACGGCUGUACUCAGUUGUUGGCCCG |
| | | | | CUUGGACGACAGAGACUCAUCAUCGAA |
| | | | | GAGCUGACACUGGAGACACAGGGGAUG |
| | | | | UACUACUGGGUGUGGGGCCGUACUGAC |
| | | | | CGCCCUUCCGCAUAUGGCACUUGGGUGA |
| | | | | GAGUUCGCGUCUUUCGGCCCCCUUCUCU |
| | | | | CACCAUCCAUCCUCAUGCCGUGCUCGAA |
| | | | | GGCCAGCCCUUUAAGGCCACAUGCACUG |
| | | | | CUGCGACCUACUACCCUGGCAACAGAGC |
| | | | | CGAGUUUGUCUGGUUUGAGGAUGGUCG |
| | | | | GCGAGUAUUCGAUCCAGCCCAGAUUCAC |
| | | | | ACACAAACGCAGGAAAAUCCGGACGGC |
| | | | | UUCAGCACAGUGUCCACGGUGACCUCUG |
| | | | | CUGCAGUUGGUGGACAAGGACCCCCUCG |
| | | | | AACCUUCACCUGUCAGCUGACCUGGCAC |
| | | | | AGAGACUCCGUAAGCUUCAGCCGUAGA |
| | | | | AACGCCUCUGGAACCGCCAGUGUGUUGC |
| | | | | CGAGGCCGACUAUCACGAUGGAAUUCA |
| | | | | CAGGCGAUCAUGCCGUCUGUACUGCCGG |
| | | | | CUGUGUGCCAGAAGGCGUAACCUUCGC |
| | | | | UUGGUUUCUCGGGGAUGACUCAAGUCC |
| | | | | UGCAGAGAAAGUGGCUGUGGCCUCUCA |
| | | | | GACGAGCUGCGGUCGACCAGGAACAGC |
| | | | | UACCAUUCGCAGCACUCUGCCCGUGUCC |
| | | | | UACGAGCAGACGGAGUACAUCUGCAGG |
| | | | | CUGGCCGGCUAUCCCGAUGGGAUUCCAG |
| | | | | UCCUGGAGCACCACUGAUAA |
| 211 | HSV-1 gD- | HSV-2 gC | Version 3 | AUGGGAGGAGCUGCUGCUAGAUUGGGA |
| | KY | (28-426) | | GCUGUGAUUCUGUUUGUGGUGAUUGUG |
| | | | | GGACUGCAUGGAGUGAGAGGAAAAUAC |
| | | | | UCUCCUGGAAGAACCAUCACAGUGGGA |
| | | | | CCAAGAGGAAAUGCAAGCAAUGCAGCA |
| | | | | CCUUCUGCUUCUCCAAGAAAUGCUUCUG |
| | | | | CUCCAAGAACCACCCCAACCCCUCCUCA |
| | | | | GCCAAGAAAAGCAACCAAAUCCAAAGC |
| | | | | AUCCACAGCAAAACCUGCACCUCCUCCA |
| | | | | AAAACAGGACCUCCAAAAACCUCCUCUG |
| | | | | AACCUGUGAGAUGCAACAGACAUGAUC |
| | | | | CUCUGGCAAGAUAUGGAUCAAGAGUGC |
| | | | | AGAUCAGAUGCAGAUUUCCAAAUUCCA |
| | | | | CCAGAACAGAAUUCAGACUCCAGAUCU |
| | | | | GGAGAUAUGCAACAGCAACAGAUGCAG |
| | | | | AAAUUGGAACAGCACCAUCUCUGGAAG |
| | | | | AAGUGAUGGUGAAUGUGUCUGCUCCUC |
| | | | | CUGGAGGACAGCUGGUGUAUGAUUCUG |
| | | | | CUCCAAACAGAACAGAUCCUCAUGUGA |
| | | | | UCUGGGCUGAAGGAGCUGGACCUGGAG |
| | | | | CUUCUCCAAGACUGUACUCUGUGGUGG |
| | | | | GACCUCUGGGAAGACAGAGACUGAUCA |
| | | | | UUGAAGAACUGACCCUGGAAACCCAGG |
| | | | | GAAUGUACUACUGGGUGUGGGGAAGAA |
| | | | | CAGACAGACCUUCUGCUUAUGGAACCU |
| | | | | GGGUGAGAGUGAGAGUGUUCAGACCUC |
| | | | | CUUCUCUGACCAUCCACCCUCAUGCUGU |
| | | | | GCUGGAAGGACAGCCUUUCAAAGCAAC |
| | | | | CUGCACAGCAGCAACCUACUACCCUGGA |
| | | | | AACAGAGCUGAAUUUGUGUGGUUUGAA |
| | | | | GAUGGAAGAAGGGUGUUUGAUCCUGCU |
| | | | | CAGAUCCACACCCAGACCCAGGAAAAUC |
| | | | | CUGAUGGAUUUUCCACAGUGUCCACAG |
| | | | | UGACAUCUGCUGCUGUGGGAGGACAGG |
| | | | | GACCUCCAAGAACCUUCACCUGCCAGCU |
| | | | | GACCUGGCACAGAGAUUCUGUGUCUUU |
| | | | | UUCAAGAAGAAAUGCUUCUGGAACAGC |
| | | | | UUCUGUGCUGCCAAGACCAACCAUCACC |
| | | | | AUGGAAUUCACAGGAGAUCAUGCUGUG |
| | | | | UGCACAGCUGGAUGUGUGCCUGAAGGA |
| | | | | GUGACCUUUGCUUGGUUUCUGGGAGAU |
| | | | | GAUUCUUCUCCAGCUGAAAAAGUGGCU |
| | | | | GUGGCUUCCCAGACCUCUUGUGGAAGA |
| | | | | CCUGGAACAGCAACCAUCAGAUCCACCC |
| | | | | UGCCUGUGUCUUAUGAACAGACAGAAU |
| | | | | ACAUUUGCAGACUGGCUGGAUACCCUG |
| | | | | AUGGAAUCCCUGUGCUGGAACACCACU |
| | | | | GAUAA |
| 212 | HSV-1 gD- | HSV-2 gC | Version 4 | AUGGGCGGCGCAGCCGCUAGACUGGGU |
| | KY | (28-426) | | GCAGUCAUCCUCUUUGUGGUGAUCGUG |
| | | | | GGGCUGCAUGGGGUCAGAGGGAAGUAU |
| | | | | UCUCCGGGACGGACUAUAACCGUAGGU |
| | | | | CCAAGAGGAAACGCCUCUAACGCAGCCC |
| | | | | CGUCUGCCUCACCACGAAACGCCUCAGC |
| | | | | UCCCAGAACCACUCCUACUCCACCCCAG |
| | | | | CCUAGGAAGGCGACGAAAUCCAAGGCU |
| | | | | UCCACGGCCAAACCCGCCCCUCCACCCA |
| | | | | AAACCGGACCUCCUAAGACCAGCUCUGA |
| | | | | ACCGGUGCGGUGUAAUAGGCACGACCC |
| | | | | AUUGGCGCGAUAUGGCAGUAGGGUCCA |
| | | | | GAUACGGUGCAGAUUCCCAAACAGCAC |
| | | | | AAGAACAGAAUUCCGGCUGCAAAUCUG |
| | | | | GCGAUAUGCAACGGCCACCGAUGCCGAA |
| | | | | AUCGGAACAGCACCCAGUCUGGAAGAA |
| | | | | GUGAUGGUGAACGUCAGUGCUCCACCU |
| | | | | GGCGGACAACUGGUGUACGACUCUGCA |
| | | | | CCCAAUCGCACAGAUCCCCACGUGAUUU |
| | | | | GGGCCGAGGGUGCUGGACCUGGGGCUU |
| | | | | CACCCAGGCUGUAUAGCGUUGUAGGGC |
| | | | | CACUUGGGAGGCAGAGACUCAUCAUUG |
| | | | | AGGAACUGACCCUGGAAACUCAGGGCA |
| | | | | UGUACUACUGGGUAUGGGGCCGCACAG |
| | | | | AUCGCCCCAGCGCUUAUGGCACCUGGGU |
| | | | | GCGGGUGCGGGUGUUUCGCCCACCCUCC |
| | | | | CUCACCAUUCACCCUCAUGCGGUUCUGG |
| | | | | AGGGACAGCCUUUCAAGGCAACUUGUA |
| | | | | CCGCAGCCACCUACUAUCCCGGCAAUAG |
| | | | | AGCGGAGUUCGUCUGGUUUGAGGACGG |
| | | | | CCGUAGGGUGUUCGAUCCUGCCCAGAU |
| | | | | UCACACCCAGACACAGGAGAAUCCCGAC |
| | | | | GGCUUUAGCACAGUGAGCACUGUGACG |
| | | | | UCUGCUGCCGUUGGUGGUCAAGGGCCU |
| | | | | CCUCGUACCUUCACAUGCCAAUUGACCU |
| | | | | GGCACCGCGACUCAGUUAGCUUUAGCCG |
| | | | | CCGGAAUGCCAGUGGGACCGCCAGUGU |
| | | | | UCUCCCAAGGCCGACAAUCACCAUGGAG |
| | | | | UUCACUGGCGACCAUGCAGUGUGCACA |
| | | | | GCUGGGUGUGUCCCAGAAGGCGUGACU |
| | | | | UUCGCCUGGUUUCUGGGUGAUGACUCC |
| | | | | UCACCCGCCGAGAAAGUAGCUGUCGCUU |
| | | | | CCCAGACUUCCUGUGGACGUCCUGGAAC |
| | | | | UGCGACAAUCCGAAGCACACUGCCGGUU |
| | | | | UCCUACGAGCAGACGGAGUACAUAUGC |
| | | | | CGCCUUGCAGGCUACCCCGAUGGAAUUC |
| | | | | CAGUCCUUGAGCACCAUUGA |
| 213 | HSV-1 | HSV-2 gC | Version 1 | AUGCAUCAGGGCGCUCCAUCUUGGGGU |
| | gB-AP | (28-426) | | AGACGUUGGUUCGUUGUGUGGGCCCUG |
| | | | | CUGGGACUGACACUGGGAGUUCUGGUU |
| | | | | GCCUCUGCUGCUCCUAGCCCUGGCAGAA |
| | | | | CCAUCACAGUGGGCCCUAGAGGCAACGC |
| | | | | CUCUAAUGCCGCUCCUAGCGCCUCUCCU |
| | | | | AGAAACGCCUCUGCUCCCAGAACCACAC |
| | | | | CUACACCUCCACAGCCUAGAAAGGCCAC |
| | | | | CAAGAGCAAGGCCAGCACAGCCAAACCU |
| | | | | GCUCCUCCACCUAAGACAGGCCCUCCAA |
| | | | | AGACAAGCUCUGAGCCCGUGCGGUGCA |
| | | | | ACAGACACGAUCCACUGGCCAGAUACGG |
| | | | | CAGCCGGGUGCAGAUCAGAUGCAGAUU |
| | | | | CCCCAACAGCACCCGGACCGAGUUCCGG |
| | | | | CUCCAGAUUUGGAGAUACGCCACCGCCA |
| | | | | CAGAUGCCGAGAUUGGAACAGCCCCUA |
| | | | | GCCUGGAAGAAGUGAUGGUCAACGUUU |
| | | | | CAGCCCCUCCUGGCGGCCAGCUGGUGUA |
| | | | | UGAUUCUGCCCCUAACCGGACCGAUCCU |
| | | | | CACGUGAUAUGGGCUGAAGGUGCUGGC |
| | | | | CCUGGCGCUUCCCCUAGACUGUAUUCUG |
| | | | | UUGUGGGCCCUCUGGGCAGACAGCGGC |
| | | | | UGAUCAUUGAGGAACUGACCCUGGAAA |
| | | | | CCCAGGGCAUGUACUACUGGGUCUGGG |
| | | | | GCAGAACCGAUAGACCAAGCGCCUAUG |
| | | | | GCACCUGGGUUCGAGUGCGAGUGUUCA |
| | | | | GACCUCCUAGCCUGACCAUCCAUCCUCA |
| | | | | CGCCGUUCUGGAAGGCCAGCCUUUCAAG |
| | | | | GCCACAUGUACCGCCGCCACCUACUAUC |
| | | | | CCGGAAACAGAGCCGAGUUCGUUUGGU |
| | | | | UCGAGGACGGCAGAAGGGUGUUCGACC |
| | | | | CCGCUCAGAUCCACACACAGACCCAAGA |
| | | | | GAACCCCGACGGCUUUAGCACCGUGUCC |
| | | | | ACAGUGACAUCUGCCGCCGUUGGAGGA |
| | | | | CAGGGCCCUCCUAGAACCUUUACCUGCC |
| | | | | AGCUGACCUGGCACAGAGACAGCGUGU |
| | | | | CCUUCAGCAGAAGAAACGCCAGCGGCAC |
| | | | | AGCCAGCGUUCUGCCUAGACCUACCAUC |
| | | | | ACCAUGGAAUUCACCGGCGACCACGCCG |
| | | | | UGUGUACAGCUGGAUGUGUUCCUGAGG |
| | | | | GCGUGACCUUCGCUUGGUUUCUGGGCG |
| | | | | ACGAUAGCAGCCCUGCCGAAAAAGUGG |
| | | | | CUGUGGCCAGCCAGACAAGCUGUGGCA |
| | | | | GACCUGGAACCGCCACCAUCAGAAGCAC |
| | | | | ACUGCCUGUCAGCUACGAGCAGACCGAG |
| | | | | UACAUCUGUCGGCUGGCCGGCUAUCCUG |
| | | | | AUGGCAUCCCUGUGCUGGAACACCACUG |
| | | | | AUAA |
| 214 | HSV-1 gB- | HSV-2 gC | Version 2 | AUGCACCAGGGUGCCCCUUCCUGGGGCA |
| | AP | (28-426) | | GAAGGUGGUUCGUGGUGUGGGCCCUUC |
| | | | | UGGGGCUGACCCUCGGAGUCCUGGUUG |
| | | | | CGAGCGCAGCUCCCAGCCCCGGCAGAAC |
| | | | | CAUAACAGUAGGGCCACGGGGGAAUGC |
| | | | | UUCCAAUGCUGCACCUUCCGCUUCACCG |
| | | | | AGGAAUGCUUCUGCCCCAAGAACUACCC |
| | | | | CCACUCCUCCUCAACCCAGGAAAGCGAC |
| | | | | AAAGUCCAAGGCCAGCACCGCAAAACCC |
| | | | | GCUCCUCCUCCAAAGACUGGGCCCCCAA |
| | | | | AGACAAGUAGCGAACCAGUUCGGUGCA |
| | | | | ACAGGCAUGACCCACUUGCACGCUAUGG |
| | | | | GUCAAGAGUCCAGAUACGGUGUCGCUU |
| | | | | CCCUAACAGUACAAGGACUGAGUUUCG |
| | | | | GCUGCAGAUCUGGCGUUAUGCCACAGC |
| | | | | UACUGACGCAGAGAUUGGUACCGCCCCC |
| | | | | AGUUUGGAAGAGGUGAUGGUCAACGUG |
| | | | | UCCGCACCCCCAGGAGGACAGCUGGUCU |
| | | | | AUGACUCAGCGCCCAAUAGGACCGAUCC |
| | | | | CCACGUGAUCUGGGCAGAAGGAGCCGG |
| | | | | UCCUGGGGCCUCUCCACGGCUGUACUCA |
| | | | | GUUGUUGGCCCGCUUGGACGACAGAGA |
| | | | | CUCAUCAUCGAAGAGCUGACACUGGAG |
| | | | | ACACAGGGGAUGUACUACUGGGUGUGG |
| | | | | GGCCGUACUGACCGCCCUUCCGCAUAUG |
| | | | | GCACUUGGGUGAGAGUUCGCGUCUUUC |
| | | | | GGCCCCCUUCUCUCACCAUCCAUCCUCA |
| | | | | UGCCGUGCUCGAAGGCCAGCCCUUUAAG |
| | | | | GCCACAUGCACUGCUGCGACCUACUACC |
| | | | | CUGGCAACAGAGCCGAGUUUGUCUGGU |
| | | | | UUGAGGAUGGUCGGCGAGUAUUCGAUC |
| | | | | CAGCCCAGAUUCACACACAAACGCAGGA |
| | | | | AAAUCCGGACGGCUUCAGCACAGUGUCC |
| | | | | ACGGUGACCUCUGCUGCAGUUGGUGGA |
| | | | | CAAGGACCCCCUCGAACCUUCACCUGUC |
| | | | | AGCUGACCUGGCACAGAGACUCCGUAA |
| | | | | GCUUCAGCCGUAGAAACGCCUCUGGAAC |
| | | | | CGCCAGUGUGUUGCCGAGGCCGACUAUC |
| | | | | ACGAUGGAAUUCACAGGCGAUCAUGCC |
| | | | | GUCUGUACUGCCGGCUGUGUGCCAGAA |
| | | | | GGCGUAACCUUCGCUUGGUUUCUCGGG |
| | | | | GAUGACUCAAGUCCUGCAGAGAAAGUG |
| | | | | GCUGUGGCCUCUCAGACGAGCUGCGGUC |
| | | | | GACCAGGAACAGCUACCAUUCGCAGCAC |
| | | | | UCUGCCCGUGUCCUACGAGCAGACGGAG |
| | | | | UACAUCUGCAGGCUGGCCGGCUAUCCCG |
| | | | | AUGGGAUUCCAGUCCUGGAGCACCACU |
| | | | | GAUAA |
| 215 | HSV-1 gB | HSV-2 gC | Version 3 | AUGCACCAGGGAGCACCUUCUUGGGGA |
| | -AP | (28-426) | | AGAAGAUGGUUUGUGGUGUGGGCUCUG |
| | | | | CUGGGACUGACCCUGGGAGUGCUGGUG |
| | | | | GCUUCUGCUGCUCCUUCUCCUGGAAGAA |
| | | | | CCAUCACAGUGGGACCAAGAGGAAAUG |
| | | | | CAAGCAAUGCAGCACCUUCUGCUUCUCC |
| | | | | AAGAAAUGCUUCUGCUCCAAGAACCACC |
| | | | | CCAACCCCUCCUCAGCCAAGAAAAGCAA |
| | | | | CCAAAUCCAAAGCAUCCACAGCAAAACC |
| | | | | UGCACCUCCUCCAAAAACAGGACCUCCA |
| | | | | AAAACCUCCUCUGAACCUGUGAGAUGC |
| | | | | AACAGACAUGAUCCUCUGGCAAGAUAU |
| | | | | GGAUCAAGAGUGCAGAUCAGAUGCAGA |
| | | | | UUUCCAAAUUCCACCAGAACAGAAUUC |
| | | | | AGACUCCAGAUCUGGAGAUAUGCAACA |
| | | | | GCAACAGAUGCAGAAAUUGGAACAGCA |
| | | | | CCAUCUCUGGAAGAAGUGAUGGUGAAU |
| | | | | GUGUCUGCUCCUCCUGGAGGACAGCUG |
| | | | | GUGUAUGAUUCUGCUCCAAACAGAACA |
| | | | | GAUCCUCAUGUGAUCUGGGCUGAAGGA |
| | | | | GCUGGACCUGGAGCUUCUCCAAGACUG |
| | | | | UACUCUGUGGUGGGACCUCUGGGAAGA |
| | | | | CAGAGACUGAUCAUUGAAGAACUGACC |
| | | | | CUGGAAACCCAGGGAAUGUACUACUGG |
| | | | | GUGUGGGGAAGAACAGACAGACCUUCU |
| | | | | GCUUAUGGAACCUGGGUGAGAGUGAGA |
| | | | | GUGUUCAGACCUCCUUCUCUGACCAUCC |
| | | | | ACCCUCAUGCUGUGCUGGAAGGACAGCC |
| | | | | UUUCAAAGCAACCUGCACAGCAGCAACC |
| | | | | UACUACCCUGGAAACAGAGCUGAAUUU |
| | | | | GUGUGGUUUGAAGAUGGAAGAAGGGUG |
| | | | | UUUGAUCCUGCUCAGAUCCACACCCAGA |
| | | | | CCCAGGAAAAUCCUGAUGGAUUUUCCA |
| | | | | CAGUGUCCACAGUGACAUCUGCUGCUG |
| | | | | UGGGAGGACAGGGACCUCCAAGAACCU |
| | | | | UCACCUGCCAGCUGACCUGGCACAGAGA |
| | | | | UUCUGUGUCUUUUUCAAGAAGAAAUGC |
| | | | | UUCUGGAACAGCUUCUGUGCUGCCAAG |
| | | | | ACCAACCAUCACCAUGGAAUUCACAGGA |
| | | | | GAUCAUGCUGUGUGCACAGCUGGAUGU |
| | | | | GUGCCUGAAGGAGUGACCUUUGCUUGG |
| | | | | UUUCUGGGAGAUGAUUCUUCUCCAGCU |
| | | | | GAAAAAGUGGCUGUGGCUUCCCAGACC |
| | | | | UCUUGUGGAAGACCUGGAACAGCAACC |
| | | | | AUCAGAUCCACCCUGCCUGUGUCUUAUG |
| | | | | AACAGACAGAAUACAUUUGCAGACUGG |
| | | | | CUGGAUACCCUGAUGGAAUCCCUGUGC |
| | | | | UGGAACACCACUGAUAA |
| 216 | HSV-1 gB- | HSV-2 gC | Version 4 | AUGCAUCAAGGCGCACCCAGUUGGGGC |
| | AP | (28-426) | | AGACGGUGGUUCGUAGUGUGGGCCUUG |
| | | | | CUGGGACUGACUCUCGGUGUCCUCGUU |
| | | | | GCGUCUGCUGCACCGAGUCCAGGAAGG |
| | | | | ACGAUUACGGUGGGACCCAGAGGUAAU |
| | | | | GCGUCCAAUGCUGCGCCAUCCGCUUCUC |
| | | | | CACGGAACGCAUCUGCACCCAGGACUAC |
| | | | | ACCGACACCACCUCAGCCGCGCAAAGCC |
| | | | | ACCAAGAGCAAGGCCAGCACAGCCAAAC |
| | | | | CCGCUCCUCCACCUAAAACCGGACCACC |
| | | | | UAAGACCAGCUCUGAACCCGUCAGAUGC |
| | | | | AACAGGCACGAUCCGUUGGCCAGAUAU |
| | | | | GGCAGUCGCGUCCAGAUCAGGUGUCGC |
| | | | | UUCCCUAACAGCACACGGACCGAGUUCA |
| | | | | GGCUGCAAAUUUGGCGCUACGCUACAG |
| | | | | CCACUGACGCAGAGAUUGGCACUGCUCC |
| | | | | CAGUCUGGAGGAGGUCAUGGUGAACGU |
| | | | | GUCUGCUCCACCAGGCGGUCAGCUGGUC |
| | | | | UAUGACUCAGCCCCUAAUCGCACAGAUC |
| | | | | CUCACGUGAUUUGGGCAGAAGGUGCGG |
| | | | | GGCCUGGGGCCUCCCCAAGGCUCUACUC |
| | | | | AGUGGUUGGACCCCUUGGGAGACAGCG |
| | | | | GCUGAUCAUCGAGGAACUGACUCUCGA |
| | | | | AACCCAAGGUAUGUACUACUGGGUAUG |
| | | | | GGGCAGAACAGACAGACCUUCAGCUUA |
| | | | | UGGCACCUGGGUGCGGGUGAGAGUGUU |
| | | | | UAGGCCUCCCUCCCUGACGAUCCAUCCC |
| | | | | CAUGCUGUGCUGGAAGGACAGCCGUUC |
| | | | | AAGGCAACAUGCACAGCAGCCACUUACU |
| | | | | AUCCCGGAAACCGUGCUGAGUUUGUGU |
| | | | | GGUUCGAGGAUGGGCGACGUGUAUUCG |
| | | | | ACCCUGCCCAGAUUCACACCCAGACACA |
| | | | | GGAGAAUCCCGACGGGUUUUCCACUGU |
| | | | | GAGCACCGUGACAUCAGCGGCAGUAGG |
| | | | | AGGGCAGGGCCCACCCCGAACGUUCACU |
| | | | | UGCCAGCUUACUUGGCAUCGGGACAGU |
| | | | | GUUAGCUUUAGCCGCCGGAAUGCCUCU |
| | | | | GGCACCGCAUCCGUCCUUCCUCGCCCAA |
| | | | | CCAUCACCAUGGAAUUCACUGGCGAUCA |
| | | | | CGCCGUUUGUACAGCCGGGUGUGUUCCC |
| | | | | GAGGGAGUGACCUUUGCUUGGUUUCUG |
| | | | | GGCGAUGACUCAAGCCCAGCCGAAAAG |
| | | | | GUGGCCGUCGCCUCCCAAACGAGCUGUG |
| | | | | GGCGACCUGGCACCGCUACCAUACGUAG |
| | | | | CACUCUGCCCGUUUCCUACGAACAGACC |
| | | | | GAGUAUAUCUGCCGAUUGGCCGGUUAC |
| | | | | CCCGAUGGGAUACCAGUCCUGGAGCACC |
| | | | | ACUGA |
| 217 | HSV-2 gI- | HSV-2 gC | Version 1 | AUGCCUGGCAGAUCUCUGCAAGGACUG |
| | L | (28-426) | | GCCAUCCUCGGACUGUGGGUUUGCGCA |
| | | | | ACAGGACUGAGCCCUGGCAGAACCAUCA |
| | | | | CAGUGGGCCCUAGAGGCAACGCCUCUAA |
| | | | | UGCCGCUCCUAGCGCCUCUCCUAGAAAC |
| | | | | GCCUCUGCUCCCAGAACCACACCUACAC |
| | | | | CUCCACAGCCUAGAAAGGCCACCAAGAG |
| | | | | CAAGGCCAGCACAGCCAAACCUGCUCCU |
| | | | | CCACCUAAGACAGGCCCUCCAAAGACAA |
| | | | | GCUCUGAGCCCGUGCGGUGCAACAGACA |
| | | | | CGAUCCACUGGCCAGAUACGGCAGCCGG |
| | | | | GUGCAGAUCAGAUGCAGAUUCCCCAAC |
| | | | | AGCACCCGGACCGAGUUCCGGCUCCAGA |
| | | | | UUUGGAGAUACGCCACCGCCACAGAUGC |
| | | | | CGAGAUUGGAACAGCCCCUAGCCUGGA |
| | | | | AGAAGUGAUGGUCAACGUUUCAGCCCC |
| | | | | UCCUGGCGGCCAGCUGGUGUAUGAUUC |
| | | | | UGCCCCUAACCGGACCGAUCCUCACGUG |
| | | | | AUAUGGGCUGAAGGUGCUGGCCCUGGC |
| | | | | GCUUCCCCUAGACUGUAUUCUGUUGUG |
| | | | | GGCCCUCUGGGCAGACAGCGGCUGAUCA |
| | | | | UUGAGGAACUGACCCUGGAAACCCAGG |
| | | | | GCAUGUACUACUGGGUCUGGGGCAGAA |
| | | | | CCGAUAGACCAAGCGCCUAUGGCACCUG |
| | | | | GGUUCGAGUGCGAGUGUUCAGACCUCC |
| | | | | UAGCCUGACCAUCCAUCCUCACGCCGUU |
| | | | | CUGGAAGGCCAGCCUUUCAAGGCCACAU |
| | | | | GUACCGCCGCCACCUACUAUCCCGGAAA |
| | | | | CAGAGCCGAGUUCGUUUGGUUCGAGGA |
| | | | | CGGCAGAAGGGUGUUCGACCCCGCUCAG |
| | | | | AUCCACACACAGACCCAAGAGAACCCCG |
| | | | | ACGGCUUUAGCACCGUGUCCACAGUGAC |
| | | | | AUCUGCCGCCGUUGGAGGACAGGGCCCU |
| | | | | CCUAGAACCUUUACCUGCCAGCUGACCU |
| | | | | GGCACAGAGACAGCGUGUCCUUCAGCA |
| | | | | GAAGAAACGCCAGCGGCACAGCCAGCGU |
| | | | | UCUGCCUAGACCUACCAUCACCAUGGAA |
| | | | | UUCACCGGCGACCACGCCGUGUGUACAG |
| | | | | CUGGAUGUGUUCCUGAGGGCGUGACCU |
| | | | | UCGCUUGGUUUCUGGGCGACGAUAGCA |
| | | | | GCCCUGCCGAAAAAGUGGCUGUGGCCA |
| | | | | GCCAGACAAGCUGUGGCAGACCUGGAA |
| | | | | CCGCCACCAUCAGAAGCACACUGCCUGU |
| | | | | CAGCUACGAGCAGACCGAGUACAUCUG |
| | | | | UCGGCUGGCCGGCUAUCCUGAUGGCAUC |
| | | | | CCUGUGCUGGAACACCACUGAUAA |
| 218 | HSV-2 gI- | HSV-2 gC | Version 2 | AUGCCCGGCAGAAGCCUCCAGGGACUGG |
| | L | (28-426) | | CUAUCCUGGGGCUGUGGGUGUGCGCCA |
| | | | | CCGGUCUUAGCCCCGGCAGAACCAUAAC |
| | | | | AGUAGGGCCACGGGGGAAUGCUUCCAA |
| | | | | UGCUGCACCUUCCGCUUCACCGAGGAAU |
| | | | | GCUUCUGCCCCAAGAACUACCCCCACUC |
| | | | | CUCCUCAACCCAGGAAAGCGACAAAGUC |
| | | | | CAAGGCCAGCACCGCAAAACCCGCUCCU |
| | | | | CCUCCAAAGACUGGGCCCCCAAAGACAA |
| | | | | GUAGCGAACCAGUUCGGUGCAACAGGC |
| | | | | AUGACCCACUUGCACGCUAUGGGUCAA |
| | | | | GAGUCCAGAUACGGUGUCGCUUCCCUA |
| | | | | ACAGUACAAGGACUGAGUUUCGGCUGC |
| | | | | AGAUCUGGCGUUAUGCCACAGCUACUG |
| | | | | ACGCAGAGAUUGGUACCGCCCCCAGUUU |
| | | | | GGAAGAGGUGAUGGUCAACGUGUCCGC |
| | | | | ACCCCCAGGAGGACAGCUGGUCUAUGAC |
| | | | | UCAGCGCCCAAUAGGACCGAUCCCCACG |
| | | | | UGAUCUGGGCAGAAGGAGCCGGUCCUG |
| | | | | GGGCCUCUCCACGGCUGUACUCAGUUGU |
| | | | | UGGCCCGCUUGGACGACAGAGACUCAUC |
| | | | | AUCGAAGAGCUGACACUGGAGACACAG |
| | | | | GGGAUGUACUACUGGGUGUGGGGCCGU |
| | | | | ACUGACCGCCCUUCCGCAUAUGGCACUU |
| | | | | GGGUGAGAGUUCGCGUCUUUCGGCCCCC |
| | | | | UUCUCUCACCAUCCAUCCUCAUGCCGUG |
| | | | | CUCGAAGGCCAGCCCUUUAAGGCCACAU |
| | | | | GCACUGCUGCGACCUACUACCCUGGCAA |
| | | | | CAGAGCCGAGUUUGUCUGGUUUGAGGA |
| | | | | UGGUCGGCGAGUAUUCGAUCCAGCCCA |
| | | | | GAUUCACACACAAACGCAGGAAAAUCC |
| | | | | GGACGGCUUCAGCACAGUGUCCACGGU |
| | | | | GACCUCUGCUGCAGUUGGUGGACAAGG |
| | | | | ACCCCCUCGAACCUUCACCUGUCAGCUG |
| | | | | ACCUGGCACAGAGACUCCGUAAGCUUCA |
| | | | | GCCGUAGAAACGCCUCUGGAACCGCCAG |
| | | | | UGUGUUGCCGAGGCCGACUAUCACGAU |
| | | | | GGAAUUCACAGGCGAUCAUGCCGUCUG |
| | | | | UACUGCCGGCUGUGUGCCAGAAGGCGU |
| | | | | AACCUUCGCUUGGUUUCUCGGGGAUGA |
| | | | | CUCAAGUCCUGCAGAGAAAGUGGCUGU |
| | | | | GGCCUCUCAGACGAGCUGCGGUCGACCA |
| | | | | GGAACAGCUACCAUUCGCAGCACUCUGC |
| | | | | CCGUGUCCUACGAGCAGACGGAGUACA |
| | | | | UCUGCAGGCUGGCCGGCUAUCCCGAUGG |
| | | | | GAUUCCAGUCCUGGAGCACCACUGAUA |
| | | | | A |
| 219 | HSV-2 gI- | HSV-2 gC | Version 3 | AUGCCUGGAAGAUCUCUCCAGGGACUG |
| | L | (28-426) | | GCAAUCCUGGGACUGUGGGUGUGUGCA |
| | | | | ACAGGACUGUCUCCUGGAAGAACCAUC |
| | | | | ACAGUGGGACCAAGAGGAAAUGCAAGC |
| | | | | AAUGCAGCACCUUCUGCUUCUCCAAGAA |
| | | | | AUGCUUCUGCUCCAAGAACCACCCCAAC |
| | | | | CCCUCCUCAGCCAAGAAAAGCAACCAAA |
| | | | | UCCAAAGCAUCCACAGCAAAACCUGCAC |
| | | | | CUCCUCCAAAAACAGGACCUCCAAAAAC |
| | | | | CUCCUCUGAACCUGUGAGAUGCAACAG |
| | | | | ACAUGAUCCUCUGGCAAGAUAUGGAUC |
| | | | | AAGAGUGCAGAUCAGAUGCAGAUUUCC |
| | | | | AAAUUCCACCAGAACAGAAUUCAGACU |
| | | | | CCAGAUCUGGAGAUAUGCAACAGCAAC |
| | | | | AGAUGCAGAAAUUGGAACAGCACCAUC |
| | | | | UCUGGAAGAAGUGAUGGUGAAUGUGUC |
| | | | | UGCUCCUCCUGGAGGACAGCUGGUGUA |
| | | | | UGAUUCUGCUCCAAACAGAACAGAUCC |
| | | | | UCAUGUGAUCUGGGCUGAAGGAGCUGG |
| | | | | ACCUGGAGCUUCUCCAAGACUGUACUCU |
| | | | | GUGGUGGGACCUCUGGGAAGACAGAGA |
| | | | | CUGAUCAUUGAAGAACUGACCCUGGAA |
| | | | | ACCCAGGGAAUGUACUACUGGGUGUGG |
| | | | | GGAAGAACAGACAGACCUUCUGCUUAU |
| | | | | GGAACCUGGGUGAGAGUGAGAGUGUUC |
| | | | | AGACCUCCUUCUCUGACCAUCCACCCUC |
| | | | | AUGCUGUGCUGGAAGGACAGCCUUUCA |
| | | | | AAGCAACCUGCACAGCAGCAACCUACUA |
| | | | | CCCUGGAAACAGAGCUGAAUUUGUGUG |
| | | | | GUUUGAAGAUGGAAGAAGGGUGUUUGA |
| | | | | UCCUGCUCAGAUCCACACCCAGACCCAG |
| | | | | GAAAAUCCUGAUGGAUUUUCCACAGUG |
| | | | | UCCACAGUGACAUCUGCUGCUGUGGGA |
| | | | | GGACAGGGACCUCCAAGAACCUUCACCU |
| | | | | GCCAGCUGACCUGGCACAGAGAUUCUG |
| | | | | UGUCUUUUUCAAGAAGAAAUGCUUCUG |
| | | | | GAACAGCUUCUGUGCUGCCAAGACCAAC |
| | | | | CAUCACCAUGGAAUUCACAGGAGAUCA |
| | | | | UGCUGUGUGCACAGCUGGAUGUGUGCC |
| | | | | UGAAGGAGUGACCUUUGCUUGGUUUCU |
| | | | | GGGAGAUGAUUCUUCUCCAGCUGAAAA |
| | | | | AGUGGCUGUGGCUUCCCAGACCUCUUG |
| | | | | UGGAAGACCUGGAACAGCAACCAUCAG |
| | | | | AUCCACCCUGCCUGUGUCUUAUGAACAG |
| | | | | ACAGAAUACAUUUGCAGACUGGCUGGA |
| | | | | UACCCUGAUGGAAUCCCUGUGCUGGAA |
| | | | | CACCACUGAUAA |
| 220 | HSV-2 gI- | HSV-2 gC | Version 4 | AUGCCUGGGAGAAGCCUGCAAGGGCUC |
| | L | (28-426) | | GCAAUCUUGGGCCUGUGGGUUUGUGCC |
| | | | | ACAGGCUUGUCCCCGGGUCGAACAAUCA |
| | | | | CUGUUGGGCCCAGGGGAAAUGCCAGCA |
| | | | | AUGCUGCACCUUCAGCAAGCCCACGAAA |
| | | | | CGCUUCAGCACCCAGGACAACACCCACU |
| | | | | CCACCUCAACCGCGGAAAGCCACCAAGA |
| | | | | GCAAGGCAAGUACCGCCAAACCCGCUCC |
| | | | | UCCUCCCAAGACAGGGCCACCCAAGACC |
| | | | | UCUAGUGAGCCAGUGAGGUGUAACCGC |
| | | | | CAUGAUCCCCUUGCCAGAUACGGGAGCA |
| | | | | GAGUGCAGAUUAGGUGCCGGUUUCCAA |
| | | | | ACUCCACGAGAACCGAAUUUCGCCUCCA |
| | | | | GAUUUGGCGGUAUGCGACUGCCACAGA |
| | | | | CGCAGAGAUUGGUACCGCUCCCAGCCUG |
| | | | | GAGGAGGUCAUGGUGAACGUGUCAGCG |
| | | | | CCUCCGGGUGGCCAGCUGGUCUACGACU |
| | | | | CUGCCCCAAAUCGAACCGACCCUCACGU |
| | | | | CAUCUGGGCUGAAGGAGCGGGACCAGG |
| | | | | AGCCUCUCCACGCUUGUAUAGCGUAGU |
| | | | | UGGCCCUCUGGGGAGACAGCGCCUGAUC |
| | | | | AUUGAGGAACUGACCCUUGAGACACAG |
| | | | | GGGAUGUACUACUGGGUGUGGGGCAGG |
| | | | | ACUGACAGGCCCAGUGCCUAUGGAACU |
| | | | | UGGGUUAGGGUCCGCGUCUUUCGGCCA |
| | | | | CCCAGUCUGACCAUCCAUCCACAUGCCG |
| | | | | UGCUGGAAGGCCAGCCCUUCAAAGCGAC |
| | | | | UUGCACUGCCGCCACGUACUAUCCAGGG |
| | | | | AAUAGAGCCGAGUUCGUUUGGUUCGAG |
| | | | | GAUGGCCGGAGAGUAUUCGAUCCAGCU |
| | | | | CAGAUCCACACCCAGACGCAGGAAAACC |
| | | | | CGGACGGCUUUAGCACGGUGAGUACCG |
| | | | | UCACCUCUGCUGCCGUCGGAGGCCAAGG |
| | | | | ACCUCCCCGUACCUUCACAUGCCAGCUU |
| | | | | ACAUGGCACCGGGACUCAGUAAGCUUU |
| | | | | UCACGUCGUAAUGCAUCCGGUACUGCU |
| | | | | UCUGUGCUGCCUCGACCCACCAUCACCA |
| | | | | UGGAGUUCACAGGGGAUCACGCAGUGU |
| | | | | GUACGGCAGGCUGCGUGCCUGAAGGCG |
| | | | | UGACAUUCGCCUGGUUUCUCGGUGAUG |
| | | | | ACUCCUCUCCUGCUGAAAAGGUGGCUG |
| | | | | UAGCCUCCCAAACAAGCUGUGGUCGGCC |
| | | | | UGGAACUGCCACUAUACGCUCCACUCUC |
| | | | | CCGGUGUCCUACGAACAGACCGAGUACA |
| | | | | UAUGCAGACUGGCUGGAUAUCCCGAUG |
| | | | | GCAUUCCCGUGCUGGAGCAUCACUGA |
| 221 | HSV-2 gE- | HSV-2 gC | Version 1 | AUGGCUAGAGGUGCCGGCCUGGUGUUC |
| | RTS | (28-426) | | UUUGUUGGCGUGUGGGUCGUGUCCUGU |
| | | | | CUGGCCGCUGCUCCUAGAACAUCUAGCC |
| | | | | CUGGCAGAACCAUCACAGUGGGCCCUAG |
| | | | | AGGCAACGCCUCUAAUGCCGCUCCUAGC |
| | | | | GCCUCUCCUAGAAACGCCUCUGCUCCCA |
| | | | | GAACCACACCUACACCUCCACAGCCUAG |
| | | | | AAAGGCCACCAAGAGCAAGGCCAGCACA |
| | | | | GCCAAACCUGCUCCUCCACCUAAGACAG |
| | | | | GCCCUCCAAAGACAAGCUCUGAGCCCGU |
| | | | | GCGGUGCAACAGACACGAUCCACUGGCC |
| | | | | AGAUACGGCAGCCGGGUGCAGAUCAGA |
| | | | | UGCAGAUUCCCCAACAGCACCCGGACCG |
| | | | | AGUUCCGGCUCCAGAUUUGGAGAUACG |
| | | | | CCACCGCCACAGAUGCCGAGAUUGGAAC |
| | | | | AGCCCCUAGCCUGGAAGAAGUGAUGGU |
| | | | | CAACGUUUCAGCCCCUCCUGGCGGCCAG |
| | | | | CUGGUGUAUGAUUCUGCCCCUAACCGG |
| | | | | ACCGAUCCUCACGUGAUAUGGGCUGAA |
| | | | | GGUGCUGGCCCUGGCGCUUCCCCUAGAC |
| | | | | UGUAUUCUGUUGUGGGCCCUCUGGGCA |
| | | | | GACAGCGGCUGAUCAUUGAGGAACUGA |
| | | | | CCCUGGAAACCCAGGGCAUGUACUACUG |
| | | | | GGUCUGGGGCAGAACCGAUAGACCAAG |
| | | | | CGCCUAUGGCACCUGGGUUCGAGUGCG |
| | | | | AGUGUUCAGACCUCCUAGCCUGACCAUC |
| | | | | CAUCCUCACGCCGUUCUGGAAGGCCAGC |
| | | | | CUUUCAAGGCCACAUGUACCGCCGCCAC |
| | | | | CUACUAUCCCGGAAACAGAGCCGAGUUC |
| | | | | GUUUGGUUCGAGGACGGCAGAAGGGUG |
| | | | | UUCGACCCCGCUCAGAUCCACACACAGA |
| | | | | CCCAAGAGAACCCCGACGGCUUUAGCAC |
| | | | | CGUGUCCACAGUGACAUCUGCCGCCGUU |
| | | | | GGAGGACAGGGCCCUCCUAGAACCUUU |
| | | | | ACCUGCCAGCUGACCUGGCACAGAGACA |
| | | | | GCGUGUCCUUCAGCAGAAGAAACGCCA |
| | | | | GCGGCACAGCCAGCGUUCUGCCUAGACC |
| | | | | UACCAUCACCAUGGAAUUCACCGGCGAC |
| | | | | CACGCCGUGUGUACAGCUGGAUGUGUU |
| | | | | CCUGAGGGCGUGACCUUCGCUUGGUUU |
| | | | | CUGGGCGACGAUAGCAGCCCUGCCGAAA |
| | | | | AAGUGGCUGUGGCCAGCCAGACAAGCU |
| | | | | GUGGCAGACCUGGAACCGCCACCAUCAG |
| | | | | AAGCACACUGCCUGUCAGCUACGAGCAG |
| | | | | ACCGAGUACAUCUGUCGGCUGGCCGGCU |
| | | | | AUCCUGAUGGCAUCCCUGUGCUGGAAC |
| | | | | ACCACUGAUAA |
| 222 | HSV-2 gE- | HSV-2 gC | Version 2 | AUGGCGAGAGGAGCCGGGCUCGUGUUC |
| | RTS | (28-426) | | UUUGUGGGCGUAUGGGUCGUUUCCUGC |
| | | | | CUGGCUGCCGCACCCAGGACCAGCAGCC |
| | | | | CCGGCAGAACCAUAACAGUAGGGCCACG |
| | | | | GGGGAAUGCUUCCAAUGCUGCACCUUCC |
| | | | | GCUUCACCGAGGAAUGCUUCUGCCCCAA |
| | | | | GAACUACCCCCACUCCUCCUCAACCCAG |
| | | | | GAAAGCGACAAAGUCCAAGGCCAGCACC |
| | | | | GCAAAACCCGCUCCUCCUCCAAAGACUG |
| | | | | GGCCCCCAAAGACAAGUAGCGAACCAGU |
| | | | | UCGGUGCAACAGGCAUGACCCACUUGCA |
| | | | | CGCUAUGGGUCAAGAGUCCAGAUACGG |
| | | | | UGUCGCUUCCCUAACAGUACAAGGACU |
| | | | | GAGUUUCGGCUGCAGAUCUGGCGUUAU |
| | | | | GCCACAGCUACUGACGCAGAGAUUGGU |
| | | | | ACCGCCCCCAGUUUGGAAGAGGUGAUG |
| | | | | GUCAACGUGUCCGCACCCCCAGGAGGAC |
| | | | | AGCUGGUCUAUGACUCAGCGCCCAAUA |
| | | | | GGACCGAUCCCCACGUGAUCUGGGCAGA |
| | | | | AGGAGCCGGUCCUGGGGCCUCUCCACGG |
| | | | | CUGUACUCAGUUGUUGGCCCGCUUGGA |
| | | | | CGACAGAGACUCAUCAUCGAAGAGCUG |
| | | | | ACACUGGAGACACAGGGGAUGUACUAC |
| | | | | UGGGUGUGGGGCCGUACUGACCGCCCU |
| | | | | UCCGCAUAUGGCACUUGGGUGAGAGUU |
| | | | | CGCGUCUUUCGGCCCCCUUCUCUCACCA |
| | | | | UCCAUCCUCAUGCCGUGCUCGAAGGCCA |
| | | | | GCCCUUUAAGGCCACAUGCACUGCUGCG |
| | | | | ACCUACUACCCUGGCAACAGAGCCGAGU |
| | | | | UUGUCUGGUUUGAGGAUGGUCGGCGAG |
| | | | | UAUUCGAUCCAGCCCAGAUUCACACACA |
| | | | | AACGCAGGAAAAUCCGGACGGCUUCAG |
| | | | | CACAGUGUCCACGGUGACCUCUGCUGCA |
| | | | | GUUGGUGGACAAGGACCCCCUCGAACCU |
| | | | | UCACCUGUCAGCUGACCUGGCACAGAGA |
| | | | | CUCCGUAAGCUUCAGCCGUAGAAACGCC |
| | | | | UCUGGAACCGCCAGUGUGUUGCCGAGG |
| | | | | CCGACUAUCACGAUGGAAUUCACAGGC |
| | | | | GAUCAUGCCGUCUGUACUGCCGGCUGU |
| | | | | GUGCCAGAAGGCGUAACCUUCGCUUGG |
| | | | | UUUCUCGGGGAUGACUCAAGUCCUGCA |
| | | | | GAGAAAGUGGCUGUGGCCUCUCAGACG |
| | | | | AGCUGCGGUCGACCAGGAACAGCUACCA |
| | | | | UUCGCAGCACUCUGCCCGUGUCCUACGA |
| | | | | GCAGACGGAGUACAUCUGCAGGCUGGC |
| | | | | CGGCUAUCCCGAUGGGAUUCCAGUCCUG |
| | | | | GAGCACCACUGAUAA |
| 223 | HSV-2 gE- | HSV-2 gC | Version 3 | AUGGCAAGAGGAGCAGGACUGGUGUUU |
| | RTS | (28-426) | | UUCGUGGGAGUUUGGGUGGUGUCUUGC |
| | | | | CUGGCUGCUGCUCCAAGAACCUCUUCUC |
| | | | | CUGGAAGAACCAUCACAGUGGGACCAA |
| | | | | GAGGAAAUGCAAGCAAUGCAGCACCUU |
| | | | | CUGCUUCUCCAAGAAAUGCUUCUGCUCC |
| | | | | AAGAACCACCCCAACCCCUCCUCAGCCA |
| | | | | AGAAAAGCAACCAAAUCCAAAGCAUCC |
| | | | | ACAGCAAAACCUGCACCUCCUCCAAAAA |
| | | | | CAGGACCUCCAAAAACCUCCUCUGAACC |
| | | | | UGUGAGAUGCAACAGACAUGAUCCUCU |
| | | | | GGCAAGAUAUGGAUCAAGAGUGCAGAU |
| | | | | CAGAUGCAGAUUUCCAAAUUCCACCAG |
| | | | | AACAGAAUUCAGACUCCAGAUCUGGAG |
| | | | | AUAUGCAACAGCAACAGAUGCAGAAAU |
| | | | | UGGAACAGCACCAUCUCUGGAAGAAGU |
| | | | | GAUGGUGAAUGUGUCUGCUCCUCCUGG |
| | | | | AGGACAGCUGGUGUAUGAUUCUGCUCC |
| | | | | AAACAGAACAGAUCCUCAUGUGAUCUG |
| | | | | GGCUGAAGGAGCUGGACCUGGAGCUUC |
| | | | | UCCAAGACUGUACUCUGUGGUGGGACC |
| | | | | UCUGGGAAGACAGAGACUGAUCAUUGA |
| | | | | AGAACUGACCCUGGAAACCCAGGGAAU |
| | | | | GUACUACUGGGUGUGGGGAAGAACAGA |
| | | | | CAGACCUUCUGCUUAUGGAACCUGGGU |
| | | | | GAGAGUGAGAGUGUUCAGACCUCCUUC |
| | | | | UCUGACCAUCCACCCUCAUGCUGUGCUG |
| | | | | GAAGGACAGCCUUUCAAAGCAACCUGC |
| | | | | ACAGCAGCAACCUACUACCCUGGAAACA |
| | | | | GAGCUGAAUUUGUGUGGUUUGAAGAUG |
| | | | | GAAGAAGGGUGUUUGAUCCUGCUCAGA |
| | | | | UCCACACCCAGACCCAGGAAAAUCCUGA |
| | | | | UGGAUUUUCCACAGUGUCCACAGUGAC |
| | | | | AUCUGCUGCUGUGGGAGGACAGGGACC |
| | | | | UCCAAGAACCUUCACCUGCCAGCUGACC |
| | | | | UGGCACAGAGAUUCUGUGUCUUUUUCA |
| | | | | AGAAGAAAUGCUUCUGGAACAGCUUCU |
| | | | | GUGCUGCCAAGACCAACCAUCACCAUGG |
| | | | | AAUUCACAGGAGAUCAUGCUGUGUGCA |
| | | | | CAGCUGGAUGUGUGCCUGAAGGAGUGA |
| | | | | CCUUUGCUUGGUUUCUGGGAGAUGAUU |
| | | | | CUUCUCCAGCUGAAAAAGUGGCUGUGG |
| | | | | CUUCCCAGACCUCUUGUGGAAGACCUGG |
| | | | | AACAGCAACCAUCAGAUCCACCCUGCCU |
| | | | | GUGUCUUAUGAACAGACAGAAUACAUU |
| | | | | UGCAGACUGGCUGGAUACCCUGAUGGA |
| | | | | AUCCCUGUGCUGGAACACCACUGAUAA |
| 224 | HSV-2 gE- | HSV-2 gC | Version 4 | AUGGCGAGAGGAGCCGGACUCGUGUUC |
| | RTS | (28-426) | | UUUGUGGGAGUCUGGGUUGUGAGCUGC |
| | | | | CUGGCAGCAGCUCCACGCACUAGCUCUC |
| | | | | CAGGCAGAACUAUCACAGUGGGACCCA |
| | | | | GAGGGAAUGCCAGCAAUGCAGCCCCGA |
| | | | | GUGCCAGCCCUCGUAACGCCAGCGCUCC |
| | | | | CAGAACAACCCCAACUCCACCGCAGCCU |
| | | | | AGAAAGGCGACCAAGUCCAAAGCAUCC |
| | | | | ACUGCAAAACCAGCCCCACCUCCCAAAA |
| | | | | CGGGACCUCCCAAGACCAGCUCCGAGCC |
| | | | | UGUAAGGUGCAAUCGGCAUGACCCCUU |
| | | | | GGCCCGAUAUGGCAGUCGCGUGCAGAU |
| | | | | UCGAUGUCGGUUUCCCAACUCUACCCGG |
| | | | | ACUGAGUUCCGGUUGCAGAUCUGGAGG |
| | | | | UAUGCGACCGCCACUGACGCUGAGAUCG |
| | | | | GCACAGCACCAAGCCUGGAAGAAGUGA |
| | | | | UGGUGAACGUUAGUGCUCCUCCGGGCG |
| | | | | GGCAACUCGUGUAUGACUCCGCACCCAA |
| | | | | CCGCACAGAUCCUCACGUGAUUUGGGCC |
| | | | | GAAGGAGCCGGACCCGGUGCGUCACCUA |
| | | | | GGCUCUACUCUGUCGUAGGACCACUGG |
| | | | | GCCGUCAACGCCUGAUAAUCGAGGAGC |
| | | | | UGACUCUGGAGACACAGGGUAUGUACU |
| | | | | ACUGGGUCUGGGGCAGAACCGACAGGC |
| | | | | CAUCUGCUUACGGGACAUGGGUCCGCG |
| | | | | UUCGAGUAUUUCGGCCACCCUCACUGAC |
| | | | | CAUACAUCCCCAUGCCGUUCUUGAAGGG |
| | | | | CAGCCUUUCAAGGCAACCUGUACUGCUG |
| | | | | CCACAUACUAUCCCGGGAAUAGGGCCGA |
| | | | | GUUCGUCUGGUUUGAAGAUGGCCGAAG |
| | | | | GGUGUUUGACCCGGCUCAGAUCCACACC |
| | | | | CAGACACAGGAGAACCCCGAUGGCUUCA |
| | | | | GUACGGUGUCUACCGUCACAAGCGCCGC |
| | | | | UGUGGGUGGCCAAGGUCCUCCCAGAAC |
| | | | | UUUCACCUGUCAGCUGACGUGGCACAG |
| | | | | GGAUUCCGUGAGCUUUUCCCGCCGCAAU |
| | | | | GCGUCAGGGACCGCCUCCGUGCUUCCUC |
| | | | | GGCCAACCAUCACAAUGGAAUUCACGG |
| | | | | GUGAUCACGCUGUCUGCACAGCUGGCU |
| | | | | GCGUUCCUGAGGGCGUGACAUUCGCAU |
| | | | | GGUUUCUUGGUGACGACUCAUCUCCCGC |
| | | | | AGAGAAGGUGGCUGUUGCCUCACAAAC |
| | | | | GAGUUGUGGGCGUCCAGGCACUGCCACC |
| | | | | AUUCGGUCCACCCUCCCCGUAAGCUACG |
| | | | | AACAGACUGAGUAUAUUUGCAGACUGG |
| | | | | CUGGAUACCCGGAUGGGAUUCCUGUCC |
| | | | | UGGAACAUCACUGA |
| 225 | EboZ | HSV-2 gC | Version 1 | AUGGGAGUGACCGGCAUUCUCCAGCUG |
| | | (28-426) | | CCUCGGGACAGAUUCAAGCGGACCAGCU |
| | | | | UCUUCCUGUGGGUCAUCAUCCUGUUCCA |
| | | | | GCGGACCUUCAGCAUCCCCAGCCCUGGC |
| | | | | AGAACCAUCACAGUGGGCCCUAGAGGC |
| | | | | AACGCCUCUAAUGCCGCUCCUAGCGCCU |
| | | | | CUCCUAGAAACGCCUCUGCUCCCAGAAC |
| | | | | CACACCUACACCUCCACAGCCUAGAAAG |
| | | | | GCCACCAAGAGCAAGGCCAGCACAGCCA |
| | | | | AACCUGCUCCUCCACCUAAGACAGGCCC |
| | | | | UCCAAAGACAAGCUCUGAGCCCGUGCGG |
| | | | | UGCAACAGACACGAUCCACUGGCCAGAU |
| | | | | ACGGCAGCCGGGUGCAGAUCAGAUGCA |
| | | | | GAUUCCCCAACAGCACCCGGACCGAGUU |
| | | | | CCGGCUCCAGAUUUGGAGAUACGCCACC |
| | | | | GCCACAGAUGCCGAGAUUGGAACAGCCC |
| | | | | CUAGCCUGGAAGAAGUGAUGGUCAACG |
| | | | | UUUCAGCCCCUCCUGGCGGCCAGCUGGU |
| | | | | GUAUGAUUCUGCCCCUAACCGGACCGAU |
| | | | | CCUCACGUGAUAUGGGCUGAAGGUGCU |
| | | | | GGCCCUGGCGCUUCCCCUAGACUGUAUU |
| | | | | CUGUUGUGGGCCCUCUGGGCAGACAGC |
| | | | | GGCUGAUCAUUGAGGAACUGACCCUGG |
| | | | | AAACCCAGGGCAUGUACUACUGGGUCU |
| | | | | GGGGCAGAACCGAUAGACCAAGCGCCU |
| | | | | AUGGCACCUGGGUUCGAGUGCGAGUGU |
| | | | | UCAGACCUCCUAGCCUGACCAUCCAUCC |
| | | | | UCACGCCGUUCUGGAAGGCCAGCCUUUC |
| | | | | AAGGCCACAUGUACCGCCGCCACCUACU |
| | | | | AUCCCGGAAACAGAGCCGAGUUCGUUU |
| | | | | GGUUCGAGGACGGCAGAAGGGUGUUCG |
| | | | | ACCCCGCUCAGAUCCACACACAGACCCA |
| | | | | AGAGAACCCCGACGGCUUUAGCACCGUG |
| | | | | UCCACAGUGACAUCUGCCGCCGUUGGAG |
| | | | | GACAGGGCCCUCCUAGAACCUUUACCUG |
| | | | | CCAGCUGACCUGGCACAGAGACAGCGUG |
| | | | | UCCUUCAGCAGAAGAAACGCCAGCGGCA |
| | | | | CAGCCAGCGUUCUGCCUAGACCUACCAU |
| | | | | CACCAUGGAAUUCACCGGCGACCACGCC |
| | | | | GUGUGUACAGCUGGAUGUGUUCCUGAG |
| | | | | GGCGUGACCUUCGCUUGGUUUCUGGGC |
| | | | | GACGAUAGCAGCCCUGCCGAAAAAGUG |
| | | | | GCUGUGGCCAGCCAGACAAGCUGUGGC |
| | | | | AGACCUGGAACCGCCACCAUCAGAAGCA |
| | | | | CACUGCCUGUCAGCUACGAGCAGACCGA |
| | | | | GUACAUCUGUCGGCUGGCCGGCUAUCCU |
| | | | | GAUGGCAUCCCUGUGCUGGAACACCACU |
| | | | | GAUAA |
| 226 | EboZ | HSV-2 gC | Version 2 | AUGGGAGUGACUGGCAUACUCCAGCUU |
| | | (28-426) | | CCUAGAGACAGGUUUAAGCGCACAUCC |
| | | | | UUCUUUCUGUGGGUCAUCAUCCUGUUC |
| | | | | CAACGGACCUUCAGCAUUCCCAGCCCCG |
| | | | | GCAGAACCAUAACAGUAGGGCCACGGG |
| | | | | GGAAUGCUUCCAAUGCUGCACCUUCCGC |
| | | | | UUCACCGAGGAAUGCUUCUGCCCCAAGA |
| | | | | ACUACCCCCACUCCUCCUCAACCCAGGA |
| | | | | AAGCGACAAAGUCCAAGGCCAGCACCGC |
| | | | | AAAACCCGCUCCUCCUCCAAAGACUGGG |
| | | | | CCCCCAAAGACAAGUAGCGAACCAGUUC |
| | | | | GGUGCAACAGGCAUGACCCACUUGCACG |
| | | | | CUAUGGGUCAAGAGUCCAGAUACGGUG |
| | | | | UCGCUUCCCUAACAGUACAAGGACUGA |
| | | | | GUUUCGGCUGCAGAUCUGGCGUUAUGC |
| | | | | CACAGCUACUGACGCAGAGAUUGGUAC |
| | | | | CGCCCCCAGUUUGGAAGAGGUGAUGGU |
| | | | | CAACGUGUCCGCACCCCCAGGAGGACAG |
| | | | | CUGGUCUAUGACUCAGCGCCCAAUAGG |
| | | | | ACCGAUCCCCACGUGAUCUGGGCAGAAG |
| | | | | GAGCCGGUCCUGGGGCCUCUCCACGGCU |
| | | | | GUACUCAGUUGUUGGCCCGCUUGGACG |
| | | | | ACAGAGACUCAUCAUCGAAGAGCUGAC |
| | | | | ACUGGAGACACAGGGGAUGUACUACUG |
| | | | | GGUGUGGGGCCGUACUGACCGCCCUUCC |
| | | | | GCAUAUGGCACUUGGGUGAGAGUUCGC |
| | | | | GUCUUUCGGCCCCCUUCUCUCACCAUCC |
| | | | | AUCCUCAUGCCGUGCUCGAAGGCCAGCC |
| | | | | CUUUAAGGCCACAUGCACUGCUGCGACC |
| | | | | UACUACCCUGGCAACAGAGCCGAGUUU |
| | | | | GUCUGGUUUGAGGAUGGUCGGCGAGUA |
| | | | | UUCGAUCCAGCCCAGAUUCACACACAAA |
| | | | | CGCAGGAAAAUCCGGACGGCUUCAGCAC |
| | | | | AGUGUCCACGGUGACCUCUGCUGCAGU |
| | | | | UGGUGGACAAGGACCCCCUCGAACCUUC |
| | | | | ACCUGUCAGCUGACCUGGCACAGAGACU |
| | | | | CCGUAAGCUUCAGCCGUAGAAACGCCUC |
| | | | | UGGAACCGCCAGUGUGUUGCCGAGGCC |
| | | | | GACUAUCACGAUGGAAUUCACAGGCGA |
| | | | | UCAUGCCGUCUGUACUGCCGGCUGUGU |
| | | | | GCCAGAAGGCGUAACCUUCGCUUGGUU |
| | | | | UCUCGGGGAUGACUCAAGUCCUGCAGA |
| | | | | GAAAGUGGCUGUGGCCUCUCAGACGAG |
| | | | | CUGCGGUCGACCAGGAACAGCUACCAUU |
| | | | | CGCAGCACUCUGCCCGUGUCCUACGAGC |
| | | | | AGACGGAGUACAUCUGCAGGCUGGCCG |
| | | | | GCUAUCCCGAUGGGAUUCCAGUCCUGG |
| | | | | AGCACCACUGAUAA |
| 227 | EboZ | HSV-2 gC | Version 3 | AUGGGAGUAACCGGAAUUCUCCAGCUG |
| | | (28-426) | | CCAAGAGAUCGAUUCAAAAGAACAUCA |
| | | | | UUUUUCCUUUGGGUAAUUAUUCUGUUU |
| | | | | CAGAGAACAUUUUCCAUCCCUUCUCCUG |
| | | | | GAAGAACCAUCACAGUGGGACCAAGAG |
| | | | | GAAAUGCAAGCAAUGCAGCACCUUCUG |
| | | | | CUUCUCCAAGAAAUGCUUCUGCUCCAAG |
| | | | | AACCACCCCAACCCCUCCUCAGCCAAGA |
| | | | | AAAGCAACCAAAUCCAAAGCAUCCACAG |
| | | | | CAAAACCUGCACCUCCUCCAAAAACAGG |
| | | | | ACCUCCAAAAACCUCCUCUGAACCUGUG |
| | | | | AGAUGCAACAGACAUGAUCCUCUGGCA |
| | | | | AGAUAUGGAUCAAGAGUGCAGAUCAGA |
| | | | | UGCAGAUUUCCAAAUUCCACCAGAACA |
| | | | | GAAUUCAGACUCCAGAUCUGGAGAUAU |
| | | | | GCAACAGCAACAGAUGCAGAAAUUGGA |
| | | | | ACAGCACCAUCUCUGGAAGAAGUGAUG |
| | | | | GUGAAUGUGUCUGCUCCUCCUGGAGGA |
| | | | | CAGCUGGUGUAUGAUUCUGCUCCAAAC |
| | | | | AGAACAGAUCCUCAUGUGAUCUGGGCU |
| | | | | GAAGGAGCUGGACCUGGAGCUUCUCCA |
| | | | | AGACUGUACUCUGUGGUGGGACCUCUG |
| | | | | GGAAGACAGAGACUGAUCAUUGAAGAA |
| | | | | CUGACCCUGGAAACCCAGGGAAUGUAC |
| | | | | UACUGGGUGUGGGGAAGAACAGACAGA |
| | | | | CCUUCUGCUUAUGGAACCUGGGUGAGA |
| | | | | GUGAGAGUGUUCAGACCUCCUUCUCUG |
| | | | | ACCAUCCACCCUCAUGCUGUGCUGGAAG |
| | | | | GACAGCCUUUCAAAGCAACCUGCACAGC |
| | | | | AGCAACCUACUACCCUGGAAACAGAGCU |
| | | | | GAAUUUGUGUGGUUUGAAGAUGGAAGA |
| | | | | AGGGUGUUUGAUCCUGCUCAGAUCCAC |
| | | | | ACCCAGACCCAGGAAAAUCCUGAUGGA |
| | | | | UUUUCCACAGUGUCCACAGUGACAUCU |
| | | | | GCUGCUGUGGGAGGACAGGGACCUCCA |
| | | | | AGAACCUUCACCUGCCAGCUGACCUGGC |
| | | | | ACAGAGAUUCUGUGUCUUUUUCAAGAA |
| | | | | GAAAUGCUUCUGGAACAGCUUCUGUGC |
| | | | | UGCCAAGACCAACCAUCACCAUGGAAUU |
| | | | | CACAGGAGAUCAUGCUGUGUGCACAGC |
| | | | | UGGAUGUGUGCCUGAAGGAGUGACCUU |
| | | | | UGCUUGGUUUCUGGGAGAUGAUUCUUC |
| | | | | UCCAGCUGAAAAAGUGGCUGUGGCUUC |
| | | | | CCAGACCUCUUGUGGAAGACCUGGAAC |
| | | | | AGCAACCAUCAGAUCCACCCUGCCUGUG |
| | | | | UCUUAUGAACAGACAGAAUACAUUUGC |
| | | | | AGACUGGCUGGAUACCCUGAUGGAAUC |
| | | | | CCUGUGCUGGAACACCACUGAUAA |
| 228 | EboZ | HSV-2 gC | Version 4 | AUGGGAGUUACCGGAAUUCUGCAAUUG |
| | | (28-426) | | CCCAGGGAUCGGUUCAAGCGCACAUCCU |
| | | | | UCUUCCUGUGGGUCAUCAUCCUCUUUCA |
| | | | | GCGUACUUUCUCCAUACCCUCUCCCGGA |
| | | | | CGAACUAUCACUGUAGGCCCAAGGGGC |
| | | | | AACGCUAGUAAUGCCGCUCCCAGUGCUU |
| | | | | CACCACGCAAUGCGAGCGCACCCAGAAC |
| | | | | UACACCCACACCUCCUCAGCCGAGGAAA |
| | | | | GCCACCAAGUCCAAAGCCAGCACCGCCA |
| | | | | AACCCGCUCCUCCACCUAAAACAGGGCC |
| | | | | UCCCAAGACCUCAAGCGAGCCCGUUAGA |
| | | | | UGCAAUCGGCAUGAUCCACUGGCUCGU |
| | | | | UACGGUUCUCGGGUCCAGAUACGCUGU |
| | | | | AGGUUUCCUAACUCCACACGAACCGAGU |
| | | | | UCAGAUUGCAGAUCUGGAGAUAUGCCA |
| | | | | CAGCCACUGACGCUGAGAUUGGCACUGC |
| | | | | ACCUAGUCUGGAGGAGGUGAUGGUGAA |
| | | | | CGUGAGCGCCCCUCCUGGCGGUCAGCUU |
| | | | | GUGUAUGACUCAGCACCGAAUCGCACA |
| | | | | GAUCCGCACGUCAUAUGGGCCGAAGGU |
| | | | | GCAGGGCCCGGUGCAUCCCCUCGGCUGU |
| | | | | AUUCCGUGGUCGGACCACUCGGGCGCCA |
| | | | | GAGGCUUAUCAUUGAGGAACUGACCCU |
| | | | | CGAAACCCAGGGUAUGUACUACUGGGU |
| | | | | AUGGGGCCGUACCGACCGGCCUAGCGCC |
| | | | | UACGGAACUUGGGUGAGAGUUCGGGUG |
| | | | | UUCAGACCGCCAAGUCUUACAAUUCACC |
| | | | | CUCAUGCCGUGCUCGAAGGUCAGCCAUU |
| | | | | UAAGGCCACGUGUACUGCGGCUACGUA |
| | | | | CUAUCCCGGGAAUCGGGCUGAAUUCGU |
| | | | | GUGGUUUGAGGAUGGCAGGAGAGUGUU |
| | | | | CGACCCAGCCCAAAUCCACACCCAAACA |
| | | | | CAGGAGAACCCAGACGGGUUUUCCACCG |
| | | | | UGUCAACGGUCACAUCUGCCGCCGUCGG |
| | | | | AGGACAAGGGCCACCCAGAACCUUCACA |
| | | | | UGCCAGCUGACCUGGCAUAGGGAUAGC |
| | | | | GUAAGCUUUAGCCGGCGAAACGCAUCU |
| | | | | GGAACGGCGAGCGUUCUGCCUCGACCAA |
| | | | | CAAUCACCAUGGAGUUCACCGGCGAUCA |
| | | | | CGCAGUGUGCACUGCUGGGUGUGUACC |
| | | | | CGAAGGCGUGACAUUUGCCUGGUUUCU |
| | | | | GGGAGAUGACUCCUCACCCGCAGAAAA |
| | | | | GGUCGCCGUUGCAUCUCAGACCAGUUG |
| | | | | UGGCAGGCCCGGGACUGCUACCAUCCGC |
| | | | | AGCACUCUGCCGGUGUCUUACGAACAG |
| | | | | ACGGAGUACAUUUGCCGCUUGGCGGGC |
| | | | | UAUCCAGACGGCAUUCCAGUUCUGGAG |
| | | | | CAUCACUGA |
| 229 | HSV-2 gD | HSV-2 gD | | AUGGGCCGCCUGACCUCCGGCGUGGGCA |
| | | (26-331) | | CCGCCGCCCUGCUGGUGGUGGCCGUGGG |
| | | | | CCUGCGCGUGGUGUGCGCCAAGUACGCC |
| | | | | CUGGCCGACCCCUCCCUGAAGAUGGCCG |
| | | | | ACCCCAACCGCUUCCGCGGCAAGAACCU |
| | | | | GCCCGUGCUGGACCAGCUGACCGACCCC |
| | | | | CCCGGCGUGAAGCGCGUGUACCACAUCC |
| | | | | AGCCCUCCCUGGAGGACCCCUUCCAGCC |
| | | | | CCCCUCCAUCCCCAUCACCGUGUACUAC |
| | | | | GCCGUGCUGGAGCGCGCCUGCCGCUCCG |
| | | | | UGCUGCUGCACGCCCCCUCCGAGGCCCC |
| | | | | CCAGAUCGUGCGCGGCGCCUCCGACGAG |
| | | | | GCCCGCAAGCACACCUACAACCUGACCA |
| | | | | UCGCCUGGUACCGCAUGGGCGACAACUG |
| | | | | CGCCAUCCCCAUCACCGUGAUGGAGUAC |
| | | | | ACCGAGUGCCCCUACAACAAGUCCCUGG |
| | | | | GCGUGUGCCCCAUCCGCACCCAGCCCCG |
| | | | | CUGGUCCUACUACGACUCCUUCUCCGCC |
| | | | | GUGUCCGAGGACAACCUGGGCUUCCUG |
| | | | | AUGCACGCCCCCGCCUUCGAGACCGCCG |
| | | | | GCACCUACCUGCGCCUGGUGAAGAUCAA |
| | | | | CGACUGGACCGAGAUCACCCAGUUCAUC |
| | | | | CUGGAGCACCGCGCCCGCGCCUCCUGCA |
| | | | | AGUACGCCCUGCCCCUGCGCAUCCCCCC |
| | | | | CGCCGCCUGCCUGACCUCCAAGGCCUAC |
| | | | | CAGCAGGGCGUGACCGUGGACUCCAUCG |
| | | | | GCAUGCUGCCCCGCUUCAUCCCCGAGAA |
| | | | | CCAGCGCACCGUGGCCCUGUACUCCCUG |
| | | | | AAGAUCGCCGGCUGGCACGGCCCCAAGC |
| | | | | CCCCCUACACCUCCACCCUGCUGCCCCC |
| | | | | CGAGCUGUCCGACACCACCAACGCCACC |
| | | | | CAGCCCGAGCUGGUGCCCGAGGACCCCG |
| | | | | AGGACUCCGCCCUGCUGGAGGACCCCGC |
| | | | | CGGCACCGUGUCCUCCCAGAUCCCCCCC |
| | | | | AACUGGCACAUCCCCUCCAUCCAGGACG |
| | | | | UGGCCCCCCACCACUAA |
| 230 | HSV-2 gD | HSV-2 gD | Version 1 | AUGGGCAGACUGACAUCUGGCGUGGGA |
| | | (26-331) | | ACAGCUGCUCUGCUGGUGGUUGCUGUG |
| | | | | GGCCUGAGAGUCGUGUGUGCCAAAUAC |
| | | | | GCCCUGGCCGAUCCUAGCCUGAAGAUGG |
| | | | | CUGACCCCAACCGGUUCCGGGGCAAGAA |
| | | | | UCUGCCUGUUCUGGACCAGCUGACCGAU |
| | | | | CCUCCUGGCGUGAAACGGGUGUACCACA |
| | | | | UCCAGCCAAGCCUGGAAGAUCCCUUCCA |
| | | | | GCCUCCUAGCAUCCCCAUCACCGUGUAC |
| | | | | UACGCCGUGCUGGAAAGGGCCUGUAGA |
| | | | | AGCGUGCUGCUGCACGCCCCAUCUGAAG |
| | | | | CCCCUCAAAUCGUCAGAGGCGCUUCCGA |
| | | | | CGAGGCCAGAAAGCACACCUACAACCUG |
| | | | | ACAAUCGCCUGGUACAGAAUGGGCGAC |
| | | | | AACUGCGCCAUUCCUAUCACCGUGAUGG |
| | | | | AGUACACCGAGUGUCCCUACAACAAGA |
| | | | | GCCUGGGCGUGUGCCCCAUCAGAACACA |
| | | | | GCCUAGAUGGUCCUACUACGACAGCUUC |
| | | | | AGCGCCGUGUCCGAGGACAAUCUGGGC |
| | | | | UUCCUGAUGCAUGCCCCUGCCUUUGAGA |
| | | | | CAGCCGGCACCUAUCUGCGGCUGGUCAA |
| | | | | GAUCAACGACUGGACCGAGAUCACCCAG |
| | | | | UUCAUCCUGGAACACAGAGCCAGAGCCA |
| | | | | GCUGCAAAUACGCUCUGCCCCUGAGAAU |
| | | | | UCCUCCUGCCGCCUGUCUGACAAGCAAG |
| | | | | GCCUAUCAGCAGGGCGUGACCGUGGAU |
| | | | | AGCAUCGGCAUGCUGCCCAGAUUCAUCC |
| | | | | CCGAGAACCAGAGAACAGUGGCCCUGU |
| | | | | ACUCCCUGAAGAUCGCCGGAUGGCACGG |
| | | | | ACCCAAGCCUCCAUACACAAGCACACUG |
| | | | | CUGCCUCCAGAGCUGAGCGACACCACCA |
| | | | | AUGCCACACAGCCUGAACUGGUGCCUGA |
| | | | | GGACCCAGAGGAUUCUGCCCUGCUUGA |
| | | | | AGAUCCUGCCGGCACCGUGUCUAGCCAG |
| | | | | AUUCCUCCUAACUGGCACAUCCCCAGCA |
| | | | | UCCAGGAUGUGGCCCCUCAUCAUUGA |
| 231 | HSV-2 gD | HSV-2 gD | Version 2 | AUGGGGAGACUCACAUCAGGCGUAGGA |
| | | (26-331) | | ACCGCUGCCCUGUUGGUCGUGGCCGUUG |
| | | | | GUCUGAGAGUUGUGUGUGCCAAAUAUG |
| | | | | CUCUCGCUGAUCCGAGCCUCAAGAUGGC |
| | | | | AGAUCCCAACCGAUUUCGGGGAAAGAA |
| | | | | UCUGCCAGUACUGGACCAGCUGACGGAC |
| | | | | CCACCUGGCGUCAAACGCGUCUACCACA |
| | | | | UACAGCCUAGUCUUGAGGACCCUUUUC |
| | | | | AGCCACCGUCUAUCCCCAUUACCGUGUA |
| | | | | CUAUGCCGUGCUGGAACGCGCGUGUAG |
| | | | | GUCAGUUCUGCUGCAUGCCCCAUCCGAA |
| | | | | GCCCCCCAGAUCGUCAGAGGAGCUUCUG |
| | | | | AUGAAGCACGCAAACACACCUACAACCU |
| | | | | CACAAUAGCGUGGUAUCGAAUGGGCGA |
| | | | | UAACUGCGCAAUUCCCAUCACAGUCAUG |
| | | | | GAGUACACGGAGUGCCCCUACAACAAG |
| | | | | AGCCUCGGUGUUUGCCCUAUCAGGACAC |
| | | | | AACCCAGGUGGAGCUAUUACGACAGUU |
| | | | | UCAGCGCCGUGUCUGAGGACAAUCUGG |
| | | | | GGUUUCUGAUGCACGCACCCGCCUUCGA |
| | | | | GACUGCCGGCACCUACUUGCGGCUGGUG |
| | | | | AAGAUCAACGACUGGACUGAGAUCACC |
| | | | | CAGUUCAUCCUGGAACAUAGGGCCAGA |
| | | | | GCCAGCUGCAAGUAUGCUCUUCCCCUGC |
| | | | | GGAUUCCGCCUGCAGCAUGUCUGACCUC |
| | | | | AAAAGCCUACCAGCAAGGGGUGACUGU |
| | | | | GGACAGCAUUGGCAUGCUGCCUCGUUU |
| | | | | CAUUCCCGAGAAUCAACGGACAGUGGC |
| | | | | UCUGUAUUCCCUGAAGAUCGCAGGAUG |
| | | | | GCAUGGGCCCAAACCACCUUAUACCUCU |
| | | | | ACGUUGCUUCCACCAGAACUCAGUGACA |
| | | | | CCACUAAUGCGACACAGCCAGAACUUGU |
| | | | | GCCUGAGGAUCCUGAAGAUAGCGCUCU |
| | | | | GUUGGAGGAUCCAGCCGGUACUGUGUC |
| | | | | CUCCCAGAUACCACCCAAUUGGCACAUU |
| | | | | CCUUCCAUUCAGGACGUAGCUCCGCAUC |
| | | | | ACUGA |
| 232 | HSV-2 gD | HSV-2 gD | Version | AUGGGGAGACUCACAUCAGGCGUAGGA |
| | | (26-331) | 2.1 | ACCGCUGCCCUGUUGGUCGUGGCCGUUG |
| | | | | GUCUGAGAGUUGUGUGUGCCAAAUAUG |
| | | | | CUCUCGCUGAUCCGAGCCUCAAGAUGGC |
| | | | | AGAUCCCAACCGAUUUCGGGGAAAGAA |
| | | | | UCUGCCAGUACUGGACCAGCUGACGGAC |
| | | | | CCACCUGGCGUCAAACGCGUCUACCACA |
| | | | | UACAGCCUAGUCUUGAGGACCCUUUUC |
| | | | | AGCCACCGUCUAUCCCCAUUACCGUGUA |
| | | | | CUAUGCCGUGCUGGAACGCGCGUGUAG |
| | | | | GUCAGUUCUGCUGCAUGCCCCAUCCGAA |
| | | | | GCCCCCCAGAUCGUCAGAGGAGCUUCUG |
| | | | | AUGAAGCACGCAAACACACCUACAACCU |
| | | | | CACAAUAGCGUGGUAUCGAAUGGGCGA |
| | | | | UAACUGCGCAAUUCCCAUCACAGUCAUG |
| | | | | GAGUACACGGAGUGCCCCUACAACAAG |
| | | | | AGCCUCGGUGUUUGCCCUAUCAGGACAC |
| | | | | AACCCAGGUGGAGCUAUUACGACAGUU |
| | | | | UCAGCGCCGUGUCUGAGGACAAUCUGG |
| | | | | GGUUUCUGAUGCACGCACCCGCCUUCGA |
| | | | | GACUGCCGGCACCUACUUGCGGCUGGUG |
| | | | | AAGAUCAACGACUGGACUGAGAUCACC |
| | | | | CAGUUCAUCCUGGAACAUAGGGCCAGA |
| | | | | GCCAGCUGCAAGUAUGCCCUUCCCCUGC |
| | | | | GGAUUCCGCCUGCAGCAUGUCUGACCUC |
| | | | | AAAAGCCUACCAGCAAGGGGUGACUGU |
| | | | | GGACAGCAUUGGCAUGCUGCCUCGUUU |
| | | | | CAUUCCCGAGAAUCAACGGACAGUGGC |
| | | | | UCUGUAUUCCCUGAAGAUCGCAGGAUG |
| | | | | GCAUGGGCCCAAACCACCUUAUACCUCU |
| | | | | ACGUUGCUUCCACCAGAACUCAGUGACA |
| | | | | CCACUAAUGCGACACAGCCAGAACUUGU |
| | | | | GCCUGAGGAUCCUGAAGAUAGCGCUCU |
| | | | | GUUGGAGGAUCCAGCCGGUACUGUGUC |
| | | | | CUCCCAGAUACCACCCAAUUGGCACAUU |
| | | | | CCUUCCAUUCAGGACGUAGCUCCGCAUC |
| | | | | ACUGA |
| 233 | HSV-2 gD | HSV-2 gD | Version 3 | AUGGGCAGACUGACCUCCGGCGUGGGC |
| | | (26-331) | | ACCGCCGCCCUGCUGGUGGUGGCCGUGG |
| | | | | GCCUGAGAGUGGUGUGCGCCAAAUACG |
| | | | | CCCUGGCCGAUCCCUCCCUGAAAAUGGC |
| | | | | CGAUCCCAACAGGUUCAGAGGCAAAAA |
| | | | | CCUGCCCGUGCUGGAUCAGCUGACCGAU |
| | | | | CCCCCUGGCGUGAAAAGAGUGUACCACA |
| | | | | UCCAGCCCUCCCUGGAAGAUCCCUUCCA |
| | | | | GCCCCCUUCCAUCCCCAUCACCGUGUAC |
| | | | | UACGCCGUGCUGGAAAGAGCUUGCAGA |
| | | | | UCCGUGCUGCUGCACGCCCCCUCCGAAG |
| | | | | CCCCUCAGAUCGUGAGAGGCGCCUCCGA |
| | | | | UGAAGCCAGAAAACACACCUACAACCUG |
| | | | | ACCAUCGCCUGGUACAGAAUGGGCGAU |
| | | | | AACUGCGCCAUCCCCAUCACCGUGAUGG |
| | | | | AAUACACCGAAUGCCCCUACAACAAAUC |
| | | | | CCUGGGCGUGUGCCCCAUCAGAACCCAG |
| | | | | CCCAGAUGGUCCUACUACGAUUCCUUCU |
| | | | | CCGCCGUGUCCGAAGAUAACCUGGGCUU |
| | | | | CCUGAUGCACGCCCCCGCCUUCGAAACC |
| | | | | GCCGGCACCUACCUGAGACUGGUGAAA |
| | | | | AUCAACGAUUGGACCGAAAUCACCCAG |
| | | | | UUCAUCCUGGAACACAGAGCCAGAGCCU |
| | | | | CCUGCAAAUACGCCCUGCCCCUGAGAAU |
| | | | | CCCUCCCGCCGCCUGCCUGACCUCCAAA |
| | | | | GCCUACCAGCAGGGCGUGACCGUGGAU |
| | | | | UCCAUCGGCAUGCUGCCCAGAUUCAUCC |
| | | | | CCGAAAACCAGAGAACCGUGGCCCUGUA |
| | | | | CUCCCUGAAAAUCGCCGGCUGGCACGGC |
| | | | | CCCAAACCCCCUUACACCUCCACCCUGC |
| | | | | UGCCCCCUGAACUGUCCGAUACCACCAA |
| | | | | CGCCACCCAGCCCGAACUGGUGCCCGAA |
| | | | | GAUCCCGAAGAUUCCGCCCUGCUGGAAG |
| | | | | AUCCCGCCGGCACCGUGUCCUCCCAGAU |
| | | | | CCCUCCCAACUGGCACAUCCCCUCCAUC |
| | | | | CAGGAUGUGGCCCCUCACCACUGA |
| 234 | IL2 | HSV-2 gE | | AUGCGCAUGCAGCUGCUGCUGCUGAUC |
| | | (24-405) | | GCCCUGUCCCUGGCCCUGGUGACCAACU |
| | | | | CCCGCACCUCCUGGAAGCGCGUGACCUC |
| | | | | CGGCGAGGACGUGGUGCUGCUGCCCGCC |
| | | | | CCCGCCGGCCCCGAGGAGCGCACCCGCG |
| | | | | CCCACAAGCUGCUGUGGGCCGCCGAGCC |
| | | | | CCUGGACGCCUGCGGCCCCCUGCGCCCC |
| | | | | UCCUGGGUGGCCCUGUGGCCCCCCCGCC |
| | | | | GCGUGCUGGAGACCGUGGUGGACGCCG |
| | | | | CCUGCAUGCGCGCCCCCGAGCCCCUGGC |
| | | | | CAUCGCCUACUCCCCCCCCUUCCCCGCC |
| | | | | GGCGACGAGGGCCUGUACUCCGAGCUG |
| | | | | GCCUGGCGCGACCGCGUGGCCGUGGUGA |
| | | | | ACGAGUCCCUGGUGAUCUACGGCGCCCU |
| | | | | GGAGACCGACUCCGGCCUGUACACCCUG |
| | | | | UCCGUGGUGGGCCUGUCCGACGAGGCCC |
| | | | | GCCAGGUGGCCUCCGUGGUGCUGGUGG |
| | | | | UGGAGCCCGCCCCCGUGCCCACCCCCAC |
| | | | | CCCCGACGACUACGACGAGGAGGACGAC |
| | | | | GCCGGCGUGUCCGAGCGCACCCCCGUGU |
| | | | | CCGUGCCCCCCCCCACCCCCCCCCGCCGC |
| | | | | CCCCCCGUGGCC- |
| | | | | CCCCCCACCCACCCCCGCGUGAUCCCCG |
| | | | | AGGUGUCCCACGUGCGCGGCGUGACCGU |
| | | | | GCACAUGGAGACCCCCGAGGCCAUCCUG |
| | | | | UUCGCCCCCGGCGAGACCUUCGGCACCA |
| | | | | ACGUGUCCAUCCACGCCAUCGCCCACGA |
| | | | | CGACGGCCCCUACGCCAUGGACGUGGUG |
| | | | | UGGAUGCGCUUCGACGUGCCCUCCUCCU |
| | | | | GCGCCGAGAUGCGCAUCUACGAGGCCUG |
| | | | | CCUGUACCACCCCCAGCUGCCCGAGUGC |
| | | | | CUGUCCCCCGCCGACGCCCCCUGCGCCG |
| | | | | UGUCCUCCUGGGCCUACCGCCUGGCCGU |
| | | | | GCGCUCCUACGCCGGCUGCUCCCGCACC |
| | | | | ACCCCCCCCCCCCGCUGCUUCGCCGAGG |
| | | | | CCCGCAUGGAGCCCGUGCCCGGCCUGGC |
| | | | | CUGGCUGGCCUCCACCGUGAACCUGGAG |
| | | | | UUCCAGCACGCCUCCCCCCAGCACGCCG |
| | | | | GCCUGUACCUGUGCGUGGUGUACGUGG |
| | | | | ACGACCACAUCCACGCCUGGGGCCACAU |
| | | | | GACCAUCUCCACCGCCGCCCAGUACCGC |
| | | | | AACGCCGUGGUGGAGCAGCACCUGCCCC |
| | | | | AGCGCCAGCCCGAGCCCGUGGAGCCCAC |
| | | | | CCGCCCCCACGUGCGCGCCUAA |
| 235 | IL2 | HSV-2 gE | Version 1 | AUGAGAAUGCAGCUGCUGCUCCUGAUC |
| | | (24-405) | | GCCCUGUCUCUGGCCCUGGUCACCAAUA |
| | | | | GCAGAACCAGCUGGAAAAGAGUGACCA |
| | | | | GCGGCGAGGAUGUGGUGCUGCUUCCUG |
| | | | | CUCCUGCUGGCCCCGAGGAAAGAACAAG |
| | | | | AGCCCACAAACUGCUGUGGGCCGCUGAG |
| | | | | CCUCUUGAUGCCUGUGGACCUCUCAGAC |
| | | | | CUAGCUGGGUUGCACUGUGGCCACCUCG |
| | | | | GAGAGUGCUGGAAACAGUGGUGGAUGC |
| | | | | CGCCUGCAUGAGAGCCCCUGAACCUCUG |
| | | | | GCCAUUGCCUACUCUCCACCAUUUCCAG |
| | | | | CCGGCGACGAGGGCCUGUAUUCUGAGC |
| | | | | UUGCUUGGAGAGACAGAGUGGCCGUGG |
| | | | | UCAACGAGAGCCUGGUUAUCUAUGGCG |
| | | | | CCCUGGAAACCGACAGCGGCCUGUACAC |
| | | | | ACUGUCUGUCGUGGGCCUGUCUGACGA |
| | | | | GGCUAGACAGGUGGCAUCUGUGGUCCU |
| | | | | GGUGGUGGAACCUGCUCCAGUGCCUAC |
| | | | | ACCUACACCUGACGACUACGACGAGGAA |
| | | | | GAUGACGCUGGCGUCAGCGAGAGAACC |
| | | | | CCUGUUUCUGUGCCUCCUCCUACGCCUC |
| | | | | CUCGUAGACCUCCUGUUGCUCCUCCAAC |
| | | | | ACACCCCAGAGUGAUCCCUGAAGUGUCU |
| | | | | CACGUGCGGGGCGUGACCGUGCACAUG |
| | | | | GAAACACCUGAGGCCAUCCUGUUCGCCC |
| | | | | CUGGCGAGACAUUUGGCACCAACGUGU |
| | | | | CCAUCCACGCUAUCGCCCACGACGAUGG |
| | | | | CCCUUACGCCAUGGAUGUCGUGUGGAU |
| | | | | GAGAUUCGACGUGCCCAGCAGCUGUGCC |
| | | | | GAGAUGAGAAUCUAUGAGGCCUGCCUG |
| | | | | UAUCACCCUCAGCUGCCCGAAUGUCUGA |
| | | | | GCCCUGCUGAUGCCCCUUGUGCCGUUAG |
| | | | | CAGCUGGGCCUAUAGACUGGCCGUGCG |
| | | | | GUCUUAUGCCGGCUGCUCUAGAACAACC |
| | | | | CCUCCUCCUCGGUGUUUCGCCGAGGCCA |
| | | | | GAAUGGAACCUGUUCCUGGACUGGCCU |
| | | | | GGCUGGCCUCCACAGUGAACCUGGAAU |
| | | | | UUCAGCACGCCUCUCCACAGCACGCCGG |
| | | | | CCUGUAUCUGUGUGUGGUGUACGUGGA |
| | | | | CGAUCACAUCCACGCCUGGGGCCACAUG |
| | | | | ACCAUCUCUACAGCCGCUCAGUACCGGA |
| | | | | ACGCCGUGGUUGAACAGCAUCUGCCUCA |
| | | | | GAGACAGCCCGAGCCUGUGGAACCUACA |
| | | | | AGACCUCAUGUUCGGGCCUGA |
| 236 | IL2 | HSV-2 gE | Version 2 | AUGCGAAUGCAGCUUCUGCUGCUCAUU |
| | | (24-405) | | GCCUUGUCCCUUGCCUUGGUGACCAACU |
| | | | | CACGCACCUCUUGGAAACGCGUUACUUC |
| | | | | CGGGGAGGACGUUGUCCUCCUUCCAGCA |
| | | | | CCCGCAGGACCUGAAGAGAGGACUAGG |
| | | | | GCCCACAAGCUGCUGUGGGCCGCUGAAC |
| | | | | CUCUGGAUGCCUGUGGUCCUCUGAGACC |
| | | | | UAGCUGGGUCGCCCUUUGGCCACCUAGA |
| | | | | CGCGUUCUGGAGACGGUCGUGGAUGCC |
| | | | | GCGUGCAUGCGUGCACCCGAACCUCUGG |
| | | | | CCAUCGCCUAUAGUCCCCCUUUUCCCGC |
| | | | | UGGCGACGAGGGGCUUUACUCCGAACU |
| | | | | GGCCUGGCGGGAUAGGGUGGCGGUGGU |
| | | | | GAACGAGAGCCUCGUCAUCUACGGUGC |
| | | | | UCUGGAAACCGACUCAGGACUGUAUAC |
| | | | | GCUCAGCGUUGUUGGCCUCUCCGAUGA |
| | | | | GGCUCGACAGGUUGCCUCCGUAGUGCU |
| | | | | GGUCGUAGAACCAGCCCCCGUACCAACA |
| | | | | CCCACACCCGACGACUACGACGAAGAGG |
| | | | | ACGACGCUGGAGUUAGCGAAAGAACAC |
| | | | | CGGUGAGUGUGCCACCUCCCACACCGCC |
| | | | | AAGGAGACCCCCAGUAGCACCUCCAACC |
| | | | | CAUCCGAGAGUGAUUCCCGAGGUCAGCC |
| | | | | AUGUGCGCGGCGUAACUGUGCACAUGG |
| | | | | AGACGCCCGAAGCGAUACUGUUUGCCCC |
| | | | | UGGAGAGACAUUCGGCACCAAUGUGUC |
| | | | | CAUACACGCAAUUGCGCACGAUGAUGG |
| | | | | CCCAUACGCUAUGGACGUCGUCUGGAU |
| | | | | GAGGUUCGAUGUGCCUUCUUCUUGCGC |
| | | | | CGAGAUGAGGAUCUACGAGGCAUGCCU |
| | | | | GUAUCACCCCCAAUUGCCGGAGUGUCUG |
| | | | | UCUCCCGCAGAUGCACCGUGUGCAGUGA |
| | | | | GUAGCUGGGCUUAUCGGUUGGCUGUCC |
| | | | | GGAGUUAUGCUGGGUGUUCACGGACCA |
| | | | | CCCCACCUCCACGUUGCUUUGCUGAAGC |
| | | | | CAGAAUGGAACCCGUGCCUGGUCUGGC |
| | | | | UUGGCUGGCAUCAACUGUCAACCUGGA |
| | | | | GUUCCAGCAUGCCUCUCCACAGCACGCA |
| | | | | GGCCUGUAUCUCUGCGUGGUGUACGUU |
| | | | | GACGAUCACAUCCAUGCGUGGGGGCAU |
| | | | | AUGACCAUCAGCACAGCUGCCCAGUACC |
| | | | | GCAAUGCCGUCGUGGAGCAGCACCUCCC |
| | | | | CCAACGGCAGCCAGAACCAGUGGAGCCC |
| | | | | ACUCGGCCUCAUGUGCGAGCCUGA |
| 237 | IL2 | HSV-2 gE | Version 3 | AUGAGAAUGCAGCUGCUGCUGCUGAUC |
| | | (24-405) | | GCCCUGUCCCUGGCCCUGGUGACCAACU |
| | | | | CCAGAACCUCCUGGAAAAGAGUGACCUC |
| | | | | CGGCGAAGAUGUGGUGCUGCUGCCCGCC |
| | | | | CCCGCCGGCCCCGAAGAAAGAACCAGAG |
| | | | | CCCACAAACUGCUGUGGGCCGCCGAACC |
| | | | | CCUGGAUGCCUGCGGCCCCCUCAGACCC |
| | | | | UCCUGGGUGGCCCUGUGGCCCCCUAGAA |
| | | | | GGGUGCUGGAAACCGUGGUGGAUGCCG |
| | | | | CCUGCAUGAGAGCCCCCGAACCCCUGGC |
| | | | | CAUCGCCUACUCCCCUCCCUUCCCCGCC |
| | | | | GGCGAUGAAGGCCUGUACUCCGAACUG |
| | | | | GCCUGGAGAGAUAGAGUGGCCGUGGUG |
| | | | | AACGAAUCCCUGGUGAUCUACGGCGCCC |
| | | | | UGGAAACCGAUUCCGGCCUGUACACCCU |
| | | | | GUCCGUGGUGGGCCUGUCCGAUGAAGC |
| | | | | CAGACAGGUGGCCUCCGUGGUGCUGGU |
| | | | | GGUGGAACCCGCCCCCGUGCCCACCCCC |
| | | | | ACCCCCGAUGAUUACGAUGAAGAAGAU |
| | | | | GAUGCCGGCGUGUCCGAAAGAACCCCCG |
| | | | | UGUCCGUGCCCCCUCCCACCCCUCCCCG |
| | | | | CAGACCCCCUGUGGCCCCUCCCACCCAC |
| | | | | CCCAGAGUGAUCCCCGAAGUGUCCCACG |
| | | | | UGAGAGGCGUGACCGUGCACAUGGAAA |
| | | | | CCCCCGAAGCCAUCCUGUUCGCCCCCGG |
| | | | | CGAAACCUUCGGCACCAACGUGUCCAUC |
| | | | | CACGCCAUCGCCCACGAUGAUGGCCCCU |
| | | | | ACGCCAUGGAUGUGGUGUGGAUGAGAU |
| | | | | UCGAUGUGCCCUCCUCCUGCGCCGAAAU |
| | | | | GAGAAUCUACGAAGCCUGCCUGUACCAC |
| | | | | CCCCAGCUGCCCGAAUGCCUGUCCCCCG |
| | | | | CCGAUGCCCCCUGCGCCGUGUCCUCCUG |
| | | | | GGCCUACAGACUGGCCGUGAGAUCCUAC |
| | | | | GCCGGCUGCUCCAGAACCACCCCUCCCC |
| | | | | CUAGAUGCUUCGCCGAAGCCAGAAUGG |
| | | | | AACCCGUGCCCGGCCUGGCCUGGCUGGC |
| | | | | CUCCACCGUGAACCUGGAAUUCCAGCAC |
| | | | | GCCAGCCCCCAGCACGCCGGCCUGUACC |
| | | | | UGUGCGUGGUGUACGUGGAUGAUCACA |
| | | | | UCCACGCCUGGGGCCACAUGACCAUCUC |
| | | | | CACCGCCGCCCAGUACAGAAACGCCGUG |
| | | | | GUGGAACAGCACCUGCCCCAGAGACAGC |
| | | | | CCGAACCCGUGGAACCCACCAGACCCCA |
| | | | | CGUGAGAGCCUGA |
| 237 | HSV-2 gD- | HSV-2 gE | | AUGGGCCGCCUGACCUCCGGCGUGGGCA |
| | KYA | (24-405) | | CCGCCGCCCUGCUGGUGGUGGCCGUGGG |
| | | | | CCUGCGCGUGGUGUGCGCCAAGUACGCC |
| | | | | CGCACCUCCUGGAAGCGCGUGACCUCCG |
| | | | | GCGAGGACGUGGUGCUGCUGCCCGCCCC |
| | | | | CGCCGGCCCCGAGGAGCGCACCCGCGCC |
| | | | | CACAAGCUGCUGUGGGCCGCCGAGCCCC |
| | | | | UGGACGCCUGCGGCCCCCUGCGCCCCUC |
| | | | | CUGGGUGGCCCUGUGGCCCCCCCGCCGC |
| | | | | GUGCUGGAGACCGUGGUGGACGCCGCC |
| | | | | UGCAUGCGCGCCCCCGAGCCCCUGGCCA |
| | | | | UCGCCUACUCCCCCCCCUUCCCCGCCGG |
| | | | | CGACGAGGGCCUGUACUCCGAGCUGGCC |
| | | | | UGGCGCGACCGCGUGGCCGUGGUGAAC |
| | | | | GAGUCCCUGGUGAUCUACGGCGCCCUGG |
| | | | | AGACCGACUCCGGCCUGUACACCCUGUC |
| | | | | CGUGGUGGGCCUGUCCGACGAGGCCCGC |
| | | | | CAGGUGGCCUCCGUGGUGCUGGUGGUG |
| | | | | GAGCCCGCCCCCGUGCCCACCCCCACCC |
| | | | | CCGACGACUACGACGAGGAGGACGACGC |
| | | | | CGGCGUGUCCGAGCGCACCCCCGUGUCC |
| | | | | GUGCCCCCCCCCACCCCCCCCCGCCGCCC |
| | | | | CCCCGUGGCCCCCCCCACCCACCCCCGC |
| | | | | GUGAUCCCCGAGGUGUCCCACGUGCGCG |
| | | | | GCGUGACCGUGCACAUGGAGACCCCCGA |
| | | | | GGCCAUCCUGUUCGCCCCCGGCGAGACC |
| | | | | UUCGGCACCAACGUGUCCAUCCACGCCA |
| | | | | UCGCCCACGACGACGGCCCCUACGCCAU |
| | | | | GGACGUGGUGUGGAUGCGCUUCGACGU |
| | | | | GCCCUCCUCCUGCGCCGAGAUGCGCAUC |
| | | | | UACGAGGCCUGCCUGUACCACCCCCAGC |
| | | | | UGCCCGAGUGCCUGUCCCCCGCCGACGC |
| | | | | CCCCUGCGCCGUGUCCUCCUGGGCCUAC |
| | | | | CGCCUGGCCGUGCGCUCCUACGCCGGCU |
| | | | | GCUCCCGCACCACCCCCCCCCCCCGCUG |
| | | | | CUUCGCCGAGGCCCGCAUGGAGCCCGUG |
| | | | | CCCGGCCUGGCCUGGCUGGCCUCCACCG |
| | | | | UGAACCUGGAGUUCCAGCACGCCUCCCC |
| | | | | CCAGCACGCCGGCCUGUACCUGUGCGUG |
| | | | | GUGUACGUGGACGACCACAUCCACGCCU |
| | | | | GGGGCCACAUGACCAUCUCCACCGCCGC |
| | | | | CCAGUACCGCAACGCCGUGGUGGAGCAG |
| | | | | CACCUGCCCCAGCGCCAGCCCGAGCCCG |
| | | | | UGGAGCCCACCCGCCCCCACGUGCGCGC |
| | | | | CUAA |
| 239 | HSV-2 gD- | HSV-2 gE | Version 1 | AUGGGCAGACUGACAUCUGGCGUGGGA |
| | KYA | (24-405) | | ACAGCUGCUCUGCUGGUGGUUGCUGUG |
| | | | | GGCCUGAGAGUCGUGUGUGCCAAAUAC |
| | | | | GCCAGAACCAGCUGGAAAAGAGUGACC |
| | | | | AGCGGCGAGGAUGUGGUGCUGCUUCCU |
| | | | | GCUCCUGCUGGCCCCGAGGAAAGAACAA |
| | | | | GAGCCCACAAACUGCUGUGGGCCGCUGA |
| | | | | GCCUCUUGAUGCCUGUGGACCUCUCAGA |
| | | | | CCUAGCUGGGUUGCACUGUGGCCACCUC |
| | | | | GGAGAGUGCUGGAAACAGUGGUGGAUG |
| | | | | CCGCCUGCAUGAGAGCCCCUGAACCUCU |
| | | | | GGCCAUUGCCUACUCUCCACCAUUUCCA |
| | | | | GCCGGCGACGAGGGCCUGUAUUCUGAG |
| | | | | CUUGCUUGGAGAGACAGAGUGGCCGUG |
| | | | | GUCAACGAGAGCCUGGUUAUCUAUGGC |
| | | | | GCCCUGGAAACCGACAGCGGCCUGUACA |
| | | | | CACUGUCUGUCGUGGGCCUGUCUGACG |
| | | | | AGGCUAGACAGGUGGCAUCUGUGGUCC |
| | | | | UGGUGGUGGAACCUGCUCCAGUGCCUA |
| | | | | CACCUACACCUGACGACUACGACGAGGA |
| | | | | AGAUGACGCUGGCGUCAGCGAGAGAAC |
| | | | | CCCUGUUUCUGUGCCUCCUCCUACGCCU |
| | | | | CCUCGUAGACCUCCUGUUGCUCCUCCAA |
| | | | | CACACCCCAGAGUGAUCCCUGAAGUGUC |
| | | | | UCACGUGCGGGGCGUGACCGUGCACAU |
| | | | | GGAAACACCUGAGGCCAUCCUGUUCGCC |
| | | | | CCUGGCGAGACAUUUGGCACCAACGUG |
| | | | | UCCAUCCACGCUAUCGCCCACGACGAUG |
| | | | | GCCCUUACGCCAUGGAUGUCGUGUGGA |
| | | | | UGAGAUUCGACGUGCCCAGCAGCUGUG |
| | | | | CCGAGAUGAGAAUCUAUGAGGCCUGCC |
| | | | | UGUAUCACCCUCAGCUGCCCGAAUGUCU |
| | | | | GAGCCCUGCUGAUGCCCCUUGUGCCGUU |
| | | | | AGCAGCUGGGCCUAUAGACUGGCCGUG |
| | | | | CGGUCUUAUGCCGGCUGCUCUAGAACA |
| | | | | ACCCCUCCUCCUCGGUGUUUCGCCGAGG |
| | | | | CCAGAAUGGAACCUGUUCCUGGACUGG |
| | | | | CCUGGCUGGCCUCCACAGUGAACCUGGA |
| | | | | AUUUCAGCACGCCUCUCCACAGCACGCC |
| | | | | GGCCUGUAUCUGUGUGUGGUGUACGUG |
| | | | | GACGAUCACAUCCACGCCUGGGGCCACA |
| | | | | UGACCAUCUCUACAGCCGCUCAGUACCG |
| | | | | GAACGCCGUGGUUGAACAGCAUCUGCC |
| | | | | UCAGAGACAGCCCGAGCCUGUGGAACCU |
| | | | | ACAAGACCUCAUGUUCGGGCCUGA |
| 240 | HSV-2 gD- | HSV-2 gE | Version 2 | AUGGGGAGACUCACAUCAGGCGUAGGA |
| | KYA | (24-405) | | ACCGCUGCCCUGUUGGUCGUGGCCGUUG |
| | | | | GUCUGAGAGUUGUGUGUGCCAAAUAUG |
| | | | | CUCGCACCUCUUGGAAACGCGUUACUUC |
| | | | | CGGGGAGGACGUUGUCCUCCUUCCAGCA |
| | | | | CCCGCAGGACCUGAGGAAAGGACUAGG |
| | | | | GCCCACAAGCUGCUGUGGGCCGCUGAAC |
| | | | | CUCUGGAUGCCUGUGGUCCUCUGAGACC |
| | | | | UAGCUGGGUCGCCCUUUGGCCACCUAGA |
| | | | | CGCGUUCUGGAGACGGUCGUGGAUGCC |
| | | | | GCGUGCAUGCGUGCACCCGAACCUCUGG |
| | | | | CCAUCGCCUAUAGUCCCCCUUUUCCCGC |
| | | | | UGGCGACGAGGGGCUUUACUCCGAACU |
| | | | | GGCCUGGCGGGAUAGGGUGGCGGUGGU |
| | | | | GAACGAGAGCCUCGUCAUCUACGGUGC |
| | | | | UCUGGAAACCGACUCAGGACUGUAUAC |
| | | | | GCUCAGCGUUGUUGGCCUCUCCGAUGA |
| | | | | GGCUCGACAGGUUGCCUCCGUAGUGCU |
| | | | | GGUCGUAGAACCAGCCCCCGUACCAACA |
| | | | | CCCACACCCGACGACUACGACGAGGAGG |
| | | | | ACGACGCUGGAGUUAGCGAAAGAACAC |
| | | | | CGGUGAGUGUGCCACCUCCCACACCGCC |
| | | | | AAGGAGACCCCCAGUAGCACCUCCAACC |
| | | | | CAUCCGAGAGUGAUUCCCGAGGUCAGCC |
| | | | | AUGUGCGCGGCGUAACUGUGCACAUGG |
| | | | | AGACGCCCGAAGCGAUACUGUUUGCCCC |
| | | | | UGGAGAGACAUUCGGCACCAAUGUGUC |
| | | | | CAUACACGCAAUUGCGCACGAUGAUGG |
| | | | | CCCAUACGCUAUGGACGUCGUCUGGAU |
| | | | | GAGGUUCGAUGUGCCUUCUUCUUGCGC |
| | | | | CGAGAUGAGGAUCUACGAGGCAUGCCU |
| | | | | GUAUCACCCCCAAUUGCCGGAGUGUCUG |
| | | | | UCUCCCGCAGAUGCACCGUGUGCAGUGA |
| | | | | GUAGCUGGGCUUAUCGGUUGGCUGUCC |
| | | | | GGAGUUAUGCUGGGUGUUCACGGACCA |
| | | | | CCCCACCUCCACGUUGCUUUGCUGAAGC |
| | | | | CAGAAUGGAACCCGUGCCUGGUCUGGC |
| | | | | UUGGCUGGCAUCAACUGUCAACCUGGA |
| | | | | GUUCCAGCAUGCCUCUCCACAGCACGCA |
| | | | | GGCCUGUAUCUCUGCGUGGUGUACGUU |
| | | | | GACGAUCACAUCCAUGCGUGGGGGCAU |
| | | | | AUGACCAUCAGCACAGCUGCCCAGUACC |
| | | | | GCAAUGCCGUCGUGGAGCAGCACCUCCC |
| | | | | CCAACGGCAGCCAGAACCAGUGGAGCCC |
| | | | | ACUCGGCCUCAUGUGCGAGCCUGA |
| 241 | HSV-2 gD- | HSV-2 gE | Version | AUGGGGAGACUCACAUCAGGCGUAGGA |
| | KYA | (24-405) | 2.1 | ACCGCUGCCCUGUUGGUCGUGGCCGUUG |
| | | | | GUCUGAGAGUUGUGUGUGCCAAAUAUG |
| | | | | CUCGCACCUCUUGGAAACGCGUUACUUC |
| | | | | CGGGGAGGACGUUGUCCUCCUUCCAGCA |
| | | | | CCCGCAGGACCUGAAGAGAGGACUAGG |
| | | | | GCCCACAAGCUGCUGUGGGCCGCUGAAC |
| | | | | CUCUGGAUGCCUGUGGUCCUCUGAGACC |
| | | | | UAGCUGGGUCGCCCUUUGGCCACCUAGA |
| | | | | CGCGUUCUGGAGACGGUCGUGGAUGCC |
| | | | | GCGUGCAUGCGUGCACCCGAACCUCUGG |
| | | | | CCAUCGCCUAUAGUCCCCCUUUUCCCGC |
| | | | | UGGCGACGAGGGGCUUUACUCCGAACU |
| | | | | GGCCUGGCGGGAUAGGGUGGCGGUGGU |
| | | | | GAACGAGAGCCUCGUCAUCUACGGUGC |
| | | | | UCUGGAAACCGACUCAGGACUGUAUAC |
| | | | | GCUCAGCGUUGUUGGCCUCUCCGAUGA |
| | | | | GGCUCGACAGGUUGCCUCCGUAGUGCU |
| | | | | GGUCGUAGAACCAGCCCCCGUACCAACA |
| | | | | CCCACACCCGACGACUACGACGAAGAGG |
| | | | | ACGACGCUGGAGUUAGCGAAAGAACAC |
| | | | | CGGUGAGUGUGCCACCUCCCACACCGCC |
| | | | | AAGGAGACCCCCAGUAGCACCUCCAACC |
| | | | | CAUCCGAGAGUGAUUCCCGAGGUCAGCC |
| | | | | AUGUGCGCGGCGUAACUGUGCACAUGG |
| | | | | AGACGCCCGAAGCGAUACUGUUUGCCCC |
| | | | | UGGAGAGACAUUCGGCACCAAUGUGUC |
| | | | | CAUACACGCAAUUGCGCACGAUGAUGG |
| | | | | CCCAUACGCUAUGGACGUCGUCUGGAU |
| | | | | GAGGUUCGAUGUGCCUUCUUCUUGCGC |
| | | | | CGAGAUGAGGAUCUACGAGGCAUGCCU |
| | | | | GUAUCACCCCCAAUUGCCGGAGUGUCUG |
| | | | | UCUCCCGCAGAUGCACCGUGUGCAGUGA |
| | | | | GUAGCUGGGCUUAUCGGUUGGCUGUCC |
| | | | | GGAGUUAUGCUGGGUGUUCACGGACCA |
| | | | | CCCCACCUCCACGUUGCUUUGCUGAAGC |
| | | | | CAGAAUGGAACCCGUGCCUGGUCUGGC |
| | | | | UUGGCUGGCAUCAACUGUCAACCUGGA |
| | | | | GUUCCAGCAUGCCUCUCCACAGCACGCA |
| | | | | GGCCUGUAUCUCUGCGUGGUGUACGUU |
| | | | | GACGAUCACAUCCAUGCGUGGGGGCAU |
| | | | | AUGACCAUCAGCACAGCUGCCCAGUACC |
| | | | | GCAAUGCCGUCGUGGAGCAGCACCUCCC |
| | | | | CCAACGGCAGCCAGAACCAGUGGAGCCC |
| | | | | ACUCGGCCUCAUGUGCGAGCCUGA |
| 242 | HSV-2 gD- | HSV-2 gE | Version 3 | AUGGGCAGACUGACCUCCGGCGUGGGC |
| | KYA | (24-405) | | ACCGCCGCCCUGCUGGUGGUGGCCGUGG |
| | | | | GCCUGAGAGUGGUGUGCGCCAAAUACG |
| | | | | CCAGAACCUCCUGGAAAAGAGUGACCUC |
| | | | | CGGCGAAGAUGUGGUGCUGCUGCCCGCC |
| | | | | CCCGCCGGCCCCGAAGAAAGAACCAGAG |
| | | | | CCCACAAACUGCUGUGGGCCGCCGAACC |
| | | | | CCUGGAUGCCUGCGGCCCCCUCAGACCC |
| | | | | UCCUGGGUGGCCCUGUGGCCCCCUAGAA |
| | | | | GGGUGCUGGAAACCGUGGUGGAUGCCG |
| | | | | CCUGCAUGAGAGCCCCCGAACCCCUGGC |
| | | | | CAUCGCCUACUCCCCUCCCUUCCCCGCC |
| | | | | GGCGAUGAAGGCCUGUACUCCGAACUG |
| | | | | GCCUGGAGAGAUAGAGUGGCCGUGGUG |
| | | | | AACGAAUCCCUGGUGAUCUACGGCGCCC |
| | | | | UGGAAACCGAUUCCGGCCUGUACACCCU |
| | | | | GUCCGUGGUGGGCCUGUCCGAUGAAGC |
| | | | | CAGACAGGUGGCCUCCGUGGUGCUGGU |
| | | | | GGUGGAACCCGCCCCCGUGCCCACCCCC |
| | | | | ACCCCCGAUGAUUACGAUGAAGAAGAU |
| | | | | GAUGCCGGCGUGUCCGAAAGAACCCCCG |
| | | | | UGUCCGUGCCCCCUCCCACCCCUCCCCG |
| | | | | CAGACCCCCUGUGGCCCCUCCCACCCAC |
| | | | | CCCAGAGUGAUCCCCGAAGUGUCCCACG |
| | | | | UGAGAGGCGUGACCGUGCACAUGGAAA |
| | | | | CCCCCGAAGCCAUCCUGUUCGCCCCCGG |
| | | | | CGAAACCUUCGGCACCAACGUGUCCAUC |
| | | | | CACGCCAUCGCCCACGAUGAUGGCCCCU |
| | | | | ACGCCAUGGAUGUGGUGUGGAUGAGAU |
| | | | | UCGAUGUGCCCUCCUCCUGCGCCGAAAU |
| | | | | GAGAAUCUACGAAGCCUGCCUGUACCAC |
| | | | | CCCCAGCUGCCCGAAUGCCUGUCCCCCG |
| | | | | CCGAUGCCCCCUGCGCCGUGUCCUCCUG |
| | | | | GGCCUACAGACUGGCCGUGAGAUCCUAC |
| | | | | GCCGGCUGCUCCAGAACCACCCCUCCCC |
| | | | | CUAGAUGCUUCGCCGAAGCCAGAAUGG |
| | | | | AACCCGUGCCCGGCCUGGCCUGGCUGGC |
| | | | | CUCCACCGUGAACCUGGAAUUCCAGCAC |
| | | | | GCCAGCCCCCAGCACGCCGGCCUGUACC |
| | | | | UGUGCGUGGUGUACGUGGAUGAUCACA |
| | | | | UCCACGCCUGGGGCCACAUGACCAUCUC |
| | | | | CACCGCCGCCCAGUACAGAAACGCCGUG |
| | | | | GUGGAACAGCACCUGCCCCAGAGACAGC |
| | | | | CCGAACCCGUGGAACCCACCAGACCCCA |
| | | | | CGUGAGAGCCUGA |
| 243 | HSV-2 gE | HSV-2 gE | Version 2 | AUGGCACGGGGAGCCGGAUUGGUGUUC |
| | | (24-405) | | UUUGUGGGCGUGUGGGUGGUGAGCUGC |
| | | | | UUGGCAGCCGCACCACGCACCUCUUGGA |
| | | | | AACGCGUUACUUCCGGGGAGGACGUUG |
| | | | | UCCUCCUUCCAGCACCCGCAGGACCUGA |
| | | | | GGAAAGGACUAGGGCCCACAAGCUGCU |
| | | | | GUGGGCCGCUGAACCUCUGGAUGCCUG |
| | | | | UGGUCCUCUGAGACCUAGCUGGGUCGCC |
| | | | | CUUUGGCCACCUAGACGCGUUCUGGAG |
| | | | | ACGGUCGUGGAUGCCGCGUGCAUGCGU |
| | | | | GCACCCGAACCUCUGGCCAUCGCCUAUA |
| | | | | GUCCCCCUUUUCCCGCUGGCGACGAGGG |
| | | | | GCUUUACUCCGAACUGGCCUGGCGGGA |
| | | | | UAGGGUGGCGGUGGUGAACGAGAGCCU |
| | | | | CGUCAUCUACGGUGCUCUGGAAACCGAC |
| | | | | UCAGGACUGUAUACGCUCAGCGUUGUU |
| | | | | GGCCUCUCCGAUGAGGCUCGACAGGUU |
| | | | | GCCUCCGUAGUGCUGGUCGUAGAACCA |
| | | | | GCCCCCGUACCAACACCCACACCCGACG |
| | | | | ACUACGACGAGGAGGACGACGCUGGAG |
| | | | | UUAGCGAAAGAACACCGGUGAGUGUGC |
| | | | | CACCUCCCACACCGCCAAGGAGACCCCC |
| | | | | AGUAGCACCUCCAACCCAUCCGAGAGUG |
| | | | | AUUCCCGAGGUCAGCCAUGUGCGCGGCG |
| | | | | UAACUGUGCACAUGGAGACGCCCGAAG |
| | | | | CGAUACUGUUUGCCCCUGGAGAGACAU |
| | | | | UCGGCACCAAUGUGUCCAUACACGCAAU |
| | | | | UGCGCACGAUGAUGGCCCAUACGCUAU |
| | | | | GGACGUCGUCUGGAUGAGGUUCGAUGU |
| | | | | GCCUUCUUCUUGCGCCGAGAUGAGGAU |
| | | | | CUACGAGGCAUGCCUGUAUCACCCCCAA |
| | | | | UUGCCGGAGUGUCUGUCUCCCGCAGAU |
| | | | | GCACCGUGUGCAGUGAGUAGCUGGGCU |
| | | | | UAUCGGUUGGCUGUCCGGAGUUAUGCU |
| | | | | GGGUGUUCACGGACCACCCCACCUCCAC |
| | | | | GUUGCUUUGCUGAAGCCAGAAUGGAAC |
| | | | | CCGUGCCUGGUCUGGCUUGGCUGGCAUC |
| | | | | AACUGUCAACCUGGAGUUCCAGCAUGCC |
| | | | | UCUCCACAGCACGCAGGCCUGUAUCUCU |
| | | | | GCGUGGUGUACGUUGACGAUCACAUCC |
| | | | | AUGCGUGGGGGCAUAUGACCAUCAGCA |
| | | | | CAGCUGCCCAGUACCGCAAUGCCGUCGU |
| | | | | GGAGCAGCACCUCCCCCAACGGCAGCCA |
| | | | | GAACCAGUGGAGCCCACUCGGCCUCAUG |
| | | | | UGCGAGCCUGA |
| 244 | HSV-2 gE | HSV-2 gE | Version | AUGGCACGGGGCGCAGGUUUGGUCUUU |
| | | (24-405) | 2.1 | UUCGUGGGCGUGUGGGUGGUGAGCUGC |
| | | | | UUGGCAGCCGCACCACGCACCUCUUGGA |
| | | | | AACGCGUUACUUCCGGGGAGGACGUUG |
| | | | | UCCUCCUUCCAGCACCCGCAGGACCUGA |
| | | | | GGAAAGGACUAGGGCCCACAAGCUGCU |
| | | | | GUGGGCCGCUGAACCUCUGGAUGCCUG |
| | | | | UGGUCCUCUGAGACCUAGCUGGGUCGCC |
| | | | | CUUUGGCCACCUAGACGCGUUCUGGAG |
| | | | | ACGGUCGUGGAUGCCGCGUGCAUGCGU |
| | | | | GCACCCGAACCUCUGGCCAUCGCCUAUA |
| | | | | GUCCCCCUUUUCCCGCUGGCGACGAGGG |
| | | | | GCUUUACUCCGAACUGGCCUGGCGGGA |
| | | | | UAGGGUGGCGGUGGUGAACGAGAGCCU |
| | | | | CGUCAUCUACGGUGCUCUGGAAACCGAC |
| | | | | UCAGGACUGUAUACGCUCAGCGUUGUU |
| | | | | GGCCUCUCCGAUGAGGCUCGACAGGUU |
| | | | | GCCUCCGUAGUGCUGGUCGUAGAACCA |
| | | | | GCCCCCGUACCAACACCCACACCCGACG |
| | | | | ACUACGACGAGGAGGACGACGCUGGAG |
| | | | | UUAGCGAAAGAACACCGGUGAGUGUGC |
| | | | | CACCUCCCACACCGCCAAGGAGACCCCC |
| | | | | AGUAGCACCUCCAACCCAUCCGAGAGUG |
| | | | | AUUCCCGAGGUCAGCCAUGUGCGCGGCG |
| | | | | UAACUGUGCACAUGGAGACGCCCGAAG |
| | | | | CGAUACUGUUUGCCCCUGGAGAGACAU |
| | | | | UCGGCACCAAUGUGUCCAUACACGCAAU |
| | | | | UGCGCACGAUGAUGGCCCAUACGCUAU |
| | | | | GGACGUCGUCUGGAUGAGGUUCGAUGU |
| | | | | GCCUUCUUCUUGCGCCGAGAUGAGGAU |
| | | | | CUACGAGGCAUGCCUGUAUCACCCCCAA |
| | | | | UUGCCGGAGUGUCUGUCUCCCGCAGAU |
| | | | | GCACCGUGUGCAGUGAGUAGCUGGGCU |
| | | | | UAUCGGUUGGCUGUCCGGAGUUAUGCU |
| | | | | GGGUGUUCACGGACCACCCCACCUCCAC |
| | | | | GUUGCUUUGCUGAAGCCAGAAUGGAAC |
| | | | | CCGUGCCUGGUCUGGCUUGGCUGGCAUC |
| | | | | AACUGUCAACCUGGAGUUCCAGCAUGCC |
| | | | | UCUCCACAGCACGCAGGCCUGUAUCUCU |
| | | | | GCGUGGUGUACGUUGACGAUCACAUCC |
| | | | | AUGCGUGGGGGCAUAUGACCAUCAGCA |
| | | | | CAGCUGCCCAGUACCGCAAUGCCGUCGU |
| | | | | GGAGCAGCACCUCCCCCAACGGCAGCCA |
| | | | | GAACCAGUGGAGCCCACUCGGCCUCAUG |
| | | | | UGCGAGCCUGA |
| 245 | HSV-2 gE | HSV-2 gE | Version | AUGGCGAGAGGAGCCGGACUCGUGUUC |
| | | (24-405) | 2.2 | UUUGUGGGAGUCUGGGUUGUGAGCUGC |
| | | | | CUGGCAGCAGCUCCACGCACCUCUUGGA |
| | | | | AACGCGUUACUUCCGGGGAGGACGUUG |
| | | | | UCCUCCUUCCAGCACCCGCAGGACCUGA |
| | | | | AGAGAGGACUAGGGCCCACAAGCUGCU |
| | | | | GUGGGCCGCUGAACCUCUGGAUGCCUG |
| | | | | UGGUCCUCUGAGACCUAGCUGGGUCGCC |
| | | | | CUUUGGCCACCUAGACGCGUUCUGGAG |
| | | | | ACGGUCGUGGAUGCCGCGUGCAUGCGU |
| | | | | GCACCCGAACCUCUGGCCAUCGCCUAUA |
| | | | | GUCCCCCUUUUCCCGCUGGCGACGAGGG |
| | | | | GCUUUACUCCGAACUGGCCUGGCGGGA |
| | | | | UAGGGUGGCGGUGGUGAACGAGAGCCU |
| | | | | CGUCAUCUACGGUGCUCUGGAAACCGAC |
| | | | | UCAGGACUGUAUACGCUCAGCGUUGUU |
| | | | | GGCCUCUCCGAUGAGGCUCGACAGGUU |
| | | | | GCCUCCGUAGUGCUGGUCGUAGAACCA |
| | | | | GCCCCCGUACCAACACCCACACCCGACG |
| | | | | ACUACGACGAAGAGGACGACGCUGGAG |
| | | | | UUAGCGAAAGAACACCGGUGAGUGUGC |
| | | | | CACCUCCCACACCGCCAAGGAGACCCCC |
| | | | | AGUAGCACCUCCAACCCAUCCGAGAGUG |
| | | | | AUUCCCGAGGUCAGCCAUGUGCGCGGCG |
| | | | | UAACUGUGCACAUGGAGACGCCCGAAG |
| | | | | CGAUACUGUUUGCCCCUGGAGAGACAU |
| | | | | UCGGCACCAAUGUGUCCAUACACGCAAU |
| | | | | UGCGCACGAUGAUGGCCCAUACGCUAU |
| | | | | GGACGUCGUCUGGAUGAGGUUCGAUGU |
| | | | | GCCUUCUUCUUGCGCCGAGAUGAGGAU |
| | | | | CUACGAGGCAUGCCUGUAUCACCCCCAA |
| | | | | UUGCCGGAGUGUCUGUCUCCCGCAGAU |
| | | | | GCACCGUGUGCAGUGAGUAGCUGGGCU |
| | | | | UAUCGGUUGGCUGUCCGGAGUUAUGCU |
| | | | | GGGUGUUCACGGACCACCCCACCUCCAC |
| | | | | GUUGCUUUGCUGAAGCCAGAAUGGAAC |
| | | | | CCGUGCCUGGUCUGGCUUGGCUGGCAUC |
| | | | | AACUGUCAACCUGGAGUUCCAGCAUGCC |
| | | | | UCUCCACAGCACGCAGGCCUGUAUCUCU |
| | | | | GCGUGGUGUACGUUGACGAUCACAUCC |
| | | | | AUGCGUGGGGGCAUAUGACCAUCAGCA |
| | | | | CAGCUGCCCAGUACCGCAAUGCCGUCGU |
| | | | | GGAGCAGCACCUCCCCCAACGGCAGCCA |
| | | | | GAACCAGUGGAGCCCACUCGGCCUCAUG |
| | | | | UGCGAGCCUGA |
| 246 | HSV-2 gE | HSV-2 gE | Version | AUGGCGAGAGGAGCCGGGCUCGUGUUC |
| | | (24-405) | 2.3 | UUUGUGGGCGUAUGGGUCGUUUCCUGC |
| | | | | CUGGCUGCCGCACCCCGCACCUCUUGGA |
| | | | | AACGCGUUACUUCCGGGGAGGACGUUG |
| | | | | UCCUCCUUCCAGCACCCGCAGGACCUGA |
| | | | | GGAAAGGACUAGGGCCCACAAGCUGCU |
| | | | | GUGGGCCGCUGAACCUCUGGAUGCCUG |
| | | | | UGGUCCUCUGAGACCUAGCUGGGUCGCC |
| | | | | CUUUGGCCACCUAGACGCGUUCUGGAG |
| | | | | ACGGUCGUGGAUGCCGCGUGCAUGCGU |
| | | | | GCACCCGAACCUCUGGCCAUCGCCUAUA |
| | | | | GUCCCCCUUUUCCCGCUGGCGACGAGGG |
| | | | | GCUUUACUCCGAACUGGCCUGGCGGGA |
| | | | | UAGGGUGGCGGUGGUGAACGAGAGCCU |
| | | | | CGUCAUCUACGGUGCUCUGGAAACCGAC |
| | | | | UCAGGACUGUAUACGCUCAGCGUUGUU |
| | | | | GGCCUCUCCGAUGAGGCUCGACAGGUU |
| | | | | GCCUCCGUAGUGCUGGUCGUAGAACCA |
| | | | | GCCCCCGUACCAACACCCACACCCGACG |
| | | | | ACUACGACGAGGAGGACGACGCUGGAG |
| | | | | UUAGCGAAAGAACACCGGUGAGUGUGC |
| | | | | CACCUCCCACACCGCCAAGGAGACCCCC |
| | | | | AGUAGCACCUCCAACCCAUCCGAGAGUG |
| | | | | AUUCCCGAGGUCAGCCAUGUGCGCGGCG |
| | | | | UAACUGUGCACAUGGAGACGCCCGAAG |
| | | | | CGAUACUGUUUGCCCCUGGAGAGACAU |
| | | | | UCGGCACCAAUGUGUCCAUACACGCAAU |
| | | | | UGCGCACGAUGAUGGCCCAUACGCUAU |
| | | | | GGACGUCGUCUGGAUGAGGUUCGAUGU |
| | | | | GCCUUCUUCUUGCGCCGAGAUGAGGAU |
| | | | | CUACGAGGCAUGCCUGUAUCACCCCCAA |
| | | | | UUGCCGGAGUGUCUGUCUCCCGCAGAU |
| | | | | GCACCGUGUGCAGUGAGUAGCUGGGCU |
| | | | | UAUCGGUUGGCUGUCCGGAGUUAUGCU |
| | | | | GGGUGUUCACGGACCACCCCACCUCCAC |
| | | | | GUUGCUUUGCUGAAGCCAGAAUGGAAC |
| | | | | CCGUGCCUGGUCUGGCUUGGCUGGCAUC |
| | | | | AACUGUCAACCUGGAGUUCCAGCAUGCC |
| | | | | UCUCCACAGCACGCAGGCCUGUAUCUCU |
| | | | | GCGUGGUGUACGUUGACGAUCACAUCC |
| | | | | AUGCGUGGGGGCAUAUGACCAUCAGCA |
| | | | | CAGCUGCCCAGUACCGCAAUGCCGUCGU |
| | | | | GGAGCAGCACCUCCCCCAACGGCAGCCA |
| | | | | GAACCAGUGGAGCCCACUCGGCCUCAUG |
| | | | | UGCGAGCCUGA |
| 247 | HSV-2 gE- | HSV-2 gE | Version 2 | AUGGCGAGAGGAGCCGGACUCGUGUUC |
| | LVVVP | (24-405) | | UUUGUGGGAGUCUGGGUUGUGAGCUGC |
| | | | | CUGGUAGUAGUUCCACGCACCUCUUGG |
| | | | | AAACGCGUUACUUCCGGGGAGGACGUU |
| | | | | GUCCUCCUUCCAGCACCCGCAGGACCUG |
| | | | | AAGAGAGGACUAGGGCCCACAAGCUGC |
| | | | | UGUGGGCCGCUGAACCUCUGGAUGCCU |
| | | | | GUGGUCCUCUGAGACCUAGCUGGGUCG |
| | | | | CCCUUUGGCCACCUAGACGCGUUCUGGA |
| | | | | GACGGUCGUGGAUGCCGCGUGCAUGCG |
| | | | | UGCACCCGAACCUCUGGCCAUCGCCUAU |
| | | | | AGUCCCCCUUUUCCCGCUGGCGACGAGG |
| | | | | GGCUUUACUCCGAACUGGCCUGGCGGG |
| | | | | AUAGGGUGGCGGUGGUGAACGAGAGCC |
| | | | | UCGUCAUCUACGGUGCUCUGGAAACCG |
| | | | | ACUCAGGACUGUAUACGCUCAGCGUUG |
| | | | | UUGGCCUCUCCGAUGAGGCUCGACAGG |
| | | | | UUGCCUCCGUAGUGCUGGUCGUAGAAC |
| | | | | CAGCCCCCGUACCAACACCCACACCCGA |
| | | | | CGACUACGACGAAGAGGACGACGCUGG |
| | | | | AGUUAGCGAAAGAACACCGGUGAGUGU |
| | | | | GCCACCUCCCACACCGCCAAGGAGACCC |
| | | | | CCAGUAGCACCUCCAACCCAUCCGAGAG |
| | | | | UGAUUCCCGAGGUCAGCCAUGUGCGCG |
| | | | | GCGUAACUGUGCACAUGGAGACGCCCG |
| | | | | AAGCGAUACUGUUUGCCCCUGGAGAGA |
| | | | | CAUUCGGCACCAAUGUGUCCAUACACGC |
| | | | | AAUUGCGCACGAUGAUGGCCCAUACGC |
| | | | | UAUGGACGUCGUCUGGAUGAGGUUCGA |
| | | | | UGUGCCUUCUUCUUGCGCCGAGAUGAG |
| | | | | GAUCUACGAGGCAUGCCUGUAUCACCCC |
| | | | | CAAUUGCCGGAGUGUCUGUCUCCCGCAG |
| | | | | AUGCACCGUGUGCAGUGAGUAGCUGGG |
| | | | | CUUAUCGGUUGGCUGUCCGGAGUUAUG |
| | | | | CUGGGUGUUCACGGACCACCCCACCUCC |
| | | | | ACGUUGCUUUGCUGAAGCCAGAAUGGA |
| | | | | ACCCGUGCCUGGUCUGGCUUGGCUGGCA |
| | | | | UCAACUGUCAACCUGGAGUUCCAGCAU |
| | | | | GCCUCUCCACAGCACGCAGGCCUGUAUC |
| | | | | UCUGCGUGGUGUACGUUGACGAUCACA |
| | | | | UCCAUGCGUGGGGGCAUAUGACCAUCA |
| | | | | GCACAGCUGCCCAGUACCGCAAUGCCGU |
| | | | | CGUGGAGCAGCACCUCCCCCAACGGCAG |
| | | | | CCAGAACCAGUGGAGCCCACUCGGCCUC |
| | | | | AUGUGCGAGCCUGA |
| 248 | None | HSV-2 gE | Version 2 | AUGCGCACCUCUUGGAAACGCGUUACU |
| | (N- | (24-405) | | UCCGGGGAGGACGUUGUCCUCCUUCCAG |
| | terminal | | | CACCCGCAGGACCUGAGGAAAGGACUA |
| | Met) | | | GGGCCCACAAGCUGCUGUGGGCCGCUGA |
| | | | | ACCUCUGGAUGCCUGUGGUCCUCUGAG |
| | | | | ACCUAGCUGGGUCGCCCUUUGGCCACCU |
| | | | | AGACGCGUUCUGGAGACGGUCGUGGAU |
| | | | | GCCGCGUGCAUGCGUGCACCCGAACCUC |
| | | | | UGGCCAUCGCCUAUAGUCCCCCUUUUCC |
| | | | | CGCUGGCGACGAGGGGCUUUACUCCGA |
| | | | | ACUGGCCUGGCGGGAUAGGGUGGCGGU |
| | | | | GGUGAACGAGAGCCUCGUCAUCUACGG |
| | | | | UGCUCUGGAAACCGACUCAGGACUGUA |
| | | | | UACGCUCAGCGUUGUUGGCCUCUCCGAU |
| | | | | GAGGCUCGACAGGUUGCCUCCGUAGUG |
| | | | | CUGGUCGUAGAACCAGCCCCCGUACCAA |
| | | | | CACCCACACCCGACGACUACGACGAGGA |
| | | | | GGACGACGCUGGAGUUAGCGAAAGAAC |
| | | | | ACCGGUGAGUGUGCCACCUCCCACACCG |
| | | | | CCAAGGAGACCCCCAGUAGCACCUCCAA |
| | | | | CCCAUCCGAGAGUGAUUCCCGAGGUCAG |
| | | | | CCAUGUGCGCGGCGUAACUGUGCACAU |
| | | | | GGAGACGCCCGAAGCGAUACUGUUUGC |
| | | | | CCCUGGAGAGACAUUCGGCACCAAUGU |
| | | | | GUCCAUACACGCAAUUGCGCACGAUGA |
| | | | | UGGCCCAUACGCUAUGGACGUCGUCUG |
| | | | | GAUGAGGUUCGAUGUGCCUUCUUCUUG |
| | | | | CGCCGAGAUGAGGAUCUACGAGGCAUG |
| | | | | CCUGUAUCACCCCCAAUUGCCGGAGUGU |
| | | | | CUGUCUCCCGCAGAUGCACCGUGUGCAG |
| | | | | UGAGUAGCUGGGCUUAUCGGUUGGCUG |
| | | | | UCCGGAGUUAUGCUGGGUGUUCACGGA |
| | | | | CCACCCCACCUCCACGUUGCUUUGCUGA |
| | | | | AGCCAGAAUGGAACCCGUGCCUGGUCU |
| | | | | GGCUUGGCUGGCAUCAACUGUCAACCU |
| | | | | GGAGUUCCAGCAUGCCUCUCCACAGCAC |
| | | | | GCAGGCCUGUAUCUCUGCGUGGUGUAC |
| | | | | GUUGACGAUCACAUCCAUGCGUGGGGG |
| | | | | CAUAUGACCAUCAGCACAGCUGCCCAGU |
| | | | | ACCGCAAUGCCGUCGUGGAGCAGCACCU |
| | | | | CCCCCAACGGCAGCCAGAACCAGUGGAG |
| | | | | CCCACUCGGCCUCAUGUGCGAGCCUGA |
| 249 | HSV-1 gD- | HSV-2 gE | Version 1 | AUGGGCGGAGCUGCUGCUAGACUGGGA |
| | KY | (24-405) | | GCCGUGAUCCUGUUCGUGGUUAUCGUG |
| | | | | GGACUGCAUGGCGUGCGGGGCAAGUAU |
| | | | | AGAACCUCUUGGAAGCGCGUGACAAGC |
| | | | | GGCGAGGAUGUGGUUCUGCUUCCUGCU |
| | | | | CCUGCCGGACCUGAGGAAAGAACAAGA |
| | | | | GCCCACAAGCUGCUGUGGGCCGCUGAAC |
| | | | | CUUUGGAUGCCUGUGGACCUCUGAGGC |
| | | | | CUUCUUGGGUUGCACUGUGGCCACCUCG |
| | | | | GAGAGUGCUGGAAACAGUGGUGGAUGC |
| | | | | CGCCUGCAUGAGAGCCCCUGAACCUCUG |
| | | | | GCCAUUGCCUACUCUCCACCUUUUCCAG |
| | | | | CCGGCGACGAGGGCCUGUAUUCUGAGC |
| | | | | UUGCUUGGAGAGACAGAGUGGCCGUGG |
| | | | | UCAACGAGAGCCUGGUUAUCUAUGGCG |
| | | | | CCCUGGAAACCGACAGCGGCCUGUACAC |
| | | | | ACUGUCUGUCGUGGGCCUGUCUGACGA |
| | | | | GGCUAGACAGGUGGCAUCUGUGGUGCU |
| | | | | GGUGGUGGAACCUGCUCCAGUGCCUAC |
| | | | | ACCUACACCUGACGACUACGACGAGGAA |
| | | | | GAUGACGCUGGCGUCAGCGAGAGAACC |
| | | | | CCUGUUUCUGUGCCUCCUCCUACGCCUC |
| | | | | CUCGUAGACCUCCUGUUGCUCCUCCAAC |
| | | | | ACACCCCAGAGUGAUCCCUGAAGUGUCU |
| | | | | CACGUGCGGGGCGUGACCGUGCACAUG |
| | | | | GAAACACCUGAGGCCAUCCUGUUCGCCC |
| | | | | CUGGCGAGACAUUUGGCACCAACGUGU |
| | | | | CCAUCCACGCUAUCGCCCACGACGAUGG |
| | | | | CCCUUACGCCAUGGAUGUCGUGUGGAU |
| | | | | GAGAUUCGACGUGCCCAGCAGCUGCGCC |
| | | | | GAGAUGAGAAUCUACGAGGCCUGCCUG |
| | | | | UAUCACCCUCAGCUGCCCGAAUGUCUGA |
| | | | | GCCCUGCUGAUGCCCCUUGUGCCGUUAG |
| | | | | CAGCUGGGCCUAUAGACUGGCCGUGCG |
| | | | | GUCUUAUGCCGGCUGCUCUAGAACAACC |
| | | | | CCUCCUCCUCGGUGUUUCGCCGAGGCCA |
| | | | | GAAUGGAACCUGUUCCUGGACUGGCCU |
| | | | | GGCUGGCCUCCACAGUGAACCUGGAAU |
| | | | | UUCAGCACGCCUCUCCACAGCACGCCGG |
| | | | | CCUGUAUCUGUGUGUGGUGUACGUGGA |
| | | | | CGAUCACAUCCACGCCUGGGGCCACAUG |
| | | | | ACCAUCUCUACAGCCGCUCAGUACCGGA |
| | | | | ACGCCGUGGUUGAACAGCAUCUGCCCCA |
| | | | | GAGACAGCCCGAGCCUGUGGAACCUACA |
| | | | | AGACCUCAUGUUCGGGCCUGAUAA |
| 250 | HSV-1 gD- | HSV-2 gE | Version 2 | AUGGGCGGAGCUGCGGCUCGCCUCGGA |
| | KY | (24-405) | | GCCGUCAUUCUGUUCGUGGUGAUCGUG |
| | | | | GGUCUGCAUGGGGUCAGAGGAAAGUAC |
| | | | | CGGACCAGCUGGAAAAGGGUGACAUCU |
| | | | | GGGGAAGAUGUCGUGCUCCUUCCUGCG |
| | | | | CCUGCUGGCCCAGAAGAACGCACUAGGG |
| | | | | CCCACAAGCUUCUGUGGGCCGCGGAGCC |
| | | | | ACUGGAUGCGUGUGGUCCCCUGCGACCC |
| | | | | UCUUGGGUGGCAUUGUGGCCACCCCGAC |
| | | | | GCGUACUCGAAACGGUCGUUGACGCCGC |
| | | | | CUGUAUGAGAGCGCCAGAGCCCCUCGCC |
| | | | | AUUGCCUACAGCCCGCCUUUUCCCGCCG |
| | | | | GUGACGAAGGACUGUACAGUGAGCUGG |
| | | | | CCUGGCGCGAUAGGGUCGCCGUAGUUA |
| | | | | ACGAGUCCCUGGUGAUAUACGGCGCUC |
| | | | | UGGAAACCGACAGUGGCUUGUACACCC |
| | | | | UGUCCGUUGUAGGCCUGUCCGAUGAAG |
| | | | | CACGGCAAGUGGCUUCCGUGGUACUGG |
| | | | | UCGUAGAGCCCGCACCAGUUCCCACACC |
| | | | | CACCCCGGAUGACUACGAUGAGGAGGA |
| | | | | CGAUGCCGGUGUGAGUGAGCGUACACC |
| | | | | UGUUAGCGUACCUCCACCAACUCCUCCA |
| | | | | CGCCGCCCUCCUGUUGCACCGCCUACAC |
| | | | | AUCCCCGUGUGAUUCCUGAAGUGUCAC |
| | | | | ACGUUAGAGGGGUGACAGUCCACAUGG |
| | | | | AGACUCCCGAAGCCAUCCUCUUUGCACC |
| | | | | AGGCGAGACUUUUGGGACCAAUGUGAG |
| | | | | CAUUCACGCCAUAGCUCACGAUGACGGG |
| | | | | CCCUAUGCCAUGGACGUGGUGUGGAUG |
| | | | | AGGUUCGAUGUGCCCUCAUCAUGCGCU |
| | | | | GAGAUGCGGAUCUACGAAGCUUGCCUG |
| | | | | UAUCACCCACAGCUUCCCGAGUGCUUGU |
| | | | | CUCCCGCUGACGCCCCUUGUGCUGUUAG |
| | | | | CUCUUGGGCUUAUCGGCUUGCCGUCAG |
| | | | | GAGCUAUGCUGGAUGCUCCAGAACCAC |
| | | | | ACCUCCACCGAGGUGUUUCGCCGAGGCC |
| | | | | AGAAUGGAGCCUGUGCCAGGACUGGCC |
| | | | | UGGCUGGCAAGUACUGUGAACCUGGAA |
| | | | | UUCCAGCACGCAUCACCUCAGCAUGCAG |
| | | | | GCCUGUACCUCUGCGUUGUCUAUGUCG |
| | | | | ACGACCAUAUCCACGCAUGGGGCCAUAU |
| | | | | GACCAUCAGCACGGCAGCACAGUAUCGG |
| | | | | AAUGCUGUGGUCGAGCAGCACUUGCCCC |
| | | | | AGCGACAACCCGAACCAGUGGAGCCAAC |
| | | | | CAGACCGCAUGUGCGGGCCUGA |
| 251 | HSV-1 gD- | HSV-2 gE | Version 3 | AUGGGAGGAGCUGCUGCUAGAUUGGGA |
| | KY | (24-405) | | GCUGUGAUUCUGUUUGUGGUGAUUGUG |
| | | | | GGACUGCAUGGAGUGAGAGGAAAAUAC |
| | | | | AGAACCUCUUGGAAAAGAGUGACCUCU |
| | | | | GGAGAAGAUGUGGUGCUGCUGCCAGCU |
| | | | | CCAGCUGGACCAGAAGAAAGAACCAGA |
| | | | | GCACACAAACUGCUGUGGGCUGCUGAA |
| | | | | CCUCUGGAUGCUUGUGGACCUCUGAGA |
| | | | | CCUUCUUGGGUGGCUCUGUGGCCACCAA |
| | | | | GAAGGGUGCUGGAAACAGUGGUGGAUG |
| | | | | CUGCUUGCAUGAGAGCACCUGAACCUCU |
| | | | | GGCAAUUGCAUACUCUCCUCCUUUUCCA |
| | | | | GCUGGAGAUGAAGGACUGUAUUCUGAA |
| | | | | CUGGCUUGGAGAGACAGAGUGGCUGUG |
| | | | | GUGAAUGAAUCUCUGGUGAUCUAUGGA |
| | | | | GCACUGGAAACAGAUUCUGGACUGUAC |
| | | | | ACCCUGUCUGUGGUGGGACUGUCUGAU |
| | | | | GAAGCAAGACAGGUGGCAUCUGUGGUG |
| | | | | CUGGUGGUGGAACCAGCUCCAGUGCCA |
| | | | | ACCCCAACCCCAGAUGAUUAUGAUGAA |
| | | | | GAAGAUGAUGCUGGAGUGUCUGAAAGA |
| | | | | ACCCCAGUGUCUGUGCCACCACCAACCC |
| | | | | CACCAAGAAGACCACCAGUGGCUCCACC |
| | | | | AACCCACCCAAGAGUGAUCCCAGAAGUG |
| | | | | UCUCAUGUGAGAGGAGUGACAGUGCAC |
| | | | | AUGGAAACCCCUGAAGCAAUCCUGUUU |
| | | | | GCACCUGGAGAAACCUUUGGAACCAAU |
| | | | | GUGUCCAUCCAUGCAAUUGCACAUGAU |
| | | | | GAUGGACCUUAUGCAAUGGAUGUGGUG |
| | | | | UGGAUGAGAUUUGAUGUGCCUUCUUCU |
| | | | | UGUGCUGAAAUGAGAAUCUAUGAAGCU |
| | | | | UGCCUGUACCACCCUCAGCUGCCUGAAU |
| | | | | GCCUGUCUCCUGCUGAUGCUCCUUGUGC |
| | | | | UGUGUCUUCUUGGGCUUACAGACUGGC |
| | | | | UGUGAGAUCUUAUGCUGGAUGCUCCAG |
| | | | | AACCACCCCACCACCAAGAUGCUUUGCU |
| | | | | GAAGCAAGAAUGGAACCUGUGCCUGGA |
| | | | | CUGGCAUGGCUGGCAUCCACAGUGAAU |
| | | | | CUGGAAUUUCAGCAUGCUUCUCCUCAGC |
| | | | | AUGCUGGACUGUACCUGUGUGUGGUGU |
| | | | | AUGUGGACGAUCACAUCCAUGCUUGGG |
| | | | | GACACAUGACCAUCAGCACAGCUGCUCA |
| | | | | GUACAGAAAUGCUGUGGUGGAACAGCA |
| | | | | CCUGCCACAGAGACAGCCAGAACCAGUG |
| | | | | GAACCAACCAGACCACAUGUGAGAGCU |
| | | | | UGAUAA |
| 252 | HSV-1 gD- | HSV-2 gE | Version 4 | AUGGGCGGAGCUGCGGCUCGCCUCGGA |
| | KY | (24-405) | | GCCGUCAUUCUGUUCGUGGUGAUCGUG |
| | | | | GGUCUGCAUGGGGUCAGAGGAAAGUAC |
| | | | | CGGACCAGCUGGAAAAGGGUGACAUCU |
| | | | | GGGGAAGAUGUCGUGCUCCUUCCUGCG |
| | | | | CCUGCUGGCCCAGAAGAACGCACUAGGG |
| | | | | CCCACAAGCUUCUGUGGGCCGCGGAGCC |
| | | | | ACUGGAUGCGUGUGGUCCCCUGCGACCC |
| | | | | UCUUGGGUGGCAUUGUGGCCACCCCGAC |
| | | | | GCGUACUCGAAACGGUCGUUGACGCCGC |
| | | | | CUGUAUGAGAGCGCCAGAGCCCCUCGCC |
| | | | | AUUGCCUACAGCCCGCCUUUUCCCGCCG |
| | | | | GUGACGAAGGACUGUACAGUGAGCUGG |
| | | | | CCUGGCGCGAUAGGGUCGCCGUAGUUA |
| | | | | ACGAGUCCCUGGUGAUAUACGGCGCUC |
| | | | | UGGAAACCGACAGUGGCUUGUACACCC |
| | | | | UGUCCGUUGUAGGCCUGUCCGAUGAAG |
| | | | | CACGGCAAGUGGCUUCCGUGGUACUGG |
| | | | | UCGUAGAGCCCGCACCAGUUCCCACACC |
| | | | | CACCCCGGAUGACUACGAUGAGGAGGA |
| | | | | CGAUGCCGGUGUGAGUGAGCGUACACC |
| | | | | UGUUAGCGUACCUCCACCAACUCCUCCA |
| | | | | CGCCGCCCUCCUGUUGCACCGCCUACAC |
| | | | | AUCCCCGUGUGAUUCCUGAAGUGUCAC |
| | | | | ACGUUAGAGGGGUGACAGUCCACAUGG |
| | | | | AGACUCCCGAAGCCAUCCUCUUUGCACC |
| | | | | AGGCGAGACUUUUGGGACCAAUGUGAG |
| | | | | CAUUCACGCCAUAGCUCACGAUGACGGG |
| | | | | CCCUAUGCCAUGGACGUGGUGUGGAUG |
| | | | | AGGUUCGAUGUGCCCUCAUCAUGCGCU |
| | | | | GAGAUGCGGAUCUACGAAGCUUGCCUG |
| | | | | UAUCACCCACAGCUUCCCGAGUGCUUGU |
| | | | | CUCCCGCUGACGCCCCUUGUGCUGUUAG |
| | | | | CUCUUGGGCUUAUCGGCUUGCCGUCAG |
| | | | | GAGCUAUGCUGGAUGCUCCAGAACCAC |
| | | | | ACCUCCACCGAGGUGUUUCGCCGAGGCC |
| | | | | AGAAUGGAGCCUGUGCCAGGACUGGCC |
| | | | | UGGCUGGCAAGUACUGUGAACCUGGAA |
| | | | | UUCCAGCACGCAUCACCUCAGCAUGCAG |
| | | | | GCCUGUACCUCUGCGUUGUCUAUGUCG |
| | | | | ACGACCAUAUCCACGCAUGGGGCCAUAU |
| | | | | GACCAUCAGCACGGCAGCACAGUAUCGG |
| | | | | AAUGCUGUGGUCGAGCAGCACUUGCCCC |
| | | | | AGCGACAACCCGAACCAGUGGAGCCAAC |
| | | | | CAGACCGCAUGUGCGGGCCUGA |

### Exemplary Nucleotide Sequence Features

In some embodiments, an RNA provided herein comprises a nucleotide sequence that encodes an HSV-2 gC protein or immunogenic fragment thereof and a signal sequence. In some embodiments, an RNA provided herein comprises a nucleotide sequence that encodes an HSV-2 gD protein or immunogenic fragment thereof and a signal sequence. In some embodiments, an RNA provided herein comprises a nucleotide sequence that encodes an HSV-2 gE protein or immunogenic fragment thereof and a signal sequence.

In some embodiments, nucleotide sequences described herein can comprise a nucleotide sequence that encodes a 5′UTR and/or a 3′ UTR. In some embodiments, polynucleotides described herein can comprise a nucleotide sequence that encodes a polyA tail. In some embodiments, nucleotide sequences described herein may comprise a 5′ cap, which may be incorporated during transcription, or joined to a nucleotide sequence post-transcription.

In some embodiments, a nucleotide sequence provided herein encodes one or more glycoproteins (e.g., gC, gD, gE, or a combination thereof) or an immunogenic fragment thereof. In some embodiments, an RNA comprises a 5′ cap, a 5′UTR, a nucleotide sequence that encodes one or more glycoproteins (e.g., gC, gD, gE, or a combination thereof), or immunogenic fragment thereof, a 3′ UTR, and a polyA tail.

1. 5′ Cap

A structural feature of messenger RNA (mRNA) is a cap structure at the five-prime end (5′). Natural eukaryotic mRNA comprise a 7-methylguanosine cap linked to the mRNA via a 5′ to 5′-triphosphate bridge resulting in a cap0 structure (m7GpppN). In most eukaryotic mRNA and some viral mRNA, further modifications can occur at the 2′-hydroxyl-group (2′-OH) (e.g., the 2′-hydroxyl group may be methylated to form 2′-O-Me) of the first and subsequent nucleotides producing “cap1” and “cap2” five-prime ends, respectively). Diamond, et al., (2014) Cytokine & growth Factor Reviews, 25:543-550, which is incorporated herein by reference in its entirety, reported that cap0-mRNA cannot be translated as efficiently as cap1-mRNA in which the role of 2′-O-Me in the penultimate position at the mRNA 5′ end is determinant. Lack of the 2′-O-met has been shown to trigger innate immunity and activate an interferon (IFN) response. Daffis, et al. (2010) Nature, 468:452-456; and Züst et al. (2011) Nature Immunology, 12:137-143, each of which is incorporated herein by reference in its entirety.

RNA capping is well researched and is described, e.g., in Decroly E et al. (2012) Nature Reviews 10: 51-65; and in Ramanathan A. et al., (2016) Nucleic Acids Res; 44(16): 7511-7526, the entire contents of each of which are hereby incorporated by reference. For example, in some embodiments, a 5′-cap structure which may be suitable in the context of the present disclosure is a cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (“anti-reverse cap analogue”), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

The term “5′-cap” as used herein refers to a structure found on the 5′-end of an RNA, e.g., mRNA, and generally includes a guanosine nucleotide connected to an RNA, e.g., mRNA, via a 5′- to 5′-triphosphate linkage (also referred to as Gppp or G(5′)ppp(5′)). In some embodiments, a guanosine nucleoside included in a 5′ cap may be modified, for example, by methylation at one or more positions (e.g., at the 7-position) on a base (guanine), and/or by methylation at one or more positions of a ribose. In some embodiments, a guanosine nucleoside included in a 5′ cap comprises a 3′-O methylation at a ribose (3′-OMeG). In some embodiments, a guanosine nucleoside included in a 5′ cap comprises methylation at the 7-position of guanine (m7G). In some embodiments, a guanosine nucleoside included in a 5′ cap comprises methylation at the 7′-position of guanine and a 3′ 0 methylation at a ribose (m7(3′-OMeG)). It will be understood that the notation used in the above paragraph, e.g., “(m27,3′-O)G” or “m7(3′-OMeG)”, applies to other structures described herein.

In some embodiments, providing an RNA with a 5′-cap disclosed herein may be achieved by in vitro transcription, in which a 5′-cap is co-transcriptionally incorporated into an RNA strand, or may be attached to an RNA post-transcriptionally using capping enzymes. In some embodiments, co-transcriptional capping with a cap disclosed herein improves the capping efficiency of an RNA compared to co-transcriptional capping with an appropriate reference comparator. In some embodiments, improving capping efficiency can increase a translation efficiency and/or translation rate of an RNA, and/or increase expression of an encoded protein. In some embodiments, alterations to polynucleotides generate a non-hydrolyzable cap structure which can, for example, prevent decapping and increase RNA half-life.

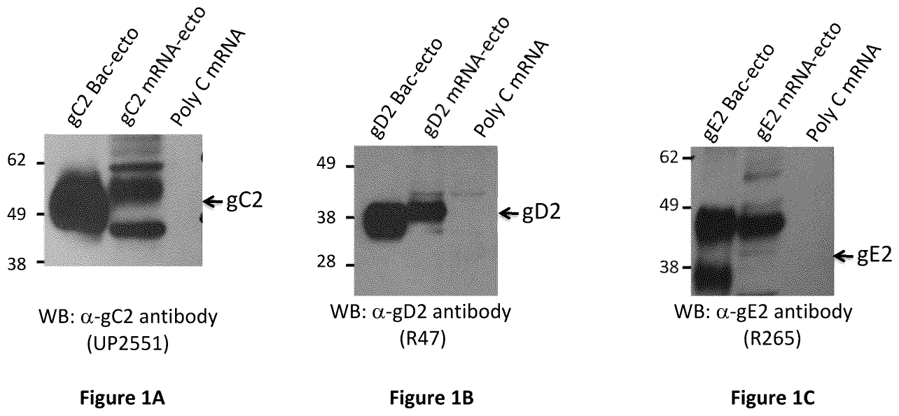

In some embodiments, a utilized 5′ cap is a cap0, a cap1, or cap2 structure. See, e.g., FIG. 1 of Ramanathan A et al., and FIG. 1 of Decroly E et al., each of which is incorporated herein by reference in its entirety. In some embodiments, an RNA described herein comprises a cap1 structure. In some embodiments, an RNA described herein comprises a cap2 structure.

In some embodiments, an RNA described herein comprises a cap0 structure. In some embodiments, a cap0 structure comprises a guanosine nucleoside methylated at the 7-position of guanine ((m7)G). In some embodiments, such a cap0 structure is connected to an RNA via a 5′- to 5′-triphosphate linkage and is also referred to herein as (m7)Gppp. In some embodiments, a cap0 structure comprises a guanosine nucleoside methylated at the 2′-position of the ribose of guanosine. In some embodiments, a cap0 structure comprises a guanosine nucleoside methylated at the 3′-position of the ribose of guanosine. In some embodiments, a guanosine nucleoside included in a 5′ cap comprises methylation at the 7-position of guanine and at the 2′-position of the ribose ((m27,2′-O)G). In some embodiments, a guanosine nucleoside included in a 5′ cap comprises methylation at the 7-position of guanine and at the 2′-position of the ribose ((m27,3′-O)G).

In some embodiments, a cap1 structure comprises a guanosine nucleoside methylated at the 7-position of guanine ((m7)G) and optionally methylated at the 2′ or 3′ position of the ribose, and a 2′O methylated first nucleotide in an RNA ((m2′-O)N1). In some embodiments, a cap1 structure comprises a guanosine nucleoside methylated at the 7-position of guanine ((m7)G) and the 3′ position of the ribose, and a 2′O methylated first nucleotide in an RNA ((m2′-O)N1). In some embodiments, a cap1 structure is connected to an RNA via a 5′- to linkage and is also referred to herein as, e.g., ((m7)Gppp(2′-O)N1) or (m27,3′-O)Gpp(2′-O)N1), wherein N1 is as defined and described herein. In some embodiments, a cap1 structure comprises a second nucleotide, N2, which is at position 2 and is chosen from A, G, C, or U, e.g., (m7)Gppp(2′-O)N1pN2 or (m27,3′-O)Gppp(2′-O)N1pN2, wherein each of N1 and N2 is as defined and described herein.

In some embodiments, a cap2 structure comprises a guanosine nucleoside methylated at the 7-position of guanine ((m7)G) and optionally methylated at the 2′ or 3′ position of the ribose, and 2′O methylated first and second nucleotides in an RNA ((m2′-O)N1p(m2′-O)N2. In some embodiments, a cap2 structure comprises a guanosine nucleoside methylated at the 7-position of guanine ((m7)G) and the 3′ position of the ribose, and 2′O methylated first and second nucleotides in an RNA. In some embodiments, a cap2 structure is connected to an RNA via a 5′- to 5′-triphosphate linkage and is also referred to herein as, e.g., ((m7)Gppp(2′-N1p(2′-O)N2) or (m27,3′-O)Gppp(2′-O)N1p(2′-O)N2), wherein each of N1 and N2 is as defined and described herein.

In some embodiments, the 5′ cap is a dinucleotide cap structure. In some embodiments, the 5′ cap is a dinucleotide cap structure comprising N1, wherein N1 is as defined and described herein. In some embodiments, the 5′ cap is a dinucleotide cap G*N1, wherein N1 is as defined above and herein, and G* comprises a structure of formula (I):

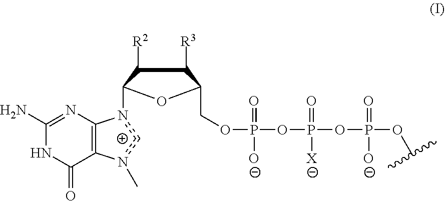

or a salt thereof,
wherein each R2 and R3 is —OH or —OCH3; and X is O or S.

In some embodiments, R2 is —OH. In some embodiments, R2 is —OCH3. In some embodiments, R3 is —OH. In some embodiments, R3 is —OCH3. In some embodiments, R2 is —OH and R3 is —OH. In some embodiments, R2 is —OH and R3 is —CH3. In some embodiments, R2 is —CH3 and R3 is —OH. In some embodiments, R2 is —CH3 and R3 is —CH3.

In some embodiments, X is 0. In some embodiments, X is S.

In some embodiments, the 5′ cap is a dinucleotide cap0 structure (e.g., (m7)GpppN1, (m27,2′-O)GpppN1, (m27,3′-O)GpppN1, (m7)GppSpN1, (m27,2′-O)GppSpN1, or (m27,3′-O)GppSpN1), wherein N1 is as defined and described herein. In some embodiments, the 5′ cap is a dinucleotide cap0 structure (e.g., (m7)GpppN1, (m27,2′-O)GpppN1, (m27,3′-(m7)GppSpN1, (m27,2′-O)GppSpN1, or (m27,3′-O)GppSpN1), wherein N1 is G. In some embodiments, the 5′ cap is a dinucleotide cap0 structure (e.g., (m7)GpppN1, (m27,2′-(m27,3′-O)GpppN1, (m7)GppSpN1, (m27,2′-O)GppSpN1, or (m27,3′-O)GppSp(m2′-O)N1), wherein N1 is A, U, or C. In some embodiments, the 5′ cap is a dinucleotide cap1 structure (e.g., (m7)Gppp(m2′-O)N1, (m27,2′-O)Gppp(m2′-O)N1, (m27,3′-O)Gppp(m2′-O)N1, (m7)GppSp(m2′-O)N1, (m27,2′-O)GppSp(m2′-O)N1, or (m27,3′-O)GppSp(m2′-O)N1), wherein N1 is as defined and described herein. In some embodiments, the 5′ cap is selected from the group consisting of (m7)GpppG (“Ecap0”), (m7)Gppp(m2′-O)G (“Ecap1”), (m27,3′-O)GpppG (“ARCA” or “Dl”), and (m27,2′-O)GppSpG (“beta-S-ARCA”). In some embodiments, the 5′ cap is (m7)GpppG (“Ecap0”), having a structure of formula (II):

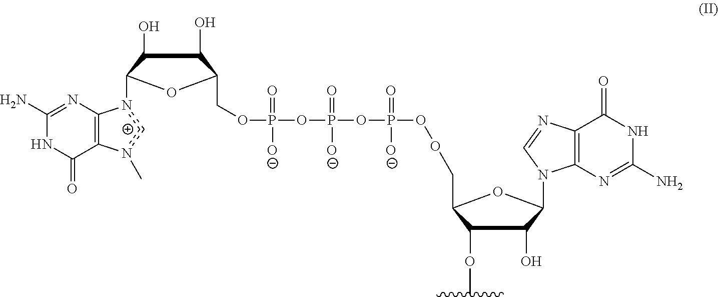

or a salt thereof.

In some embodiments, the 5′ cap is (m7)Gppp(m2′-O)G (“Ecap1”), having a structure of formula (III):

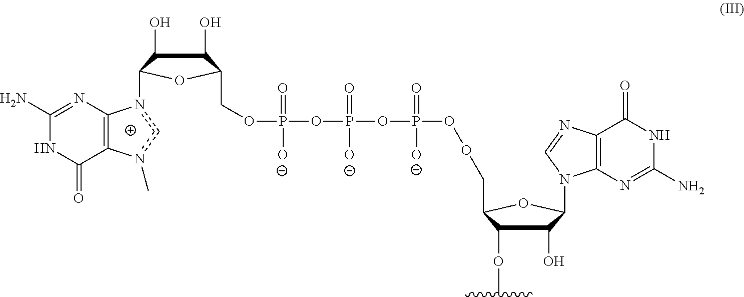

or a salt thereof.

In some embodiments, the 5′ cap is (m27,3′-O)GpppG (“ARCA” or “Dl”), having a structure of formula (IV):

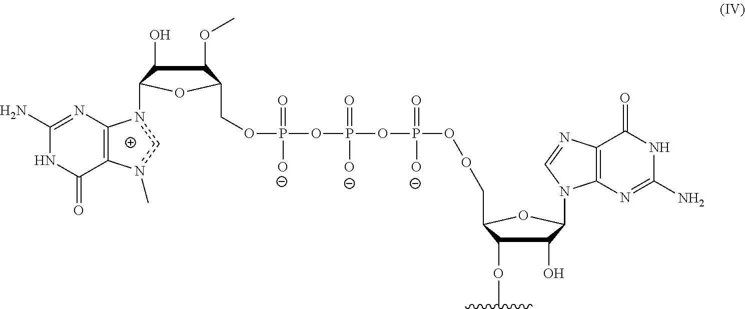

or a salt thereof.

In some embodiments, the 5′ cap is (m27,2′-O)GppSpG (“beta-S-ARCA”), having a structure of formula (V):

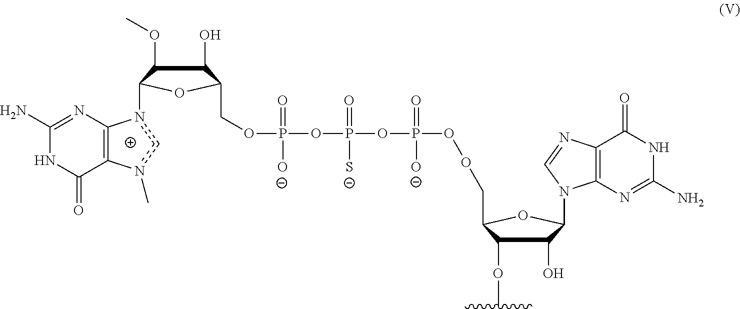

or a salt thereof.

In some embodiments, the 5′ cap is a trinucleotide cap structure. In some embodiments, the 5′ cap is a trinucleotide cap structure comprising N1pN2, wherein N1 and N2 are as defined and described herein. In some embodiments, the 5′ cap is a dinucleotide cap G*N1pN2, wherein N1 and N2 are as defined above and herein, and G* comprises a structure of formula (VI):

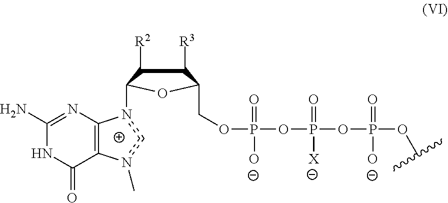

or a salt thereof, wherein R², R³, and X are as defined and described herein.

In some embodiments, the 5′ cap is a trinucleotide cap0 structure (e.g., (m7)GpppN1pN2, (m27,2′-O)GpppN1pN2, or (m27,3′-O)GpppN1pN2), wherein N1 and N2 are as defined and described herein). In some embodiments, the 5′ cap is a trinucleotide cap1 structure (e.g., (m7)Gppp(m2′-O)N1pN2, (m27,2′-O)Gppp(m2′-O)N1pN2, (m27,3′-O)Gppp(m2′-O)N1pN2wherein N1 and N2 are as defined and described herein. In some embodiments, the 5′ cap is a trinucleotide cap2 structure (e.g., (m7)Gppp(m2′-O)N1p(m2′-(m27,2′-O)Gppp(m2′-O)N1p(m2′-O)N2, (m27,3′-O)Gppp(m2 ‘-0)N1p(m2’-0)N2), wherein N1 and N2 are as defined and described herein. In some embodiments, the 5′ cap is selected from the group consisting of (m27,3′-O)Gppp(m2′-O)ApG (“CleanCap AG”, “CC413”), (m27,3′-O)Gppp(m2′-O)GpG (“CleanCap GG”), (m7)Gppp(m2′-O)ApG, (m7)Gppp(m2′-O)GpG, (m27,3′-O)Gppp(m26,2′-O)ApG, and (m7)Gppp(m2′-O)ApU.

In some embodiments, the 5′ cap is (m27,3′-O)Gppp(m2′-O)ApG (“CleanCap AG”, “CC413”), having a structure of formula (VII):

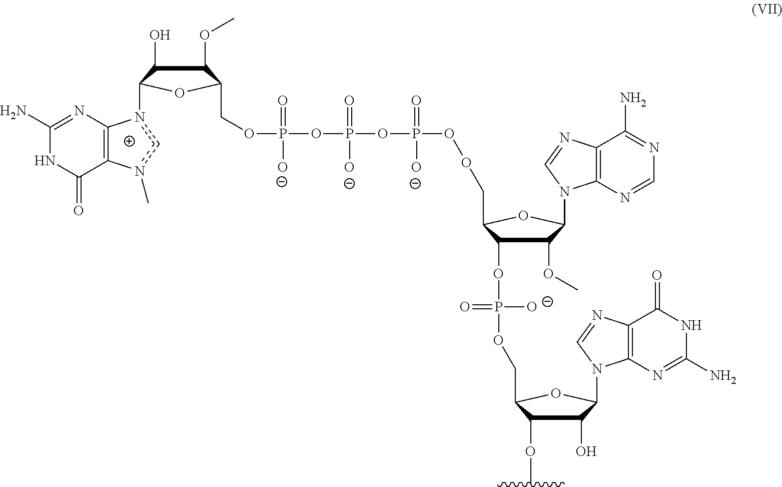

or a salt thereof.

In some embodiments, the 5′ cap is (m27,3′-O)Gppp(m2′-O)GpG (“CleanCap GG”), having a structure of formula (VIII):

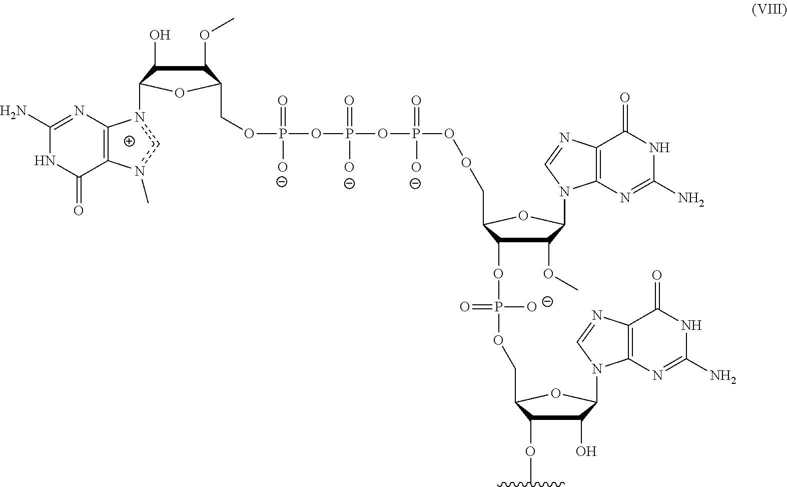

or a salt thereof.

In some embodiments, the 5′ cap is (m7)Gppp(m2′-O)ApG, having a structure of formula (IX):

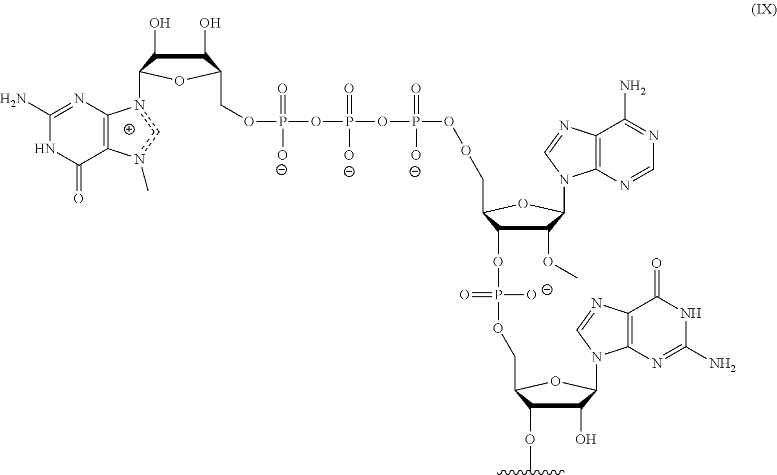

or a salt thereof.

In some embodiments, the 5′ cap is (m7)Gppp(m2′-O)GpG, having a structure of formula (X):

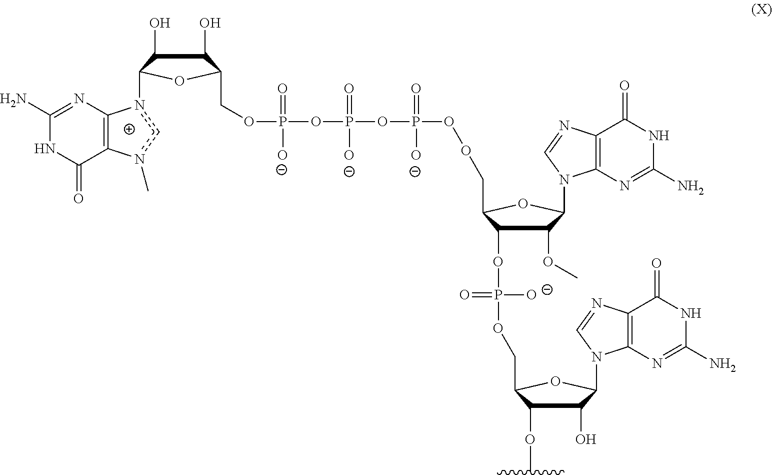

or a salt thereof.

In some embodiments, the 5′ cap is (m27,3′-O)Gppp(m26,2′-O)ApG, having a structure of formula (XI):

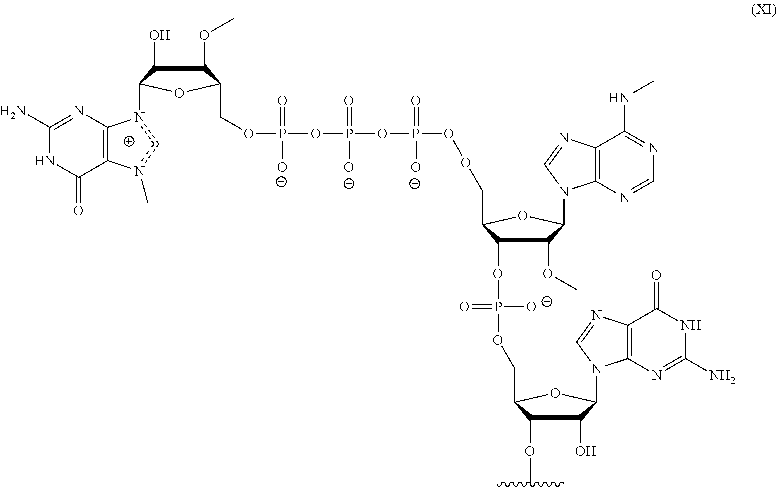

or a salt thereof.

In some embodiments, the 5′ cap is (m7)Gppp(m2′-O)ApU, having a structure of formula (XII):

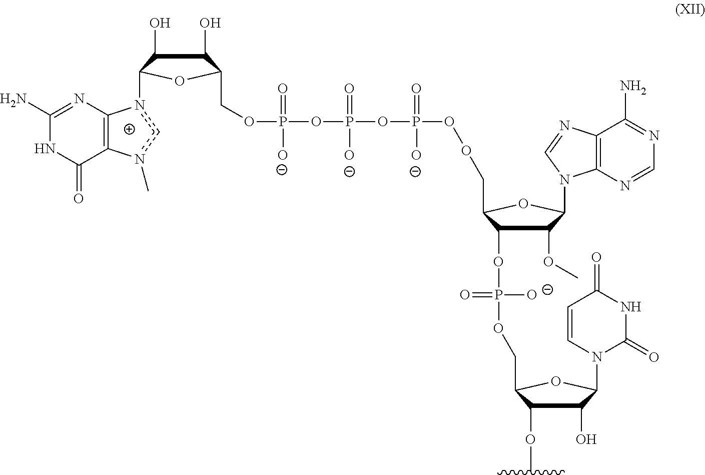

or a salt thereof.

In some embodiments, the 5′ cap is a tetranucleotide cap structure. In some embodiments, the 5′ cap is a tetranucleotide cap structure comprising N1pN2pN3, wherein N1, N2, and N3 are as defined and described herein. In some embodiments, the 5′ cap is a tetranucleotide cap G*N1pN2pN3, wherein N1, N2, and N3 are as defined above and herein, and G* comprises a structure of formula (XIII):

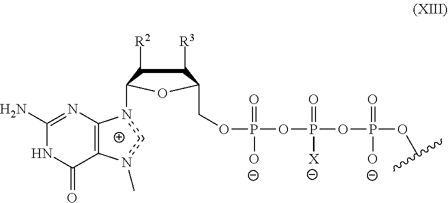

or a salt thereof, wherein R², R³, and X are as defined and described herein.

In some embodiments, the 5′ cap is a tetranucleotide cap0 structure (e.g. (m⁷)GpppN1pN2pN3, (m₂)^{7,2′-O})GpppN₁pN₂pN₃, or (m₂^{7,3′-O})GpppN₁N₂pN₃), wherein N₁, N₂, and N₃ are as defined and described herein). In some embodiments, the 5′ cap is a tetranucleotide Cap1 structure (e.g., (m⁷)Gppp(m^{2′-O})N₁pN₂pN₃, (m₂^{7,3′-O})Gppp(m^{2′-O})N₁pN₂pN₃, (m₂^{7,3′-O})Gppp(m^{2′-O})N₁pN₂N3), wherein N₁, N₂, and N₃ are as defined and described herein. In some embodiments, the 5′ cap is a tetranucleotide Cap2 structure (e.g., (m⁷)Gppp(m^{2′-O})N₁p(m^{2′-O})N₂pN₃, (m₂^{7,2′-O}(Gppp(m^{2′-O})Nip(m^{2′-O})N₂pN₃, (m₂^{7,3′-O})Gppp(m^{2′-O})Nip(m^{2′-O})N₂pN₃), wherein N₁, N₂, and N₃ are as defined and described herein. In some embodiments, the 5′ cap is selected from the group consisting of (m₂^{7,3′-O})Gppp(m^{2′-O})Ap(m^{2′-O})GpG, (m₂^{7,3′-O})Gppp(m^{2′-O})Gp(m^{2′-O})GpC, (m⁷)Gppp(m^{2′-O})Ap(m^{2′-O})UpA, and (m⁷)Gppp(m^{2′-O})Ap(m^{2′-O})GpG.

In some embodiments, the 5′ cap is (m₂^{7,3′-O})Gppp(m^{2′-O})Ap(m^{2′-O})GpG, having a structure of formula (XIV):

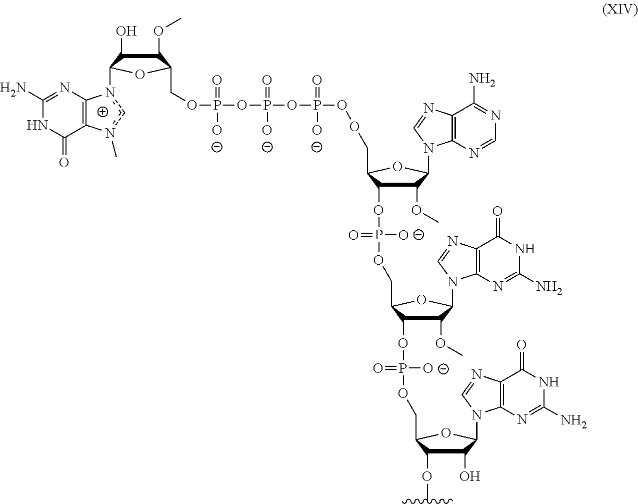

or a salt thereof.

In some embodiments, the 5′ cap is (m₂^{7,3′-O})Gppp(m^{2′-O})Gp(m^{2′-O})GpC, having a structure of formula (XV):

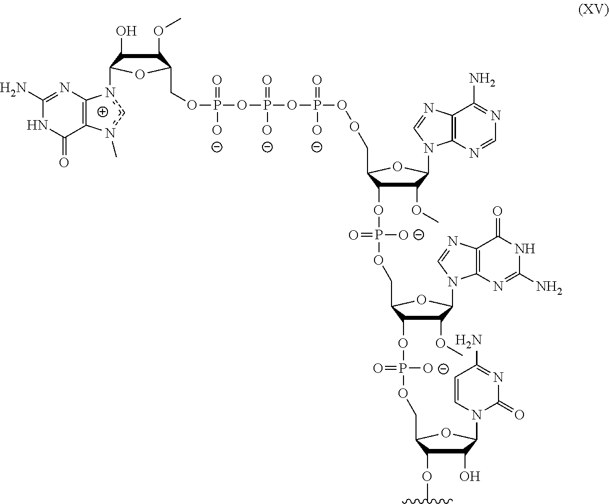

or a salt thereof.

In some embodiments, the 5′ cap is (m⁷)Gppp(m^{2′-O})Ap(m^{2′-O})UpA, having a structure of formula (XVI):

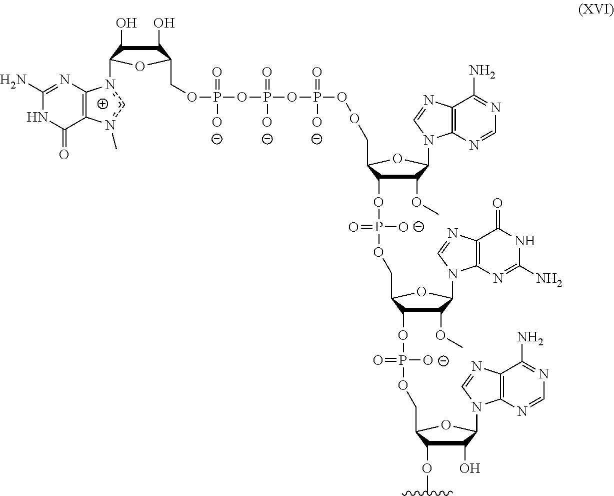

or a salt thereof.

In some embodiments, the 5′ cap is (m⁷)Gppp(m^{2′-O})Ap(m^{2′-O})GpG, having a structure of formula (XVII):

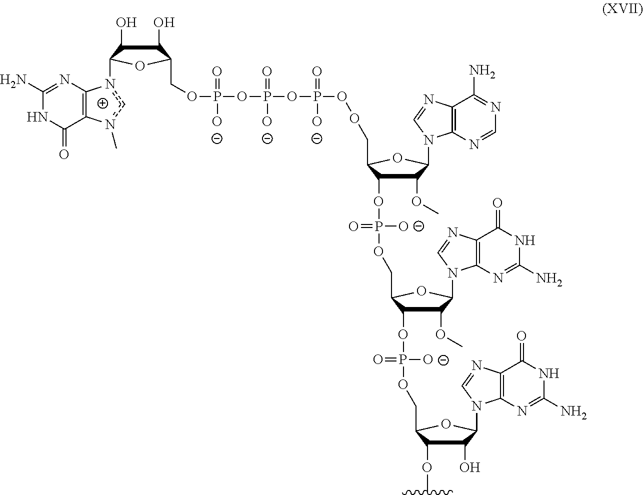

or a salt thereof.

### 2. Cap Proximal Sequences

In some embodiments, a 5′ UTR utilized in accordance with the present disclosure comprises a cap proximal sequence, e.g., as disclosed herein. In some embodiments, a cap proximal sequence comprises a sequence adjacent to a 5′ cap. In some embodiments, a cap proximal sequence comprises nucleotides in positions +1, +2, +3, +4, and/or +5 of an RNA polynucleotide.

In some embodiments, a cap structure comprises one or more polynucleotides of a cap proximal sequence. In some embodiments, a cap structure comprises an m⁷ guanosine cap and nucleotide +1 (N₁) of an RNA polynucleotide. In some embodiments, a cap structure comprises an m⁷ guanosine cap and nucleotide +2 (N₂) of an RNA polynucleotide. In some embodiments, a cap structure comprises an m⁷ guanosine cap and nucleotides +1 and +2 (N₁ and N₂) of an RNA polynucleotide. In some embodiments, a cap structure comprises an m⁷ guanosine cap and nucleotides +1, +2, and +3 (N₁, N₂, and N₃) of an RNA polynucleotide.

Those skilled in the art, reading the present disclosure, will appreciate that, in some embodiments, one or more residues of a cap proximal sequence (e.g., one or more of residues +1, +2, +3, +4, and/or +5) may be included in an RNA by virtue of having been included in a cap entity (e.g., a cap1 or cap2 structure, etc.); alternatively, in some embodiments, at least some of the residues in a cap proximal sequence may be enzymatically added (e.g., by a polymerase such as a T7 polymerase). For example, in certain exemplified embodiments where a m₂^{7,3′-O}Gppp(m₁^{2′-O})ApG cap is utilized, +1 (i.e., N₁) and +2 (i.e. N₂) are the (m₁^{2′-O})A and G residues of the cap, and +3, +4, and +5 are added by a polymerase (e.g., T7 polymerase).

In some embodiments, the 5″ cap is a dinucleotide cap structure, wherein the cap proximal sequence comprises N1 of the 5′ cap, where N₁ is any nucleotide, e.g., A, C, G or U. In some embodiments, the 5′ cap is a trinucleotide cap structure (e.g., the trinucleotide cap structures described above and herein), wherein the cap proximal sequence comprises N₁ and N₂ of the 5′ cap, wherein N₁ and N₂ are independently any nucleotide, e.g., A, C, G or U. In some embodiments, the 5′ cap is a tetranucleotide cap structure (e.g., the trinucleotide cap structures described above and herein), wherein the cap proximal sequence comprises N₁, N₂, and N₃ of the 5′ cap, wherein N₁, N₂, and N₃ are any nucleotide, e.g., A, C, G or U.

In some embodiments, e.g., where the 5′ cap is a dinucleotide cap structure, a cap proximal sequence comprises N1 of a the 5′ cap, and N2, N3, N4 and N5, wherein N1 to N5 correspond to positions +1, +2, +3, +4, and/or +5 of an RNA polynucleotide. In some embodiments, e.g., where the 5′ cap is a trinucleotide cap structure, a cap proximal sequence comprises N1 and N2 of a the 5′ cap, and N3, N4 and N5, wherein N1 to N5 correspond to positions +1, +2, +3, +4, and/or +5 of an RNA polynucleotide. In some embodiments, e.g., where the 5′ cap is a tetranucleotide cap structure, a cap proximal sequence comprises N1, N2, and N3 of a the 5′ cap, and N4 and N5, wherein N1 to N5 correspond to positions +1, +2, +3, +4, and/or +5 of an RNA polynucleotide.

In some embodiments, N1 is A. In some embodiments, N1 is C. In some embodiments, N1 is G. In some embodiments, N1 is U. In some embodiments, N2 is A. In some embodiments, N2 is C. In some embodiments, N2 is G. In some embodiments, N2 is U. In some embodiments, N3 is A. In some embodiments, N3 is C. In some embodiments, N3 is G. In some embodiments, N3 is U. In some embodiments, N4 is A. In some embodiments, N4 is C. In some embodiments, N4 is G. In some embodiments, N4 is U. In some embodiments, N5 is A. In some embodiments, N5 is C. In some embodiments, N5 is G. In some embodiments, N5 is U. It will be understood that, each of the embodiments described above and herein (e.g., for N1 through N5) may be taken singly or in combination and/or may be combined with other embodiments of variables described above and herein (e.g., 5′ caps).

In some embodiments, a cap proximal sequence comprises A1 and G2 of the Cap1 structure, and a sequence comprising: A3A4U5 (SEQ ID NO: 256) at positions +3, +4 and +5 respectively of the nucleotide sequence.

### 3. 5′ UTR

In some embodiments, an RNA utilized in accordance with the present disclosure comprises a 5′-UTR. In some embodiments, a 5′-UTR may comprise a plurality of distinct sequence elements; in some embodiments, such plurality may be or comprise multiple copies of one or more particular sequence elements (e.g., as may be from a particular source or otherwise known as a functional or characteristic sequence element). In some embodiments, a 5′ UTR comprises multiple different sequence elements.

The term “untranslated region” or “UTR” is commonly used in the art to refer to a region in a DNA molecule which is transcribed but is not translated into an amino acid sequence, or to the corresponding region in an RNA polynucleotide, such as an mRNA molecule. An untranslated region (UTR) can be present 5′ (upstream) of an open reading frame (5′-UTR) and/or 3′ (downstream) of an open reading frame (3′-UTR). As used herein, the terms “five prime untranslated region” or “5′ UTR” refer to a sequence of a nucleotide sequence between the 5′ end of the nucleotide sequence (e.g., a transcription start site) and a start codon of a coding region of the nucleotide sequence. In some embodiments, “5′ UTR” refers to a sequence of a nucleotide sequence that begins at the 5′ end of the nucleotide sequence (e.g., a transcription start site) and ends one nucleotide (nt) before a start codon (usually AUG) of a coding region of the nucleotide sequence, e.g., in its natural context. In some embodiments, a 5′ UTR comprises a Kozak sequence. A 5′-UTR is downstream of the 5′-cap (if present), e.g., directly adjacent to the 5′-cap. In some embodiments, a 5′ UTR disclosed herein comprises a cap proximal sequence, e.g., as defined and described herein. In some embodiments, a cap proximal sequence comprises a sequence adjacent to a 5′ cap.

Exemplary 5′ UTRs include a human alpha globin (hAg) 5′UTR or a fragment thereof, a TEV 5′ UTR or a fragment thereof, a HSP70 5′ UTR or a fragment thereof, or a c-Jun 5′ UTR or a fragment thereof.

In some embodiments, an RNA disclosed herein comprises a hAg 5′ UTR or a fragment thereof.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AGAATAAACTAGTATTCTTCTGGTCCCCACAGACTCAGAGAGAACCCGCCACC (SEQ ID NO: 257). In some embodiments, an RNA disclosed herein comprises a 5′ UTR having the sequence

(SEQ ID NO: 257)
AGAATAAACTAGTATTCTTCTGGTCCCCACAGACTCAGAGAGAACCCGCC
ACC.

In some embodiments, an RNA disclosed herein comprises a 5′ UTR having at least 80%, at least 85%, at least 90%, at least 95%, at least 96%, at least 97%, at least 98%, or at least 99% identity to a 5′ UTR with the sequence AACUAGUAUUCUUCUGGUCCCCACAGACUCAGAGAGAACCCGCCACC (SEQ ID NO: 258)(hAg-Kozak/5′UTR). In some embodiments, an RNA disclosed herein comprises a 5′ UTR having the sequence

(hAg-Kozak/5′UTR)
(SEQ ID NO: 258)
AACUAGUAUUCUUCUGGUCCCCACAGACUCAGAGAGAACCCGCCACC.

### 4. PolyA Tail

In some embodiments, a polynucleotide (e.g., DNA, RNA) disclosed herein comprises a polyadenylate (polyA) sequence, e.g., as described herein. In some embodiments, a polyA sequence is situated downstream of a 3′-UTR, e.g., adjacent to a 3′-UTR.

As used herein, the term “poly(A) sequence” or “poly-A tail” refers to an uninterrupted or interrupted sequence of adenylate residues which is typically located at the 3′-end of an RNA polynucleotide. Poly(A) sequences are known to those of skill in the art and may follow the 3′-UTR in the RNAs described herein. An uninterrupted poly(A) sequence is characterized by consecutive adenylate residues. In nature, an uninterrupted poly(A) sequence is typical. In some embodiments, polynucleotides disclosed herein comprise an uninterrupted poly(A) sequence. In some embodiments, polynucleotides disclosed herein comprise interrupted poly(A) sequence. In some embodiments, RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A nucleotides has a beneficial influence on the levels of RNA in transfected eukaryotic cells, as well as on the levels of protein that are translated from an open reading frame that is present upstream (5′) of the poly(A) sequence (Holtkamp et al., 2006, Blood, vol. 108, pp. 4009-4017, which is herein incorporated by reference).

In some embodiments, a poly(A) sequence in accordance with the present disclosure is not limited to a particular length; in some embodiments, a poly(A) sequence is any length. In some embodiments, a poly(A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as a poly(A) cassette.

In some embodiments, the poly(A) cassette present in the coding strand of DNA essentially consists of dA nucleotides, but is interrupted by a random sequence of the four nucleotides (dA, dC, dG, and dT). Such a random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1, which is incorporated herein by reference in its entirety, may be used in accordance with the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, at the DNA level, constant propagation of plasmid DNA in E. coli and is still associated, at the RNA level, with the beneficial properties with respect to supporting RNA stability and translational efficiency is encompassed. In some embodiments, the poly(A) sequence contained in an RNA polynucleotide described herein essentially consists of A nucleotides, but is interrupted by a random sequence of the four nucleotides (A, C, G, U). Such a random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, a poly(A) sequence comprises a specific number of adenosines, such as about 50 or more, about 60 or more, about 70 or more, about 80 or more, about 90 or more, about 100 or more, about 120, or about 150 or about 200. In some embodiments a poly(A) sequence of an RNA may comprise 200 A residues or less. In some embodiments, a poly(A) sequence of an RNA may comprise about 200 A residues. In some embodiments, a poly(A) sequence of an RNA may comprise 180 A residues or less. In some embodiments, a poly(A) sequence 1 of an RNA may comprise about 180 A residues. In some embodiments, a poly(A) sequence may comprise 150 residues or less.

In some embodiments, RNA comprises a poly(A) sequence comprising the nucleotide sequence of: AAAAAAAAAAAAAAAAAAAAAAAAAAAAAAGCATATGACTAAAAAAAAAAAAAA AAAAAAAAAAAAAAAAAAAAAAAAAAAAAAAAAAAAAAAAAAAAAAAAAAAAAA AA (SEQ ID NO: 259), or a nucleotide sequence having at least 99%, 98%, 97%, 96%, 95%, 90%, 85%, or 80% identity to the nucleotide sequence of: AAAAAAAAAAAAAAAAAAAAAAAAAAAAAAGCATATGACTAAAAAAAAAAAAAA AAAAAAAAAAAAAAAAAAAAAAAAAAAAAAAAAAAAAAAAAAAAAAAAAAAAAA AA (SEQ ID NO: 259). In some embodiments, a poly(A) sequence comprises a plurality of A residues interrupted by a linker. In some embodiments, a linker comprises the nucleotide sequence GCATATGAC (SEQ ID NO: 260).

In some embodiments, an RNA comprises a poly(A) sequence comprising the nucleotide sequence of: AAAAAAAAAAAAAAAAAAAAAAAAAAAAAAGCAUAUGACUAAAAAAAAAAAAAA AAAAAAAAAAAAAAAAAAAAAAAAAAAAAAAAAAAAAAAAAAAAAAAAAAAAAA AA (SEQ ID NO: 261), or a nucleotide sequence having at least 99%, 98%, 97%, 96%, 95%, 90%, 85%, or 80% identity to the nucleotide sequence of: AAAAAAAAAAAAAAAAAAAAAAAAAAAAAAGCAUAUGACUAAAAAAAAAAAAAA AAAAAAAAAAAAAAAAAAAAAAAAAAAAAAAAAAAAAAAAAAAAAAAAAAAAAA AA (SEQ ID NO: 261). In some embodiments, a poly(A) sequence comprises a plurality of A residues interrupted by a linker. In some embodiments, a linker comprises the nucleotide sequence GCAUAUGAC (SEQ ID NO: 262).

### 5. 3′ UTR

In some embodiments, an RNA utilized in accordance with the present disclosure comprises a 3′-UTR. As used herein, the terms “three prime untranslated region,” “3′ untranslated region,” or “3′ UTR” refer to a sequence of an mRNA molecule that begins following a stop codon of a coding region of an open reading frame sequence. In some embodiments, the 3′ UTR begins immediately after a stop codon of a coding region of an open reading frame sequence, e.g., in its natural context. In other embodiments, the 3′ UTR does not begin immediately after stop codon of the coding region of an open reading frame sequence, e.g., in its natural context. The term “3′-UTR” preferably does not include the poly(A) sequence. Thus, the 3′-UTR is upstream of the poly(A) sequence (if present), e.g. directly adjacent to the poly(A) sequence.

In some embodiments, an RNA disclosed herein comprises a 3′TR comprising an F element and/or an I element. In some embodiments, a 3′ UTR or a proximal sequence thereto comprises a restriction site. In some embodiments, a restriction site is a BamHI site. In some embodiments, a restriction site is an XhoI site.

In some embodiments, an RNA construct comprises an F element. In some embodiments, an F element sequence is a 3′ UTR of amino-terminal enhancer of split (AES).

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CTGGTACTGCATGCACGCAATGCTAGCTGCCCCTTTCCCGTCCTGGGTACCCCGAGT CTCCCCCGACCTCGGGTCCCAGGTATGCTCCCACCTCCACCTGCCCCACTCACCACC TCTGCTAGTTCCAGACACCTCCCAAGCACGCAGCAATGCAGCTCAAAACGCTTAGCC TAGCCACACCCCCACGGGAAACAGCAGTGATTAACCTTTAGCAATAAACGAAAGTT TAACTAAGCTATACTAACCCCAGGGTTGGTCAATTTCGTGCCAGCCACACC (SEQ ID NO: 263). In some embodiments, an RNA disclosed herein comprises a 3′ UTR with the sequence of

(SEQ ID NO: 263)
CTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTG
CCCCACTCACCACCTCTGCTAGTTCCAGACACCTCCCAAGCACGCAGCAA
TGCAGCTCAAAACGCTTAGCCTAGCCACACCCCCACGGGAAACAGCAGTG
ATTAACCTTTAGCAATAAACGAAAGTTTAACTAAGCTATACTAACCCCAG
GGTTGGTCAATTTCGTGCCAGCCACACC.

In some embodiments, an RNA disclosed herein comprises a 3′ UTR having at least 80%, at least 85%, at least 90%, at least 95%, at least 96%, at least 97%, at least 98%, or at least 99% identity to a 3′ UTR with the sequence of CUGGUACUGCAUGCACGCAAUGCUAGCUGCCCCUUUCCCGUCCUGGGUACCCCGA GUCUCCCCCGACCUCGGGUCCCAGGUAUGCUCCCACCUCCACCUGCCCCACUCACC ACCUCUGCUAGUUCCAGACACCUCCCAAGCACGCAGCAAUGCAGCUCAAAACGCU UAGCCUAGCCACACCCCCACGGGAAACAGCAGUGAUUAACCUUUAGCAAUAAACG AAAGUUUAACUAAGCUAUACUAACCCCAGGGUUGGUCAAUUUCGUGCCAGCCACA CC (SEQ ID NO: 264). In some embodiments, an RNA disclosed herein comprises a 3′ UTR with the sequence of

(SEQ ID NO: 264)
CUGGUACUGCAUGCACGCAAUGCUAGCUGCCCCUUUCCCGUCCUGGGUAC
CCCGAGUCUCCCCCGACCUCGGGUCCCAGGUAUGCUCCCACCUCCACCUG
CCCCACUCACCACCUCUGCUAGUUCCAGACACCUCCCAAGCACGCAGCAA
UGCAGCUCAAAACGCUUAGCCUAGCCACACCCCCACGGGAAACAGCAGUG
AUUAACCUUUAGCAAUAAACGAAAGUUUAACUAAGCUAUACUAACCCCAG
GGUUGGUCAAUUUCGUGCCAGCCACACC.

In some embodiments, a 3′ UTR is an FI element as described in WO2017/060314, which is herein incorporated by reference in its entirety.

### Modified RNAs

In some embodiments, the present disclosure provides compositions comprising modified RNAs and methods of use thereof. In some embodiments, the modified RNA comprises one or more modified nucleoside residues. For example, in some embodiments, an RNA comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 2, Table 4, or Table 6, comprises one or more modified nucleoside residues.

In some embodiments, an RNA as described herein refers to a messenger RNA.

In some embodiments, all uridine residues are modified as described herein. In some embodiments, one or more of the RNAs as described herein are nucleoside-modified RNAs. In other embodiments, two or more of the RNAs as described herein are nucleoside-modified RNAs. In other embodiments, three or more of the RNAs as described herein are nucleoside-modified RNAs.

In another embodiment, the modified nucleoside of the methods and compositions of the present disclosure is m5C (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine), m²A (2-methyladenosine), m⁶A (N6-methyladenosine), Am (2′-O-methyladenosine), ms²m₆A (2-methylthio-N6-methyladenosine), i⁶A (N6-isopentenyladenosine), ms²io⁶A (2-methylthio-N6-isopentenyladenosine), io⁶A (N6-(cis-hydroxyisopentenyl)adenosine), ms²io⁶A (2-methylthio-N6-(cis-hydroxyisopentenyl) adenosine), g⁶A (N6-glycinylcarbamoyladenosine), t⁶A (N6-threonylcarbamoyladenosine), ms²t⁶A (2-methylthio-N6-threonyl carbamoyladenosine), m⁶t⁶A (N6-methyl-N6-threonylcarbamoyladenosine), hn⁶A (N6-hydroxynorvalylcarbamoyladenosine), ms²hn⁶A (2-methylthio-N6-hydroxynorvalyl carbamoyladenosine), Ar(p) (2′-O-ribosyladenosine (phosphate)), I (inosine), m¹I (1-methylinosine), m¹Im (1,2′-O-dimethylinosine), m³C (3-methylcytidine), m⁵C (5-methylcytidine), Cm (2′-O-methylcytidine), s²C (2-thiocytidine), ac⁴C (N4-acetylcytidine), f⁵C (5-formylcytidine), m⁵Cm (5,2′-O-dimethylcytidine), ac⁴Cm (N4-acetyl-2′-O-methylcytidine), k²C (lysidine), m¹G (1-methylguanosine), m²G (N2-methylguanosine), m⁷G (7-methylguanosine), Gm (2′-O-methylguanosine), m²₂G (N2,N2-dimethylguanosine), m²Gm (N2,2′-O-dimethylguanosine), m²₂Gm (N2,N2,2′-O-trimethylguanosine), Gr(p) (2′-O-ribosylguanosine (phosphate)), yW (wybutosine), o₂yW (peroxywybutosine), OHyW (hydroxywybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQ0 (7-cyano-7-deazaguanosine), preQ1 (7-aminomethyl-7-deazaguanosine), G+(archaeosine), Ψ (pseudouridine), D (dihydrouridine), m⁵U (5-methyluridine), Um (2′-O-methyluridine), m⁵Um (5,2′-O-dimethyluridine), m¹Ψ (1-methylpseudouridine), Ψm (2′-O-methylpseudouridine), s²U (2-thiouridine), s⁴U (4-thiouridine), m⁵s2U (5-methyl-2-thiouridine), s²Um (2-thio-2′-O-methyluridine), acp³U (3-(3-amino-3-carboxypropyl)uridine), ho⁵U (5-hydroxyuridine), mo⁵U (5-methoxyuridine), cmo⁵U (uridine 5-oxyacetic acid), mcmo⁵U (uridine 5-oxyacetic acid methyl ester), chm⁵U (5-(carboxyhydroxymethyl)uridine), mchm⁵U (5-(carboxyhydroxymethyl)uridine methyl ester), mcm⁵U (5-methoxycarbonylmethyluridine), mcm⁵Um (5-methoxycarbonylmethyl-2′-O-methyluridine), mcm⁵s2U (5-methoxycarbonylmethyl-2-thiouridine), nm⁵s2U (5-aminomethyl-2-thiouridine), mnm⁵U (5-methylaminomethyluridine), mnm⁵s2U (5-methylaminomethyl-2-thiouridine), mnm⁵se²U (5-methylaminomethyl-2-selenouridine), nCm⁵U (5-carbamoylmethyluridine), nCm⁵Um (5-carbamoylmethyl-2′-O-methyluridine), cmnm⁵U (5-carboxymethylaminomethyluridine), cmnm⁵Um (5-carboxymethylaminomethyl-2′-O-methyluridine), cmnm⁵s2U (5-carboxymethylaminomethyl-2-thiouridine), m⁶₂A (N6,N6-dimethyladenosine), Im (2′-O-methylinosine), m⁴C (N4-methylcytidine), m⁴Cm (N4,2′-O-dimethylcytidine), hm⁵C (5-hydroxymethylcytidine), m³U (3-methyluridine), m¹acp³Ψ (1-methyl-3-(3-amino-3-carboxypropyl) pseudouridine), cm⁵U (5-carboxymethyluridine), m⁶Am (N6,2′-O-dimethyladenosine), m⁶₂Am (N6,N6,2′-O-trimethyladenosine), m^{2,7}G (N2,7-dimethylguanosine), m^{2,2,7}G (N2,N2,7-trimethylguanosine), m³Um (3,2′-O-dimethyluridine), m⁵D (5-methyldihydrouridine), m³Ψ (3-methylpseudouridine), f⁵Cm (5-formyl-2′-O-methylcytidine), m¹Gm (1,2′-O-dimethylguanosine), m¹Am (1,2′-O-dimethyladenosine), τm⁵U (5-taurinomethyluridine), τm⁵s2U (5-taurinomethyl-2-thiouridine), imG-14 (4-demethylwyosine), imG2 (isowyosine), ac⁶A (N6-acetyladenosine), inm⁵U (5-(isopentenylaminomethyl)uridine), inm⁵s2U (5-(isopentenylaminomethyl)-2-thiouridine), inm⁵Um (5-(isopentenylaminomethyl)-2′-O-methyluridine), m^{2,7}Gm (N2,7,2′-O-trimethylguanosine), m⁴²Cm (N4,N4,2′-O-trimethylcytidine), C⁺ (agmatidine), m⁸A (8-methyladenosine), gmnm⁵s²U (geranylated 5-methylaminomethyl-2-thiouridine), gcmnm⁵s²U (geranylated 5-carboxymethylaminomethyl-2-thiouridine), or cnm⁵U (5-cyanomethyl-uridine).

In some embodiments, the modified nucleoside residues are pseudouridine or pseudouridine family residues.

In some embodiments, the modified RNA comprises pseudouridine residues. In some embodiments, pseudouridine refers to the C-glycoside isomer of the nucleoside uridine. In some embodiments, pseudouridine residues comprise m¹acp³Ψ (1-methyl-3-(3-amino-5-carboxypropyl)pseudouridine, m¹Ψ (1-methylpseudouridine), Ψm (2′-O-methylpseudouridine, m⁵D (5-methyldihydrouridine), m³ Ψ (3-methylpseudouridine), or a combination thereof. In some embodiments, said pseudouridine residues comprise 1-methylpseudouridine residues instead of uridine.

In some embodiments, the modified nucleoside residues are pseudouridine analogues. In some embodiments, a “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹Ψ), 1-methyl-4-thio-pseudouridine (m¹s⁴Ψ), 4-thio-1-methyl-pseudouridine, 3-methyl-pseudouridine (m³T), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³Ψ), and 2′-O-methyl-pseudouridine (Ψm).

In some embodiments, the modified nucleobase is a modified uracil. Exemplary nucleobases and nucleosides having a modified uracil include pseudouridine (Ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τcm⁵U), 1-taurinomethyl-pseudouridine, 5-taurinomethyl-2-thio-uridine (τrm⁵s²U), 1-taurinomethyl-4-thio-pseudouridine, 5-methyl-uridine (m⁵U, i.e., having the nucleobase deoxythymine), 1-methylpseudouridine (m¹Ψ), 5-methyl-2-thio-uridine (M⁵s²U), 1-methyl-4-thio-pseudouridine (m¹s⁴Ψ), 4-thio-1-methyl-pseudouridine, 3-methyl-pseudouridine (m³T), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹Ψ), 3-(3-amino-3-carboxypropyl)uridine (acp³U), 1-methyl-3-(3-amino-3-carboxypropyl)pseudouridine (acp³Ψ), 5-(isopentenylaminomethyl)uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (Ψm), 2-thio-2′-O-methyl-uridine (s²Um), 5-methoxycarbonylmethyl-2′-O-methyl-uridine (mcm⁵Um), 5-carbamoylmethyl-2′-β-methyl-uridine (ncm⁵Um), 5-carboxymethylaminomethyl-2′-O-methyl-uridine (cmnmsUm), 3,2′-O-dimethyl-uridine (m³Um), 5-(isopentenylaminomethyl)-2′-β-methyl-uridine (inm⁵Um), 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁶Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m₆A), N6-isopentenyl-adenosine (i⁶A), 2-methylthio-N6-isopentenyl-adenosine (ms²hn⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac^{6A}), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-β-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²2G), N2,7-dimethyl-guanosine (m^{2,7}G), N2,N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m²′7Gm), 2′-O-methyl-inosine (Im), 1,2′-O-dimethyl-inosine (m¹Im), and 2′-O-ribosylguanosine (phosphate) (Gr(p)).

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

### Modifications on the Internucleoside Linkage

The modified nucleotides, which may be incorporated into a polynucleotide, primary construct, or RNA molecule, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked polynucleotides, primary constructs, or modified RNA molecules are expected to also reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein below.

### Combinations of Modified Sugars, Nucleobases, and Internucleoside Linkages

The polynucleotides, primary constructs, and modified RNA of the disclosure can include a combination of modifications to the sugar, the nucleobase, and/or the internucleoside linkage.

In another embodiment, the purified preparation of RNA, oligoribonucleotide, or polyribonucleotide of the methods and compositions of the present disclosure comprises a combination of two or more of the above-described modifications. In another embodiment, the purified preparation of the RNA or oligoribonucleotide comprises a combination of three or more of the above-described modifications. In another embodiment, the purified preparation of the RNA or oligoribonucleotide comprises a combination of more than three of the above-described modifications.

In some embodiments, the modified RNAs comprise in vitro-synthesized modified RNAs.

In some embodiments, the present disclosure comprises one or more modified RNAs encoding an HSV glycoprotein. In some embodiments, the modified RNA comprises pseudouridine or pseudouridine family residues. In another embodiment, the modified RNAs of the present disclosure are capable of directing protein expression of HSV glycoproteins encoded thereon.

In another embodiment, the present disclosure provides an in vitro-transcribed RNA molecule encoding an HSV glycoprotein, comprising a pseudouridine. In another embodiment, the present disclosure provides a synthetic RNA molecule encoding an HSV glycoprotein, comprising a pseudouridine.

In another embodiment, an in vitro-transcribed RNA molecule of the methods and compositions of the present disclosure is synthesized by T7 phage RNA polymerase. In another embodiment, the molecule is synthesized by SP6 phage RNA polymerase. In another embodiment, the molecule is synthesized by T3 phage RNA polymerase. In another embodiment, the molecule is synthesized by a polymerase selected from the above polymerases. In another embodiment, the RNA is synthesized chemically on a column similar to DNA.

In another embodiment, the nucleoside that is modified in an RNA, oligoribonucleotide, or polyribonucleotide of the methods and compositions of the present disclosure is uridine (U). In another embodiment, the modified nucleoside is cytidine (C). In another embodiment, the modified nucleoside is adenine (A). In another embodiment the modified nucleoside is guanine (G).

In another embodiment, the RNA of the methods and compositions of the present disclosure further comprises a poly-A tail. In another embodiment, the RNA of the methods and compositions of the present disclosure does not comprise a poly-A tail. Each possibility represents a separate embodiment of the present disclosure.

In another embodiment, the RNA of the methods and compositions of the present disclosure comprises a cap. In some embodiments, the cap is a 5′ cap. In some embodiments, the 5′ cap comprises a trinucleotide cap. In some embodiments, the 5′ cap generate a Cap 1 structure.

In some embodiments, the cap comprises an m7GpppG cap. In another embodiment, the RNA of the methods and compositions of the present disclosure does not comprise an m7GpppG cap. In another embodiment, the RNA of the methods and compositions of the present disclosure comprises a 3′-O-methyl-m7GpppG. In another embodiment, the RNA of methods and composition of the present disclosure comprise a non-reversible cap analog, which, in some embodiments, is added during transcription of the RNA. In another embodiment, the RNA of methods and composition of the present disclosure comprise an anti-reverse cap analog. Each possibility represents a separate embodiment of the present disclosure.

In another embodiment, the RNA of the methods and compositions of the present disclosure further comprises a cap-independent translational enhancer. In another embodiment, the RNA of the methods and compositions of the present disclosure does not comprise a cap-independent translational enhancer. In another embodiment, the cap-independent translational enhancer is a tobacco etch virus (TEV) cap-independent translational enhancer. In another embodiment, the cap-independent translational enhancer is any other cap-independent translational enhancer known in the art. Each possibility represents a separate embodiment of the present disclosure.

In some embodiments, “pseudouridine” refers to m¹acp³T (1-methyl-3-(3-amino-5-carboxypropyl)pseudouridine. In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the modified nucleoside is 4′ (pseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present disclosure.

In another embodiment, the modified RNA comprises a modified nucleoside, which in some embodiments, comprises m⁵C, m5U, m⁶A, s²U, T, 2′-O-methyl-U, 2′-O-methylpseudouridine, or a combination thereof.

In another embodiment, the present disclosure provides a method for delivering a recombinant protein to a subject, the method comprising the step of contacting the subject with an RNA of the methods and compositions of the present disclosure, thereby delivering a recombinant protein to a subject.

In another embodiment, a method of the present disclosure comprises increasing the number, percentage, or frequency of modified uridine nucleosides in the RNA molecule to decrease immunogenicity or increase efficiency of translation. In some embodiments, the number of modified uridine residues in an RNA, oligoribonucleotide, or polyribonucleotide molecule determines the magnitude of the effects observed in the present disclosure.

In another embodiment, between 0.1% and 100% of the uridine residues in the modified RNAs of the methods and compositions of the present disclosure are modified (e.g. by the presence of pseudouridine). In another embodiment, 0.1% of the residues are modified. In another embodiment, 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%

In another embodiment, 0.1% of the residues of a given uridine nucleotide are modified. In another embodiment, the fraction of the nucleotide is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given uridine nucleotide is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, the terms “ribonucleotide,” “oligoribonucleotide,” and polyribonucleotide refers to, in some embodiments, compounds comprising nucleotides in which the sugar moiety is ribose. In another embodiment, the term includes both RNA and RNA derivates in which the backbone is modified. Numerous RNA backbone modifications are known in the art and contemplated in the present disclosure. In some embodiments, modified RNA is a PNA (peptide nucleic acid). PNA contain peptide backbones and nucleotide bases and are able to bind, in another embodiment, to both DNA and RNA molecules. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. Each nucleic acid derivative represents a separate embodiment of the present disclosure.

Methods for production of nucleic acids having modified backbones are well known in the art, and are described, for example in U.S. Pat. Nos. 5,723,335 and 5,663,153 issued to Hutcherson et al. and related PCT publication WO95/26204. Each method represents a separate embodiment of the present disclosure.

The nucleic acid of interest can be purified by any method known in the art, or any method to be developed, so long as the method of purification removes contaminants from the nucleic acid preparation and thereby substantially reduces the immunogenicity potential of the nucleic acid preparation. In some embodiments, the nucleic acid of interest is purified using high-performance liquid chromatography (HPLC). In another embodiment, the nucleic acid of interest is purified by contacting the nucleic acid of interest with the bacterial enzyme RNase III. In other various embodiments, any method of nucleic acid purification that substantially reduces the immunogenicity of the nucleic acid preparation can be used. Non-limiting examples of purification methods that can be used with the compositions and methods of the disclosure include liquid chromatography separation and enzyme digestion, each used alone or in any combination, simultaneously or in any order. Non-limiting examples of liquid chromatography separation include HPLC and fast protein liquid chromatography (FPLC). Materials useful in the HPLC and FPLC methods of the disclosure include, but are not limited to, cross-linked polystyrene/divinylbenzene (PS/DVB), PS/DVB-C18, PS/DVB-alkylated, Helix DNA columns (Varian), Eclipse dsDNA Analysis Columns (Agilent Technologies), Reverse-phase 5 (RPC-5) exchange material, DNAPac, ProSwift, and bio-inert UltiMate.RTM. 3000 Titanium columns (Dionex). Enzymes useful in the enzyme digestion methods of the disclosure include any enzyme able to digest any contaminant in a nucleic acid preparation of the disclosure, such as, for example a dsRNA contaminant, and include but are not limited to, RNase III, RNase V1, Dicer, and Chipper (see Fruscoloni et al., 2002, PNAS 100:1639) Non-limiting examples of assays for assessing the purity of the nucleic acid of interest include a dot-blot assay, a Northern blot assay, and a dendritic cell activation assay, as described elsewhere herein.

In another embodiment, the modified RNA of the methods and compositions of the present disclosure is significantly less immunogenic than an unmodified in vitro-synthesized RNA molecule with the same sequence. In another embodiment, the modified RNA molecule is 2-fold less immunogenic than its unmodified counterpart. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

In another embodiment, “significantly less immunogenic” refers to a detectable decrease in immunogenicity. In another embodiment, the term refers to a fold decrease in immunogenicity (e.g. 1 of the fold decreases enumerated above). In another embodiment, the term refers to a decrease such that an effective amount of the modified RNA can be administered without triggering a detectable immune response. In another embodiment, the term refers to a decrease such that the modified RNA can be repeatedly administered without eliciting an immune response sufficient to detectably reduce expression of the recombinant protein. In another embodiment, the decrease is such that the modified RNA can be repeatedly administered without eliciting an immune response sufficient to eliminate detectable expression of the recombinant protein.

Methods of determining immunogenicity are well known in the art, and described in detail in U.S. Pat. No. 8,278,036 which is hereby incorporated by reference herein.

In another embodiment, the modified RNA of the methods and compositions of the present disclosure is translated in the cell more efficiently than an unmodified RNA molecule with the same sequence. In another embodiment, the modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts. Each possibility represents a separate embodiment of the present disclosure.

Methods of determining translation efficiency are well known in the art, and include, e.g. measuring the activity of an encoded reporter protein (e.g luciferase or renilla or green fluorescent protein [Wall A A, Phillips A M et al., Effective translation of the second cistron in two Drosophila dicistronic transcripts is determined by the absence of in-frame AUG codons in the first cistron. J Biol Chem 2005; 280(30): 27670-8]), or measuring radioactive label incorporated into the translated protein (Ngosuwan J, Wang N M et al, Roles of cytosolic Hsp70 and Hsp40 molecular chaperones in post-translational translocation of pre-secretory proteins into the endoplasmic reticulum. J Biol Chem 2003; 278(9): 7034-42). Each method represents a separate embodiment of the present disclosure.

In another embodiment, the target cell of the method of the present disclosure is a dendritic cell. In another embodiment, the target cell of the method of the present disclosure is a macrophage. In another embodiment, the target cell of the method of the present disclosure is a B cell. In another embodiment, the target cell of the method of the present disclosure is another antigen presenting cell. In another embodiment, the target cell of methods of the present disclosure is a mucosal cell. In another embodiment, the target cell of methods of the present disclosure is an epithelial cell. In another embodiment, the cell is a skin cell. In another embodiment, the cell is an epidermal cell. In another embodiment, the cell is a keratinocyte. In another embodiment, the cell is a Merkel cell, melanocyte or Langerhans cell. Each possibility represents a separate embodiment of the present disclosure.

### Codon Optimization and GC Enrichment

The present disclosure also provides codon optimized nucleotide sequences.

As used herein, the term “codon-optimized” refers to alteration of codons in a coding region of a nucleic acid molecule (e.g., a nucleotide sequence) to reflect the typical codon usage of a host organism (e.g., a subject receiving a nucleic acid molecule (e.g., a nucleotide sequence)) preferably without altering the amino acid sequence encoded by the nucleic acid molecule. Within the context of the present disclosure, in some embodiments, coding regions are codon-optimized for optimal expression in a subject to be treated using the RNA molecules described herein. In some embodiments, codon-optimization may be performed such that codons for which frequently occurring tRNAs are available are inserted in place of “rare codons.” In some embodiments, codon-optimization may include increasing guanosine/cytosine (G/C) content of a coding region of RNA described herein as compared to the G/C content of the corresponding coding sequence of a wild type RNA, wherein the amino acid sequence encoded by the RNA is preferably not modified compared to the amino acid sequence.

In some embodiments, a coding sequence (also referred to as a “coding region”) is codon optimized for expression in the subject to whom a composition (e.g., a pharmaceutical composition) is to be administered (e.g., a human). Thus, in some embodiments, sequences in such a polynucleotide (e.g., a nucleotide sequence) may differ from wild type sequences encoding the relevant antigen, fragment or epitope thereof, even when the amino acid sequence of the antigen, fragment or epitope thereof is wild type.

In some embodiments, strategies for codon optimization for expression in a relevant subject (e.g., a human), and even, in some cases, for expression in a particular cell or tissue.

Various species exhibit particular bias for certain codons of a particular amino acid. Without wishing to be bound by any one theory, codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally be a reflection of the codons used most frequently in peptide synthesis. Accordingly, genes may be tailored for optimal gene expression in a given organism based on codon optimization. Codon usage tables are available, for example, at the “Codon Usage Database” available at www.kazusa.orjp/codon/ and these tables may be adapted in a number of ways. Computer algorithms for codon optimizing a particular sequence for expression in a particular subject or its cells are also available, such as Gene Forge (Aptagen; Jacobus, PA), are also available.

In some embodiments, a polynucleotide (e.g., a polyribonucleotide or a nucleotide sequence) of the present disclosure is codon optimized, wherein the codons in the polynucleotide (e.g., the polyribonucleotide) are adapted to human codon usage (herein referred to as “human codon optimized polynucleotide”). In some embodiments, a portion of a nucleotide sequence is codon optimized (e.g., a portion of or the portion encoding a glycoprotein or a portion of or the portion encoding a signal sequence). In some embodiments, the entire nucleotide sequence is codon optimized. Codons encoding the same amino acid occur at different frequencies in a subject, e.g., a human. Accordingly, in some embodiments, the coding sequence of a polynucleotide of the present disclosure is modified such that the frequency of the codons encoding the same amino acid corresponds to the naturally occurring frequency of that codon according to the human codon usage, e.g., as shown in Table 7. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG. is used with 30 a frequency of 0.10 etc. (see Table 7). Accordingly, in some embodiments, such a procedure (as exemplified for Ala) is applied for each amino acid encoded by the coding sequence of a polynucleotide to obtain sequences adapted to human codon usage.

TABLE 7
Human codon usage with
frequencies indicated
for each amino acid.

| Amino | | |
|---|---|---|
| Acid | Codon | Frequency |
| Ala | GCG | 0.10 |
| Ala | GCA | 0.22 |
| Ala | GCT | 0.28 |
| Ala | GCC* | 0.40 |
| Cys | TGT | 0.42 |
| Cys | TGC* | 0.58 |
| Asp | GAT | 0.44 |
| Asp | GAC* | 0.56 |
| Glu | GAG* | 0.59 |
| Glu | GAA | 0.41 |
| Phe | TTT | 0.43 |
| Phe | TTC* | 0.57 |
| Gly | GGG | 0.23 |
| Gly | GGA | 0.26 |
| Gly | GGT | 0.18 |
| Gly | GGC* | 0.33 |
| His | CAT | 0.41 |
| His | CAC* | 0.59 |
| lle | ATA | 0.14 |
| lle | ATT | 0.35 |
| lle | ATC* | 0.52 |
| Lys | AAG* | 0.60 |
| Lys | AAA | 0.40 |
| Leu | TTG | 0.12 |
| Leu | TTA | 0.06 |
| Leu | CTG* | 0.43 |
| Leu | CTA | 0.07 |
| Leu | CTT | 0.12 |
| Leu | CTC | 0.20 |
| Met | ATG* | 1 |
| Asn | AAT | 0.44 |
| Asn | AAC* | 0.56 |
| Pro | CCG | 0.11 |
| Pro | CCA | 0.27 |
| Pro | CCT | 0.29 |
| Pro | CCC* | 0.33 |
| Gln | CAG* | 0.73 |
| Gln | CAA | 0.27 |
| Arg | AGG | 0.22 |
| Arg | AGA* | 0.21 |
| Arg | CGG | 0.19 |
| Arg | CGA | 0.10 |
| Arg | CGT | 0.09 |
| Arg | CGC | 0.19 |
| Ser | AGT | 0.14 |
| Ser | AGC* | 0.25 |
| Ser | TCG | 0.06 |
| Ser | TCA | 0.15 |
| Ser | TCT | 0.18 |
| Ser | TCC | 0.23 |
| Thr | ACG | 0.12 |
| Thr | ACA | 0.27 |
| Thr | ACT | 0.23 |
| Thr | ACC* | 0.38 |
| Val | GTG* | 0.48 |
| Val | GTA | 0.10 |
| Val | GTT | 0.17 |
| Val | GTC | 0.25 |
| Trp | TGG* | 1 |
| Tyr | TAT | 0.42 |
| Tyr | TAC* | 0.58 |
| Stop | TGA* | 0.61 |
| Stop | TAG | 0.17 |
| Stop | TAA | 0.22 |

Certain strategies for codon optimization and/or G/C enrichment for human expression are described in WO2002/098443, which is incorporated by reference herein in its entirety. In some embodiments, a coding sequence may be optimized using a multiparametric optimization strategy. In some embodiments, optimization parameters may include parameters that influence protein expression, which can be, for example, impacted on a transcription level, an RNA level, and/or a translational level. In some embodiments, exemplary optimization parameters include, but are not limited to transcription-level parameters (including, e.g., GC content, consensus splice sites, cryptic splice sites, SD sequences, TATA boxes, termination signals, artificial recombination sites, and combinations thereof); RNA-level parameters (including, e.g., RNA instability motifs, ribosomal entry sites, repetitive sequences, and combinations thereof); translation-level parameters (including, e.g., codon usage, premature poly(A) sites, ribosomal entry sites, secondary structures, and combinations thereof); or combinations thereof. In some embodiments, a coding sequence may be optimized by a GeneOptimizer algorithm as described in Fath et al. “Multiparameter RNA and Codon Optimization: A Standardized Tool to Assess and Enhance Autologous Mammalian Gene Expression” PLoS ONE 6(3): e17596; Rabb et al., which is incorporated herein by reference in its entirety, “The GeneOptimizer Algorithm: using a sliding window approach to cope with the vast sequence space in multiparameter DNA sequence optimization” Systems and Synthetic Biology (2010) 4:215-225; and Graft et al. “Codon-optimized genes that enable increased heterologous expression in mammalian cells and elicit efficient immune responses in mice after vaccination of naked DNA” Methods Mol Med (2004) 94:197-210, the entire content of each of which is incorporated herein for the purposes described herein. In some embodiments, a coding sequence may be optimized by Eurofins' adaption and optimization algorithm “GENEius” as described in Eurofins' Application Notes: Eurofins' adaption and optimization software “GENEius” in comparison to other optimization algorithms, the entire content of which is incorporated by reference for the purposes described herein.

In some embodiments, a coding sequence utilized in accordance with the present disclosure has G/C content that is increased compared to a coding sequence for an HSV gC, gD, and/or gE (or immunogenic fragment thereof) construct described herein. In some embodiments, guanosine/cytidine (G/C) content of a coding region is modified relative to a comparable coding sequence for an HSV gC, gD, and/or gE (or immunogenic fragment thereof) construct described herein, but the amino acid sequence encoded by the nucleotide sequence is not modified.

Without wishing to be bound by any particular theory, it is proposed that GC enrichment may improve translation of a payload sequence. Typically, sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability can be determined (so-called alternative codon usage). Depending on the amino acid to be encoded by a nucleotide sequence, there are various possibilities for modification of the ribonucleic acid sequence, compared to its wild type sequence. In particular, codons which contain A and/or U nucleosides can be modified by substituting these codons by other codons, which code for the same amino acids but contain no A and/or U or contain a lower content of A and/or U nucleosides.

In some embodiments, G/C content of a coding region of a nucleotide sequence described herein is increased by at least 1%, at least 2%, at least 3%, at least 4%, at least 5%, at least 6%, or even more compared to the G/C content of the coding region prior to codon optimization, e.g., of the wild type RNA. In some embodiments, G/C content of a coding region of a nucleotide sequence described herein is decreased by at least 1%, at least 2%, at least 3%, at least 4%, at least 5%, at least 6%, or even more compared to the G/C content of the coding region prior to codon optimization, e.g., of the wild type RNA.

In some embodiments, stability and translation efficiency of a nucleotide sequence may incorporate one or more elements established to contribute to stability and/or translation efficiency of the nucleotide sequence; exemplary such elements are described, for example, in PCT/EP2006/009448 incorporated herein by reference. In some embodiments, to increase expression of a nucleotide sequence used according to the present disclosure, a nucleotide sequence may be modified within the coding region, i.e., the sequence encoding the expressed peptide or protein, without altering the sequence of the expressed peptide or protein, for example so as to increase the GC-content to increase RNA stability and/or to perform a codon optimization and, thus, enhance translation in cells.

### Methods of Treatment and Uses of the Compositions

In some embodiments, the present disclosure provides methods of vaccinating a subject against HSV and treating, impeding, inhibiting, reducing the incidence of, or suppressing an HSV infection or a symptom or manifestation thereof, comprising administration of a composition of the present disclosure.

In some embodiments, the present disclosure provides a method for treating an HSV infection in a subject, comprising contacting said subject with a composition comprising one or more RNAs, wherein each of said RNAs encodes an HSV glycoprotein or immunogenic fragment thereof. For example, said RNAs can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6. In another embodiment, the present disclosure provides a method for suppressing an HSV infection in a subject, comprising contacting said subject with a composition comprising one or more RNAs, wherein each of said RNAs encodes an HSV glycoprotein or immunogenic fragment thereof. For example, said RNAs can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

In another embodiment, the present disclosure provides a method for inhibiting an HSV infection in a subject, comprising contacting said subject with a composition comprising one or more RNAs, wherein each of said RNAs encodes an HSV glycoprotein or immunogenic fragment thereof. For example, said RNAs can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

In another embodiment, the present disclosure provides a method for reducing the incidence of HSV infection in a subject, comprising contacting said subject with a composition comprising one or more RNAs, wherein each of said RNAs encodes an HSV glycoprotein or immunogenic fragment thereof. For example, said RNAs can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

In some embodiments, the HSV infection is an HSV-1 infection. In another embodiment, the HSV infection is an HSV-2 infection.

In some embodiments, the subject is administered HSV-1 glycoproteins for methods of treating, inhibiting, suppressing, etc. an HSV-1 infection. In another embodiment, the subject is administered HSV-2 glycoproteins for methods of treating, inhibiting, suppressing, etc. an HSV-2 infection. For example, said HSV-2 glycoproteins can be one or more glycoproteins comprising an amino acid sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 5, or immunogenic fragments thereof.

In another embodiment, the subject is administered HSV-1 glycoproteins, or immunogenic fragments thereof, for methods of treating, inhibiting, suppressing, etc. an HSV-1 infection, HSV-2 infection, or a combination thereof. In another embodiment, the subject is administered HSV-2 glycoproteins, or immunogenic fragments thereof, for methods of treating, inhibiting, suppressing, etc. an HSV-1 infection, HSV-2 infection, or a combination thereof. In some embodiments, administration of HSV-1 glycoproteins (e.g., gC1, gD1, gE1, or a combination thereof), or immunogenic fragments thereof, treats or prevents HSV-1 and HSV-2 infection. In another embodiment, administration of HSV-2 glycoproteins (e.g., gC2, gD2 and gE2, or a combination thereof), or immunogenic fragments thereof, treats or prevents HSV-1 and HSV-2 infection.

In some embodiments, the present disclosure provides a method for treating, suppressing, inhibiting, or reducing the incidence of HSV-1 infection in a subject, comprising contacting said subject with a composition comprising one or more RNAs, wherein each of said RNAs encodes an HSV-1 glycoprotein or immunogenic fragment thereof.

In other embodiments, the present disclosure provides methods of inhibiting an HSV-1 infection in a subject comprising the step of administering a composition comprising one or more RNA encoding HSV-2 proteins, or immunogenic fragments thereof. In other embodiments, the present disclosure provides methods of treating an HSV-1 infection in a subject comprising the step of administering a composition comprising one or more RNA encoding HSV-2 proteins. In other embodiments, the present disclosure provides methods of suppressing an HSV-1 infection in a subject comprising the step of administering a composition comprising one or more RNA encoding HSV-2 proteins. In other embodiments, the present disclosure provides methods of reducing the incidence of an HSV-1 infection in a subject comprising the step of administering a composition comprising one or more RNA encoding HSV-2 proteins.

In some embodiments, the HSV-2 protein comprises an HSV-2 glycoprotein. In some embodiments, the HSV-2 glycoprotein comprises and HSV-2 gE. In some embodiments, the HSV-2 glycoprotein comprises an HSV-2 gD or an immunogenic fragment thereof, an HSV gC or an immunogenic fragment thereof, and an HSV gE or an immunogenic fragment thereof.

In some embodiments, the present disclosure provides a method for treating, suppressing, inhibiting, or reducing the incidence of HSV-2 infection in a subject, comprising contacting said subject with a composition comprising one or more RNAs, wherein each of said RNAs encodes an HSV-2 glycoprotein or immunogenic fragment thereof.

In some embodiments, said contacting is via administration to said subject.

In another embodiment, the present disclosure provides a method of treating, suppressing, inhibiting, or reducing the incidence of an HSV infection in a subject, the method comprising the step of administering to said subject an RNA encoding an HSV gE or immunogenic fragment thereof as described herein.

In another embodiment, the present disclosure provides a method of treating, suppressing, inhibiting, or reducing the incidence of an HSV infection in a subject, the method comprising the step of administering to said subject an RNA encoding an HSV-2 gE or immunogenic fragment thereof as described herein.

In another embodiment, the present disclosure provides a method of treating, suppressing, inhibiting, or reducing the incidence of an HSV infection in a subject, the method comprising the step of administering to said subject an RNA encoding an HSV-1 gE or immunogenic fragment thereof as described herein.

In some embodiments, an RNA is part of a composition, which in some embodiments, is an immunogenic composition.

In another embodiment, the present disclosure provides a method of treating, suppressing, inhibiting, or reducing the incidence of an HSV infection in a subject, the method comprising the step of administering to said subject an immunogenic composition comprising RNAs encoding: (a) an HSV gD or immunogenic fragment thereof; (b) an HSV gC or fragment thereof as described herein; (c) an HSV gE or fragment thereof as described herein, or a combination thereof.

In another embodiment, the present disclosure provides a method of treating, suppressing, inhibiting, or reducing the incidence of an HSV infection in a subject, the method comprising the step of administering to said subject an immunogenic composition comprising RNAs encoding: (a) an HSV-2 gD or immunogenic fragment thereof; (b) an HSV-2 gC or fragment thereof as described herein; and (c) an HSV-2 gE or fragment thereof as described herein, or a combination thereof.

In another embodiment, the present disclosure provides a method of treating, suppressing, inhibiting, or reducing the incidence of an HSV infection in a subject, the method comprising the step of administering to said subject an immunogenic composition comprising RNAs encoding: (a) an HSV-1 gD or immunogenic fragment thereof; (b) an HSV-1 gC or fragment thereof as described herein; and (c) an HSV-1 gE or fragment thereof as described herein, or a combination thereof.

In another embodiment, the present disclosure provides a method of inducing an anti-HSV immune response in a subject, the method comprising the step of administering to said subject an RNA encoding an HSV gE or immunogenic fragment thereof as described herein.

In another embodiment, the present disclosure provides a method of inducing an anti-HSV immune response in a subject, the method comprising the step of administering to said subject an RNA encoding an HSV-2 gE immunogenic or fragment thereof as described herein.

In another embodiment, the present disclosure provides a method of inducing an anti-HSV immune response in a subject, the method comprising the step of administering to said subject an RNA encoding an HSV-1 gE immunogenic or fragment thereof as described herein.

In another embodiment, the present disclosure provides a method of inducing an anti-HSV immune response in a subject, the method comprising the step of administering to said subject an immunogenic composition comprising RNAs encoding: (a) an HSV gD or immunogenic fragment thereof as described herein; (b) an HSV gC or immunogenic fragment thereof as described herein; (c) an HSV gE or immunogenic fragment thereof as described herein, or a combination thereof.

In another embodiment, the present disclosure provides a method of inducing an anti-HSV immune response in a subject, the method comprising the step of administering to said subject an immunogenic composition comprising RNAs encoding: (a) an HSV-2 gD or immunogenic fragment thereof as described herein; (b) an HSV-2 gC or immunogenic fragment thereof as described herein; and (c) an HSV-2 gE or immunogenic fragment thereof as described herein, or a combination thereof.

In another embodiment, the present disclosure provides a method of inducing an anti-HSV immune response in a subject, the method comprising the step of administering to said subject an immunogenic composition comprising RNAs encoding: (a) an HSV-1 gD or immunogenic fragment thereof as described herein; (b) an HSV-1 gC or immunogenic fragment thereof as described herein; and (c) an HSV-1 gE or immunogenic fragment thereof as described herein, or a combination thereof.

In another embodiment, the present disclosure provides a method of inhibiting a primary HSV infection in a subject, the method comprising the step of administering to the subject a composition of the present disclosure. In another embodiment, the present disclosure provides a method of treating an HSV infection in a subject, the method comprising the step of administering to said subject a composition of the present disclosure. In another embodiment, the present disclosure provides a method of reducing the incidence of an HSV infection in a subject, the method comprising the step of administering to said subject a composition of the present disclosure. In another embodiment, the present disclosure provides a method of inhibiting a flare following a primary HSV infection in a subject, the method comprising the step of administering to said subject a composition of the present disclosure.

In some embodiments, the present disclosure provides methods of treating and/or suppressing a primary HSV infection and/or a secondary HSV infection. In some embodiments, a “primary” infection refers to a first-time infection. In some embodiments, a “secondary” infection refers to a recurrence of an HSV infection.

In some embodiments, a “flare” or “recurrence” refers to reinfection of skin tissue following latent neuronal HSV infection. In another embodiment, the terms refer to reactivation of HSV after a latency period. In another embodiment, the terms refer to symptomatic HSV lesions following a non-symptomatic latency period.

In another embodiment, the present disclosure provides a method of inhibiting spread of HSV. In some embodiments, the spread from dorsal root ganglia (DRG) to skin is inhibited. In some embodiments, cell-to-cell spread of HSV is inhibited. In some embodiments, anterograde spread is inhibited. In some embodiments, retrograde spread is inhibited. “DRG” refers, in some embodiments, to a neuronal cell body and in another embodiment, contain the neuron cell bodies of nerve fibers. In another embodiment, the term refers to any other definition of “DRG” used in the art. In another embodiment, spread of HSV to neural tissue is inhibited.

In another embodiment, the present disclosure provides a method of inhibiting a recurrence following a primary HSV infection in a subject, the method comprising the step of administering to said subject a composition of the present disclosure. In another embodiment, the present disclosure provides a method of preventing a recurrence following a primary HSV infection in a subject, the method comprising the step of administering to said subject a composition of the present disclosure.

In another embodiment, the present disclosure provides a method of inhibiting an HSV labialis following a primary HSV infection in a subject, the method comprising the step of administering to said subject a composition of the present disclosure.

In another embodiment, the present disclosure provides a method of preventing a recurrence of an HSV infection, the method comprising the step of administering to said subject a composition of the present disclosure. In another embodiment, the present disclosure provides a method of diminishing the severity of a recurrence of an HSV infection, the method comprising the step of administering to said subject a composition of the present disclosure. In another embodiment, the present disclosure provides a method of reducing the frequency of a recurrence of an HSV infection, the method comprising the step of administering to said subject a composition of the present disclosure. In some embodiments, the present disclosure provides any of the described methods in an HIV-infected subject.

In another embodiment, the present disclosure provides a method of treating HSV encephalitis in a subject, the method comprising the step of administering to said subject a composition of the present disclosure. In another embodiment, the present disclosure provides a method of reducing the incidence of HSV encephalitis in a subject, the method comprising the step of administering to said subject a composition of the present disclosure. “HSV encephalitis” refers, in some embodiments, to encephalitis caused by HSV. In another embodiment, the term refers to encephalitis associated with HSV. In another embodiment, the term refers to any other type of HSV-mediated encephalitis known in the art.

In another embodiment, the present disclosure provides a method of treating or reducing an HSV neonatal infection in a subject, the method comprising the step of administering to said subject a composition of the present disclosure.

In another embodiment, the present disclosure provides a method for introducing an HSV glycoprotein to a cell of a subject, comprising contacting said cell with an in vitro-transcribed RNA molecule encoding the recombinant protein, or fragment thereof, wherein said in vitro-transcribed RNA molecule further comprises a modified nucleoside, thereby introducing said HSV glycoprotein, or immunogenic fragment thereof, into said cell of said subject.

In another embodiment, the present disclosure provides a method for inducing a mammalian cell to produce an HSV glycoprotein, or immunogenic fragment thereof, comprising contacting said mammalian cell with an in vitro-synthesized RNA molecule encoding the HSV glycoprotein, or immunogenic fragment thereof, the in vitro-synthesized RNA molecule comprising a pseudouridine, thereby inducing said mammalian cell to produce said HSV glycoprotein, or immunogenic fragment thereof.

It is to be understood that reference to HSV herein refers in some embodiments, to HSV-1, while in another embodiment, to HSV-2, while in another embodiment, to HSV-1 and HSV-2.

“HSV-1” refers, in another embodiment, to a Herpes Simplex Virus-1. In another embodiment, the term refers to a KOS strain. In another embodiment, the term refers to an F strain. In another embodiment, the term refers to an NS strain. In another embodiment, the term refers to a CL101 strain. In another embodiment, the term refers to a “17” strain. In another embodiment, the term refers to a “17+syn” strain. In another embodiment, the term refers to a MacIntyre strain. In another embodiment, the term refers to an MP strain. In another embodiment, the term refers to an HF strain. In another embodiment, the term refers to any other HSV-1 strain known in the art.

“HSV-2” refers, in another embodiment, to a Herpes Simplex Virus-2. In another embodiment, the term refers to an HSV-2 333 strain. In another embodiment, the term refers to a 2.12 strain. In another embodiment, the term refers to an HG52 strain. In another embodiment, the term refers to an MS strain. In another embodiment, the term refers to a G strain. In another embodiment, the term refers to a 186 strain. In another embodiment, the term refers to any other HSV-2 strain known in the art.

In another embodiment, the present disclosure provides a method of vaccinating a subject against an HSV infection, the method comprising the step of administering to said subject a composition of the present disclosure. In another embodiment, the present disclosure provides a method of suppressing an HSV infection in a subject, the method comprising the step of administering to said subject a composition of the present disclosure. In another embodiment, the present disclosure provides a method of impeding an HSV infection in a subject, the method comprising the step of administering to said subject a composition of the present disclosure. In another embodiment, the present disclosure provides a method of impeding a primary HSV infection in a subject, the method comprising the step of administering to said subject a composition of the present disclosure. In another embodiment, the present disclosure provides a method of impeding neuronal HSV spread in a subject, the method comprising the step of administering to said subject a composition of the present disclosure.

The terms “impeding an HSV infection” and “impeding a primary HSV infection” refer, in another embodiment, to decreasing the titer of infectious virus. In another embodiment, the terms refer to decreasing the extent of viral replication.

In another embodiment, the present disclosure provides a method of treating an HSV-mediated herpetic ocular disease in a subject, the method comprising the step of administering to said subject a composition of the present disclosure. In another embodiment, the present disclosure provides a method of reducing the incidence of an HSV-mediated herpetic ocular disease in a subject, the method comprising the step of administering to said subject a composition of the present disclosure. In some embodiments, the herpetic ocular disease comprises a corneal infection. In another embodiment, the present disclosure provides a method of treating an HSV-1 corneal infection or herpes keratitis in a subject, the method comprising the step of administering to said subject a composition of the present disclosure. In another embodiment, the present disclosure provides a method of reducing the incidence of an HSV-1 corneal infection or herpes keratitis in a subject, the method comprising the step of administering to said subject a composition of the present disclosure. In some embodiments, the HSV-1 corneal infection or herpes keratitis is an HSV-1 corneal infection or herpes keratitis.

In another embodiment, the present disclosure provides a method of treating herpetic stomatitis in a subject, the method comprising the step of administering to said subject a composition of the present disclosure. In another embodiment, the present disclosure provides a method of reducing the incidence of herpetic stomatitis in a subject, the method comprising the step of administering to said subject a composition of the present disclosure. In some embodiments, the stomatitis is an HSV-1 stomatitis.

In another embodiment, the present disclosure provides a method of treating, suppressing or inhibiting an HSV genital infection, the method comprising the step of administering to said subject a composition of the present disclosure. In another embodiment, the present disclosure provides a method of treating, suppressing or inhibiting any manifestation of recurrent HSV infection, the method comprising the step of administering to said subject a composition of the present disclosure.

In another embodiment, the present disclosure provides a method of reducing the incidence of an HSV-mediated genital ulcer disease in a subject, the method comprising the step of administering to said subject a composition of the present disclosure.

In another embodiment, the present disclosure provides a method of impeding establishment of a latent HSV infection in a subject, the method comprising the step of administering to said subject a composition of the present disclosure. In another embodiment, the present disclosure provides a method of preventing establishment of a latent HSV infection in a subject, the method comprising the step of administering to said subject a composition of the present disclosure. In another embodiment, the present disclosure provides a method of suppressing the establishment of a latent HSV infection in a subject, the method comprising the step of administering to said subject a composition of the present disclosure. In some embodiments, establishment of a latent HSV infection in a subject is through HSV infection of the DRG. In another embodiment, establishment of a latent HSV infection in a subject is through HSV infection of the trigeminal nerve or trigeminal ganglia.

In other embodiments, the present disclosure provides methods of preventing establishment of a latent HSV infection in a subject comprising administering a composition of the present disclosure. In other embodiments, the present disclosure provides methods of treating a latent HSV infection in a subject comprising administering a composition of the present disclosure.

In some embodiments, the present disclosure provides a method of treating, suppressing or inhibiting a genital herpes infection in a subject, comprising the step of administering to said subject a composition of the present disclosure. In another embodiment, the present disclosure provides a method of treating, suppressing or inhibiting an oral herpes infection in a subject, comprising the step of administering to said subject a composition of the present disclosure.

In another embodiment, the present disclosure provides a method of treating or inhibiting an orolabial herpes infection in a subject, comprising the step of administering to said subject a composition of the present disclosure. In some embodiment, the HSV infection is on a lip of the subject.

In another embodiment, the present disclosure provides a method of inhibiting an oral mucosal infection in a subject comprising administering a composition of the present disclosure. In other embodiments, the present disclosure provides methods of suppressing an oral mucosal infection in a subject comprising administering a composition of the present disclosure. In other embodiments, the present disclosure provides methods of reducing the incidence of an oral mucosal infection in a subject comprising administering a composition of the present disclosure. In other embodiments, the present disclosure provides methods of preventing an oral mucosal infection in a subject comprising administering a composition of the present disclosure. In other embodiments, the present disclosure provides methods of treating an oral mucosal infection in a subject comprising administering a composition of the present disclosure.

In some embodiments, an oral mucosal infection is an HSV-1 infection. In another embodiment, an oral mucosal infection is an HSV-2 infection.

In another embodiment, the present disclosure provides a method of reducing the incidence of an HSV-mediated encephalitis in a subject, the method comprising the step of administering to said subject a composition of the present disclosure.

In another embodiment, the herpes-mediated encephalitis treated or prevented by a method of the present disclosure is a focal herpes encephalitis. In another embodiment, the herpes-mediated encephalitis is a neonatal herpes encephalitis. In another embodiment, the herpes-mediated encephalitis is any other type of herpes-mediated encephalitis known in the art.

In another embodiment, the present disclosure provides a method of treating or reducing the incidence of a disease, disorder, or symptom associated with or secondary to an HSV-mediated encephalitis in a subject, the method comprising the step of administering to said subject a composition of the present disclosure.

In another embodiment, the present disclosure provides a method of treating, reducing the pathogenesis of, ameliorating the symptoms of, ameliorating the secondary symptoms of, reducing the incidence of, prolonging the latency to a relapse of an HSV infection in a subject, comprising the step of administering to the subject a composition of the present disclosure.

In another embodiment, the present disclosure provides a method of protecting a subject against formation of a zosteriform lesion or an analogous outbreak in a human subject. In another embodiment, the present disclosure provides a method of inhibiting the formation of an HSV zosteriform lesion or an analogous outbreak in a human subject.

“Zosteriform” refers, in some embodiments, to skin lesions characteristic of an HSV infection, particularly during reactivation infection, which, in some embodiments, begin as a rash and follow a distribution near dermatomes, commonly occurring in a strip or belt-like pattern. In some embodiments, the rash evolves into vesicles or small blisters filled with serous fluid. In some embodiments, zosteriform lesions form in mice as a result of contact with HSV. In another embodiment, zosteriform lesions form in humans as a result of contact with HSV. “Zosteriform spread” refers, in some embodiments, to an HSV infection that spreads from the ganglia to secondary skin sites within the dermatome. In another embodiment, the term refers to spread within the same dermatome as the initial site of infection. In another embodiment, the term refers to any other definition of “zosteriform spread” known in the art. “Outbreak”, in another embodiment, refers to a sudden increase in symptoms of a disease or in the spread or prevalence of a disease, and in some embodiments, refers to a sudden increase in zosteriform lesions, while in another embodiment, “outbreak” refers to a sudden eruption of zosteriform lesions.

In some embodiments, the present disclosure provides a method of impeding the formation of a dermatome lesion or an analogous condition in a subject. In some embodiments, dermatome lesions form as a result of contact with HSV. In another embodiment, dermatome lesions most often develop when the virus reactivates from latency in the ganglia and in some embodiments, spreads down nerves, in some embodiments, causing a recurrent infection.

It is to be understood that the methods of the present disclosure may be used to treat, inhibit, suppress, etc. an HSV infection or primary or secondary symptoms related to such an infection following exposure of the subject to HSV. In another embodiment, the subject has been infected with HSV before vaccination. In another embodiment, the subject is at risk for HSV infection. In another embodiment, whether or not the subject has been infected with HSV at the time of vaccination, vaccination by a method of the present disclosure is efficacious in treating, inhibiting, suppressing, etc. an HSV infection or primary or secondary symptoms related to such an infection.

In some embodiments, “treating” refers to either therapeutic treatment or prophylactic or preventative measures, wherein the object is to prevent or lessen the targeted pathologic condition or disorder as described hereinabove. Thus, in some embodiments, treating may include directly affecting or curing, suppressing, inhibiting, preventing, reducing the severity of, delaying the onset of, reducing symptoms associated with the disease, disorder or condition, or a combination thereof. Thus, in some embodiments, “treating” refers inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, the compositions and methods of the present disclosure are effective in lowering HSV acquisition rates, duration of HSV infection, frequency of HSV reactivation, or a combination thereof. In another embodiment, the compositions and methods of the present disclosure are effective in treating or inhibiting genital ulcer disease, which in some embodiments, entails decreasing the severity or frequency of HSV genital ulcer disease. In some embodiments, the compositions and methods of the present disclosure block immune evasion from complement. In some embodiments, vaccination with RNA-encoded HSV subunits may produce high titers of neutralizing antibodies or potent T-cell responses; however, upon subsequent infection, HSV immune evasion molecules may block the activities of antibodies or T cells, thereby reducing composition efficacy. In some embodiments, the compositions and methods of the present disclosure incorporate strategies to block virus mediated immune evasion by, in some embodiments, enhancing the effectiveness of e.g., a HSV-1 gD, or immunogenic fragment thereof, composition using HSV-1 gC to prevent immune evasion from complement.

In some embodiments, studies in guinea pigs and mice suggest that viral load in ganglia correlates with the frequency of recurrent HSV infections. Thus, in some embodiments, the compositions and methods of the present disclosure are useful for preventing or inhibiting recurrent HSV infections. In some embodiments, antibodies to e.g., HSV-1 gC block domains involved in immune evasion, which enhances complement activity, improves neutralizing activity of anti-HSV-1 gD IgG, increases antibody- and complement-dependent cellular cytotoxicity, and augments complement-mediated neutralization and lysis of infected cells.

In some embodiments, symptoms are primary, while in another embodiment, symptoms are secondary. In some embodiments, “primary” refers to a symptom that is a direct result of the subject viral infection, while in some embodiments, “secondary” refers to a symptom that is derived from or consequent to a primary cause. In some embodiments, the compositions and strains for use in the present disclosure treat primary or secondary symptoms or secondary complications related to HSV infection.

In another embodiment, “symptoms” may be any manifestation of an HSV infection, comprising blisters, ulcerations, or lesions on the urethra, cervix, upper thigh, and/or anus in women and on the penis, urethra, scrotum, upper thigh, and anus in men, inflammation, swelling, fever, flu-like symptoms, sore mouth, sore throat, pharyngitis, pain, blisters on tongue, mouth or lips, ulcers, cold sores, neck pain, enlarged lymph nodes, reddening, bleeding, itching, dysuria, headache, muscle pain, etc., or a combination thereof.

In another embodiment, the disease, disorder, or symptom is fever. In another embodiment, the disease, disorder, or symptom is headache. In another embodiment, the disease, disorder, or symptom is stiff neck. In another embodiment, the disease, disorder, or symptom is seizures. In another embodiment, the disease, disorder, or symptom is partial paralysis. In another embodiment, the disease, disorder, or symptom is stupor. In another embodiment, the disease, disorder, or symptom is coma. In another embodiment, the disease, disorder, or symptom is any other disease, disorder, or symptom known in the art that is associated with or secondary to a herpes-mediated encephalitis.

Methods of determining the presence and severity of herpes-mediated encephalitis are well known in the art, and are described, for example, in Bonkowsky J L et al. (Herpes simplex virus central nervous system relapse during treatment of infantile spasms with corticotropin. Pediatrics. 2006 May; 117(5):e1045-8) and Khan O A, et al. (Herpes encephalitis presenting as mild aphasia: case report. BMC Fam Pract. 2006 Mar. 24; 7:22). Each method represents a separate embodiment of the present disclosure.

In another embodiment, the present disclosure provides a method of treating or reducing the incidence of a disease, disorder, or symptom associated with an HSV infection in a subject, the method comprising the step of administering to said subject a composition of the present disclosure.

In another embodiment, the disease, disorder, or symptom secondary to an HSV infection is oral lesions. In another embodiment, the disease, disorder, or symptom is genital lesions. In another embodiment, the disease, disorder, or symptom is oral ulcers. In another embodiment, the disease, disorder, or symptom is genital ulcers. In another embodiment, the disease, disorder, or symptom is fever. In another embodiment, the disease, disorder, or symptom is headache. In another embodiment, the disease, disorder, or symptom is muscle ache. In another embodiment, the disease, disorder, or symptom is swollen glands in the groin area. In another embodiment, the disease, disorder, or symptom is painful urination. In another embodiment, the disease, disorder, or symptom is vaginal discharge. In another embodiment, the disease, disorder, or symptom is blistering. In another embodiment, the disease, disorder, or symptom is flu-like malaise. In another embodiment, the disease, disorder, or symptom is keratitis. In another embodiment, the disease, disorder, or symptom is herpetic whitlow. In another embodiment, the disease, disorder, or symptom is Bell's palsy. In another embodiment, the disease, disorder, or symptom is herpetic erythema multiforme. In another embodiment, the disease, disorder, or symptom is a lower back symptom (e.g. numbness, tingling of the buttocks or the area around the anus, urinary retention, constipation, and impotence). In another embodiment, the disease, disorder, or symptom is a localized eczema herpeticum. In another embodiment, the disease, disorder, or symptom is a disseminated eczema herpeticum. In another embodiment, the disease, disorder, or symptom is a herpes gladiatorum. In another embodiment, the disease, disorder, or symptom is a herpetic sycosis. In another embodiment, the disease, disorder, or symptom is an esophageal symptom (e.g. difficulty swallowing or burning, squeezing throat pain while swallowing, weight loss, pain in or behind the upper chest while swallowing). In another embodiment, the disease, disorder, or symptom is any other disease, disorder, or symptom that is known in the art. Each disease, disorder, and symptom represents a separate embodiment of the present disclosure.

Thus, in some embodiments, the compositions and methods of the instant disclosure treat, suppress, inhibit, or reduce the incidence of the infection itself, while in another embodiment, the compositions and methods of the instant disclosure treat, suppress, inhibit, or reduce the incidence of primary symptoms of the infection, while in another embodiment, the compositions and methods of the instant disclosure treat, suppress, inhibit, or reduce the incidence of secondary symptoms of the infection. It is to be understood that the compositions and methods of the instant disclosure may affect any combination of the infection, the primary symptoms caused by the infection, and secondary symptoms related to the infection.

The HSV infection that is treated or ameliorated by methods and compositions of the present disclosure is, in another embodiment, a genital HSV infection. In another embodiment, the HSV infection is an oral HSV infection. In another embodiment, the HSV infection is an ocular HSV infection. In another embodiment, the HSV infection is a dermatologic HSV infection.

In another embodiment, the present disclosure provides a method of reducing the incidence of a disseminated HSV infection in a subject, the method comprising the step of administering to said subject a composition of the present disclosure.

In another embodiment, the present disclosure provides a method of reducing the incidence of a neonatal HSV infection in an offspring of a subject, the method comprising the step of administering to said subject a composition of the present disclosure.

In another embodiment, the present disclosure provides a method of reducing a transmission of an HSV infection from a subject to an offspring thereof, the method comprising the step of administering to said subject a composition of the present disclosure.

In another embodiment, the offspring is an infant. In another embodiment, the transmission that is reduced or inhibited is transmission during birth. In another embodiment, transmission during breastfeeding is reduced or inhibited. In another embodiment, the transmission that is reduced or inhibited is any other type of parent-to-offspring transmission known in the art.

In another embodiment, the present disclosure provides a method of reducing a severity of a neonatal HSV infection in an offspring of a subject, the method comprising the step of administering to said subject a composition of the present disclosure.

In some embodiments, the composition for use in the methods of the present disclosure elicits an immune response against HSV. In another embodiment, the composition for use in the methods of the present disclosure elicits an immune response against HSV-1. In another embodiment, the composition for use in the methods of the present disclosure elicits an immune response against HSV-2. In another embodiment, the composition comprises RNAs encoding gD and gC proteins, or immunogenic fragments thereof. In another embodiment, the composition comprises RNAs encoding gE and gD proteins, or immunogenic fragments thereof. In another embodiment, the composition comprises RNAs encoding gC and gE proteins, or immunogenic fragments thereof. In another embodiment, the composition comprises RNAs encoding gE, gD, and gC proteins, or immunogenic fragments thereof. In another embodiment, the composition comprises RNAs encoding gE, gD, or gC protein, or immunogenic fragments thereof. In another embodiment, the proteins encoded by the RNAs are HSV-1 proteins, or immunogenic fragments thereof. In another embodiment, the proteins encoded by the RNAs are HSV-2 proteins, or immunogenic fragments thereof. In another embodiment, the proteins encoded by the RNAs comprise both HSV-1 and HSV-2 proteins, or immunogenic fragments thereof.

It is to be understood that, in some embodiments, a subject according to any of the embodiments described herein may be a subject infected with, or in another embodiment, susceptible to infection with HSV. In some embodiments, a subject may be infected with, or in another embodiment, susceptible to infection with at least one other pathogen. In some embodiments, a subject may be immunocompromised. In some embodiments, the subject is infected by HSV, while in another embodiment, the subject is at risk for infection by HSV, which in some embodiments, is a subject who is a neonate, in another embodiment, immunocompromised, in another embodiment, elderly, and in another embodiment, an immunocompromised neonate or an immunocompromised elderly subject.

In another embodiment, the compositions of the present disclosure and their related uses may suppress, inhibit, prevent or treat an HIV infection in a subject. In some embodiments, the compositions of the present disclosure and their related uses may treat secondary complications of HIV infection, which in some embodiments, are opportunistic infections, neoplasms, neurologic abnormalities, or progressive immunologic deterioration. In another embodiment, the methods comprise treating acquired immunodeficiency syndrome (AIDS). In another embodiment, the methods comprise treating a decline in the number of CD4+T lymphocytes.

In another embodiment, the present disclosure provides a method of reducing HIV-1 transmission to an offspring, the method comprising the step of administering to a subject a composition of the present disclosure. As is known in the art, HSV-2 infection increases HIV-1 viral shedding in genital secretions (Nagot N et al., Reduction of HIV-1 RNA levels with therapy to suppress herpes simplex virus. N Engl J Med. 2007 Feb. 22; 356(8):790-9). Thus, methods of the present disclosure of inhibiting HSV-2 infection are also efficacious for reducing HIV-1 transmission to an offspring. In another embodiment, the mutant HSV strain is an HSV-1 strain. In another embodiment, the mutant HSV strain is an HSV-2 strain.

In another embodiment, the present disclosure provides a method of reducing HIV-1 transmission to a sexual partner, the method comprising the step of administering to a subject a composition of the present disclosure. As is known in the art, HSV-2 infection increases HIV-1 viral shedding in genital secretions. Thus, methods of the present disclosure of inhibiting HSV-2 infection are also efficacious for reducing HIV-1 transmission to a sexual partner. In another embodiment, the mutant HSV strain is an HSV-1 strain. In another embodiment, the mutant HSV strain is an HSV-2 strain.

In another embodiment, the present disclosure provides a method of reducing susceptibility to HIV-1, the method comprising the step of administering to a subject a composition of the present disclosure. As is known in the art, HSV-2 infection increases HIV-1 replication (Ouedraogo A et al., Impact of suppressive herpes therapy on genital HIV-1 RNA among women taking antiretroviral therapy: a randomized controlled trial. AIDS. 2006 Nov. 28; 20(18):2305-13). Thus, methods of the present disclosure of inhibiting HSV-2 infection are also efficacious for reducing susceptibility to HIV-1. In another embodiment, the mutant HSV strain is an HSV-1 strain. In another embodiment, the mutant HSV strain is an HSV-2 strain.

Thus, in some embodiments, the present disclosure provides a method of inhibiting a primary HSV infection in an HIV-infected subject, comprising the step of administering to said subject a composition of the present disclosure. In another embodiment, the present disclosure provides a method of treating or reducing the incidence of an HSV infection in an HIV-infected subject, comprising the step of administering to said subject a composition of the present disclosure. In another embodiment, the present disclosure provides a method of inhibiting a flare, recurrence, or HSV labialis following a primary HSV infection in an HIV-infected subject, the method comprising the step of administering to said subject a composition of the present disclosure. In some embodiments, administration of a composition of the present disclosure an anti-HSV immune response.

In another embodiment, the present disclosure provides a method for inducing an immune response in a subject, the method comprising the step of administering to said subject a nucleoside RNA composition of the present disclosure. In another embodiment, the immune response comprises a CD4 immune response. In another embodiment, the immune response comprises a CD8 immune response. In another embodiment, the immune response comprises a T follicular helper cell immune response. In another embodiment, the immune response comprises a germinal center B cell immune response. In another embodiment, the immune response comprises an IgG antibody response to gC2, gD2, gE2, or immunogenic fragments thereof, or a combination thereof.

In another embodiment, the present disclosure provides a method of treating a Herpes Simplex Virus (HSV) infection in a subject, the method comprising the step of intramuscularly administering to said subject a nucleoside RNA composition of the present disclosure. In another embodiment, the disclosure provides a method of suppressing, inhibiting, or reducing the incidence of a Herpes Simplex Virus (HSV) infection in a subject, the method comprising the step of intramuscularly administering to said subject a nucleoside RNA composition of the present disclosure.

In some embodiments, the methods as described herein comprise administering a polyribonucleotide, RNA, or composition as described herein together with one or more antiviral drugs. In some embodiments, the antiviral drug comprises acyclovir, valacyclovir, famciclovir, foscarnet, 1-Docosanol, or a combination thereof.

### Administration and Pharmaceutical Regimens

Compositions of the present disclosure can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally, intra-nasally, intra-tumorally, or topically.

“Administering,” in another embodiment, refers to directly introducing into a subject by injection or other means a composition of the present disclosure. In another embodiment, “administering” refers to contacting a cell of the subject's immune system with a composition or RNA encoding HSV protein or mixture thereof.

In another embodiment of the methods and compositions of the present disclosure, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present disclosure, the active ingredient is formulated in a capsule. In accordance with this embodiment, the compositions of the present disclosure comprise, in addition to the active compound and the inert carrier or diluent, a hard gelating capsule.

In other embodiments, the pharmaceutical compositions are administered by intravenous, intra-arterial, or intramuscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intramuscularly and are thus formulated in a form suitable for intramuscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the compositions or their physiologically tolerated derivatives are prepared and applied as solutions, suspensions, or emulsions in a physiologically acceptable diluent with or without a pharmaceutical carrier.

In another embodiment, the composition is administered as a suppository, for example a rectal suppository or a urethral suppository. In another embodiment, the pharmaceutical composition is administered by subcutaneous implantation of a pellet. In another embodiment, the pellet provides for controlled release of agent over a period of time.

In a preferred embodiment, pharmaceutical compositions are administered intramuscularly, subcutaneously or intradermally.

“Effective dosage” of the polynucleotide, refers, in another embodiment, to an amount sufficient to exert a therapeutic effect. In another embodiment, the term refers to an amount sufficient to elicit expression of a detectable amount of the encoded protein. Each possibility represents a separate embodiment of the present disclosure.

Methods for measuring the dose or amount of an RNA encoding an HSV glycoprotein or an immunogenic fragment thereof (e.g. in human subjects) to be administered are well known in the art, and include, for example, dose-escalating trials. Each method represents a separate embodiment of the present disclosure.

In some embodiments, any of the HSV compositions of and for use in the methods of this disclosure will comprise an RNA encoding an HSV protein or an immunogenic fragment thereof, or combination of RNAs encoding HSV proteins, or immunogenic fragments thereof, of the present disclosure, in any form or embodiment as described herein. In some embodiments, any of the compositions of and for use in the methods will consist of an RNA encoding an HSV protein or an immunogenic fragment thereof, or combination of RNA encoding HSV proteins, or immunogenic fragments thereof, of the present disclosure, in any form or embodiment as described herein. In some embodiments, the compositions of this disclosure will consist essentially of an RNA encoding an HSV protein, or immunogenic fragment thereof, or combination of RNAs encoding HSV proteins, or immunogenic fragments thereof, of the present disclosure, in any form or embodiment as described herein. In some embodiments, the term “comprise” refers to the inclusion of RNA encoding other HSV proteins or fragments thereof, as well as inclusion of RNA encoding other proteins, or fragments thereof, that may be known in the art. In some embodiments, the term “consisting essentially of” refers to a composition, which has the RNA encoding a specific HSV protein or fragment thereof. However, other components may be included that are not involved directly in the utility of the RNA(s) encoding HSV protein(s) or fragment(s) thereof. In some embodiments, the term “consisting” refers to a composition having an RNA encoding particular HSV protein or fragment or combination of RNAs encoding HSV proteins or fragments of the present disclosure, in any form or embodiment as described herein.

In another embodiment, the present disclosure provides a composition for treating HSV-1 or a symptom or manifestation thereof, the composition comprising an RNA of the present disclosure.

In another embodiment, the present disclosure provides a composition for treating HSV-2 or a symptom or manifestation thereof, the composition comprising an RNA of the present disclosure.

It is to be understood that the compositions, and methods of the present disclosure may be used in non-HSV herpesvirus as well. In some embodiments, non-HSV herpesvirus proteins gD, gE, or gC, or immunogenic fragments thereof, may be used. In some embodiments, non-HSV herpesvirus proteins gD, gE, or gC are 70% homologous, in another embodiment, 80% homologous, in another embodiment, 85% homologous, in another embodiment, 90% homologous, in another embodiment, 95% homologous, in another embodiment, 98% homologous, and in another embodiment, 100% homologous to the gD, gE, or gC proteins of HSV-1, or in another embodiment, to the gD, gE, or gC proteins of HSV-2. In some embodiments, such compositions may be useful in suppressing, inhibiting, preventing, or treating cancers, or in another embodiment, tumors. In some embodiments, non-HSV herpesvirus comprise Varicella Zoster Virus (VZV), Epstein-Barr virus (EBV), EBNA, cytomegalovirus (CMV), and human herpesvirus-6 (HHV-6).

In another embodiment, a composition of the present disclosure is administered once in the methods of the present disclosure. In another embodiment, the composition is administered twice. In another embodiment, the composition is administered three times. In another embodiment, the composition is administered four times. In another embodiment, the composition is administered at least four times. In another embodiment, the composition is administered more than four times.

In some embodiments, a subject is immunized with a single administration of the composition. In another embodiment, a subject is immunized with a single dose. In another embodiment, a subject is immunized with two doses. In another embodiment, a subject is immunized with three doses. In another embodiment, a subject is immunized with four doses. In another embodiment, a subject is immunized with five doses.

In another embodiment, the dosage is a daily dose. In another embodiment, the dosage is a weekly dose. In another embodiment, the dosage is a monthly dose. In another embodiment, the dosage is an annual dose. In another embodiment, the dose is one is a series of a defined number of doses. In another embodiment, the dose is a one-time dose.

In some embodiments, the methods of the present disclosure include a one-time or single administration of compositions comprising one or more nucleoside RNAs of the present disclosure. In another embodiment, the methods of the present disclosure include administration of compositions comprising one or more nucleoside RNAs in a prime and boost approach. In some embodiments, the methods of the present disclosure further comprise the step of administering to said subject one or more additional administrations of said nucleoside RNA composition subsequent to the first administration.

In some embodiments, a method as described herein involves administration of a priming dose and a boosting dose. In another embodiment, a booster doses is administered following a priming dose and comprises one or modified more RNAs encoding HSV-1 proteins or immunogenic fragments thereof. In another embodiment, a booster dose is administered following a priming vaccination and comprises one or more modified more RNAs encoding HSV-2 proteins or immunogenic fragments thereof.

In another embodiment, the methods of the present disclosure comprise administering a composition comprising one or more nucleoside RNAs encoding one or more HSV glycoproteins, or immunogenic fragments thereof, as a first administration and a composition comprising one or more HSV glycoproteins, or immunogenic fragments thereof, as a second or subsequent administration. In some embodiments, the HSV glycoproteins, or immunogenic fragments thereof, encoded by the RNA in the first (or prime) administration are the same glycoproteins, or immunogenic fragments thereof, in the second or subsequent (or boost) administration. In another embodiment, a composition comprising one or more HSV glycoproteins, or immunogenic fragments thereof, is administered as a first administration, and a composition comprising one or more nucleoside RNAs encoding one or more HSV glycoproteins, or immunogenic fragments thereof, is administered as a second or subsequent administration. Each possibility represents a separate embodiment of the present disclosure.

In some embodiments, the modified RNA induces a detectably lower innate immune response than the same quantity of unmodified RNA having the same sequence.

In some embodiments, the compositions and methods of the present disclosure are for use in human subjects, while in another embodiment, they are for use in animal subjects. In another embodiment, the subject is mammalian. In another embodiment, the subject is any organism susceptible to infection by HSV. In some embodiments, the subject is murine, bovine, ovine, canine, feline, equine, porcine, etc. In some embodiments, the compositions and methods of the present disclosure are effective in male subjects. In another embodiment, the compositions and methods of the present disclosure are effective in female subjects. In some embodiments, the compositions and methods of the present disclosure are effective in seronegative subjects. In another embodiment, the compositions and methods of the present disclosure are effective in seropositive subjects.

### Pharmaceutical Formulations

In some embodiments, a method of present disclosure further comprises mixing an RNA with a transfection reagent prior to the step of contacting. In another embodiment, a method of present disclosure further comprises administering an RNA together with a transfection reagent. In another embodiment, the transfection reagent is a cationic lipid reagent. For example, the RNA can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

In another embodiment, a transfection reagent is a lipid-based transfection reagent. In another embodiment, a transfection reagent is a protein-based transfection reagent. In another embodiment, a transfection reagent is a polyethyleneimine based transfection reagent. In another embodiment, a transfection reagent is calcium phosphate. In another embodiment, a transfection reagent is Lipofectin® or Lipofectamine®. In another embodiment, a transfection reagent is any other transfection reagent known in the art.

In another embodiment, the transfection reagent forms a liposome. Liposomes, in another embodiment, increase intracellular stability, increase uptake efficiency and improve biological activity.

In another embodiment, liposomes are hollow spherical vesicles composed of lipids arranged in a similar fashion as those lipids which make up the cell membrane. They have, in another embodiment, an internal aqueous space for entrapping water soluble compounds and range in size from 0.05 to several microns in diameter. In another embodiment, liposomes can deliver RNA to cells in a biologically active form (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

Each type of transfection reagent represents a separate embodiment of the present disclosure.

In another embodiment, an RNA of the present disclosure is encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001). Each method represents a separate embodiment of the disclosure.

In some embodiments, ψRNA is encapsulated in nanoparticles to improve efficiency of delivery and expression of ψRNA. Nanoparticle packaging involves condensing and encapsulating RNA into particles that are smaller than the pore of the nuclear membrane, using chemicals including poly-L-lysine and polyethylene glycol. In some embodiments, RNA is packaged into one of four nanoparticle formulations (PEI, PLL, PAE, and CK₃₀PEG₁₀ₖ).

### Lipid Nanoparticles

In some embodiments, nanoparticles used in the compositions and methods of the present disclosure comprise lipid nanoparticles as described in Cullis, P., & Hope, M. (n.d.). Lipid Nanoparticle Systems for Enabling Gene Therapies. Molecular therapy, 25(7), which is incorporated by reference herein in its entirety.

In some embodiments, delivery of RNA (e.g., nucleoside modified RNA) comprises any suitable delivery method, including RNA transfection methods. For example, said RNAs can be one or more RNAs comprising a nucleotide sequence at least 80%, at least 85%, at least 90%, at least 91%, at least 92%, at least 93%, at least 94%, at least 95%, at least 96%, at least 97%, at least 98%, at least 99%, or 100% identical to a sequence listed in Table 6.

In certain embodiments, delivery of a nucleoside-modified RNA to a subject comprises mixing the nucleoside-modified RNA with a transfection reagent prior to the step of contacting. In another embodiment, a method of present disclosure further comprises administering nucleoside-modified RNA together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome.

Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In some embodiments, the composition comprises a lipid nanoparticle (LNP) and one or more nucleic acid molecules described herein. For example, in some embodiments, the composition comprises an LNP and one or more nucleoside-modified RNA molecules encoding one or more antigens, adjuvants, or a combination thereof.

The term “lipid nanoparticle” refers to a particle having at least one dimension on the order of nanometers (e.g., 1-1,000 nm) which includes one or more lipids, for example a lipid of Formula (I), (II) or (III), as described in WO2016176330A1, which is incorporated by reference herein in its entirety.

In some embodiments, lipid nanoparticles are included in a formulation comprising a nucleoside-modified RNA as described herein. In some embodiments, such lipid nanoparticles comprise a cationic lipid and one or more excipient selected from neutral lipids, charged lipids, steroids and polymer conjugated lipids (e.g., a pegylated lipid such as a pegylated lipid of structure (IV), such as compound IVa). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In some embodiments, the LNP comprises one or more cationic lipids, and one or more stabilizing lipids. Stabilizing lipids include neutral lipids and pegylated lipids.

In some embodiments, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N (N′,N′-dimethylaminoethane)-carbamoyl)cholesterol (DC-Choi), N-(1-(2,3-dioleoyloxy)propyl)-N-2-(sperminecarboxamido)ethyl)-N,N-dimethylammonium trifluoracetate (DOSPA), dioctadecylamidoglycyl carboxy spermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH:

DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the cationic lipid is present in the LNP in an amount from about 30 to about 95 mole percent. In some embodiments, the cationic lipid is present in the LNP in an amount from about 30 to about 70 mole percent. In some embodiments, the cationic lipid is present in the LNP in an amount from about 40 to about 60 mole percent. In some embodiments, the cationic lipid is present in the LNP in an amount of about 50 mole percent. In some embodiments, the LNP comprises only cationic lipids. In certain embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In some embodiments, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue.

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GMi). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols.

Representative polyethylene glycol-lipids include PEG-c-DOMG, PEG-c-DMA, and PEG-s-DMG. In some embodiments, the polyethylene glycol-lipid is N-[(methoxy poly(ethylene glycol)₂₀₀₀)carbamyl]-1,2-dimyristyloxlpropyl-3-amine (PEG-c-DMA). In some embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-0-(co-methoxy(polyethoxy)ethyl)butanedioate (PEG-S-DMG), a pegylated ceramide (PEG-cer), or a PEG dialkoxypropylcarbamate such as Q-methoxy(polyethoxy)ethyl-N-(2,3-di(tetradecanoxy)propyl)carbamate or 2,3-di(tetradecanoxy)propyl-N-(co-methoxy(polyethoxy)ethyl)carbamate. In various embodiments, the molar ratio of the cationic lipid to the pegylated lipid ranges from about 100:1 to about 25:1.

In certain embodiments, the additional lipid is present in the LNP in an amount from about 1 to about 10 mole percent. In some embodiments, the additional lipid is present in the LNP in an amount from about 1 to about 5 mole percent. In some embodiments, the additional lipid is present in the LNP in about 1 mole percent or about 1.5 mole percent.

In certain embodiments, the LNP comprises one or more targeting moieties which are capable of targeting the LNP to a cell or cell population. For example, in some embodiments, the targeting moiety is a ligand which directs the LNP to a receptor found on a cell surface.

In certain embodiments, the LNP comprises one or more internalization domains. For example, in some embodiments, the LNP comprises one or more domains which bind to a cell to induce the internalization of the LNP. For example, in some embodiments, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in some embodiments, the LNP binds systemic ApoE, which leads to the uptake of the LNP and associated cargo.

Other exemplary LNPs and their manufacture are described in the art, for example in WO2016176330A1, U.S. Patent Application Publication No. US20120276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tarn et al., 2013, Nanomedicine, 9(5): 665-74, each of which are incorporated by reference in their entirety.

In another embodiment, methods of the present disclosure comprise administering an RNA encoding an HSV glycoprotein or immunogenic fragment thereof, and a pharmaceutically acceptable carrier or diluent. In other embodiments, pharmaceutically acceptable carriers for liquid formulations may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

As used herein “pharmaceutically acceptable carriers or diluents” are well known to those skilled in the art.

In another embodiment, the pharmaceutical compositions provided herein are controlled-release compositions, i.e. compositions in which the compound is released over a period of time after administration. Controlled- or sustained-release compositions include formulation in lipophilic depots (e.g. fatty acids, waxes, oils). In another embodiment, the composition is an immediate-release composition, i.e. a composition in which the entire compound is released immediately after administration.

Each of the additives, excipients, formulations and methods of administration represents a separate embodiment of the present disclosure.

In another embodiment, the present disclosure provides a kit comprising a polynucleotide as described herein and a reagent utilized in performing a method of the present disclosure. In another embodiment, the present disclosure provides a kit comprising a composition, tool, or instrument of the present disclosure.

The following examples are presented in order to more fully illustrate the preferred embodiments of the disclosure. They should in no way be construed, however, as limiting the broad scope of the disclosure.

### EXPERIMENTAL DETAILS SECTION

### Example 1: Materials and Experimental Methods

Production of RNA, formulation in LNPs

Modified RNA expressing exemplary HSV-2 glycoproteins C, D and E (gC2/gD2/gE2) ectodomains. Modified RNA (encoding an immunogenic HSV-2 gC (gC2) fragment (SEQ ID NO: 65), encoding an immunogenic HSV-2 gD (gD2) fragment (SEQ ID NO: 67), and encoding an immunogenic HSV-2 gE (gE2) fragment (SEQ ID NO: 63)) was prepared based on the DNA coding sequences that encode gC amino acids 27-426 from HSV-2 strain 333 (SEQ ID NO: 9), gD amino acids 26-331 from HSV-2 strain 333 (SEQ ID NO: 18), and gE amino acids 24-405 from HSV-2 strain 2.12 (SEQ ID NO: 4). Exemplary immunogenic fragments extends from the first amino acid after the signal sequence to shortly prior to the transmembrane domain.

The RNAs encoding the same amino acids as HSV glycoproteins, or immunogenic fragments thereof, were produced using T7 RNA polymerase (Megascript, Ambion). To generate nucleoside-modified RNA, m1Ψ-5′-triphosphate (TriLink) was used instead of UTP. RNA was capped using the m7G capping kit with 2′-O-methyltransferase (ScriptCap, CellScript). The RNA contains 101 nucleotide poly(A) tail. The exemplary nucleoside-modified RNAs were purified by Fast Protein Liquid Chromatography (FPLC) (Akta Purifier GE Healthcare) and stored at −20° C. FPLC nucleoside-modified RNAs and polyC RNA (Sigma) were encapsulated in LNPs using a self-assembly process in which an aqueous solution of RNA at acidic pH 4.0 was rapidly mixed with a solution of lipids dissolved in ethanol. LNPs contain an ionizable cationic lipid/phosphatidylcholine/cholesterol/PEG-lipid (Acuitas) (50:10:38.5:1.5 mol/mol) and were encapsulated at an RNA to total lipid ratio of ˜0.05 (wt/wt). The LNPs had a diameter of ˜80 nm as measured by dynamic light scattering using a Zetasizer Nano ZS (Malvern Instruments Ltd, Malvern, UK) instrument, and were stored at −80° C. at a concentration of RNA of ˜1 μg/μl.

### CD4+ and CD8+ T Cell Responses

Spleens were harvested from 5 female BALB/c mice per group and 106 splenocytes were stimulated at 37° C. with exemplary HSV-2 gC immunogenic fragment, exemplary HSV-2 gD immunogenic fragment, or exemplary HSV-2 gE immunogenic fragment pools consisting of 15 amino acids with 11 overlapping amino acids (JPT Innovative Peptide Solutions). After 1 h, brefeldin A (10 μg/ml) (BD Pharmingen) was added for 16 h at 37° C. Splenocytes were stained with aqua blue (Invitrogen) to distinguish live-dead cells, Pacific blue-conjugated anti-CD8 mouse MAb (Biolegend) and R-phycoerythrin-cyanine 5.5 (PE-Cy5.5) anti-CD4 mouse MAb (BD Pharmingen). Cells were permeabilized with Cytofix/Cytoperm (BD Pharmingen), stained with Alexa Fluor 700-conjugated anti-IFNγ mouse MAb, PE-Cy7 anti-TNFα mouse MAb, and allophycocyanin-Cy7 anti-CD3 mouse MAb. Splenocytes were fixed with 1% paraformaldehyde and analyzed by FACS using an 18-color LSR II flow cytometer and FlowJo flow cytometry analytic software.

### Example 2: Characterization of Translational Products Produced by Modified RNA Encoding Exemplary gC2, gD2, and gE2 Immunogenic Fragments

The ability of modified RNA to express proteins of the expected molecular weight when transfected into mammalian cells was verified. 1 μg of trivalent (gC2-, gD2-, or gE2) modified RNA encoding exemplary HSV-2 gC immunogenic fragment, exemplary HSV-2 gD immunogenic fragment, or exemplary HSV-2 gE immunogenic fragment, was transfected into HEK 293 cells (American Type Culture Collection) cells using TransIT-mRNA (Mirus Bio LLC) for the transfection. Eighteen hours later, cells were harvested and extracts prepared for Western blots on denaturing SDS-PAGE gels using rabbit polyclonal anti-gC2 (UP2151), anti-gD2 (R7) or anti-gE2 (R265) antibodies. The RNAs were designed to express exemplary immunogenic fragments of the ectodomains of HSV-2 gC, HSV-2 gD and HSV-2 gE (labeled ecto). (FIG. 1A) gC2 protein probed using rabbit polyclonal gC2 antibody UP2151. Differences in bands in baculovirus and mRNA gC2 lanes likely represent glycosylation patterns for mRNA in mammalian cells and baculovirus in insect cells. (FIG. 1B) HSV-2 gD2 protein visualized using rabbit polyclonal gD2 antibody R7. (FIG. 1C) HSV-2 gE2 protein demonstrated using rabbit polyclonal gE2 antibody R265. The 38kD protein in the baculovirus gE2 lane represents a breakdown product of gE2. As controls for the expected molecular weights, purified baculovirus proteins or exemplary HSV-2 gC immunogenic fragment, exemplary HSV-2 gD immunogenic fragment, and exemplary HSV-2 gE immunogenic fragment comprising the same amino acid sequences as the exemplary HSV-2 immunogenic fragments encoded by the RNA constructs (labeled Bac-ecto) were used (FIGS. 1A-C).

Conclusion: When transfected into mammalian cells, modified RNA encoding exemplary immunogenic fragments of the ectodomains of HSV-2 gC (FIG. 1A), HSV-2 gD (FIG. 1B) and HSV-2 gE (FIG. 1C) produced proteins of the appropriate molecular weights that reacted with antibodies to the glycoproteins on Western blot.

### Example 3: CD4* and CD8* T Cell Responses in Splenocytes after Modified RNA Immunization

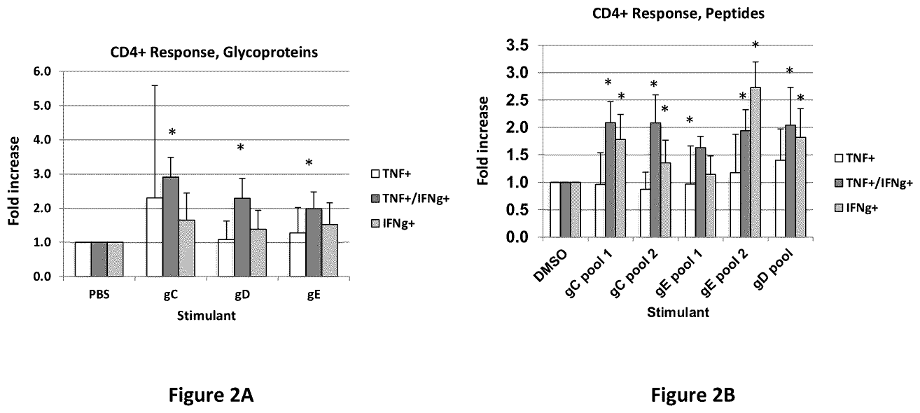

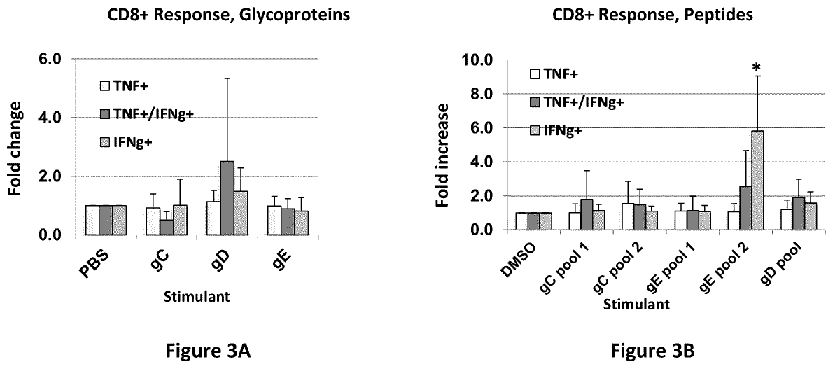

Five BALB/c mice from the exemplary trivalent modified RNA group that were immunized with each modified RNA encoding exemplary HSV-2 glycoprotein immunogenic fragment at a separate site (Trivalent-I group) were euthanized 14 days after the second immunization. Splenocytes were prepared for T cell assays. Splenocytes were stimulated with 15 amino acid peptides containing 11 overlapping amino acids of gC2, gD2 or gE2. The CD4⁺ and CD8⁺ T cell responses are shown in FIGS. 2A-2B and FIGS. 3A-3B, respectively.

CD4⁺ T cells: The exemplary HSV-2 gC (gC2) immunogenic fragment, exemplary HSV-2 gD (gD2) immunogenic fragment, and exemplary HSV-2 gE (gE2) immunogenic fragment, encoded by the modified RNA, each stimulated polyfunctional CD4⁺ T cell responses (FIGS. 2A-2B). Splenocytes harvested from immunized subjects and then stimulated with exemplary glycoprotein immunogenic fragments increased polyfunctional CD4⁺ T cell responses (FIG. 2A). Splenocytes harvested from immunized subjects and then stimulated with 15 amino acid overlapping peptides increased polyfunctional CD4⁺ T cell responses and IFN7 responses (FIG. 2B).

CD8⁺ T cells: Only subjects immunized with exemplary gE2 immunogenic fragment stimulated a significant IFN7 CD8⁺ T cell response (FIG. 3B).

### Example 4: Efficacy of Therapeutic Vaccines Comprising Exemplary HSV-2 Glycoprotein Immunogenic Fragments

Hartley strain guinea pigs were infected intravaginally with 2×10⁵ PFU HSV-2 and randomized into groups of 12 animals each based on the severity of genital lesions up to the time of randomization (on day 32 post-infection). Animals were immunized (n=12/group) on days 35 and 65 post infection (30 μg total exemplary modified RNA/immunization) with the following exemplary nucleoside-modified RNAs encapsulated in lipid nanoparticle (LNP)315:
1. gE2 alone (gE2 group): Exemplary modified RNA encoding HSV-2 gE glycoprotein E (gE2) immunogenic fragment (truncated prior to the transmembrane domain) functional joined to the human IL-2 signal peptide, was encapsulated in LNP315 (provided by Acuitas Vancouver, Canada). Modified RNA encoding exemplary gE2 immunogenic fragment was administered at 30 g at each immunization
2. Modified RNAs encoding exemplary HSV-2 gC (gC2) immunogenic fragment, exemplary HSV-2 gD (gD2) immunogenic fragment, and exemplary HSV-2 gE (gE2) immunogenic fragment, functionally joined to the human IL2 signal peptide (Exemplary gC2 immunogenic fragment, Exemplary gE2 immunogenic fragment) or the native signal peptide (Exemplary gD2 immunogenic fragment) was encapsulated in LNP315 and provided by BioNTech. 10 μg of each of modified RNA encoding exemplary gC2 immunogenic fragment, modified RNA encoding exemplary gD2 immunogenic fragment, and modified RNA encoding exemplary gE2 immunogenic fragment were administered at each immunization for a total dose of 30 μg modified RNA for each immunization.
3. PBS.

Starting one day after the first immunization, the animals were scored daily for genital lesions Monday to Friday from day 36 post-infection until day 116 post-infection (59 days). The results are summarized below. The number of days animals in each group had recurrent genital lesions starting from 1 day after the first immunization (day 36) or starting from 1 day after the second immunization (day 66) were reported. One day after the second immunization was considered as the primary endpoint for the study.

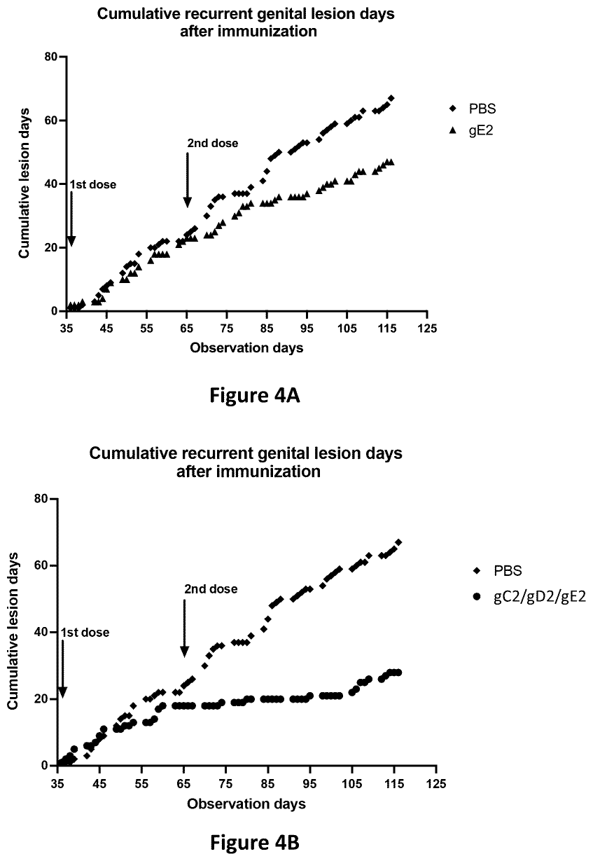

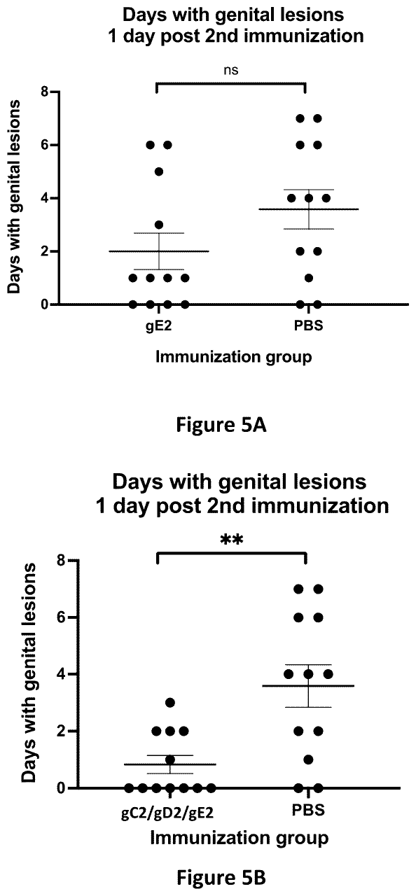

Vaccine efficacy as determined by reduction in recurrent genital lesions in guinea pigs is shown in Table 8. The cumulative recurrent genital lesion days per group are shown in FIGS. 4A-4B. FIGS. 5A-5B shows the results for each guinea pig in the study plotting the number of recurrent genital lesions from 1 day after the second immunization until the end of the study on day 116.

TABLE 8
Exemplary modified RNA Vaccines as
Immunotherapy for Recurrent Genital Lesions in Guinea Pigs

| | 1 day after 1^{st} dose | | | 1 day after 2^{nd} dose | | |
|---|---|---|---|---|---|---|
| Immunogens | Lesions over | | Vaccine | Lesions over | | Vaccine |
| n = 12/group | 708 days | | efficacy* | 444 days | | efficacy |
| HSV-2 gE | 47 | (6.6%) | 30% | 24 | (5.4%) | 44% |
| HSV-2 | 28 | (4.0%) | 58% | 10 | (2.3%) | 77% |
| gC/HSV-2 | | | | | | |
| gD/HSV-2 gE | | | | | | |
| PBS | 67 | (9.5%) | — | 43 | (9.7%) | — |

* Vaccine efficacy calculated relative to PBS group.

Conclusions: Administration of modified RNA encoding exemplary gC2 immunogenic fragment, modified RNA encoding exemplary gD2 immunogenic fragment, and modified RNA encoding exemplary gE2 immunogenic fragment, and modified RNA encoding exemplary gE2 immunogenic fragment alone, reduced the number of recurrent genital lesions compared to animals that were administered PBS control. Administration of modified RNA encoding exemplary gC2 immunogenic fragment, modified RNA encoding exemplary gD2 immunogenic fragment, and modified RNA encoding exemplary gE2 immunogenic fragment, reduced recurrent genital lesions by 77%.

These results suggest that administration of modified RNA encoding exemplary gE2 immunogenic fragment or of modified RNA encoding exemplary gC2 immunogenic fragment, modified RNA encoding exemplary gD2 immunogenic fragment, and modified RNA encoding exemplary gE2 immunogenic fragment, either alone or in combination with other glycoproteins may be effective for both preventing genital herpes and treating genital herpes. The results are very encouraging that HSV-2 glycoproteins, or immunogenic fragments thereof, will be effective for preventing and treating genital herpes.

### Example 5: T-Cell Responses to gE2 Immunization

Mice were immunized twice with 10 μg gE2 mRNA-LNP. Splenocytes from these mice were stimulated with a gE2 overlapping peptide pool. CD4⁺ and CD8⁺ T-cells producing cytokines were analyzed by flow cytometry.

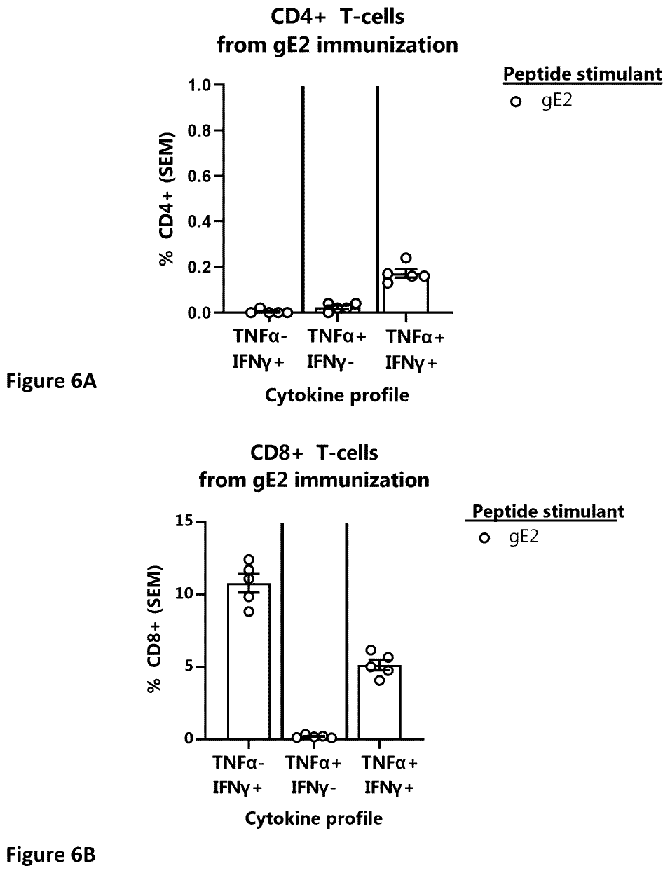

Conclusion: gE2 immunization induces a low level bifunctional cytokine CD4 response (FIG. 6A) and a high level monofunctional and bifunctional cytokine CD8⁺ T-cell response (FIG. 6B).

1. A nucleoside-modified RNA encoding the ectodomain of herpes simplex virus (HSV) glycoprotein E (gE).

2. The RNA of claim 1, wherein the nucleoside-modified RNA comprises one or more pseudouridine residues.

3. The RNA of claim 2, wherein the one or more pseudouridine residues comprise m1T (1-methylpseudouridine), m1acp3Ψ (1-methyl-3-(3-amino-5-carboxypropyl)pseudouridine, Ψm (2′-O-methylpseudouridine, m5D (5-methyldihydrouridine), m3Ψ (3-methylpseudouridine), or any combination thereof.

4. The RNA of any one of claims 1-3, wherein the nucleoside-modified RNA further comprises a signal sequence.

5. The RNA of claim 4, wherein the signal sequence comprises:
a)AUGACCCGCCUGACCGUGCUGGCCCUGCUGGCCGGCCUGCUGGCCUCCUC CCGCGCC (SEQ ID NO: 154),
b)AUGCGCAUGCAGCUGCUGCUGCUGAUCGCCCUGUCCCUGGCCCUGGUGAC CAACUCC (SEQ ID NO: 150),
c)AUGGCCAUCUCCGGCGUGCCCGUGCUGGGCUUCUUCAUCAUCGCCGUGCU GAUGUCCGCCCAGGAGUCCUGGGCC (SEQ ID NO: 155), or

6. The RNA of any one of claims 1-5, wherein the nucleoside-modified RNA further comprises:
i) a poly-A tail;
ii) an m7GpppG cap, 3′-O-methyl-m7GpppG cap, or anti-reverse cap analog;
iii) a cap-independent translational enhancer;
iv) 5′ and 3′ untranslated regions that enhance translation; or
v) a combination thereof;

7. The RNA of any one of claims 1-6, wherein the ectodomain comprises a sequence that is at least 95% identical to SEQ ID NOs: 3.

8. The RNA of any one of claims 1-7, wherein said RNA encoding the ectodomain of HSV gE consists of SEQ ID NO: 23.

9. A composition comprising the polyribonucleotide of any one of claims 1-8.

10. The composition of claim 9, further comprising a nucleoside-modified RNA encoding HSV glycoprotein I (gI).

11. The composition of claim 9, further comprising one or more nucleoside-modified RNAs encoding a) HSV glycoprotein B (gB) or immunogenic fragment thereof, b) HSV glycoprotein H (gH) or immunogenic fragment thereof, c) HSV glycoprotein L (gL) or immunogenic fragment thereof, d) or immunogenic fragment thereof, or e) any combination thereof.

12. The composition of claim 9, wherein said RNA is encapsulated in a nanoparticle, lipid, polymer, cholesterol, or cell penetrating peptide.

13. The composition of claim 12, wherein said nanoparticle is a liposomal nanoparticle.

14. A method of treating a Herpes Simplex Virus (HSV) infection or suppressing, inhibiting, or reducing the incidence of an HSV infection in a subject, the method comprising the step of administering the RNA of any one of claims 1-8 or the composition of any one of claims 9-13 to said subject.

15. The method of claim 14, wherein said HSV infection comprises an HSV-1 infection or an HSV-2 infection.

16. The method of any one of claims 14-15, wherein said HSV infection comprises a primary HSV infection; a flare, recurrence, or HSV labialis following a primary HSV infection; a reactivation of a latent HSV infection; an HSV encephalitis; an HSV neonatal infection; a genital HSV infection; or an oral HSV infection; or a combination thereof.

17. The method of any one of claims 14-16, wherein the administration step comprises intramuscular, subcutaneous, intradermal, intranasal, intravaginal, intrarectal, or topical administration.

18. A method of inducing an immune response in a subject, comprising the step of administering the RNA of any one of claims 1-8 or the composition of any one of claims 9-13 to said subject.

19. The method of claim 18, wherein the administration step comprises intramuscular, subcutaneous, intradermal, intranasal, intravaginal, intrarectal, or topical administration.

20. The method of any one of claims 18-19, wherein said immune response comprises a CD4 immune response; a CD8 immune response; a T follicular helper cell immune response; a germinal center B cell immune response; an IgG antibody response to HSV-2 glycoprotein C, HSV-2 glycoprotein D, HSV-2 gE, or combination thereof, or a combination thereof.
